(19) United States
(12) Patent Application Publication
(10) Pub. No.: US 2025/0313636 A1
(43) Pub. Date: Oct. 9, 2025

(54) ALPHA4BETA7 INTEGRIN BINDING PROTEINS AND METHODS OF USE

(71) Applicant: Paragon Therapeutics, Inc., Waltham, MA (US)

(72) Inventors: Eric Franklin Zhu, Waltham, MA (US); Hussam Hisham Shaheen, Waltham, MA (US); Jacob Cole Milligan, Waltham, MA (US)

(73) Assignee: Paragon Therapeutics, Inc., Waltham, MA (US)

(21) Appl. No.: 19/245,760

(22) Filed: Jun. 23, 2025

Related U.S. Application Data
(63) Continuation of application No. PCT/US2024/042242, filed on Aug. 14, 2024.
(60) Provisional application No. 63/519,483, filed on Aug. 14, 2023.

(51) Int. Cl. C07K 16/28

(52) U.S. Cl. CPC C07K 16/2839; C07K 2317/24; C07K 2317/565; C07K 2317/72; C07K 2317/92; C07K 2317/94

(57) ABSTRACT

Provided herein are α4β7 binding proteins (e.g., antibodies that bind α4β7) and methods of use.

### CROSS-REFERENCE TO RELATED APPLICATIONS

This application is a continuation of International Application No. PCT/US2024/042242, filed Aug. 14, 2024, which claims the benefit of and priority to U.S. Provisional Application No. 63/519,483, filed on Aug. 14, 2023, the entire content of each of which is incorporated herein by reference.

### SEQUENCE LISTING

This application contains a Sequence Listing which has been submitted electronically in XML format and is hereby incorporated by reference in its entirety. The XML copy, created on Mar. 31, 2025, is titled 220703-010302_US_SL.xml and is 1,927,278 bytes in size.

### BACKGROUND

Integrins are cell-adhesion transmembrane receptors that function as extracellular matrix (ECM)-cytoskeletal linkers and transducers of biochemical and mechanical signals between cells and their environment. Due to their exposure on the cell surface and sensitivity to molecular inhibition, integrins such as α4β7 integrin have been investigated as pharmacological targets for treating various diseases including cancer and inflammatory diseases (e.g., inflammatory bowel disease). However, current integrin therapies have been associated with serious side effects given the role of integrins in important biological processes and/or require multiple and frequent doses to maintain therapeutic efficacy. As such, improved α4β7 integrin therapies are needed.

### SUMMARY

Described herein, in certain embodiments, are α4β7 binding proteins comprising: a) a heavy chain variable region (VH) comprising (i) a CDR1 having an amino acid sequence according to SEQ ID NO: 3, (ii) a CDR2 having an amino acid sequence according to SEQ ID NO: 109, and (iii) a CDR3 having an amino acid sequence according to SEQ ID NO: 215; and a light chain variable region (VL) comprising (i) a CDR1 having an amino acid sequence according to SEQ ID NO: 321, (ii) a CDR2 having an amino acid sequence according to SEQ ID NO: 427, and (iii) a CDR3 having an amino acid sequence according to SEQ ID NO: 533; b) a heavy chain variable region (VH) comprising (i) a CDR1 having an amino acid sequence according to SEQ ID NO: 4, (ii) a CDR2 having an amino acid sequence according to SEQ ID NO: 110, and (iii) a CDR3 having an amino acid sequence according to SEQ ID NO: 216; and a light chain variable region (VL) comprising (i) a CDR1 having an amino acid sequence according to SEQ ID NO: 322, (ii) a CDR2 having an amino acid sequence according to SEQ ID NO: 428, and (iii) a CDR3 having an amino acid sequence according to SEQ ID NO: 534; c) a heavy chain variable region (VH) comprising (i) a CDR1 having an amino acid sequence according to SEQ ID NO: 5, (ii) a CDR2 having an amino acid sequence according to SEQ ID NO: 111, and (iii) a CDR3 having an amino acid sequence according to SEQ ID NO: 217; and a light chain variable region (VL) comprising (i) a CDR1 having an amino acid sequence according to SEQ ID NO: 323, (ii) a CDR2 having an amino acid sequence according to SEQ ID NO: 429, and (iii) a CDR3 having an amino acid sequence according to SEQ ID NO: 535; d) a heavy chain variable region (VH) comprising (i) a CDR1 having an amino acid sequence according to SEQ ID NO: 6, (ii) a CDR2 having an amino acid sequence according to SEQ ID NO: 112, and (iii) a CDR3 having an amino acid sequence according to SEQ ID NO: 218; and a light chain variable region (VL) comprising (i) a CDR1 having an amino acid sequence according to SEQ ID NO: 324, (ii) a CDR2 having an amino acid sequence according to SEQ ID NO: 430, and (iii) a CDR3 having an amino acid sequence according to SEQ ID NO: 536; e) a heavy chain variable region (VH) comprising (i) a CDR1 having an amino acid sequence according to SEQ ID NO: 81, (ii) a CDR2 having an amino acid sequence according to SEQ ID NO: 187, and (iii) a CDR3 having an amino acid sequence according to SEQ ID NO: 293; and a light chain variable region (VL) comprising (i) a CDR1 having an amino acid sequence according to SEQ ID NO: 399, (ii) a CDR2 having an amino acid sequence according to SEQ ID NO: 505, and (iii) a CDR3 having an amino acid sequence according to SEQ ID NO: 611; f) a heavy chain variable region (VH) comprising (i) a CDR1 having an amino acid sequence according to SEQ ID NO: 82, (ii) a CDR2 having an amino acid sequence according to SEQ ID NO: 188, and (iii) a CDR3 having an amino acid sequence according to SEQ ID NO: 294; and a light chain variable region (VL) comprising (i) a CDR1 having an amino acid sequence according to SEQ ID NO: 400, (ii) a CDR2 having an amino acid sequence according to SEQ ID NO: 506, and (iii) a CDR3 having an amino acid sequence according to SEQ ID NO: 612; or g) a heavy chain variable region (VH) comprising (i) a CDR1 having an amino acid sequence according to SEQ ID NO: 83, (ii) a CDR2 having an amino acid sequence according to SEQ ID NO: 189, and (iii) a CDR3 having an amino acid sequence according to SEQ ID NO: 295; and a light chain variable region (VL) comprising (i) a CDR1 having an amino acid sequence according to SEQ ID NO: 401, (ii) a CDR2 having an amino acid sequence according to SEQ ID NO: 507, and (iii) a CDR3 having an amino acid sequence according to SEQ ID NO: 613.

In some embodiments, the VH comprises a sequence having at least 80% sequence identity to the amino acid sequence of any one of SEQ ID NOs: 1911-1914 and 1925-1927; and the VL comprises a sequence having at least 80% sequence identity to the amino acid sequence of any one of SEQ ID NOs: 2017-2020 and 2031-2033.

In some embodiments, the VH comprises a sequence having at least 80% sequence identity to the amino acid sequence of SEQ ID NO: 1911 and the VL comprises a sequence having at least 80% sequence identity to the amino acid sequence of SEQ ID NO: 2017.

In some embodiments, the VH comprises a sequence having at least 80% sequence identity to the amino acid sequence of SEQ ID NO: 1912 and the VL comprises a sequence having at least 80% sequence identity to the amino acid sequence of SEQ ID NO: 2018.

In some embodiments, the VH comprises a sequence having at least 80% sequence identity to the amino acid sequence of SEQ ID NO: 1913 and the VL comprises a sequence having at least 80% sequence identity to the amino acid sequence of SEQ ID NO: 2019.

In some embodiments, the VH comprises a sequence having at least 80% sequence identity to the amino acid sequence of SEQ ID NO: 1914 and the VL comprises a sequence having at least 80% sequence identity to the amino acid sequence of SEQ ID NO: 2020.

In some embodiments, the VH comprises a sequence having at least 80% sequence identity to the amino acid sequence of SEQ ID NO: 1925 and the VL comprises a sequence having at least 80% sequence identity to the amino acid sequence of SEQ ID NO: 2031.

In some embodiments, the VH comprises a sequence having at least 80% sequence identity to the amino acid sequence of SEQ ID NO: 1926 and the VL comprises a sequence having at least 80% sequence identity to the amino acid sequence of SEQ ID NO: 2032.

In some embodiments, the VH comprises a sequence having at least 80% sequence identity to the amino acid sequence of SEQ ID NO: 1927 and the VL comprises a sequence having at least 80% sequence identity to the amino acid sequence of SEQ ID NO: 2033.

Described herein, in certain embodiments, are α4β7 binding proteins comprising: a) a heavy chain variable region (VH) comprising (i) a CDR1 having an amino acid sequence according to any one of SEQ ID NOs: 7-26, 32-58, 60-80, and 84-106; (ii) a CDR2 having an amino acid sequence according to any one of SEQ ID NOs: 113-132, 138-164, 166-186, and 190-212; and (iii) a CDR3 having an amino acid sequence according to any one of SEQ ID NOs: 219-238, 244-270, 272-292, and 296-318; and b) a light chain variable region (VL) comprising (i) a CDR1 having an amino acid sequence according to any one of SEQ ID NOs: 325-344, 350-376, 378-398, and 402-424; (ii) a CDR2 having an amino acid sequence according to any one of SEQ ID NOs: 431-450, 456-482, 484-504, and 508-530; and (iii) a CDR3 having an amino acid sequence according to any one of SEQ ID NOs: 537-556, 562-588, 590-610, and 614-636.

In some embodiments, the VH comprises (i) a CDR1 having an amino acid sequence according to SEQ ID NO: 7; (ii) a CDR2 having an amino acid sequence according to SEQ ID NO: 113; and (iii) a CDR3 having an amino acid sequence according to SEQ ID NO: 219; and the VL comprises (i) a CDR1 having an amino acid sequence according to SEQ ID NO: 325; (ii) a CDR2 having an amino acid sequence according to SEQ ID NO: 431; and (iii) a CDR3 having an amino acid sequence according to SEQ ID NO: 537.

In some embodiments, the VH comprises (i) a CDR1 having an amino acid sequence according to SEQ ID NO: 8; (ii) a CDR2 having an amino acid sequence according to SEQ ID NO: 114; and (iii) a CDR3 having an amino acid sequence according to SEQ ID NO: 220; and the VL comprises (i) a CDR1 having an amino acid sequence according to SEQ ID NO: 326; (ii) a CDR2 having an amino acid sequence according to SEQ ID NO: 432; and (iii) a CDR3 having an amino acid sequence according to SEQ ID NO: 538.

In some embodiments, the VH comprises (i) a CDR1 having an amino acid sequence according to SEQ ID NO: 9; (ii) a CDR2 having an amino acid sequence according to SEQ ID NO: 115; and (iii) a CDR3 having an amino acid sequence according to SEQ ID NO: 221; and the VL comprises (i) a CDR1 having an amino acid sequence according to SEQ ID NO: 327; (ii) a CDR2 having an amino acid sequence according to SEQ ID NO: 433; and (iii) a CDR3 having an amino acid sequence according to SEQ ID NO: 539.

In some embodiments, the VH comprises (i) a CDR1 having an amino acid sequence according to SEQ ID NO: 10; (ii) a CDR2 having an amino acid sequence according to SEQ ID NO: 116; and (iii) a CDR3 having an amino acid sequence according to SEQ ID NO: 222; and the VL comprises (i) a CDR1 having an amino acid sequence according to SEQ ID NO: 328; (ii) a CDR2 having an amino acid sequence according to SEQ ID NO: 434; and (iii) a CDR3 having an amino acid sequence according to SEQ ID NO: 540.

In some embodiments, the VH comprises (i) a CDR1 having an amino acid sequence according to SEQ ID NO: 11; (ii) a CDR2 having an amino acid sequence according to SEQ ID NO: 117; and (iii) a CDR3 having an amino acid sequence according to SEQ ID NO: 223; and the VL comprises (i) a CDR1 having an amino acid sequence according to SEQ ID NO: 329; (ii) a CDR2 having an amino acid sequence according to SEQ ID NO: 435; and (iii) a CDR3 having an amino acid sequence according to SEQ ID NO: 541.

In some embodiments, the VH comprises (i) a CDR1 having an amino acid sequence according to SEQ ID NO: 12; (ii) a CDR2 having an amino acid sequence according to SEQ ID NO: 118; and (iii) a CDR3 having an amino acid sequence according to SEQ ID NO: 224; and the VL comprises (i) a CDR1 having an amino acid sequence according to SEQ ID NO: 330; (ii) a CDR2 having an amino acid sequence according to SEQ ID NO: 436; and (iii) a CDR3 having an amino acid sequence according to SEQ ID NO: 542.

In some embodiments, the VH comprises (i) a CDR1 having an amino acid sequence according to SEQ ID NO: 13; (ii) a CDR2 having an amino acid sequence according to SEQ ID NO: 119; and (iii) a CDR3 having an amino acid sequence according to SEQ ID NO: 225; and the VL comprises (i) a CDR1 having an amino acid sequence according to SEQ ID NO: 331; (ii) a CDR2 having an amino acid sequence according to SEQ ID NO: 437; and (iii) a CDR3 having an amino acid sequence according to SEQ ID NO: 543.

In some embodiments, the VH comprises (i) a CDR1 having an amino acid sequence according to SEQ ID NO: 14; (ii) a CDR2 having an amino acid sequence according to SEQ ID NO: 120; and (iii) a CDR3 having an amino acid sequence according to SEQ ID NO: 226; and the VL comprises (i) a CDR1 having an amino acid sequence according to SEQ ID NO: 332; (ii) a CDR2 having an amino acid sequence according to SEQ ID NO: 438; and (iii) a CDR3 having an amino acid sequence according to SEQ ID NO: 544.

In some embodiments, the VH comprises (i) a CDR1 having an amino acid sequence according to SEQ ID NO: 15; (ii) a CDR2 having an amino acid sequence according to SEQ ID NO: 121; and (iii) a CDR3 having an amino acid sequence according to SEQ ID NO: 227; and the VL comprises (i) a CDR1 having an amino acid sequence according to SEQ ID NO: 333; (ii) a CDR2 having an amino acid sequence according to SEQ ID NO: 439; and (iii) a CDR3 having an amino acid sequence according to SEQ ID NO: 545.

In some embodiments, the VH comprises (i) a CDR1 having an amino acid sequence according to SEQ ID NO: 16; (ii) a CDR2 having an amino acid sequence according to SEQ ID NO: 122; and (iii) a CDR3 having an amino acid sequence according to SEQ ID NO: 228; and the VL comprises (i) a CDR1 having an amino acid sequence according to SEQ ID NO: 334; (ii) a CDR2 having an amino acid sequence according to SEQ ID NO: 440; and (iii) a CDR3 having an amino acid sequence according to SEQ ID NO: 546.

In some embodiments, the VH comprises (i) a CDR1 having an amino acid sequence according to SEQ ID NO: 17; (ii) a CDR2 having an amino acid sequence according to SEQ ID NO: 123; and (iii) a CDR3 having an amino acid sequence according to SEQ ID NO: 229; and the VL comprises (i) a CDR1 having an amino acid sequence according to SEQ ID NO: 335; (ii) a CDR2 having an amino acid sequence according to SEQ ID NO: 441; and (iii) a CDR3 having an amino acid sequence according to SEQ ID NO: 547.

In some embodiments, the VH comprises (i) a CDR1 having an amino acid sequence according to SEQ ID NO: 18; (ii) a CDR2 having an amino acid sequence according to SEQ ID NO: 124; and (iii) a CDR3 having an amino acid sequence according to SEQ ID NO: 230; and the VL comprises (i) a CDR1 having an amino acid sequence according to SEQ ID NO: 336; (ii) a CDR2 having an amino acid sequence according to SEQ ID NO: 442; and (iii) a CDR3 having an amino acid sequence according to SEQ ID NO: 548.

In some embodiments, the VH comprises (i) a CDR1 having an amino acid sequence according to SEQ ID NO: 19; (ii) a CDR2 having an amino acid sequence according to SEQ ID NO: 125; and (iii) a CDR3 having an amino acid sequence according to SEQ ID NO: 231; and the VL comprises (i) a CDR1 having an amino acid sequence according to SEQ ID NO: 337; (ii) a CDR2 having an amino acid sequence according to SEQ ID NO: 443; and (iii) a CDR3 having an amino acid sequence according to SEQ ID NO: 549.

In some embodiments, the VH comprises (i) a CDR1 having an amino acid sequence according to SEQ ID NO: 20; (ii) a CDR2 having an amino acid sequence according to SEQ ID NO: 126; and (iii) a CDR3 having an amino acid sequence according to SEQ ID NO: 232; and the VL comprises (i) a CDR1 having an amino acid sequence according to SEQ ID NO: 338; (ii) a CDR2 having an amino acid sequence according to SEQ ID NO: 444; and (iii) a CDR3 having an amino acid sequence according to SEQ ID NO: 550.

In some embodiments, the VH comprises (i) a CDR1 having an amino acid sequence according to SEQ ID NO: 21; (ii) a CDR2 having an amino acid sequence according to SEQ ID NO: 127; and (iii) a CDR3 having an amino acid sequence according to SEQ ID NO: 233; and the VL comprises (i) a CDR1 having an amino acid sequence according to SEQ ID NO: 339; (ii) a CDR2 having an amino acid sequence according to SEQ ID NO: 445; and (iii) a CDR3 having an amino acid sequence according to SEQ ID NO: 551.

In some embodiments, the VH comprises (i) a CDR1 having an amino acid sequence according to SEQ ID NO: 22; (ii) a CDR2 having an amino acid sequence according to SEQ ID NO: 128; and (iii) a CDR3 having an amino acid sequence according to SEQ ID NO: 234; and the VL comprises (i) a CDR1 having an amino acid sequence according to SEQ ID NO: 340; (ii) a CDR2 having an amino acid sequence according to SEQ ID NO: 446; and (iii) a CDR3 having an amino acid sequence according to SEQ ID NO: 552.

In some embodiments, the VH comprises (i) a CDR1 having an amino acid sequence according to SEQ ID NO: 23; (ii) a CDR2 having an amino acid sequence according to SEQ ID NO: 129; and (iii) a CDR3 having an amino acid sequence according to SEQ ID NO: 235; and the VL comprises (i) a CDR1 having an amino acid sequence according to SEQ ID NO: 341; (ii) a CDR2 having an amino acid sequence according to SEQ ID NO: 447; and (iii) a CDR3 having an amino acid sequence according to SEQ ID NO: 553.

In some embodiments, the VH comprises (i) a CDR1 having an amino acid sequence according to SEQ ID NO: 24; (ii) a CDR2 having an amino acid sequence according to SEQ ID NO: 130; and (iii) a CDR3 having an amino acid sequence according to SEQ ID NO: 236; and the VL comprises (i) a CDR1 having an amino acid sequence according to SEQ ID NO: 342; (ii) a CDR2 having an amino acid sequence according to SEQ ID NO: 448; and (iii) a CDR3 having an amino acid sequence according to SEQ ID NO: 554.

In some embodiments, the VH comprises (i) a CDR1 having an amino acid sequence according to SEQ ID NO: 25; (ii) a CDR2 having an amino acid sequence according to SEQ ID NO: 131; and (iii) a CDR3 having an amino acid sequence according to SEQ ID NO: 237; and the VL comprises (i) a CDR1 having an amino acid sequence according to SEQ ID NO: 343; (ii) a CDR2 having an amino acid sequence according to SEQ ID NO: 449; and (iii) a CDR3 having an amino acid sequence according to SEQ ID NO: 555.

In some embodiments, the VH comprises (i) a CDR1 having an amino acid sequence according to SEQ ID NO: 26; (ii) a CDR2 having an amino acid sequence according to SEQ ID NO: 132; and (iii) a CDR3 having an amino acid sequence according to SEQ ID NO: 238; and the VL comprises (i) a CDR1 having an amino acid sequence according to SEQ ID NO: 344; (ii) a CDR2 having an amino acid sequence according to SEQ ID NO: 450; and (iii) a CDR3 having an amino acid sequence according to SEQ ID NO: 556.

In some embodiments, the VH comprises (i) a CDR1 having an amino acid sequence according to SEQ ID NO: 32; (ii) a CDR2 having an amino acid sequence according to SEQ ID NO: 138; and (iii) a CDR3 having an amino acid sequence according to SEQ ID NO: 244; and the VL comprises (i) a CDR1 having an amino acid sequence according to SEQ ID NO: 350; (ii) a CDR2 having an amino acid sequence according to SEQ ID NO: 456; and (iii) a CDR3 having an amino acid sequence according to SEQ ID NO: 562.

In some embodiments, the VH comprises (i) a CDR1 having an amino acid sequence according to SEQ ID NO: 33; (ii) a CDR2 having an amino acid sequence according to SEQ ID NO: 139; and (iii) a CDR3 having an amino acid sequence according to SEQ ID NO: 245; and the VL comprises (i) a CDR1 having an amino acid sequence according to SEQ ID NO: 351; (ii) a CDR2 having an amino acid sequence according to SEQ ID NO: 457; and (iii) a CDR3 having an amino acid sequence according to SEQ ID NO: 563.

In some embodiments, the VH comprises (i) a CDR1 having an amino acid sequence according to SEQ ID NO: 34; (ii) a CDR2 having an amino acid sequence according to SEQ ID NO: 140; and (iii) a CDR3 having an amino acid sequence according to SEQ ID NO: 246; and the VL comprises (i) a CDR1 having an amino acid sequence according to SEQ ID NO: 352; (ii) a CDR2 having an amino acid sequence according to SEQ ID NO: 458; and (iii) a CDR3 having an amino acid sequence according to SEQ ID NO: 564.

In some embodiments, the VH comprises (i) a CDR1 having an amino acid sequence according to SEQ ID NO: 35; (ii) a CDR2 having an amino acid sequence according to SEQ ID NO: 141; and (iii) a CDR3 having an amino acid sequence according to SEQ ID NO: 247; and the VL comprises (i) a CDR1 having an amino acid sequence according to SEQ ID NO: 353; (ii) a CDR2 having an amino acid sequence according to SEQ ID NO: 459; and (iii) a CDR3 having an amino acid sequence according to SEQ ID NO: 565.

In some embodiments, the VH comprises (i) a CDR1 having an amino acid sequence according to SEQ ID NO: 36; (ii) a CDR2 having an amino acid sequence according to SEQ ID NO: 142; and (iii) a CDR3 having an amino acid sequence according to SEQ ID NO: 248; and the VL comprises (i) a CDR1 having an amino acid sequence according to SEQ ID NO: 354; (ii) a CDR2 having an amino acid sequence according to SEQ ID NO: 460; and (iii) a CDR3 having an amino acid sequence according to SEQ ID NO: 566.

In some embodiments, the VH comprises (i) a CDR1 having an amino acid sequence according to SEQ ID NO: 37; (ii) a CDR2 having an amino acid sequence according to SEQ ID NO: 143; and (iii) a CDR3 having an amino acid sequence according to SEQ ID NO: 249; and the VL comprises (i) a CDR1 having an amino acid sequence according to SEQ ID NO: 355; (ii) a CDR2 having an amino acid sequence according to SEQ ID NO: 461; and (iii) a CDR3 having an amino acid sequence according to SEQ ID NO: 567.

In some embodiments, the VH comprises (i) a CDR1 having an amino acid sequence according to SEQ ID NO: 38; (ii) a CDR2 having an amino acid sequence according to SEQ ID NO: 144; and (iii) a CDR3 having an amino acid sequence according to SEQ ID NO: 250; and the VL comprises (i) a CDR1 having an amino acid sequence according to SEQ ID NO: 356; (ii) a CDR2 having an amino acid sequence according to SEQ ID NO: 462; and (iii) a CDR3 having an amino acid sequence according to SEQ ID NO: 568.

In some embodiments, the VH comprises (i) a CDR1 having an amino acid sequence according to SEQ ID NO: 39; (ii) a CDR2 having an amino acid sequence according to SEQ ID NO: 145; and (iii) a CDR3 having an amino acid sequence according to SEQ ID NO: 251; and the VL comprises (i) a CDR1 having an amino acid sequence according to SEQ ID NO: 357; (ii) a CDR2 having an amino acid sequence according to SEQ ID NO: 463; and (iii) a CDR3 having an amino acid sequence according to SEQ ID NO: 569.

In some embodiments, the VH comprises (i) a CDR1 having an amino acid sequence according to SEQ ID NO: 40; (ii) a CDR2 having an amino acid sequence according to SEQ ID NO: 146; and (iii) a CDR3 having an amino acid sequence according to SEQ ID NO: 252; and the VL comprises (i) a CDR1 having an amino acid sequence according to SEQ ID NO: 358; (ii) a CDR2 having an amino acid sequence according to SEQ ID NO: 464; and (iii) a CDR3 having an amino acid sequence according to SEQ ID NO: 570.

In some embodiments, the VH comprises (i) a CDR1 having an amino acid sequence according to SEQ ID NO: 41; (ii) a CDR2 having an amino acid sequence according to SEQ ID NO: 147; and (iii) a CDR3 having an amino acid sequence according to SEQ ID NO: 253; and the VL comprises (i) a CDR1 having an amino acid sequence according to SEQ ID NO: 359; (ii) a CDR2 having an amino acid sequence according to SEQ ID NO: 465; and (iii) a CDR3 having an amino acid sequence according to SEQ ID NO: 571.

In some embodiments, the VH comprises (i) a CDR1 having an amino acid sequence according to SEQ ID NO: 42; (ii) a CDR2 having an amino acid sequence according to SEQ ID NO: 148; and (iii) a CDR3 having an amino acid sequence according to SEQ ID NO: 254; and the VL comprises (i) a CDR1 having an amino acid sequence according to SEQ ID NO: 360; (ii) a CDR2 having an amino acid sequence according to SEQ ID NO: 466; and (iii) a CDR3 having an amino acid sequence according to SEQ ID NO: 572.

In some embodiments, the VH comprises (i) a CDR1 having an amino acid sequence according to SEQ ID NO: 43; (ii) a CDR2 having an amino acid sequence according to SEQ ID NO: 149; and (iii) a CDR3 having an amino acid sequence according to SEQ ID NO: 255; and the VL comprises (i) a CDR1 having an amino acid sequence according to SEQ ID NO: 361; (ii) a CDR2 having an amino acid sequence according to SEQ ID NO: 467; and (iii) a CDR3 having an amino acid sequence according to SEQ ID NO: 573.

In some embodiments, the VH comprises (i) a CDR1 having an amino acid sequence according to SEQ ID NO: 44; (ii) a CDR2 having an amino acid sequence according to SEQ ID NO: 150; and (iii) a CDR3 having an amino acid sequence according to SEQ ID NO: 256; and the VL comprises (i) a CDR1 having an amino acid sequence according to SEQ ID NO: 362; (ii) a CDR2 having an amino acid sequence according to SEQ ID NO: 468; and (iii) a CDR3 having an amino acid sequence according to SEQ ID NO: 574.

In some embodiments, the VH comprises (i) a CDR1 having an amino acid sequence according to SEQ ID NO: 45; (ii) a CDR2 having an amino acid sequence according to SEQ ID NO: 151; and (iii) a CDR3 having an amino acid sequence according to SEQ ID NO: 257; and the VL comprises (i) a CDR1 having an amino acid sequence according to SEQ ID NO: 363; (ii) a CDR2 having an amino acid sequence according to SEQ ID NO: 469; and (iii) a CDR3 having an amino acid sequence according to SEQ ID NO: 575.

In some embodiments, the VH comprises (i) a CDR1 having an amino acid sequence according to SEQ ID NO: 46; (ii) a CDR2 having an amino acid sequence according to SEQ ID NO: 152; and (iii) a CDR3 having an amino acid sequence according to SEQ ID NO: 258; and the VL comprises (i) a CDR1 having an amino acid sequence according to SEQ ID NO: 364; (ii) a CDR2 having an amino acid sequence according to SEQ ID NO: 470; and (iii) a CDR3 having an amino acid sequence according to SEQ ID NO: 576.

In some embodiments, the VH comprises (i) a CDR1 having an amino acid sequence according to SEQ ID NO: 47; (ii) a CDR2 having an amino acid sequence according to SEQ ID NO: 153; and (iii) a CDR3 having an amino acid sequence according to SEQ ID NO: 259; and the VL comprises (i) a CDR1 having an amino acid sequence according to SEQ ID NO: 365; (ii) a CDR2 having an amino acid sequence according to SEQ ID NO: 471; and (iii) a CDR3 having an amino acid sequence according to SEQ ID NO: 577.

In some embodiments, the VH comprises (i) a CDR1 having an amino acid sequence according to SEQ ID NO: 48; (ii) a CDR2 having an amino acid sequence according to SEQ ID NO: 154; and (iii) a CDR3 having an amino acid sequence according to SEQ ID NO: 260; and the VL comprises (i) a CDR1 having an amino acid sequence according to SEQ ID NO: 366; (ii) a CDR2 having an amino acid sequence according to SEQ ID NO: 472; and (iii) a CDR3 having an amino acid sequence according to SEQ ID NO: 578.

In some embodiments, the VH comprises (i) a CDR1 having an amino acid sequence according to SEQ ID NO: 49; (ii) a CDR2 having an amino acid sequence according to SEQ ID NO: 155; and (iii) a CDR3 having an amino acid sequence according to SEQ ID NO: 261; and the VL comprises (i) a CDR1 having an amino acid sequence according to SEQ ID NO: 367; (ii) a CDR2 having an amino acid sequence according to SEQ ID NO: 473; and (iii) a CDR3 having an amino acid sequence according to SEQ ID NO: 579.

In some embodiments, the VH comprises (i) a CDR1 having an amino acid sequence according to SEQ ID NO: 50; (ii) a CDR2 having an amino acid sequence according to SEQ ID NO: 156; and (iii) a CDR3 having an amino acid sequence according to SEQ ID NO: 262; and the VL comprises (i) a CDR1 having an amino acid sequence according to SEQ ID NO: 368; (ii) a CDR2 having an amino acid sequence according to SEQ ID NO: 474; and (iii) a CDR3 having an amino acid sequence according to SEQ ID NO: 580.

In some embodiments, the VH comprises (i) a CDR1 having an amino acid sequence according to SEQ ID NO: 51; (ii) a CDR2 having an amino acid sequence according to SEQ ID NO: 157; and (iii) a CDR3 having an amino acid sequence according to SEQ ID NO: 263; and the VL comprises (i) a CDR1 having an amino acid sequence according to SEQ ID NO: 369; (ii) a CDR2 having an amino acid sequence according to SEQ ID NO: 475; and (iii) a CDR3 having an amino acid sequence according to SEQ ID NO: 581.

In some embodiments, the VH comprises (i) a CDR1 having an amino acid sequence according to SEQ ID NO: 52; (ii) a CDR2 having an amino acid sequence according to SEQ ID NO: 158; and (iii) a CDR3 having an amino acid sequence according to SEQ ID NO: 264; and the VL comprises (i) a CDR1 having an amino acid sequence according to SEQ ID NO: 370; (ii) a CDR2 having an amino acid sequence according to SEQ ID NO: 476; and (iii) a CDR3 having an amino acid sequence according to SEQ ID NO: 582.

In some embodiments, the VH comprises (i) a CDR1 having an amino acid sequence according to SEQ ID NO: 53; (ii) a CDR2 having an amino acid sequence according to SEQ ID NO: 159; and (iii) a CDR3 having an amino acid sequence according to SEQ ID NO: 265; and the VL comprises (i) a CDR1 having an amino acid sequence according to SEQ ID NO: 371; (ii) a CDR2 having an amino acid sequence according to SEQ ID NO: 477; and (iii) a CDR3 having an amino acid sequence according to SEQ ID NO: 583.

In some embodiments, the VH comprises (i) a CDR1 having an amino acid sequence according to SEQ ID NO: 54; (ii) a CDR2 having an amino acid sequence according to SEQ ID NO: 160; and (iii) a CDR3 having an amino acid sequence according to SEQ ID NO: 266; and the VL comprises (i) a CDR1 having an amino acid sequence according to SEQ ID NO: 372; (ii) a CDR2 having an amino acid sequence according to SEQ ID NO: 478; and (iii) a CDR3 having an amino acid sequence according to SEQ ID NO: 584.

In some embodiments, the VH comprises (i) a CDR1 having an amino acid sequence according to SEQ ID NO: 55; (ii) a CDR2 having an amino acid sequence according to SEQ ID NO: 161; and (iii) a CDR3 having an amino acid sequence according to SEQ ID NO: 267; and the VL comprises (i) a CDR1 having an amino acid sequence according to SEQ ID NO: 373; (ii) a CDR2 having an amino acid sequence according to SEQ ID NO: 479; and (iii) a CDR3 having an amino acid sequence according to SEQ ID NO: 585.

In some embodiments, the VH comprises (i) a CDR1 having an amino acid sequence according to SEQ ID NO: 56; (ii) a CDR2 having an amino acid sequence according to SEQ ID NO: 162; and (iii) a CDR3 having an amino acid sequence according to SEQ ID NO: 268; and the VL comprises (i) a CDR1 having an amino acid sequence according to SEQ ID NO: 374; (ii) a CDR2 having an amino acid sequence according to SEQ ID NO: 480; and (iii) a CDR3 having an amino acid sequence according to SEQ ID NO: 586.

In some embodiments, the VH comprises (i) a CDR1 having an amino acid sequence according to SEQ ID NO: 57; (ii) a CDR2 having an amino acid sequence according to SEQ ID NO: 163; and (iii) a CDR3 having an amino acid sequence according to SEQ ID NO: 269; and the VL comprises (i) a CDR1 having an amino acid sequence according to SEQ ID NO: 375; (ii) a CDR2 having an amino acid sequence according to SEQ ID NO: 481; and (iii) a CDR3 having an amino acid sequence according to SEQ ID NO: 587.

In some embodiments, the VH comprises (i) a CDR1 having an amino acid sequence according to SEQ ID NO: 58; (ii) a CDR2 having an amino acid sequence according to SEQ ID NO: 164; and (iii) a CDR3 having an amino acid sequence according to SEQ ID NO: 270; and the VL comprises (i) a CDR1 having an amino acid sequence according to SEQ ID NO: 376; (ii) a CDR2 having an amino acid sequence according to SEQ ID NO: 482; and (iii) a CDR3 having an amino acid sequence according to SEQ ID NO: 588.

In some embodiments, the VH comprises (i) a CDR1 having an amino acid sequence according to SEQ ID NO: 60; (ii) a CDR2 having an amino acid sequence according to SEQ ID NO: 166; and (iii) a CDR3 having an amino acid sequence according to SEQ ID NO: 272; and the VL comprises (i) a CDR1 having an amino acid sequence according to SEQ ID NO: 378; (ii) a CDR2 having an amino acid sequence according to SEQ ID NO: 484; and (iii) a CDR3 having an amino acid sequence according to SEQ ID NO: 590.

In some embodiments, the VH comprises (i) a CDR1 having an amino acid sequence according to SEQ ID NO: 61; (ii) a CDR2 having an amino acid sequence according to SEQ ID NO: 167; and (iii) a CDR3 having an amino acid sequence according to SEQ ID NO: 273; and the VL comprises (i) a CDR1 having an amino acid sequence according to SEQ ID NO: 379; (ii) a CDR2 having an amino acid sequence according to SEQ ID NO: 485; and (iii) a CDR3 having an amino acid sequence according to SEQ ID NO: 591.

In some embodiments, the VH comprises (i) a CDR1 having an amino acid sequence according to SEQ ID NO: 62; (ii) a CDR2 having an amino acid sequence according to SEQ ID NO: 168; and (iii) a CDR3 having an amino acid sequence according to SEQ ID NO: 274; and the VL comprises (i) a CDR1 having an amino acid sequence according to SEQ ID NO: 380; (ii) a CDR2 having an amino acid sequence according to SEQ ID NO: 486; and (iii) a CDR3 having an amino acid sequence according to SEQ ID NO: 592.

In some embodiments, the VH comprises (i) a CDR1 having an amino acid sequence according to SEQ ID NO: 63; (ii) a CDR2 having an amino acid sequence according to SEQ ID NO: 169; and (iii) a CDR3 having an amino acid sequence according to SEQ ID NO: 275; and the VL comprises (i) a CDR1 having an amino acid sequence according to SEQ ID NO: 381; (ii) a CDR2 having an amino acid sequence according to SEQ ID NO: 487; and (iii) a CDR3 having an amino acid sequence according to SEQ ID NO: 593.

In some embodiments, the VH comprises (i) a CDR1 having an amino acid sequence according to SEQ ID NO: 64; (ii) a CDR2 having an amino acid sequence according to SEQ ID NO: 170; and (iii) a CDR3 having an amino acid sequence according to SEQ ID NO: 276; and the VL comprises (i) a CDR1 having an amino acid sequence according to SEQ ID NO: 382; (ii) a CDR2 having an amino acid sequence according to SEQ ID NO: 488; and (iii) a CDR3 having an amino acid sequence according to SEQ ID NO: 594.

In some embodiments, the VH comprises (i) a CDR1 having an amino acid sequence according to SEQ ID NO: 65; (ii) a CDR2 having an amino acid sequence according to SEQ ID NO: 171; and (iii) a CDR3 having an amino acid sequence according to SEQ ID NO: 277; and the VL comprises (i) a CDR1 having an amino acid sequence according to SEQ ID NO: 383; (ii) a CDR2 having an amino acid sequence according to SEQ ID NO: 489; and (iii) a CDR3 having an amino acid sequence according to SEQ ID NO: 595.

In some embodiments, the VH comprises (i) a CDR1 having an amino acid sequence according to SEQ ID NO: 66; (ii) a CDR2 having an amino acid sequence according to SEQ ID NO: 172; and (iii) a CDR3 having an amino acid sequence according to SEQ ID NO: 278; and the VL comprises (i) a CDR1 having an amino acid sequence according to SEQ ID NO: 384; (ii) a CDR2 having an amino acid sequence according to SEQ ID NO: 490; and (iii) a CDR3 having an amino acid sequence according to SEQ ID NO: 596.

In some embodiments, the VH comprises (i) a CDR1 having an amino acid sequence according to SEQ ID NO: 67; (ii) a CDR2 having an amino acid sequence according to SEQ ID NO: 173; and (iii) a CDR3 having an amino acid sequence according to SEQ ID NO: 279; and the VL comprises (i) a CDR1 having an amino acid sequence according to SEQ ID NO: 385; (ii) a CDR2 having an amino acid sequence according to SEQ ID NO: 491; and (iii) a CDR3 having an amino acid sequence according to SEQ ID NO: 597. In some embodiments, the VH comprises (i) a CDR1 having an amino acid sequence according to SEQ ID NO: 68; (ii) a CDR2 having an amino acid sequence according to SEQ ID NO: 174; and (iii) a CDR3 having an amino acid sequence according to SEQ ID NO: 280; and the VL comprises (i) a CDR1 having an amino acid sequence according to SEQ ID NO: 386; (ii) a CDR2 having an amino acid sequence according to SEQ ID NO: 492; and (iii) a CDR3 having an amino acid sequence according to SEQ ID NO: 598.

In some embodiments, the VH comprises (i) a CDR1 having an amino acid sequence according to SEQ ID NO: 69; (ii) a CDR2 having an amino acid sequence according to SEQ ID NO: 175; and (iii) a CDR3 having an amino acid sequence according to SEQ ID NO: 281; and the VL comprises (i) a CDR1 having an amino acid sequence according to SEQ ID NO: 387; (ii) a CDR2 having an amino acid sequence according to SEQ ID NO: 493; and (iii) a CDR3 having an amino acid sequence according to SEQ ID NO: 599.

In some embodiments, the VH comprises (i) a CDR1 having an amino acid sequence according to SEQ ID NO: 70; (ii) a CDR2 having an amino acid sequence according to SEQ ID NO: 176; and (iii) a CDR3 having an amino acid sequence according to SEQ ID NO: 282; and the VL comprises (i) a CDR1 having an amino acid sequence according to SEQ ID NO: 388; (ii) a CDR2 having an amino acid sequence according to SEQ ID NO: 494; and (iii) a CDR3 having an amino acid sequence according to SEQ ID NO: 600.

In some embodiments, the VH comprises (i) a CDR1 having an amino acid sequence according to SEQ ID NO: 71; (ii) a CDR2 having an amino acid sequence according to SEQ ID NO: 177; and (iii) a CDR3 having an amino acid sequence according to SEQ ID NO: 283; and the VL comprises (i) a CDR1 having an amino acid sequence according to SEQ ID NO: 389; (ii) a CDR2 having an amino acid sequence according to SEQ ID NO: 495; and (iii) a CDR3 having an amino acid sequence according to SEQ ID NO: 601.

In some embodiments, the VH comprises (i) a CDR1 having an amino acid sequence according to SEQ ID NO: 72; (ii) a CDR2 having an amino acid sequence according to SEQ ID NO: 178; and (iii) a CDR3 having an amino acid sequence according to SEQ ID NO: 284; and the VL comprises (i) a CDR1 having an amino acid sequence according to SEQ ID NO: 390; (ii) a CDR2 having an amino acid sequence according to SEQ ID NO: 496; and (iii) a CDR3 having an amino acid sequence according to SEQ ID NO: 602.

In some embodiments, the VH comprises (i) a CDR1 having an amino acid sequence according to SEQ ID NO: 73; (ii) a CDR2 having an amino acid sequence according to SEQ ID NO: 179; and (iii) a CDR3 having an amino acid sequence according to SEQ ID NO: 285; and the VL comprises (i) a CDR1 having an amino acid sequence according to SEQ ID NO: 391; (ii) a CDR2 having an amino acid sequence according to SEQ ID NO: 497; and (iii) a CDR3 having an amino acid sequence according to SEQ ID NO: 603.

In some embodiments, the VH comprises (i) a CDR1 having an amino acid sequence according to SEQ ID NO: 74; (ii) a CDR2 having an amino acid sequence according to SEQ ID NO: 180; and (iii) a CDR3 having an amino acid sequence according to SEQ ID NO: 286; and the VL comprises (i) a CDR1 having an amino acid sequence according to SEQ ID NO: 392; (ii) a CDR2 having an amino acid sequence according to SEQ ID NO: 498; and (iii) a CDR3 having an amino acid sequence according to SEQ ID NO: 604.

In some embodiments, the VH comprises (i) a CDR1 having an amino acid sequence according to SEQ ID NO: 75; (ii) a CDR2 having an amino acid sequence according to SEQ ID NO: 181; and (iii) a CDR3 having an amino acid sequence according to SEQ ID NO: 287; and the VL comprises (i) a CDR1 having an amino acid sequence according to SEQ ID NO: 393; (ii) a CDR2 having an amino acid sequence according to SEQ ID NO: 499; and (iii) a CDR3 having an amino acid sequence according to SEQ ID NO: 605.

In some embodiments, the VH comprises (i) a CDR1 having an amino acid sequence according to SEQ ID NO: 76; (ii) a CDR2 having an amino acid sequence according to SEQ ID NO: 182; and (iii) a CDR3 having an amino acid sequence according to SEQ ID NO: 288; and the VL comprises (i) a CDR1 having an amino acid sequence according to SEQ ID NO: 394; (ii) a CDR2 having an amino acid sequence according to SEQ ID NO: 500; and (iii) a CDR3 having an amino acid sequence according to SEQ ID NO: 606.

In some embodiments, the VH comprises (i) a CDR1 having an amino acid sequence according to SEQ ID NO: 77; (ii) a CDR2 having an amino acid sequence according to SEQ ID NO: 183; and (iii) a CDR3 having an amino acid sequence according to SEQ ID NO: 289; and the VL comprises (i) a CDR1 having an amino acid sequence according to SEQ ID NO: 395; (ii) a CDR2 having an amino acid sequence according to SEQ ID NO: 501; and (iii) a CDR3 having an amino acid sequence according to SEQ ID NO: 607.

In some embodiments, the VH comprises (i) a CDR1 having an amino acid sequence according to SEQ ID NO: 78; (ii) a CDR2 having an amino acid sequence according to SEQ ID NO: 184; and (iii) a CDR3 having an amino acid sequence according to SEQ ID NO: 290; and the VL comprises (i) a CDR1 having an amino acid sequence according to SEQ ID NO: 396; (ii) a CDR2 having an amino acid sequence according to SEQ ID NO: 502; and (iii) a CDR3 having an amino acid sequence according to SEQ ID NO: 608.

In some embodiments, the VH comprises (i) a CDR1 having an amino acid sequence according to SEQ ID NO: 79; (ii) a CDR2 having an amino acid sequence according to SEQ ID NO: 185; and (iii) a CDR3 having an amino acid sequence according to SEQ ID NO: 291; and the VL comprises (i) a CDR1 having an amino acid sequence according to SEQ ID NO: 397; (ii) a CDR2 having an amino acid sequence according to SEQ ID NO: 503; and (iii) a CDR3 having an amino acid sequence according to SEQ ID NO: 609.

In some embodiments, the VH comprises (i) a CDR1 having an amino acid sequence according to SEQ ID NO: 80; (ii) a CDR2 having an amino acid sequence according to SEQ ID NO: 186; and (iii) a CDR3 having an amino acid sequence according to SEQ ID NO: 292; and the VL comprises (i) a CDR1 having an amino acid sequence according to SEQ ID NO: 398; (ii) a CDR2 having an amino acid sequence according to SEQ ID NO: 504; and (iii) a CDR3 having an amino acid sequence according to SEQ ID NO: 610.

In some embodiments, the VH comprises (i) a CDR1 having an amino acid sequence according to SEQ ID NO: 84; (ii) a CDR2 having an amino acid sequence according to SEQ ID NO: 190; and (iii) a CDR3 having an amino acid sequence according to SEQ ID NO: 2%; and the VL comprises (i) a CDR1 having an amino acid sequence according to SEQ ID NO: 402; (ii) a CDR2 having an amino acid sequence according to SEQ ID NO: 508; and (iii) a CDR3 having an amino acid sequence according to SEQ ID NO: 614.

In some embodiments, the VH comprises (i) a CDR1 having an amino acid sequence according to SEQ ID NO: 85; (ii) a CDR2 having an amino acid sequence according to SEQ ID NO: 191; and (iii) a CDR3 having an amino acid sequence according to SEQ ID NO: 297; and the VL comprises (i) a CDR1 having an amino acid sequence according to SEQ ID NO: 403; (ii) a CDR2 having an amino acid sequence according to SEQ ID NO: 509; and (iii) a CDR3 having an amino acid sequence according to SEQ ID NO: 615.

In some embodiments, the VH comprises (i) a CDR1 having an amino acid sequence according to SEQ ID NO: 86; (ii) a CDR2 having an amino acid sequence according to SEQ ID NO: 192; and (iii) a CDR3 having an amino acid sequence according to SEQ ID NO: 298; and the VL comprises (i) a CDR1 having an amino acid sequence according to SEQ ID NO: 404; (ii) a CDR2 having an amino acid sequence according to SEQ ID NO: 510; and (iii) a CDR3 having an amino acid sequence according to SEQ ID NO: 616.

In some embodiments, the VH comprises (i) a CDR1 having an amino acid sequence according to SEQ ID NO: 87; (ii) a CDR2 having an amino acid sequence according to SEQ ID NO: 193; and (iii) a CDR3 having an amino acid sequence according to SEQ ID NO: 299; and the VL comprises (i) a CDR1 having an amino acid sequence according to SEQ ID NO: 405; (ii) a CDR2 having an amino acid sequence according to SEQ ID NO: 511; and (iii) a CDR3 having an amino acid sequence according to SEQ ID NO: 617.

In some embodiments, the VH comprises (i) a CDR1 having an amino acid sequence according to SEQ ID NO: 88; (ii) a CDR2 having an amino acid sequence according to SEQ ID NO: 194; and (iii) a CDR3 having an amino acid sequence according to SEQ ID NO: 300; and the VL comprises (i) a CDR1 having an amino acid sequence according to SEQ ID NO: 406; (ii) a CDR2 having an amino acid sequence according to SEQ ID NO: 512; and (iii) a CDR3 having an amino acid sequence according to SEQ ID NO: 618.

In some embodiments, the VH comprises (i) a CDR1 having an amino acid sequence according to SEQ ID NO: 89; (ii) a CDR2 having an amino acid sequence according to SEQ ID NO: 195; and (iii) a CDR3 having an amino acid sequence according to SEQ ID NO: 301; and the VL comprises (i) a CDR1 having an amino acid sequence according to SEQ ID NO: 407; (ii) a CDR2 having an amino acid sequence according to SEQ ID NO: 513; and (iii) a CDR3 having an amino acid sequence according to SEQ ID NO: 619.

In some embodiments, the VH comprises (i) a CDR1 having an amino acid sequence according to SEQ ID NO: 90; (ii) a CDR2 having an amino acid sequence according to SEQ ID NO: 196; and (iii) a CDR3 having an amino acid sequence according to SEQ ID NO: 302; and the VL comprises (i) a CDR1 having an amino acid sequence according to SEQ ID NO: 408; (ii) a CDR2 having an amino acid sequence according to SEQ ID NO: 514; and (iii) a CDR3 having an amino acid sequence according to SEQ ID NO: 620.

In some embodiments, the VH comprises (i) a CDR1 having an amino acid sequence according to SEQ ID NO: 91; (ii) a CDR2 having an amino acid sequence according to SEQ ID NO: 197; and (iii) a CDR3 having an amino acid sequence according to SEQ ID NO: 303; and the VL comprises (i) a CDR1 having an amino acid sequence according to SEQ ID NO: 409; (ii) a CDR2 having an amino acid sequence according to SEQ ID NO: 515; and (iii) a CDR3 having an amino acid sequence according to SEQ ID NO: 621.

In some embodiments, the VH comprises (i) a CDR1 having an amino acid sequence according to SEQ ID NO: 92; (ii) a CDR2 having an amino acid sequence according to SEQ ID NO: 198; and (iii) a CDR3 having an amino acid sequence according to SEQ ID NO: 304; and the VL comprises (i) a CDR1 having an amino acid sequence according to SEQ ID NO: 410; (ii) a CDR2 having an amino acid sequence according to SEQ ID NO: 516; and (iii) a CDR3 having an amino acid sequence according to SEQ ID NO: 622.

In some embodiments, the VH comprises (i) a CDR1 having an amino acid sequence according to SEQ ID NO: 93; (ii) a CDR2 having an amino acid sequence according to SEQ ID NO: 199; and (iii) a CDR3 having an amino acid sequence according to SEQ ID NO: 305; and the VL comprises (i) a CDR1 having an amino acid sequence according to SEQ ID NO: 411; (ii) a CDR2 having an amino acid sequence according to SEQ ID NO: 517; and (iii) a CDR3 having an amino acid sequence according to SEQ ID NO: 623.

In some embodiments, the VH comprises (i) a CDR1 having an amino acid sequence according to SEQ ID NO: 94; (ii) a CDR2 having an amino acid sequence according to SEQ ID NO: 200; and (iii) a CDR3 having an amino acid sequence according to SEQ ID NO: 306; and the VL comprises (i) a CDR1 having an amino acid sequence according to SEQ ID NO: 412; (ii) a CDR2 having an amino acid sequence according to SEQ ID NO: 518; and (iii) a CDR3 having an amino acid sequence according to SEQ ID NO: 624.

In some embodiments, the VH comprises (i) a CDR1 having an amino acid sequence according to SEQ ID NO: 95; (ii) a CDR2 having an amino acid sequence according to SEQ ID NO: 201; and (iii) a CDR3 having an amino acid sequence according to SEQ ID NO: 307; and the VL comprises (i) a CDR1 having an amino acid sequence according to SEQ ID NO: 413; (ii) a CDR2 having an amino acid sequence according to SEQ ID NO: 519; and (iii) a CDR3 having an amino acid sequence according to SEQ ID NO: 625.

In some embodiments, the VH comprises (i) a CDR1 having an amino acid sequence according to SEQ ID NO: 96; (ii) a CDR2 having an amino acid sequence according to SEQ ID NO: 202; and (iii) a CDR3 having an amino acid sequence according to SEQ ID NO: 308; and the VL comprises (i) a CDR1 having an amino acid sequence according to SEQ ID NO: 414; (ii) a CDR2 having an amino acid sequence according to SEQ ID NO: 520; and (iii) a CDR3 having an amino acid sequence according to SEQ ID NO: 626.

In some embodiments, the VH comprises (i) a CDR1 having an amino acid sequence according to SEQ ID NO: 97; (ii) a CDR2 having an amino acid sequence according to SEQ ID NO: 203; and (iii) a CDR3 having an amino acid sequence according to SEQ ID NO: 309; and the VL comprises (i) a CDR1 having an amino acid sequence according to SEQ ID NO: 415; (ii) a CDR2 having an amino acid sequence according to SEQ ID NO: 521; and (iii) a CDR3 having an amino acid sequence according to SEQ ID NO: 627.

In some embodiments, the VH comprises (i) a CDR1 having an amino acid sequence according to SEQ ID NO: 98; (ii) a CDR2 having an amino acid sequence according to SEQ ID NO: 204; and (iii) a CDR3 having an amino acid sequence according to SEQ ID NO: 310; and the VL comprises (i) a CDR1 having an amino acid sequence according to SEQ ID NO: 416; (ii) a CDR2 having an amino acid sequence according to SEQ ID NO: 522; and (iii) a CDR3 having an amino acid sequence according to SEQ ID NO: 628.

In some embodiments, the VH comprises (i) a CDR1 having an amino acid sequence according to SEQ ID NO: 99; (ii) a CDR2 having an amino acid sequence according to SEQ ID NO: 205; and (iii) a CDR3 having an amino acid sequence according to SEQ ID NO: 311; and the VL comprises (i) a CDR1 having an amino acid sequence according to SEQ ID NO: 417; (ii) a CDR2 having an amino acid sequence according to SEQ ID NO: 523; and (iii) a CDR3 having an amino acid sequence according to SEQ ID NO: 629.

In some embodiments, the VH comprises (i) a CDR1 having an amino acid sequence according to SEQ ID NO: 100; (ii) a CDR2 having an amino acid sequence according to SEQ ID NO: 206; and (iii) a CDR3 having an amino acid sequence according to SEQ ID NO: 312; and the VL comprises (i) a CDR1 having an amino acid sequence according to SEQ ID NO: 418; (ii) a CDR2 having an amino acid sequence according to SEQ ID NO: 524; and (iii) a CDR3 having an amino acid sequence according to SEQ ID NO: 630.

In some embodiments, the VH comprises (i) a CDR1 having an amino acid sequence according to SEQ ID NO: 101; (ii) a CDR2 having an amino acid sequence according to SEQ ID NO: 207; and (iii) a CDR3 having an amino acid sequence according to SEQ ID NO: 313; and the VL comprises (i) a CDR1 having an amino acid sequence according to SEQ ID NO: 419; (ii) a CDR2 having an amino acid sequence according to SEQ ID NO: 525; and (iii) a CDR3 having an amino acid sequence according to SEQ ID NO: 631.

In some embodiments, the VH comprises (i) a CDR1 having an amino acid sequence according to SEQ ID NO: 102; (ii) a CDR2 having an amino acid sequence according to SEQ ID NO: 208; and (iii) a CDR3 having an amino acid sequence according to SEQ ID NO: 314; and the VL comprises (i) a CDR1 having an amino acid sequence according to SEQ ID NO: 420; (ii) a CDR2 having an amino acid sequence according to SEQ ID NO: 526; and (iii) a CDR3 having an amino acid sequence according to SEQ ID NO: 632.

In some embodiments, the VH comprises (i) a CDR1 having an amino acid sequence according to SEQ ID NO: 103; (ii) a CDR2 having an amino acid sequence according to SEQ ID NO: 209; and (iii) a CDR3 having an amino acid sequence according to SEQ ID NO: 315; and the VL comprises (i) a CDR1 having an amino acid sequence according to SEQ ID NO: 421; (ii) a CDR2 having an amino acid sequence according to SEQ ID NO: 527; and (iii) a CDR3 having an amino acid sequence according to SEQ ID NO: 633.

In some embodiments, the VH comprises (i) a CDR1 having an amino acid sequence according to SEQ ID NO: 104; (ii) a CDR2 having an amino acid sequence according to SEQ ID NO: 210; and (iii) a CDR3 having an amino acid sequence according to SEQ ID NO: 316; and the VL comprises (i) a CDR1 having an amino acid sequence according to SEQ ID NO: 422; (ii) a CDR2 having an amino acid sequence according to SEQ ID NO: 528; and (iii) a CDR3 having an amino acid sequence according to SEQ ID NO: 634.

In some embodiments, the VH comprises (i) a CDR1 having an amino acid sequence according to SEQ ID NO: 105; (ii) a CDR2 having an amino acid sequence according to SEQ ID NO: 211; and (iii) a CDR3 having an amino acid sequence according to SEQ ID NO: 317; and the VL comprises (i) a CDR1 having an amino acid sequence according to SEQ ID NO: 423; (ii) a CDR2 having an amino acid sequence according to SEQ ID NO: 529; and (iii) a CDR3 having an amino acid sequence according to SEQ ID NO: 635.

In some embodiments, the VH comprises (i) a CDR1 having an amino acid sequence according to SEQ ID NO: 106; (ii) a CDR2 having an amino acid sequence according to SEQ ID NO: 212; and (iii) a CDR3 having an amino acid sequence according to SEQ ID NO: 318; and the VL comprises (i) a CDR1 having an amino acid sequence according to SEQ ID NO: 424; (ii) a CDR2 having an amino acid sequence according to SEQ ID NO: 530; and (iii) a CDR3 having an amino acid sequence according to SEQ ID NO: 636.

In some embodiments, the VH comprises a sequence having at least 80% sequence identity to the amino acid sequence of any one of SEQ ID NOs: 1915-1934, 1940-1966, 1968-1988, and 1992-2014; and the VL comprises a sequence having at least 80% sequence identity to the amino acid sequence of any one of SEQ ID NOs: 2021-2040, 2046-2072, 2074-2094, and 2098-2120.

In some embodiments, the VH comprises a sequence having at least 80% sequence identity to the amino acid sequence of SEQ ID NO: 127 and the VL comprises a sequence having at least 80% sequence identity to the amino acid sequence of SEQ ID NO: 134.

In some embodiments, the VH comprises a sequence having at least 80% sequence identity to the amino acid sequence of SEQ ID NO: 1915 and the VL comprises a sequence having at least 80% sequence identity to the amino acid sequence of SEQ ID NO: 2021.

In some embodiments, the VH comprises a sequence having at least 80% sequence identity to the amino acid sequence of SEQ ID NO: 1916 and the VL comprises a sequence having at least 80% sequence identity to the amino acid sequence of SEQ ID NO: 2022.

In some embodiments, the VH comprises a sequence having at least 80% sequence identity to the amino acid sequence of SEQ ID NO: 1917 and the VL comprises a sequence having at least 80% sequence identity to the amino acid sequence of SEQ ID NO: 2023.

In some embodiments, the VH comprises a sequence having at least 80% sequence identity to the amino acid sequence of SEQ ID NO: 1918 and the VL comprises a sequence having at least 80% sequence identity to the amino acid sequence of SEQ ID NO: 2024.

In some embodiments, the VH comprises a sequence having at least 80% sequence identity to the amino acid sequence of SEQ ID NO: 1919 and the VL comprises a sequence having at least 80% sequence identity to the amino acid sequence of SEQ ID NO: 2025.

In some embodiments, the VH comprises a sequence having at least 80% sequence identity to the amino acid sequence of SEQ ID NO: 1920 and the VL comprises a sequence having at least 80% sequence identity to the amino acid sequence of SEQ ID NO: 2026.

In some embodiments, the VH comprises a sequence having at least 80% sequence identity to the amino acid sequence of SEQ ID NO: 1921 and the VL comprises a sequence having at least 80% sequence identity to the amino acid sequence of SEQ ID NO: 2027.

In some embodiments, the VH comprises a sequence having at least 80% sequence identity to the amino acid sequence of SEQ ID NO: 1922 and the VL comprises a sequence having at least 80% sequence identity to the amino acid sequence of SEQ ID NO: 2028.

In some embodiments, the VH comprises a sequence having at least 80% sequence identity to the amino acid sequence of SEQ ID NO: 1923 and the VL comprises a sequence having at least 80% sequence identity to the amino acid sequence of SEQ ID NO: 2029.

In some embodiments, the VH comprises a sequence having at least 80% sequence identity to the amino acid sequence of SEQ ID NO: 1924 and the VL comprises a sequence having at least 80% sequence identity to the amino acid sequence of SEQ ID NO: 2030.

In some embodiments, the VH comprises a sequence having at least 80% sequence identity to the amino acid sequence of SEQ ID NO: 1925 and the VL comprises a sequence having at least 80% sequence identity to the amino acid sequence of SEQ ID NO: 2031.

In some embodiments, the VH comprises a sequence having at least 80% sequence identity to the amino acid sequence of SEQ ID NO: 1926 and the VL comprises a sequence having at least 80% sequence identity to the amino acid sequence of SEQ ID NO: 2032.

In some embodiments, the VH comprises a sequence having at least 80% sequence identity to the amino acid sequence of SEQ ID NO: 1927 and the VL comprises a sequence having at least 80% sequence identity to the amino acid sequence of SEQ ID NO: 2033.

In some embodiments, the VH comprises a sequence having at least 80% sequence identity to the amino acid sequence of SEQ ID NO: 1928 and the VL comprises a sequence having at least 80% sequence identity to the amino acid sequence of SEQ ID NO: 2034.

In some embodiments, the VH comprises a sequence having at least 80% sequence identity to the amino acid sequence of SEQ ID NO: 1929 and the VL comprises a sequence having at least 80% sequence identity to the amino acid sequence of SEQ ID NO: 2035.

In some embodiments, the VH comprises a sequence having at least 80% sequence identity to the amino acid sequence of SEQ ID NO: 1930 and the VL comprises a sequence having at least 80% sequence identity to the amino acid sequence of SEQ ID NO: 2036.

In some embodiments, the VH comprises a sequence having at least 80% sequence identity to the amino acid sequence of SEQ ID NO: 1931 and the VL comprises a sequence having at least 80% sequence identity to the amino acid sequence of SEQ ID NO: 2037.

In some embodiments, the VH comprises a sequence having at least 80% sequence identity to the amino acid sequence of SEQ ID NO: 1932 and the VL comprises a sequence having at least 80% sequence identity to the amino acid sequence of SEQ ID NO: 2038.

In some embodiments, the VH comprises a sequence having at least 80% sequence identity to the amino acid sequence of SEQ ID NO: 1933 and the VL comprises a sequence having at least 80% sequence identity to the amino acid sequence of SEQ ID NO: 2039.

In some embodiments, the VH comprises a sequence having at least 80% sequence identity to the amino acid sequence of SEQ ID NO: 1934 and the VL comprises a sequence having at least 80% sequence identity to the amino acid sequence of SEQ ID NO: 2040.

In some embodiments, the VH comprises a sequence having at least 80% sequence identity to the amino acid sequence of SEQ ID NO: 1940 and the VL comprises a sequence having at least 80% sequence identity to the amino acid sequence of SEQ ID NO: 2046.

In some embodiments, the VH comprises a sequence having at least 80% sequence identity to the amino acid sequence of SEQ ID NO: 1941 and the VL comprises a sequence having at least 80% sequence identity to the amino acid sequence of SEQ ID NO: 2047.

In some embodiments, the VH comprises a sequence having at least 80% sequence identity to the amino acid sequence of SEQ ID NO: 1942 and the VL comprises a sequence having at least 80% sequence identity to the amino acid sequence of SEQ ID NO: 2048.

In some embodiments, the VH comprises a sequence having at least 80% sequence identity to the amino acid sequence of SEQ ID NO: 1943 and the VL comprises a sequence having at least 80% sequence identity to the amino acid sequence of SEQ ID NO: 2049.

In some embodiments, the VH comprises a sequence having at least 80% sequence identity to the amino acid sequence of SEQ ID NO: 1944 and the VL comprises a sequence having at least 80% sequence identity to the amino acid sequence of SEQ ID NO: 2050.

In some embodiments, the VH comprises a sequence having at least 80% sequence identity to the amino acid sequence of SEQ ID NO: 1945 and the VL comprises a sequence having at least 80% sequence identity to the amino acid sequence of SEQ ID NO: 2051.

In some embodiments, the VH comprises a sequence having at least 80% sequence identity to the amino acid sequence of SEQ ID NO: 1946 and the VL comprises a sequence having at least 80% sequence identity to the amino acid sequence of SEQ ID NO: 2052.

In some embodiments, the VH comprises a sequence having at least 80% sequence identity to the amino acid sequence of SEQ ID NO: 1947 and the VL comprises a sequence having at least 80% sequence identity to the amino acid sequence of SEQ ID NO: 2053.

In some embodiments, the VH comprises a sequence having at least 80% sequence identity to the amino acid sequence of SEQ ID NO: 1948 and the VL comprises a sequence having at least 80% sequence identity to the amino acid sequence of SEQ ID NO: 2054.

In some embodiments, the VH comprises a sequence having at least 80% sequence identity to the amino acid sequence of SEQ ID NO: 1949 and the VL comprises a sequence having at least 80% sequence identity to the amino acid sequence of SEQ ID NO: 2055.

In some embodiments, the VH comprises a sequence having at least 80% sequence identity to the amino acid sequence of SEQ ID NO: 1950 and the VL comprises a sequence having at least 80% sequence identity to the amino acid sequence of SEQ ID NO: 2056.

In some embodiments, the VH comprises a sequence having at least 80% sequence identity to the amino acid sequence of SEQ ID NO: 1951 and the VL comprises a sequence having at least 80% sequence identity to the amino acid sequence of SEQ ID NO: 2057.

In some embodiments, the VH comprises a sequence having at least 80% sequence identity to the amino acid sequence of SEQ ID NO: 1952 and the VL comprises a sequence having at least 80% sequence identity to the amino acid sequence of SEQ ID NO: 2058.

In some embodiments, the VH comprises a sequence having at least 80% sequence identity to the amino acid sequence of SEQ ID NO: 1953 and the VL comprises a sequence having at least 80% sequence identity to the amino acid sequence of SEQ ID NO: 2059.

In some embodiments, the VH comprises a sequence having at least 80% sequence identity to the amino acid sequence of SEQ ID NO: 1954 and the VL comprises a sequence having at least 80% sequence identity to the amino acid sequence of SEQ ID NO: 2060.

In some embodiments, the VH comprises a sequence having at least 80% sequence identity to the amino acid sequence of SEQ ID NO: 1955 and the VL comprises a sequence having at least 80% sequence identity to the amino acid sequence of SEQ ID NO: 2061.

In some embodiments, the VH comprises a sequence having at least 80% sequence identity to the amino acid sequence of SEQ ID NO: 1956 and the VL comprises a sequence having at least 80% sequence identity to the amino acid sequence of SEQ ID NO: 2062.

In some embodiments, the VH comprises a sequence having at least 80% sequence identity to the amino acid sequence of SEQ ID NO: 1957 and the VL comprises a sequence having at least 80% sequence identity to the amino acid sequence of SEQ ID NO: 2063.

In some embodiments, the VH comprises a sequence having at least 80% sequence identity to the amino acid sequence of SEQ ID NO: 1958 and the VL comprises a sequence having at least 80% sequence identity to the amino acid sequence of SEQ ID NO: 2064.

In some embodiments, the VH comprises a sequence having at least 80% sequence identity to the amino acid sequence of SEQ ID NO: 1959 and the VL comprises a sequence having at least 80% sequence identity to the amino acid sequence of SEQ ID NO: 2065.

In some embodiments, the VH comprises a sequence having at least 80% sequence identity to the amino acid sequence of SEQ ID NO: 1960 and the VL comprises a sequence having at least 80% sequence identity to the amino acid sequence of SEQ ID NO: 2066.

In some embodiments, the VH comprises a sequence having at least 80% sequence identity to the amino acid sequence of SEQ ID NO: 1961 and the VL comprises a sequence having at least 80% sequence identity to the amino acid sequence of SEQ ID NO: 2067.

In some embodiments, the VH comprises a sequence having at least 80% sequence identity to the amino acid sequence of SEQ ID NO: 1962 and the VL comprises a sequence having at least 80% sequence identity to the amino acid sequence of SEQ ID NO: 2068.

In some embodiments, the VH comprises a sequence having at least 80% sequence identity to the amino acid sequence of SEQ ID NO: 1963 and the VL comprises a sequence having at least 80% sequence identity to the amino acid sequence of SEQ ID NO: 2069.

In some embodiments, the VH comprises a sequence having at least 80% sequence identity to the amino acid sequence of SEQ ID NO: 1964 and the VL comprises a sequence having at least 80% sequence identity to the amino acid sequence of SEQ ID NO: 2070.

In some embodiments, the VH comprises a sequence having at least 80% sequence identity to the amino acid sequence of SEQ ID NO: 1965 and the VL comprises a sequence having at least 80% sequence identity to the amino acid sequence of SEQ ID NO: 2071.

In some embodiments, the VH comprises a sequence having at least 80% sequence identity to the amino acid sequence of SEQ ID NO: 1966 and the VL comprises a sequence having at least 80% sequence identity to the amino acid sequence of SEQ ID NO: 2072.

In some embodiments, the VH comprises a sequence having at least 80% sequence identity to the amino acid sequence of SEQ ID NO: 1968 and the VL comprises a sequence having at least 80% sequence identity to the amino acid sequence of SEQ ID NO: 2074.

In some embodiments, the VH comprises a sequence having at least 80% sequence identity to the amino acid sequence of SEQ ID NO: 1969 and the VL comprises a sequence having at least 80% sequence identity to the amino acid sequence of SEQ ID NO: 2075.

In some embodiments, the VH comprises a sequence having at least 80% sequence identity to the amino acid sequence of SEQ ID NO: 1970 and the VL comprises a sequence having at least 80% sequence identity to the amino acid sequence of SEQ ID NO: 2076.

In some embodiments, the VH comprises a sequence having at least 80% sequence identity to the amino acid sequence of SEQ ID NO: 1971 and the VL comprises a sequence having at least 80% sequence identity to the amino acid sequence of SEQ ID NO: 2077.

In some embodiments, the VH comprises a sequence having at least 80% sequence identity to the amino acid sequence of SEQ ID NO: 1972 and the VL comprises a sequence having at least 80% sequence identity to the amino acid sequence of SEQ ID NO: 2078.

In some embodiments, the VH comprises a sequence having at least 80% sequence identity to the amino acid sequence of SEQ ID NO: 1973 and the VL comprises a sequence having at least 80% sequence identity to the amino acid sequence of SEQ ID NO: 2079.

In some embodiments, the VH comprises a sequence having at least 80% sequence identity to the amino acid sequence of SEQ ID NO: 1974 and the VL comprises a sequence having at least 80% sequence identity to the amino acid sequence of SEQ ID NO: 2080.

In some embodiments, the VH comprises a sequence having at least 80% sequence identity to the amino acid sequence of SEQ ID NO: 1975 and the VL comprises a sequence having at least 80% sequence identity to the amino acid sequence of SEQ ID NO: 2081.

In some embodiments, the VH comprises a sequence having at least 80% sequence identity to the amino acid sequence of SEQ ID NO: 1976 and the VL comprises a sequence having at least 80% sequence identity to the amino acid sequence of SEQ ID NO: 2082.

In some embodiments, the VH comprises a sequence having at least 80% sequence identity to the amino acid sequence of SEQ ID NO: 1977 and the VL comprises a sequence having at least 80% sequence identity to the amino acid sequence of SEQ ID NO: 2083.

In some embodiments, the VH comprises a sequence having at least 80% sequence identity to the amino acid sequence of SEQ ID NO: 1978 and the VL comprises a sequence having at least 80% sequence identity to the amino acid sequence of SEQ ID NO: 2084.

In some embodiments, the VH comprises a sequence having at least 80% sequence identity to the amino acid sequence of SEQ ID NO: 1979 and the VL comprises a sequence having at least 80% sequence identity to the amino acid sequence of SEQ ID NO: 2085.

In some embodiments, the VH comprises a sequence having at least 80% sequence identity to the amino acid sequence of SEQ ID NO: 1980 and the VL comprises a sequence having at least 80% sequence identity to the amino acid sequence of SEQ ID NO: 2086.

In some embodiments, the VH comprises a sequence having at least 80% sequence identity to the amino acid sequence of SEQ ID NO: 1981 and the VL comprises a sequence having at least 80% sequence identity to the amino acid sequence of SEQ ID NO: 2087.

In some embodiments, the VH comprises a sequence having at least 80% sequence identity to the amino acid sequence of SEQ ID NO: 1982 and the VL comprises a sequence having at least 80% sequence identity to the amino acid sequence of SEQ ID NO: 2088.

In some embodiments, the VH comprises a sequence having at least 80% sequence identity to the amino acid sequence of SEQ ID NO: 1983 and the VL comprises a sequence having at least 80% sequence identity to the amino acid sequence of SEQ ID NO: 2089.

In some embodiments, the VH comprises a sequence having at least 80% sequence identity to the amino acid sequence of SEQ ID NO: 1984 and the VL comprises a sequence having at least 80% sequence identity to the amino acid sequence of SEQ ID NO: 2090.

In some embodiments, the VH comprises a sequence having at least 80% sequence identity to the amino acid sequence of SEQ ID NO: 1985 and the VL comprises a sequence having at least 80% sequence identity to the amino acid sequence of SEQ ID NO: 2091.

In some embodiments, the VH comprises a sequence having at least 80% sequence identity to the amino acid sequence of SEQ ID NO: 1986 and the VL comprises a sequence having at least 80% sequence identity to the amino acid sequence of SEQ ID NO: 2092.

In some embodiments, the VH comprises a sequence having at least 80% sequence identity to the amino acid sequence of SEQ ID NO: 1987 and the VL comprises a sequence having at least 80% sequence identity to the amino acid sequence of SEQ ID NO: 2093.

In some embodiments, the VH comprises a sequence having at least 80% sequence identity to the amino acid sequence of SEQ ID NO: 1992 and the VL comprises a sequence having at least 80% sequence identity to the amino acid sequence of SEQ ID NO: 2098.

In some embodiments, the VH comprises a sequence having at least 80% sequence identity to the amino acid sequence of SEQ ID NO: 1993 and the VL comprises a sequence having at least 80% sequence identity to the amino acid sequence of SEQ ID NO: 2099.

In some embodiments, the VH comprises a sequence having at least 80% sequence identity to the amino acid sequence of SEQ ID NO: 1994 and the VL comprises a sequence having at least 80% sequence identity to the amino acid sequence of SEQ ID NO: 2100.

In some embodiments, the VH comprises a sequence having at least 80% sequence identity to the amino acid sequence of SEQ ID NO: 1995 and the VL comprises a sequence having at least 80% sequence identity to the amino acid sequence of SEQ ID NO: 2101.

In some embodiments, the VH comprises a sequence having at least 80% sequence identity to the amino acid sequence of SEQ ID NO: 1996 and the VL comprises a sequence having at least 80% sequence identity to the amino acid sequence of SEQ ID NO: 2102.

In some embodiments, the VH comprises a sequence having at least 80% sequence identity to the amino acid sequence of SEQ ID NO: 1997 and the VL comprises a sequence having at least 80% sequence identity to the amino acid sequence of SEQ ID NO: 2103.

In some embodiments, the VH comprises a sequence having at least 80% sequence identity to the amino acid sequence of SEQ ID NO: 1998 and the VL comprises a sequence having at least 80% sequence identity to the amino acid sequence of SEQ ID NO: 2104.

In some embodiments, the VH comprises a sequence having at least 80% sequence identity to the amino acid sequence of SEQ ID NO: 1999 and the VL comprises a sequence having at least 80% sequence identity to the amino acid sequence of SEQ ID NO: 2105.

In some embodiments, the VH comprises a sequence having at least 80% sequence identity to the amino acid sequence of SEQ ID NO: 2000 and the VL comprises a sequence having at least 80% sequence identity to the amino acid sequence of SEQ ID NO: 2106.

In some embodiments, the VH comprises a sequence having at least 80% sequence identity to the amino acid sequence of SEQ ID NO: 2001 and the VL comprises a sequence having at least 80% sequence identity to the amino acid sequence of SEQ ID NO: 2107.

In some embodiments, the VH comprises a sequence having at least 80% sequence identity to the amino acid sequence of SEQ ID NO: 2002 and the VL comprises a sequence having at least 80% sequence identity to the amino acid sequence of SEQ ID NO: 2108.

In some embodiments, the VH comprises a sequence having at least 80% sequence identity to the amino acid sequence of SEQ ID NO: 2003 and the VL comprises a sequence having at least 80% sequence identity to the amino acid sequence of SEQ ID NO: 2109.

In some embodiments, the VH comprises a sequence having at least 80% sequence identity to the amino acid sequence of SEQ ID NO: 2004 and the VL comprises a sequence having at least 80% sequence identity to the amino acid sequence of SEQ ID NO: 2110.

In some embodiments, the VH comprises a sequence having at least 80% sequence identity to the amino acid sequence of SEQ ID NO: 2005 and the VL comprises a sequence having at least 80% sequence identity to the amino acid sequence of SEQ ID NO: 2111.

In some embodiments, the VH comprises a sequence having at least 80% sequence identity to the amino acid sequence of SEQ ID NO: 2006 and the VL comprises a sequence having at least 80% sequence identity to the amino acid sequence of SEQ ID NO: 2112.

In some embodiments, the VH comprises a sequence having at least 80% sequence identity to the amino acid sequence of SEQ ID NO: 2007 and the VL comprises a sequence having at least 80% sequence identity to the amino acid sequence of SEQ ID NO: 2113.

In some embodiments, the VH comprises a sequence having at least 80% sequence identity to the amino acid sequence of SEQ ID NO: 2008 and the VL comprises a sequence having at least 80% sequence identity to the amino acid sequence of SEQ ID NO: 2114.

In some embodiments, the VH comprises a sequence having at least 80% sequence identity to the amino acid sequence of SEQ ID NO: 2009 and the VL comprises a sequence having at least 80% sequence identity to the amino acid sequence of SEQ ID NO: 2115.

In some embodiments, the VH comprises a sequence having at least 80% sequence identity to the amino acid sequence of SEQ ID NO: 2010 and the VL comprises a sequence having at least 80% sequence identity to the amino acid sequence of SEQ ID NO: 2116.

In some embodiments, the VH comprises a sequence having at least 80% sequence identity to the amino acid sequence of SEQ ID NO: 2011 and the VL comprises a sequence having at least 80% sequence identity to the amino acid sequence of SEQ ID NO: 2117.

In some embodiments, the VH comprises a sequence having at least 80% sequence identity to the amino acid sequence of SEQ ID NO: 2012 and the VL comprises a sequence having at least 80% sequence identity to the amino acid sequence of SEQ ID NO: 2118.

In some embodiments, the VH comprises a sequence having at least 80% sequence identity to the amino acid sequence of SEQ ID NO: 2013 and the VL comprises a sequence having at least 80% sequence identity to the amino acid sequence of SEQ ID NO: 2119.

In some embodiments, the VH comprises a sequence having at least 80% sequence identity to the amino acid sequence of SEQ ID NO: 2014 and the VL comprises a sequence having at least 80% sequence identity to the amino acid sequence of SEQ ID NO: 2120.

Described herein, in certain embodiments, are α4β7 binding proteins comprising: a) a heavy chain variable region (VH) comprising the amino acid sequence according to any one of SEQ ID NOs: 1909, 1910, 1935-1939, 1967; and b) a light chain variable region (VL) comprising the amino acid sequence according to any one of SEQ ID NOs: 2015, 2016, 2041-2045, 2073.

In some embodiments, the VH comprises the amino acid sequence according to SEQ ID NO: 1909; and the VL comprises the amino acid sequence according to SEQ ID NO: 2015.

In some embodiments, the VH comprises the amino acid sequence according to SEQ ID NO: 1910; and the VL comprises the amino acid sequence according to SEQ ID NO: 2016.

In some embodiments, the VH comprises the amino acid sequence according to SEQ ID NO: 1935; and the VL comprises the amino acid sequence according to SEQ ID NO: 2041.

In some embodiments, the VH comprises the amino acid sequence according to SEQ ID NO: 1936; and the VL comprises the amino acid sequence according to SEQ ID NO: 2042.

In some embodiments, the VH comprises the amino acid sequence according to SEQ ID NO: 1937; and the VL comprises the amino acid sequence according to SEQ ID NO: 2043.

In some embodiments, the VH comprises the amino acid sequence according to SEQ ID NO: 1938; and the VL comprises the amino acid sequence according to SEQ ID NO: 2044.

In some embodiments, the VH comprises the amino acid sequence according to SEQ ID NO: 1939; and the VL comprises the amino acid sequence according to SEQ ID NO: 2045.

In some embodiments, the VH comprises the amino acid sequence according to SEQ ID NO: 1967; and the VL comprises the amino acid sequence according to SEQ ID NO: 2073.

Described herein, in certain embodiments, are α4β7 binding proteins comprising: a) a heavy chain variable region (VH) comprising (i) a CDR1 having an amino acid sequence according to any one of SEQ ID NOs: 1-106, (ii) a CDR2 having an amino acid sequence according to any one of SEQ ID NOs: 107-212, and (iii) a CDR3 having an amino acid sequence according to any one of SEQ ID NOs: 213-318; b) a light chain variable region (VL) comprising (i) a CDR1 having an amino acid sequence according to any one of SEQ ID NOs: 319-424, (ii) a CDR2 having an amino acid sequence according to any one of SEQ ID NOs: 425-530, and (iii) a CDR3 having an amino acid sequence according to any one of SEQ ID NOs: 531-636; and c) a modified Fc that comprises amino acid modifications M252Y, S254T, and T256E (YTE) and/or L234A/G237A (LAGA).

Described herein, in certain embodiments, are α4β7 binding proteins comprising: a) a heavy chain variable region (VH) comprising (i) a CDR1 having an amino acid sequence according to any one of SEQ ID NOs: 1-106, (ii) a CDR2 having an amino acid sequence according to any one of SEQ ID NOs: 107-212, and (iii) a CDR3 having an amino acid sequence according to any one of SEQ ID NOs: 213-318; b) a light chain variable region (VL) comprising (i) a CDR1 having an amino acid sequence according to any one of SEQ ID NOs: 319-424, (ii) a CDR2 having an amino acid sequence according to any one of SEQ ID NOs: 425-530, and (iii) a CDR3 having an amino acid sequence according to any one of SEQ ID NOs: 531-636; and c) a modified Fc that extends half-life of the α4β7 binding protein as compared to an α4β7 binding protein that does not comprise the modified Fc.

Described herein, in certain embodiments, are α4β7 binding proteins, wherein the α4β7 binding protein specifically binds to an epitope of α4β7 and comprises a Fc domain comprising amino acid modifications M252Y, S254T, and T256E (YTE) and/or L234A/G237A (LAGA).

In some embodiments, the Fc is an IgG1, IgG2 or IgG4 immunoglobulin Fc domain.

In some embodiments, the Fc is an IgG1 immunoglobulin domain.

In some embodiments, the Fc is an IgG2 immunoglobulin domain.

In some embodiments, the Fc is an IgG4 immunoglobulin domain.

In some embodiments, the α4β7 binding protein is an antibody.

Aspects of the disclosure relate to compositions comprising the α4β7 binding proteins described herein and a pharmaceutically acceptable carrier. Also provided herein are injectable liquid compositions comprising the α4β7 binding proteins described herein and a pharmaceutically acceptable carrier.

Aspects of the disclosure relate to nucleic acid encoding the α4β7 binding proteins described herein. In some embodiments, provided herein are recombinant host cells comprised the nucleic acid encoding the α4β7 binding proteins described herein.

Aspects of the disclosure relate to methods of treating an inflammatory bowel disease in a patient in need thereof, the method comprising subcutaneously or intravenously administering to the patient an effective amount of an α4β7 binding protein described herein. In some embodiments, the inflammatory bowel disease is Crohn's disease or ulcerative colitis. In some embodiments, the inflammatory bowel disease is ulcerative colitis. In some embodiments, the inflammatory bowel disease is Crohn's disease. In some embodiments, administration of the α4β7 binding protein is subcutaneous. In some embodiments, administration of the α4β7 binding protein is intravenous.

Aspects of the disclosure relate to methods of treating an inflammatory disease in a patient in need thereof, the method comprising subcutaneously or intravenously administering to the patient an effective amount of an α4β7 binding protein described herein. In some embodiments, the inflammatory disease is hidradenitis suppurativa. In some embodiments, administration of the α4β7 binding protein is subcutaneous. In some embodiments, administration of the α4β7 binding protein is intravenous.

### BRIEF DESCRIPTION OF THE DRAWINGS

FIG. 1 depicts binding curves for increasing concentrations of an anti-α4β7 comparator antibody, a control anti-α4 antibody, and exemplary antibodies Antibody 1 and Antibody 2 incubated with the RPMI-8866 cell line that expresses only α4β7 integrin as determined by flow cytometry analysis. X-axis depicts antibody concentration in nanomolar (nM) and y-axis depicts cell binding as mean fluorescent intensity (MFI).

FIG. 2 depicts binding curves for increasing concentrations of an anti-α4β7 comparator antibody, a control anti-α4 antibody, and exemplary antibodies Antibody 1 and Antibody 2 incubated with the Ramos cell line that expresses only α4 as determined by flow cytometry analysis. X-axis depicts antibody concentration in nanomolar (nM) and y-axis depicts cell binding as mean fluorescent intensity (MFI).

FIG. 3 depicts percent inhibition of adhesion of α4β7 integrin- and α4β1-integrin expressing HuT-78 cells mixed with increasing concentrations of comparator antibody and exemplary antibodies Antibody 1 and Antibody 2 on plates coated with MAdCAM-1. X-axis depicts antibody concentration in nanomolar (nM) and y-axis depicts percent inhibition of adhesion.

FIG. 4 depicts percent inhibition of adhesion of α4β7 integrin- and α4β1-integrin expressing HuT-78 cells mixed with increasing concentrations of comparator antibody, a control anti-α4 antibody, and exemplary antibodies Antibody 1 and Antibody 2 on plates coated with VCAM-1. X-axis depicts antibody concentration in nanomolar (nM) and y-axis depicts percent inhibition of adhesion.

### DETAILED DESCRIPTION

To facilitate an understanding of the present disclosure, a number of terms and phrases are defined below.

As used herein, unless otherwise indicated, the term “antibody” is understood to mean an intact antibody (e.g., an intact monoclonal antibody), or a fragment thereof, such as a Fc fragment of an antibody (e.g., an Fc fragment of a monoclonal antibody), or an antigen-binding fragment of an antibody (e.g., an antigen-binding fragment of a monoclonal antibody), including an intact antibody, antigen-binding fragment, or Fc fragment that has been modified, engineered, or chemically conjugated. In general, antibodies are multimeric proteins that contain four polypeptide chains. Two of the polypeptide chains are called immunoglobulin heavy chains (H chains), and two of the polypeptide chains are called immunoglobulin light chains (L chains). The immunoglobulin heavy and light chains are connected by an interchain disulfide bond. The immunoglobulin heavy chains are connected by interchain disulfide bonds. A light chain consists of one variable region (VL) and one constant region (CL). The heavy chain consists of one variable region (VH) and at least three constant regions (CH1, CH2 and CH3). The variable regions determine the binding specificity of the antibody. Each variable region contains three hypervariable regions known as complementarity determining regions (CDRs) flanked by four relatively conserved regions known as framework regions (FRs). The extent of the FRs and CDRs has been defined (Kabat, E. A., et al. (1991) Sequences of Proteins of Immunological Interest, Fifth Edition, U.S. Department of Health and Human Services, NIH Publication No. 91-3242; and Chothia, C. et al. (1987) J. Mol. Biol. 196:901-917). The three CDRs, referred to as CDR1, CDR2, and CDR3, contribute to the antibody binding specificity. Naturally occurring antibodies have been used as starting material for engineered antibodies, such as chimeric antibodies and humanized antibodies. Examples of antibody-based antigen-binding fragments include Fab, Fab′, (Fab′)2, Fv, single chain antibodies (e.g., scFv), minibodies, and diabodies. Examples of antibodies that have been modified or engineered include chimeric antibodies, humanized antibodies, and multispecific antibodies (e.g., bispecific antibodies). An example of a chemically conjugated antibody is an antibody conjugated to a toxin moiety.

The terms “variable domain” and “variable region” are used interchangeably and refer to the portions of the antibody or immunoglobulin domains that exhibit variability in their sequence and that are involved in determining the specificity and binding affinity of a particular antibody. Variability is not evenly distributed throughout the variable domains of antibodies; it is concentrated in sub-domains of each of the heavy and light chain variable regions. These sub-domains are called “hypervariable regions” or “complementarity determining regions” (CDRs). The more conserved (i.e., non-hypervariable) portions of the variable domains are called the “framework” regions (FRM or FR) and provide a scaffold for the six CDRs in three-dimensional space to form an antigen-binding surface.

An “Fc polypeptide” of a dimeric Fc as used herein refers to one of the two polypeptides forming the dimeric Fc domain, i.e. a polypeptide comprising C-terminal constant regions of an immunoglobulin heavy chain, capable of stable self-association. For example, an Fc polypeptide of a dimeric IgG Fc comprises an IgG CH2 and an IgG CH3 constant domain sequence. An Fc can be of the class IgA, IgD, IgE, IgG, and IgM. These classes are also designated α, δ, ε, γ, and μ, respectively. Several of these may be further divided into subclasses (isotypes), e.g., IgG1, IgG2, IgG3, IgG4, IgA1, and IgA2.

The terms “Fc receptor” and “FcR” are used to describe a receptor that binds to the Fc region of an antibody. For example, an FcR can be a native sequence human FcR. Generally, an FcR is one which binds an IgG antibody (a gamma receptor) and includes receptors of the FcγRI, FcγRII, and FcγRIII subclasses, including allelic variants and alternatively spliced forms of these receptors. FcγRII receptors include FcγRIIA (an “activating receptor”) and FcγRIIB (an “inhibiting receptor”), which have similar amino acid sequences that differ primarily in the cytoplasmic domains thereof. Immunoglobulins of other isotypes can also be bound by certain FcRs (see, e.g., Janeway et al., Immuno Biology: the immune system in health and disease, (Elsevier Science Ltd., NY) (4th ed., 1999)). Activating receptor FcγRIIA contains an immunoreceptor tyrosine-based activation motif (ITAM) in its cytoplasmic domain. Inhibiting receptor FcγRIIB contains an immunoreceptor tyrosine-based inhibition motif (ITIM) in its cytoplasmic domain (reviewed in Dafron, Annu. Rev. Immunol. 15:203-234 (1997)). FcRs are reviewed in Ravetch and Kinet, Annu. Rev. Immunol 9:457-92 (1991); Capel et al., Immunomethods 4:25-34 (1994); and de Haas et al., J. Lab. Clin. Med. 126:330-41 (1995). Other FcRs, including those to be identified in the future, are encompassed by the term “FcR” herein. The term also includes the neonatal receptor, FcRn, which is responsible for the transfer of maternal IgGs to the fetus (Guyer et al., J. Immunol. 117:587 (1976); and Kim et al., J. Immunol. 24:249 (1994)).

The terms “recipient”, “individual”, “subject”, “host”, and “patient”, are used interchangeably herein and in some embodiments, refer to any mammalian subject for whom diagnosis, treatment, or therapy is desired, particularly humans. “Mammal” for purposes of treatment refers to any animal classified as a mammal, including humans, domestic and farm animals, and laboratory, zoo, sports, or pet animals, such as dogs, horses, cats, cows, sheep, goats, pigs, mice, rats, rabbits, guinea pigs, monkeys etc. In some embodiments, the mammal is human. None of these terms require the supervision of medical personnel.

As used herein, the term “effective amount” refers to the amount of a compound (e.g., a compound of the present disclosure) sufficient to effect beneficial or desired results. An effective amount can be administered in one or more administrations, applications or dosages and is not intended to be limited to a particular formulation or administration route. As used herein, the term “treating” includes any effect, e.g., lessening, reducing, modulating, ameliorating or eliminating, that results in the improvement of the condition, disease, disorder, and the like, or ameliorating a symptom thereof.

As used herein, the term “pharmaceutical composition” refers to the combination of an active agent with a carrier, inert or active, making the composition especially suitable for diagnostic or therapeutic use in vivo or ex vivo.

As used herein, the term “pharmaceutically acceptable carrier” refers to any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants, see e.g., Martin, Remington's Pharmaceutical Sciences, 15th Ed., Mack Publ. Co., Easton, PA (1975).

The terms “a” and “an” as used herein mean “one or more” and include the plural unless the context is inappropriate.

As used herein, all numerical values or numerical ranges include whole integers within or encompassing such ranges and fractions of the values or the integers within or encompassing ranges unless the context clearly indicates otherwise. Thus, for example, reference to a range of 90-100%, includes 91%, 92%, 93%, 94%, 95%, 95%, 96%, 97%, etc., as well as 91.1%, 91.2%, 91.3%, 91.4%, 91.5%, etc., 92.1%, 92.2%, 92.3%, 92.4%, 92.5%, etc., and so forth. In another example, reference to a range of 1-5,000-fold includes 1, 2, 3, 4, 5, 6, 7, 8, 9, 10, 11, 12, 13, 14, 15, 16, 17, 18, 19, 20, fold, etc., as well as 1.1, 1.2, 1.3, 1.4, 1.5, fold, etc., 2.1, 2.2, 2.3, 2.4, 2.5, fold, etc., and so forth.

“About” a number, as used herein, refers to range including the number and ranging from 10% below that number to 10% above that number. “About” a range refers to 10% below the lower limit of the range, spanning to 10% above the upper limit of the range.

“Percent (%) identity” refers to the extent to which two sequences (nucleotide or amino acid) have the same residue at the same positions in an alignment. For example, “an amino acid sequence is X % identical to SEQ ID NO: Y” refers to % identity of the amino acid sequence to SEQ ID NO: Y and is elaborated as X % of residues in the amino acid sequence are identical to the residues of sequence disclosed in SEQ ID NO: Y. Generally, computer programs are employed for such calculations. Exemplary programs that compare and align pairs of sequences include ALIGN (Myers and Miller, 1988), FASTA (Pearson and Lipman, 1988; Pearson, 1990) and gapped BLAST (Altschul et al., 1997), BLASTP, BLASTN, or GCG (Devereux et al., 1984).

Throughout the description, where compositions are described as having, including, or comprising specific components, or where processes and methods are described as having, including, or comprising specific steps, it is contemplated that, additionally, there are compositions of the present disclosure that consist essentially of, or consist of, the recited components, and that there are processes and methods according to the present disclosure that consist essentially of, or consist of, the recited processing steps.

As a general matter, compositions specifying a percentage are by weight unless otherwise specified. Further, if a variable is not accompanied by a definition, then the previous definition of the variable controls.

### α4β7 Integrin Binding Proteins

Provided herein are α4β7 integrin binding proteins. In some embodiments, the α4β7 integrin binding protein is an antibody. In certain embodiments α4β7 integrin binding proteins comprise a modified Fc that extends half-life of the α4β7 binding protein as compared to an α4β7 binding protein that does not comprise the modified Fc.

Further described herein, in certain embodiments, are α4β7 integrin binding proteins, wherein the α4β7 binding protein specifically binds to an epitope of α4β7 and comprises a Fc domain comprising amino acid modifications M252Y, S254T, and T256E (YTE) and/or L234A/G237A (LAGA).

Amino acid sequences of exemplary CDRs of α4β7 integrin binding proteins are provided in Table 1.

TABLE 1
Sequences of CDRs
Kabat Numbering

| | SEQ ID | | SEQ ID | | SEQ ID | | SEQ ID | | SEQ ID | | SEQ ID | |
|---|---|---|---|---|---|---|---|---|---|---|---|---|
| Antibody | NO | CDRH1 | NO | CDRH2 | NO | CDRH3 | NO | CDRL1 | NO | CDRL2 | NO | CDRL3 |
| Antibody 1 | 1 | SYWMH | 107 | EIDPSES | 213 | GGYDG | 319 | RSSQSL | 425 | GISNRF | 531 | LQGTH |
| | | | | NTNYN | | WDYAI | | AKSYG | | S | | QPYT |
| | | | | QKFKG | | DY | | NTYLS | | | | |
| Antibody 2 | 2 | SYWMH | 108 | EIDPSES | 214 | GGYDG | 320 | RSSQSL | 426 | GISNRF | 532 | LQGTH |
| | | | | NTNYN | | WDYAI | | AKSYG | | S | | QPYT |
| | | | | QKFKG | | DY | | NTYLS | | | | |
| Antibody 3 | 3 | IYWMH | 109 | EIDPSES | 215 | GGYDG | 321 | RSSQSL | 427 | GISNRF | 533 | LQGTH |
| | | | | NTNYN | | WDYAI | | AKSYG | | S | | QPYT |
| | | | | QKFKG | | DY | | NTYLS | | | | |
| Antibody 4 | 4 | VYWMH | 110 | EIDPSES | 216 | GGYDG | 322 | RSSQSL | 428 | GISNRF | 534 | LQGTH |
| | | | | NTNYN | | WDYAI | | AKSYG | | S | | QPYT |
| | | | | QKFKG | | DY | | NTYLS | | | | |
| Antibody 5 | 5 | SMWMH | 111 | EIDPSES | 217 | GGYDG | 323 | RSSQSL | 429 | GISNRF | 535 | LQGTH |
| | | | | NTNYN | | WDYAI | | AKSYG | | S | | QPYT |
| | | | | QKFKG | | DY | | NTYLS | | | | |
| Antibody 6 | 6 | SQWMH | 112 | EIDPSES | 218 | GGYDG | 324 | RSSQSL | 430 | GISNRF | 536 | LQGTH |
| | | | | NTNYN | | WDYAI | | AKSYG | | S | | QPYT |
| | | | | QKFKG | | DY | | NTYLS | | | | |
| Antibody 7 | 7 | SYWMH | 113 | EIIPSES | 219 | GGYDG | 325 | RSSQSL | 431 | GISNRF | 537 | LQGTH |
| | | | | NTNYN | | WDYAI | | AKSYG | | S | | QPYT |
| | | | | QKFKG | | DY | | NTYLS | | | | |
| Antibody 8 | 8 | SYWMH | 114 | EIIPLES | 220 | GGYDG | 326 | RSSQSL | 432 | GISNRF | 538 | LQGTH |
| | | | | NTNYN | | WDYAI | | AKSYG | | S | | QPYT |
| | | | | QKFKG | | DY | | NTYLS | | | | |
| Antibody 9 | 9 | SYWMH | 115 | EIIPRES | 221 | GGYDG | 327 | RSSQSL | 433 | GISNRF | 539 | LQGTH |
| | | | | NTNYN | | WDYAI | | AKSYG | | S | | QPYT |
| | | | | QKFKG | | DY | | NTYLS | | | | |
| Antibody | 10 | SYWMH | 116 | EIIPVES | 222 | GGYDG | 328 | RSSQSL | 434 | GISNRF | 540 | LQGTH |
| 10 | | | | NTNYN | | WDYAI | | AKSYG | | S | | QPYT |
| | | | | QKFKG | | DY | | NTYLS | | | | |
| Antibody | 11 | SYWMH | 117 | EIRPSES | 223 | GGYDG | 329 | RSSQSL | 435 | GISNRF | 541 | LQGTH |
| 11 | | | | NTNYN | | WDYAI | | AKSYG | | S | | QPYT |
| | | | | QKFKG | | DY | | NTYLS | | | | |
| Antibody | 12 | SYWMH | 118 | EIRPLES | 224 | GGYDG | 330 | RSSQSL | 436 | GISNRF | 542 | LQGTH |
| 12 | | | | NTNYN | | WDYAI | | AKSYG | | S | | QPYT |
| | | | | QKFKG | | DY | | NTYLS | | | | |
| Antibody | 13 | SYWMH | 119 | EIRPRES | 225 | GGYDG | 331 | RSSQSL | 437 | GISNRF | 543 | LQGTH |
| 13 | | | | NTNYN | | WDYAI | | AKSYG | | S | | QPYT |
| | | | | QKFKG | | DY | | NTYLS | | | | |
| Antibody | 14 | SYWMH | 120 | EIRPVES | 226 | GGYDG | 332 | RSSQSL | 438 | GISNRF | 544 | LQGTH |
| 14 | | | | NTNYN | | WDYAI | | AKSYG | | S | | QPYT |
| | | | | QKFKG | | DY | | NTYLS | | | | |
| Antibody | 15 | SYWMH | 121 | EIDPLES | 227 | GGYDG | 333 | RSSQSL | 439 | GISNRF | 545 | LQGTH |
| 15 | | | | NTNYN | | WDYAI | | AKSYG | | S | | QPYT |
| | | | | QKFKG | | DY | | NTYLS | | | | |
| Antibody | 16 | SYWMH | 122 | EIDPME | 228 | GGYDG | 334 | RSSQSL | 440 | GISNRF | 546 | LQGTH |
| 16 | | | | SNTNYN | | WDYAI | | AKSYG | | S | | QPYT |
| | | | | QKFKG | | DY | | NTYLS | | | | |
| Antibody | 17 | SYWMH | 123 | EIDPRES | 229 | GGYDG | 335 | RSSQSL | 441 | GISNRF | 547 | LQGTH |
| 17 | | | | NTNYN | | WDYAI | | AKSYG | | S | | QPYT |
| | | | | QKFKG | | DY | | NTYLS | | | | |
| Antibody | 18 | SYWMH | 124 | EIDPVE | 230 | GGYDG | 336 | RSSQSL | 442 | GISNRF | 548 | LQGTH |
| 18 | | | | SNTNYN | | WDYAI | | AKSYG | | S | | QPYT |
| | | | | QKFKG | | DY | | NTYLS | | | | |
| Antibody | 19 | SYWMH | 125 | EIDPSES | 231 | GGYDG | 337 | RSSQSL | 443 | GISNRF | 549 | LQGTH |
| 19 | | | | NTNYN | | WDYFID | | AKSYG | | S | | QPYT |
| | | | | QKFKG | | Y | | NTYLS | | | | |
| Antibody | 20 | SYWMH | 126 | EIDPSES | 232 | GGYDG | 338 | RSSQSL | 444 | GISNRF | 550 | LQGTH |
| 20 | | | | NTNYN | | WDYFIY | | AKSYG | | S | | QPYT |
| | | | | QKFKG | | Y | | NTYLS | | | | |
| Antibody | 21 | SYWMH | 127 | EIDPSES | 233 | GGYDG | 339 | RSSQSL | 445 | GISNRF | 551 | LQGTH |
| 21 | | | | NTNYN | | WDYLID | | AKSYG | | S | | QPYT |
| | | | | QKFKG | | Y | | NTYLS | | | | |
| Antibody | 22 | SYWMH | 128 | EIDPSES | 234 | GGYDG | 340 | RSSQSL | 446 | GISNRF | 552 | LQGTH |
| 22 | | | | NTNYN | | WDYVI | | AKSYG | | S | | QPYT |
| | | | | QKFKG | | DY | | NTYLS | | | | |
| Antibody | 23 | SYWMH | 129 | EIDPSES | 235 | GGYDG | 341 | RSSQSL | 447 | GISNRF | 553 | LQGTH |
| 23 | | | | NTNYN | | WDYVI | | AKSYG | | S | | QPYT |
| | | | | QKFKG | | YY | | NTYLS | | | | |
| Antibody | 24 | SYWMH | 130 | EIDPSES | 236 | GGYDG | 342 | RSSQSL | 448 | GISNRF | 554 | LQGTH |
| 24 | | | | NTNYN | | WDYWI | | AKSYG | | S | | QPYT |
| | | | | QKFKG | | DY | | NTYLS | | | | |
| Antibody | 25 | SYWMH | 131 | EIDPSES | 237 | GGYDG | 343 | RSSQSL | 449 | GISNRF | 555 | LQGTH |
| 25 | | | | NTNYN | | WDYYI | | AKSYG | | S | | QPYT |
| | | | | QKFKG | | DY | | NTYLS | | | | |
| Antibody | 26 | SYWMH | 132 | EIDPSES | 238 | GGYDG | 344 | RSSQSL | 450 | GISNRF | 556 | LQGTH |
| 26 | | | | NTNYN | | WDYYI | | AKSYG | | S | | QPYT |
| | | | | QKFKG | | YY | | NTYLS | | | | |
| Antibody | 27 | SYWMH | 133 | EIDPSES | 239 | GGYDG | 345 | RSSQSL | 451 | GISNRF | 557 | LQGTH |
| 27 | | | | NTNYN | | WDYAI | | AKSYG | | S | | QPYT |
| | | | | QKFKG | | DY | | NTYLS | | | | |
| Antibody | 28 | SYWMH | 134 | EIDPSES | 240 | GGYDG | 346 | RSSQSL | 452 | GISNRF | 558 | LQGTH |
| 28 | | | | NTNYN | | WDYAI | | AKSYG | | S | | QPYT |
| | | | | QKFKG | | DY | | NTYLS | | | | |
| Antibody | 29 | SYWMH | 135 | EIDPSES | 241 | GGYDG | 347 | RSSQSL | 453 | GISNRF | 559 | LOGTH |
| 29 | | | | NTNYN | | WDYAI | | AKSYG | | S | | QPYT |
| | | | | QKFKG | | DY | | NTYLS | | | | |
| Antibody | 30 | SYWMH | 136 | EIDPSES | 242 | GGYDG | 348 | RSSQSL | 454 | GISNRF | 560 | LQGTH |
| 30 | | | | NTNYN | | WDYAI | | AKSYG | | S | | QPYT |
| | | | | QKFKG | | DY | | NTYLS | | | | |
| Antibody | 31 | SYWMH | 137 | EIDPSES | 243 | GGYDG | 349 | RSSQSL | 455 | GISNRF | 561 | LQGTH |
| 31 | | | | NTNYN | | WDYAI | | AKSYG | | S | | QPYT |
| | | | | QKFKG | | DY | | NTYLS | | | | |
| Antibody | 32 | SYWMH | 138 | EIDPSES | 244 | GGYWG | 350 | RSSQSL | 456 | GISNRF | 562 | LQGTH |
| 32 | | | | NTNYN | | WDYAI | | AKSYG | | S | | QPYT |
| | | | | QKFKG | | DY | | NTYLS | | | | |
| Antibody | 33 | SYWMH | 139 | EIDPSES | 245 | GGYYG | 351 | RSSQSL | 457 | GISNRF | 563 | LQGTH |
| 33 | | | | NTNYN | | WDYAI | | AKSYG | | S | | QPYT |
| | | | | QKFKG | | DY | | NTYLS | | | | |
| Antibody | 34 | SYWMH | 140 | EIDPSES | 246 | GGYDG | 352 | RSSQSL | 458 | GISNRF | 564 | LQGTH |
| 34 | | | | NTNYN | | WDYAIF | | AKSYG | | S | | QPYT |
| | | | | QKFKG | | Y | | NTYLS | | | | |
| Antibody | 35 | SYWMH | 141 | EIDPSES | 247 | GGYDG | 353 | RSSQSL | 459 | GISNRF | 565 | LQGTH |
| 35 | | | | NTNYN | | WDYAI | | AKSYG | | S | | QPYT |
| | | | | QKFKG | | MY | | NTYLS | | | | |
| Antibody | 36 | SYWMH | 142 | EIDPSES | 248 | GGYDG | 354 | RSSQSL | 460 | GISNRF | 566 | LQGTH |
| 36 | | | | NTNYN | | WDYAI | | AKSYG | | S | | QPYT |
| | | | | QKFKG | | WY | | NTYLS | | | | |
| Antibody | 37 | SYWMH | 143 | EIDPSES | 249 | GGYDG | 355 | RSSQSL | 461 | GISNRF | 567 | LQGTH |
| 37 | | | | NTNYN | | WDYAI | | AKSYG | | S | | QPYT |
| | | | | QKFKG | | YY | | NTYLS | | | | |
| Antibody | 38 | SYWMH | 144 | EIDPSES | 250 | GFYDG | 356 | RSSQSL | 462 | GISNRF | 568 | LQGTH |
| 38 | | | | NTNYN | | WDYAI | | AKSYG | | S | | QPYT |
| | | | | QKFKG | | DY | | NTYLS | | | | |
| Antibody | 39 | SYWMH | 145 | EIDPSES | 251 | GFYDG | 357 | RSSQSL | 463 | GISNRF | 569 | LQGTH |
| 39 | | | | NTNYN | | WDYLID | | AKSYG | | S | | QPYT |
| | | | | QKFKG | | Y | | NTYLS | | | | |
| Antibody | 40 | SYWMH | 146 | EIDPSES | 252 | GFYDG | 358 | RSSQSL | 464 | GISNRF | 570 | LQGTH |
| 40 | | | | NTNYN | | WDYAI | | AKSYG | | S | | QPYT |
| | | | | QKFKG | | YY | | NTYLS | | | | |
| Antibody | 41 | SYWMH | 147 | EIDPSES | 253 | GMYDG | 359 | RSSQSL | 465 | GISNRF | 571 | LQGTH |
| 41 | | | | NTNYN | | WDYAI | | AKSYG | | S | | QPYT |
| | | | | QKFKG | | DY | | NTYLS | | | | |
| Antibody | 42 | SYWMH | 148 | EIDPSES | 254 | GRYDG | 360 | RSSQSL | 466 | GISNRF | 572 | LQGTH |
| 42 | | | | NTNYN | | WDYAI | | AKSYG | | S | | QPYT |
| | | | | QKFKG | | DY | | NTYLS | | | | |
| Antibody | 43 | SYWMH | 149 | EIDPSES | 255 | GVYDG | 361 | RSSQSL | 467 | GISNRF | 573 | LQGTH |
| 43 | | | | NTNYN | | WDYAI | | AKSYG | | S | | QPYT |
| | | | | QKFKG | | DY | | NTYLS | | | | |
| Antibody | 44 | SYWMH | 150 | EIDPSES | 256 | GGYDI | 362 | RSSQSL | 468 | GISNRF | 574 | LQGTH |
| 44 | | | | NTNYN | | WDYAI | | AKSYG | | S | | QPYT |
| | | | | QKFKG | | DY | | NTYLS | | | | |
| Antibody | 45 | SYWMH | 151 | EIDPSES | 257 | GGYDL | 363 | RSSQSL | 469 | GISNRF | 575 | LQGTH |
| 45 | | | | NTNYN | | WDYAI | | AKSYG | | S | | QPYT |
| | | | | QKFKG | | DY | | NTYLS | | | | |
| Antibody | 46 | SYWMH | 152 | EIDPSES | 258 | GGYDV | 364 | RSSQSL | 470 | GISNRF | 576 | LQGTH |
| 46 | | | | NTNYN | | WDYAI | | AKSYG | | S | | QPYT |
| | | | | QKFKG | | DY | | NTYLS | | | | |
| Antibody | 47 | SYWMH | 153 | EIDPSES | 259 | FGYDG | 365 | RSSQSL | 471 | GISNRF | 577 | LQGTH |
| 47 | | | | NTNYN | | WDYAI | | AKSYG | | S | | QPYT |
| | | | | QKFKG | | DY | | NTYLS | | | | |
| Antibody | 48 | SYWMH | 154 | EIDPSES | 260 | YGYDG | 366 | RSSQSL | 472 | GISNRF | 578 | LQGTH |
| 48 | | | | NTNYN | | WDYAI | | AKSYG | | S | | QPYT |
| | | | | QKFKG | | DY | | NTYLS | | | | |
| Antibody | 49 | SYWMH | 155 | EIDPSES | 261 | GGYDG | 367 | RSSQSL | 473 | GISNRF | 579 | LQGTH |
| 49 | | | | NTNYN | | WDYAI | | AASYG | | S | | QPYT |
| | | | | QKFKG | | DY | | NTYLS | | | | |
| Antibody | 50 | SYWMH | 156 | EIDPSES | 262 | GGYDG | 368 | RSSQSL | 474 | GISNRF | 580 | LQGTH |
| 50 | | | | NTNYN | | WDYAI | | ADSYG | | S | | QPYT |
| | | | | QKFKG | | DY | | NTYLS | | | | |
| Antibody | 51 | SYWMH | 157 | EIDPSES | 263 | GGYDG | 369 | RSSQSL | 475 | GISNRF | 581 | LQGTH |
| 51 | | | | NTNYN | | WDYAI | | AISYGN | | S | | QPYT |
| | | | | QKFKG | | DY | | TYLS | | | | |
| Antibody | 52 | SYWMH | 158 | EIDPSES | 264 | GGYDG | 370 | RSSQSL | 476 | GISNRF | 582 | LQGTH |
| 52 | | | | NTNYN | | WDYAI | | AIFYGN | | S | | QPYT |
| | | | | QKFKG | | DY | | TYLS | | | | |
| Antibody | 53 | SYWMH | 159 | EIDPSES | 265 | GGYDG | 371 | RSSQSL | 477 | GISNRF | 583 | LQGTH |
| 53 | | | | NTNYN | | WDYAI | | AIFYGIT | | S | | QPYT |
| | | | | QKFKG | | DY | | YLS | | | | |
| Antibody | 54 | SYWMH | 160 | EIDPSES | 266 | GGYDG | 372 | RSSQSL | 478 | GISNRF | 584 | LQGTH |
| 54 | | | | NTNYN | | WDYAI | | AIFYGL | | S | | QPYT |
| | | | | QKFKG | | DY | | TYLS | | | | |
| Antibody | 55 | SYWMH | 161 | EIDPSES | 267 | GGYDG | 373 | RSSQSL | 479 | GISNRF | 585 | LQGTH |
| 55 | | | | NTNYN | | WDYAI | | AILYGIT | | S | | QPYT |
| | | | | QKFKG | | DY | | YLS | | | | |
| Antibody | 56 | SYWMH | 162 | EIDPSES | 268 | GGYDG | 374 | RSSQSL | 480 | GISNRF | 586 | LQGTH |
| | | | | NTNYN | | WDYAI | | AILYGL | | | | |
| 56 | | | | QKFKG | | DY | | TYLS | | S | | QPYT |
| Antibody | 57 | SYWMH | 163 | EIDPSES | 269 | GGYDG | 375 | RSSQSL | 481 | GISNRF | 587 | LQGTH |
| 57 | | | | NTNYN | | WDYAI | | ALSYGN | | S | | QPYT |
| | | | | QKFKG | | DY | | TYLS | | | | |
| Antibody | 58 | SYWMH | 164 | EIDPSES | 270 | GGYDG | 376 | RSSQSL | 482 | GISNRF | 588 | LQGTH |
| 58 | | | | NTNYN | | WDYAI | | AMSYG | | S | | QPYT |
| | | | | QKFKG | | DY | | NTYLS | | | | |
| Antibody | 59 | SYWMH | 165 | EIDPSES | 271 | GGYDG | 377 | RSSQSL | 483 | GISNRF | 589 | LQGTH |
| 59 | | | | NTNYN | | WDYAI | | ANSYG | | S | | QPYT |
| | | | | QKFKG | | DY | | NTYLS | | | | |
| Antibody | 60 | SYWMH | 166 | EIDPSES | 272 | GGYDG | 378 | RSSQSL | 484 | GISNRF | 590 | LQGTH |
| 60 | | | | NTNYN | | WDYAI | | APSYGN | | S | | QPYT |
| | | | | QKFKG | | DY | | TYLS | | | | |
| Antibody | 61 | SYWMH | 167 | EIDPSES | 273 | GGYDG | 379 | RSSQSL | 485 | GISNRF | 591 | LQGTH |
| 61 | | | | NTNYN | | WDYAI | | AQSYG | | S | | QPYT |
| | | | | QKFKG | | DY | | NTYLS | | | | |
| Antibody | 62 | SYWMH | 168 | EIDPSES | 274 | GGYDG | 380 | RSSQSL | 486 | GISNRF | 592 | LQGTH |
| 62 | | | | NTNYN | | WDYAI | | ARSYG | | S | | QPYT |
| | | | | QKFKG | | DY | | NTYLS | | | | |
| Antibody | 63 | SYWMH | 169 | EIDPSES | 275 | GGYDG | 381 | RSSQSL | 487 | GISNRF | 593 | LQGTH |
| 63 | | | | NTNYN | | WDYAI | | ARFYGL | | S | | QPYT |
| | | | | QKFKG | | DY | | TYLS | | | | |
| Antibody | 64 | SYWMH | 170 | EIDPSES | 276 | GGYDG | 382 | RSSQSL | 488 | GISNRF | 594 | LQGTH |
| 64 | | | | NTNYN | | WDYAI | | ASSYGN | | S | | QPYT |
| | | | | QKFKG | | DY | | TYLS | | | | |
| Antibody | 65 | SYWMH | 171 | EIDPSES | 277 | GGYDG | 383 | RSSQSL | 489 | GISNRF | 595 | LQGTH |
| 65 | | | | NTNYN | | WDYAI | | ATSYGN | | S | | QPYT |
| | | | | QKFKG | | DY | | TYLS | | | | |
| Antibody | 66 | SYWMH | 172 | EIDPSES | 278 | GGYDG | 384 | RSSQSL | 490 | GISNRF | 596 | LQGTH |
| 66 | | | | NTNYN | | WDYAI | | ATYYGI | | S | | QPYT |
| | | | | QKFKG | | DY | | TYLS | | | | |
| Antibody | 67 | SYWMH | 173 | EIDPSES | 279 | GGYDG | 385 | RSSQSL | 491 | GISNRF | 597 | LQGTH |
| 67 | | | | NTNYN | | WDYAI | | ATYYG | | S | | QPYT |
| | | | | QKFKG | | DY | | LTYLS | | | | |
| Antibody | 68 | SYWMH | 174 | EIDPSES | 280 | GGYDG | 386 | RSSQSL | 492 | GISNRF | 598 | LQGTH |
| 68 | | | | NTNYN | | WDYAI | | AVSYG | | S | | QPYT |
| | | | | QKFKG | | DY | | NTYLS | | | | |
| Antibody | 69 | SYWMH | 175 | EIDPSES | 281 | GGYDG | 387 | RSSQSL | 493 | GISNRF | 599 | LQGTH |
| 69 | | | | NTNYN | | WDYAI | | AYSYG | | S | | QPYT |
| | | | | QKFKG | | DY | | NTYLS | | | | |
| Antibody | 70 | SYWMH | 176 | EIDPSES | 282 | GGYDG | 388 | RSSQSL | 494 | GISNRF | 600 | LQGTH |
| 70 | | | | NTNYN | | WDYAI | | AYFYGI | | S | | QPYT |
| | | | | QKFKG | | DY | | TYLS | | | | |
| Antibody | 71 | SYWMH | 177 | EIDPSES | 283 | GGYDG | 389 | RSSQSL | 495 | GISNRF | 601 | LQGTH |
| 71 | | | | NTNYN | | WDYAI | | AYLYGI | | S | | QPYT |
| | | | | QKFKG | | DY | | TYLS | | | | |
| Antibody | 72 | SYWMH | 178 | EIDPSES | 284 | GGYDG | 390 | RSSQSL | 496 | GISNRF | 602 | LQGTH |
| 72 | | | | NTNYN | | WDYAI | | AKSYGI | | S | | QPYT |
| | | | | QKFKG | | DY | | TYLS | | | | |
| Antibody | 73 | SYWMH | 179 | EIDPSES | 285 | GGYDG | 391 | RSSQSL | 497 | GISNRF | 603 | LQGTH |
| 73 | | | | NTNYN | | WDYAI | | AKSYGL | | S | | QPYT |
| | | | | QKFKG | | DY | | TYLS | | | | |
| Antibody | 74 | SYWMH | 180 | EIDPSES | 286 | GGYDG | 392 | RSSQSL | 498 | GISNRF | 604 | LQGTH |
| 74 | | | | NTNYN | | WDYAI | | AKFYG | | S | | QPYT |
| | | | | QKFKG | | DY | | NTYLS | | | | |
| Antibody | 75 | SYWMH | 181 | EIDPSES | 287 | GGYDG | 393 | RSSQSL | 499 | GISNRF | 605 | LQGTH |
| 75 | | | | NTNYN | | WDYAI | | AKIYGN | | S | | QPYT |
| | | | | QKFKG | | DY | | TYLS | | | | |
| Antibody | 76 | SYWMH | 182 | EIDPSES | 288 | GGYDG | 394 | RSSQSL | 500 | GISNRF | 606 | LQGTH |
| 76 | | | | NTNYN | | WDYAI | | AKLYG | | S | | QPYT |
| | | | | QKFKG | | DY | | NTYLS | | | | |
| Antibody | 77 | SYWMH | 183 | EIDPSES | 289 | GGYDG | 395 | RSSQSL | 501 | GISNRF | 607 | LQGTH |
| 77 | | | | NTNYN | | WDYAI | | AKMYG | | S | | QPYT |
| | | | | QKFKG | | DY | | NTYLS | | | | |
| Antibody | 78 | SYWMH | 184 | EIDPSES | 290 | GGYDG | 396 | RSSQSL | 502 | GISNRF | 608 | LQGTH |
| 78 | | | | NTNYN | | WDYAI | | AKQYG | | S | | QPYT |
| | | | | QKFKG | | DY | | NTYLS | | | | |
| Antibody | 79 | SYWMH | 185 | EIDPSES | 291 | GGYDG | 397 | RSSQSL | 503 | GISNRF | 609 | LQGTH |
| 79 | | | | NTNYN | | WDYAI | | AKYYG | | S | | QPYT |
| | | | | QKFKG | | DY | | NTYLS | | | | |
| Antibody | 80 | SYWMH | 186 | EIDPSES | 292 | GGYDG | 398 | RSSQSL | 504 | GISNRF | 610 | LQGTH |
| 80 | | | | NTNYN | | WDYAI | | AKSHG | | S | | QPYT |
| | | | | QKFKG | | DY | | NTYLS | | | | |
| Antibody | 81 | SYWMH | 187 | EIDPSES | 293 | GGYDG | 399 | RSSQSL | 505 | FISNRFS | 611 | LQGTH |
| 81 | | | | NTNYN | | WDYAI | | AKSYG | | | | QPYT |
| | | | | QKFKG | | DY | | NTYLS | | | | |
| Antibody | 82 | SYWMH | 188 | EIDPSES | 294 | GGYDG | 400 | RSSQSL | 506 | PISNRFS | 612 | LQGTH |
| 82 | | | | NTNYN | | WDYAI | | AKSYG | | | | QPYT |
| | | | | QKFKG | | DY | | NTYLS | | | | |
| Antibody | 83 | SYWMH | 189 | EIDPSES | 295 | GGYDG | 401 | RSSQSL | 507 | YISNRF | 613 | LQGTH |
| 83 | | | | NTNYN | | WDYAI | | AKSYG | | S | | QPYT |
| | | | | QKFKG | | DY | | NTYLS | | | | |
| Antibody | 84 | SYWMH | 190 | EIDPSES | 296 | GGYDG | 402 | RSSQSL | 508 | GISNRF | 614 | LQFTHQ |
| 84 | | | | NTNYN | | WDYAI | | AKSYG | | S | | PYT |
| | | | | QKFKG | | DY | | NTYLS | | | | |
| Antibody | 85 | SYWMH | 191 | EIDPSES | 297 | GGYDG | 403 | RSSQSL | 509 | GISNRF | 615 | LQFTIQ |
| 85 | | | | NTNYN | | WDYAI | | AKSYG | | S | | FYT |
| | | | | QKFKG | | DY | | NTYLS | | | | |
| Antibody | 86 | SYWMH | 192 | EIDPSES | 298 | GGYDG | 404 | RSSQSL | 510 | GISNRF | 616 | LQFTIQ |
| 86 | | | | NTNYN | | WDYAI | | AKSYG | | S | | VYT |
| | | | | QKFKG | | DY | | NTYLS | | | | |
| Antibody | 87 | SYWMH | 193 | EIDPSES | 299 | GGYDG | 405 | RSSQSL | 511 | GISNRF | 617 | LQFTIQ |
| 87 | | | | NTNYN | | WDYAI | | AKSYG | | S | | PYI |
| | | | | QKFKG | | DY | | NTYLS | | | | |
| Antibody | 88 | SYWMH | 194 | EIDPSES | 300 | GGYDG | 406 | RSSQSL | 512 | GISNRF | 618 | LQFTHQ |
| 88 | | | | NTNYN | | WDYAI | | AKSYG | | S | | FYT |
| | | | | QKFKG | | DY | | NTYLS | | | | |
| Antibody | 89 | SYWMH | 195 | EIDPSES | 301 | GGYDG | 407 | RSSQSL | 513 | GISNRF | 619 | LQFTHQ |
| 89 | | | | NTNYN | | WDYAI | | AKSYG | | S | | IYI |
| | | | | QKFKG | | DY | | NTYLS | | | | |
| Antibody | 90 | SYWMH | 196 | EIDPSES | 302 | GGYDG | 408 | RSSQSL | 514 | GISNRF | 620 | LQFTHQ |
| 90 | | | | NTNYN | | WDYAI | | AKSYG | | S | | PYI |
| | | | | QKFKG | | DY | | NTYLS | | | | |
| Antibody | 91 | SYWMH | 197 | EIDPSES | 303 | GGYDG | 409 | RSSQSL | 515 | GISNRF | 621 | LQRTHQ |
| 91 | | | | NTNYN | | WDYAI | | AKSYG | | S | | PYT |
| | | | | QKFKG | | DY | | NTYLS | | | | |
| Antibody | 92 | SYWMH | 198 | EIDPSES | 304 | GGYDG | 410 | RSSQSL | 516 | GISNRF | 622 | LQRTIQ |
| 92 | | | | NTNYN | | WDYAI | | AKSYG | | S | | VYI |
| | | | | QKFKG | | DY | | NTYLS | | | | |
| Antibody | 93 | SYWMH | 199 | EIDPSES | 305 | GGYDG | 411 | RSSQSL | 517 | GISNRF | 623 | LQRTIQ |
| 93 | | | | NTNYN | | WDYAI | | AKSYG | | S | | YYT |
| | | | | QKFKG | | DY | | NTYLS | | | | |
| Antibody | 94 | SYWMH | 200 | EIDPSES | 306 | GGYDG | 412 | RSSQSL | 518 | GISNRF | 624 | LQVTH |
| 94 | | | | NTNYN | | WDYAI | | AKSYG | | S | | QPYT |
| | | | | QKFKG | | DY | | NTYLS | | | | |
| Antibody | 95 | SYWMH | 201 | EIDPSES | 307 | GGYDG | 413 | RSSQSL | 519 | GISNRF | 625 | LQWTH |
| 95 | | | | NTNYN | | WDYAI | | AKSYG | | S | | QPYT |
| | | | | QKFKG | | DY | | NTYLS | | | | |
| Antibody | 96 | SYWMH | 202 | EIDPSES | 308 | GGYDG | 414 | RSSQSL | 520 | GISNRF | 626 | LQYTH |
| 96 | | | | NTNYN | | WDYAI | | AKSYG | | S | | QPYT |
| | | | | QKFKG | | DY | | NTYLS | | | | |
| Antibody | 97 | SYWMH | 203 | EIDPSES | 309 | GGYDG | 415 | RSSQSL | 521 | GISNRF | 627 | LQYTIQ |
| 97 | | | | NTNYN | | WDYAI | | AKSYG | | S | | FYI |
| | | | | QKFKG | | DY | | NTYLS | | | | |
| Antibody | 98 | SYWMH | 204 | EIDPSES | 310 | GGYDG | 416 | RSSQSL | 522 | GISNRF | 628 | LQYTH |
| 98 | | | | NTNYN | | WDYAI | | AKSYG | | S | | QFYI |
| | | | | QKFKG | | DY | | NTYLS | | | | |
| Antibody | 99 | SYWMH | 205 | EIDPSES | 311 | GGYDG | 417 | RSSQSL | 523 | GISNRF | 629 | LQGTIQ |
| 99 | | | | NTNYN | | WDYAI | | AKSYG | | S | | PYT |
| | | | | QKFKG | | DY | | NTYLS | | | | |
| Antibody | 100 | SYWMH | 206 | EIDPSES | 312 | GGYDG | 418 | RSSQSL | 524 | GISNRF | 630 | LQGTW |
| 100 | | | | NTNYN | | WDYAI | | AKSYG | | S | | QPYT |
| | | | | QKFKG | | DY | | NTYLS | | | | |
| Antibody | 101 | SYWMH | 207 | EIDPSES | 313 | GGYDG | 419 | RSSQSL | 525 | GISNRF | 631 | LOGTH |
| 101 | | | | NTNYN | | WDYAI | | AKSYG | | S | | QFYT |
| | | | | QKFKG | | DY | | NTYLS | | | | |
| Antibody | 102 | SYWMH | 208 | EIDPSES | 314 | GGYDG | 420 | RSSQSL | 526 | GISNRF | 632 | LQGTH |
| 102 | | | | NTNYN | | WDYAI | | AKSYG | | S | | QIYT |
| | | | | QKFKG | | DY | | NTYLS | | | | |
| Antibody | 103 | SYWMH | 209 | EIDPSES | 315 | GGYDG | 421 | RSSQSL | 527 | GISNRF | 633 | LQGTH |
| 103 | | | | NTNYN | | WDYAI | | AKSYG | | S | | QVYT |
| | | | | QKFKG | | DY | | NTYLS | | | | |
| Antibody | 104 | SYWMH | 210 | EIDPSES | 316 | GGYDG | 422 | RSSQSL | 528 | GISNRF | 634 | LQGTH |
| 104 | | | | NTNYN | | WDYAI | | AKSYG | | S | | QYYT |
| | | | | QKFKG | | DY | | NTYLS | | | | |
| Antibody | 105 | SYWMH | 211 | EIDPSES | 317 | GGYDG | 423 | RSSQSL | 529 | GISNRF | 635 | LQGTH |
| 105 | | | | NTNYN | | WDYAI | | AKSYG | | S | | QPYI |
| | | | | QKFKG | | DY | | NTYLS | | | | |
| Antibody | 106 | SYWMH | 212 | EIDPSES | 318 | GGYDG | 424 | RSSQSL | 530 | GISNRF | 636 | LQGTH |
| 106 | | | | NTNYN | | WDYAI | | AKSYG | | S | | QPYR |
| | | | | QKFKG | | DY | | NTYLS | | | | |

Chothia Numbering

| | | | | | | | | | | | | |
|---|---|---|---|---|---|---|---|---|---|---|---|---|
| Antibody 1 | 637 | GGTFTS | 743 | DPSESN | 849 | GGYDG | 955 | RSSQSL | 1061 | GISNRF | 1167 | LOGTH |
| | | YWMH | | | | WDYAI | | AKSYG | | S | | QPYT |
| | | | | | | DY | | NTYLS | | | | |
| Antibody 2 | 638 | GYTFTS | 744 | DPSESN | 850 | GGYDG | 956 | RSSQSL | 1062 | GISNRF | 1168 | LOGTH |
| | | YWMH | | | | WDYAI | | AKSYG | | S | | QPYT |
| | | | | | | DY | | NTYLS | | | | |
| Antibody 3 | 639 | GYTFTI | 745 | DPSESN | 851 | GGYDG | 957 | RSSQSL | 1063 | GISNRF | 1169 | LQGTH |
| | | | | | | WDYAI | | AKSYG | | | | |
| | | YWMH | | | | DY | | NTYLS | | S | | QPYT |
| Antibody 4 | 640 | GYTFTV | 746 | DPSESN | 852 | GGYDG | 958 | RSSQSL | 1064 | GISNRF | 1170 | LQGTH |
| | | YWMH | | | | WDYAI | | AKSYG | | S | | QPYT |
| | | | | | | DY | | NTYLS | | | | |
| Antibody 5 | 641 | GYTFTS | 747 | DPSESN | 853 | GGYDG | 959 | RSSQSL | 1065 | GISNRF | 1171 | LQGTH |
| | | MWMH | | | | WDYAI | | AKSYG | | S | | QPYT |
| | | | | | | DY | | NTYLS | | | | |
| Antibody 6 | 642 | GYTFTS | 748 | DPSESN | 854 | GGYDG | 960 | RSSQSL | 1066 | GISNRF | 1172 | LQGTH |
| | | QWMH | | | | WDYAI | | AKSYG | | S | | QPYT |
| | | | | | | DY | | NTYLS | | | | |
| Antibody 7 | 643 | GYTFTS | 749 | IPSESN | 855 | GGYDG | 961 | RSSQSL | 1067 | GISNRF | 1173 | LQGTH |
| | | YWMH | | | | WDYAI | | AKSYG | | S | | QPYT |
| | | | | | | DY | | NTYLS | | | | |
| Antibody 8 | 644 | GYTFTS | 750 | IPLESN | 856 | GGYDG | 962 | RSSQSL | 1068 | GISNRF | 1174 | LQGTH |
| | | YWMH | | | | WDYAI | | AKSYG | | S | | QPYT |
| | | | | | | DY | | NTYLS | | | | |
| Antibody 9 | 645 | GYTFTS | 751 | IPRESN | 857 | GGYDG | 963 | RSSQSL | 1069 | GISNRF | 1175 | LQGTH |
| | | YWMH | | | | WDYAI | | AKSYG | | S | | QPYT |
| | | | | | | DY | | NTYLS | | | | |
| Antibody | 646 | GYTFTS | 752 | IPVESN | 858 | GGYDG | 964 | RSSQSL | 1070 | GISNRF | 1176 | LQGTH |
| 10 | | YWMH | | | | WDYAI | | AKSYG | | S | | QPYT |
| | | | | | | DY | | NTYLS | | | | |
| Antibody | 647 | GYTFTS | 753 | RPSESN | 859 | GGYDG | 965 | RSSQSL | 1071 | GISNRF | 1177 | LQGTH |
| 11 | | YWMH | | | | WDYAI | | AKSYG | | S | | QPYT |
| | | | | | | DY | | NTYLS | | | | |
| Antibody | 648 | GYTFTS | 754 | RPLESN | 860 | GGYDG | 966 | RSSQSL | 1072 | GISNRF | 1178 | LOGTH |
| 12 | | YWMH | | | | WDYAI | | AKSYG | | S | | QPYT |
| | | | | | | DY | | NTYLS | | | | |
| Antibody | 649 | GYTFTS | 755 | RPRESN | 861 | GGYDG | 967 | RSSQSL | 1073 | GISNRF | 1179 | LQGTH |
| 13 | | YWMH | | | | WDYAI | | AKSYG | | S | | QPYT |
| | | | | | | DY | | NTYLS | | | | |
| Antibody | 650 | GYTFTS | 756 | RPVESN | 862 | GGYDG | 968 | RSSQSL | 1074 | GISNRF | 1180 | LQGTH |
| 14 | | YWMH | | | | WDYAI | | AKSYG | | S | | QPYT |
| | | | | | | DY | | NTYLS | | | | |
| Antibody | 651 | GYTFTS | 757 | DPLESN | 863 | GGYDG | 969 | RSSQSL | 1075 | GISNRF | 1181 | LQGTH |
| 15 | | YWMH | | | | WDYAI | | AKSYG | | S | | QPYT |
| | | | | | | DY | | NTYLS | | | | |
| Antibody | 652 | GYTFTS | 758 | DPMESN | 864 | GGYDG | 970 | RSSQSL | 1076 | GISNRF | 1182 | LQGTH |
| 16 | | YWMH | | | | WDYAI | | AKSYG | | S | | QPYT |
| | | | | | | DY | | NTYLS | | | | |
| Antibody | 653 | GYTFTS | 759 | DPRESN | 865 | GGYDG | 971 | RSSQSL | 1077 | GISNRF | 1183 | LQGTH |
| 17 | | YWMH | | | | WDYAI | | AKSYG | | S | | QPYT |
| | | | | | | DY | | NTYLS | | | | |
| Antibody | 654 | GYTFTS | 760 | DPVESN | 866 | GGYDG | 972 | RSSQSL | 1078 | GISNRF | 1184 | LQGTH |
| 18 | | YWMH | | | | WDYAI | | AKSYG | | S | | QPYT |
| | | | | | | DY | | NTYLS | | | | |
| Antibody | 655 | GYTFTS | 761 | DPSESN | 867 | GGYDG | 973 | RSSQSL | 1079 | GISNRF | 1185 | LQGTH |
| 19 | | YWMH | | | | WDYFID | | AKSYG | | S | | QPYT |
| | | | | | | Y | | NTYLS | | | | |
| Antibody | 656 | GYTFTS | 762 | DPSESN | 868 | GGYDG | 974 | RSSQSL | 1080 | GISNRF | 1186 | LQGTH |
| 20 | | YWMH | | | | WDYFIY | | AKSYG | | S | | QPYT |
| | | | | | | Y | | NTYLS | | | | |
| Antibody | 657 | GYTFTS | 763 | DPSESN | 869 | GGYDG | 975 | RSSQSL | 1081 | GISNRF | 1187 | LQGTH |
| 21 | | YWMH | | | | WDYLID | | AKSYG | | S | | QPYT |
| | | | | | | Y | | NTYLS | | | | |
| Antibody | 658 | GYTFTS | 764 | DPSESN | 870 | GGYDG | 976 | RSSQSL | 1082 | GISNRF | 1188 | LQGTH |
| 22 | | YWMH | | | | WDYVI | | AKSYG | | S | | QPYT |
| | | | | | | DY | | NTYLS | | | | |
| Antibody | 659 | GYTFTS | 765 | DPSESN | 871 | GGYDG | 977 | RSSQSL | 1083 | GISNRF | 1189 | LQGTH |
| 23 | | YWMH | | | | WDYVI | | AKSYG | | S | | QPYT |
| | | | | | | YY | | NTYLS | | | | |
| Antibody | 660 | GYTFTS | 766 | DPSESN | 872 | GGYDG | 978 | RSSQSL | 1084 | GISNRF | 1190 | LQGTH |
| 24 | | YWMH | | | | WDYWI | | AKSYG | | S | | QPYT |
| | | | | | | DY | | NTYLS | | | | |
| Antibody | 661 | GYTFTS | 767 | DPSESN | 873 | GGYDG | 979 | RSSQSL | 1085 | GISNRF | 1191 | LQGTH |
| 25 | | YWMH | | | | WDYYI | | AKSYG | | S | | QPYT |
| | | | | | | DY | | NTYLS | | | | |
| Antibody | 662 | GYTFTS | 768 | DPSESN | 874 | GGYDG | 980 | RSSQSL | 1086 | GISNRF | 1192 | LQGTH |
| 26 | | YWMH | | | | WDYYI | | AKSYG | | S | | QPYT |
| | | | | | | YY | | NTYLS | | | | |
| Antibody | 663 | GYTFTS | 769 | DPSESN | 875 | GGYDG | 981 | RSSQSL | 1087 | GISNRF | 1193 | LQGTH |
| 27 | | YWMH | | | | WDYAI | | AKSYG | | S | | QPYT |
| | | | | | | DY | | NTYLS | | | | |
| Antibody | 664 | GYTFTS | 770 | DPSESN | 876 | GGYDG | 982 | RSSQSL | 1088 | GISNRF | 1194 | LQGTH |
| 28 | | YWMH | | | | WDYAI | | AKSYG | | S | | QPYT |
| | | | | | | DY | | NTYLS | | | | |
| Antibody | 665 | GYTFTS | 771 | DPSESN | 877 | GGYDG | 983 | RSSQSL | 1089 | GISNRF | 1195 | LQGTH |
| 29 | | YWMH | | | | WDYAI | | AKSYG | | S | | QPYT |
| | | | | | | DY | | NTYLS | | | | |
| Antibody | 666 | GYTFTS | 772 | DPSESN | 878 | GGYDG | 984 | RSSQSL | 1090 | GISNRF | 1196 | LQGTH |
| 30 | | YWMH | | | | WDYAI | | AKSYG | | S | | QPYT |
| | | | | | | DY | | NTYLS | | | | |
| Antibody | 667 | GYTFTS | 773 | DPSESN | 879 | GGYDG | 985 | RSSQSL | 1091 | GISNRF | 1197 | LQGTH |
| 31 | | YWMH | | | | WDYAI | | AKSYG | | S | | QPYT |
| | | | | | | DY | | NTYLS | | | | |
| Antibody | 668 | GYTFTS | 774 | DPSESN | 880 | GGYWG | 986 | RSSQSL | 1092 | GISNRF | 1198 | LQGTH |
| 32 | | YWMH | | | | WDYAI | | AKSYG | | S | | QPYT |
| | | | | | | DY | | NTYLS | | | | |
| Antibody | 669 | GYTFTS | 775 | DPSESN | 881 | GGYYG | 987 | RSSQSL | 1093 | GISNRF | 1199 | LQGTH |
| 33 | | YWMH | | | | WDYAI | | AKSYG | | S | | QPYT |
| | | | | | | DY | | NTYLS | | | | |
| Antibody | 670 | GYTFTS | 776 | DPSESN | 882 | GGYDG | 988 | RSSQSL | 1094 | GISNRF | 1200 | LQGTH |
| 34 | | YWMH | | | | WDYAIF | | AKSYG | | S | | QPYT |
| | | | | | | Y | | NTYLS | | | | |
| Antibody | 671 | GYTFTS | 777 | DPSESN | 883 | GGYDG | 989 | RSSQSL | 1095 | GISNRF | 1201 | LQGTH |
| 35 | | YWMH | | | | WDYAI | | AKSYG | | S | | QPYT |
| | | | | | | MY | | NTYLS | | | | |
| Antibody | 672 | GYTFTS | 778 | DPSESN | 884 | GGYDG | 990 | RSSQSL | 1096 | GISNRF | 1202 | LQGTH |
| 36 | | YWMH | | | | WDYAI | | AKSYG | | S | | QPYT |
| | | | | | | WY | | NTYLS | | | | |
| Antibody | 673 | GYTFTS | 779 | DPSESN | 885 | GGYDG | 991 | RSSQSL | 1097 | GISNRF | 1203 | LQGTH |
| 37 | | YWMH | | | | WDYAI | | AKSYG | | S | | QPYT |
| | | | | | | YY | | NTYLS | | | | |
| Antibody | 674 | GYTFTS | 780 | DPSESN | 886 | GFYDG | 992 | RSSQSL | 1098 | GISNRF | 1204 | LQGTH |
| 38 | | YWMH | | | | WDYAI | | AKSYG | | S | | QPYT |
| | | | | | | DY | | NTYLS | | | | |
| Antibody | 675 | GYTFTS | 781 | DPSESN | 887 | GFYDG | 993 | RSSQSL | 1099 | GISNRF | 1205 | LQGTH |
| 39 | | YWMH | | | | WDYLID | | AKSYG | | S | | QPYT |
| | | | | | | Y | | NTYLS | | | | |
| Antibody | 676 | GYTFTS | 782 | DPSESN | 888 | GFYDG | 994 | RSSQSL | 1100 | GISNRF | 1206 | LQGTH |
| 40 | | YWMH | | | | WDYAI | | AKSYG | | S | | QPYT |
| | | | | | | YY | | NTYLS | | | | |
| Antibody | 677 | GYTFTS | 783 | DPSESN | 889 | GMYDG | 995 | RSSQSL | 1101 | GISNRF | 1207 | LQGTH |
| 41 | | YWMH | | | | WDYAI | | AKSYG | | S | | QPYT |
| | | | | | | DY | | NTYLS | | | | |
| Antibody | 678 | GYTFTS | 784 | DPSESN | 890 | GRYDG | 996 | RSSQSL | 1102 | GISNRF | 1208 | LQGTH |
| 42 | | YWMH | | | | WDYAI | | AKSYG | | S | | QPYT |
| | | | | | | DY | | NTYLS | | | | |
| Antibody | 679 | GYTFTS | 785 | DPSESN | 891 | GVYDG | 997 | RSSQSL | 1103 | GISNRF | 1209 | LQGTH |
| 43 | | YWMH | | | | WDYAI | | AKSYG | | S | | QPYT |
| | | | | | | DY | | NTYLS | | | | |
| Antibody | 680 | GYTFTS | 786 | DPSESN | 892 | GGYDI | 998 | RSSQSL | 1104 | GISNRF | 1210 | LQGTH |
| 44 | | YWMH | | | | WDYAI | | AKSYG | | S | | QPYT |
| | | | | | | DY | | NTYLS | | | | |
| Antibody | 681 | GYTFTS | 787 | DPSESN | 893 | GGYDL | 999 | RSSQSL | 1105 | GISNRF | 1211 | LQGTH |
| 45 | | YWMH | | | | WDYAI | | AKSYG | | S | | QPYT |
| | | | | | | DY | | NTYLS | | | | |
| Antibody | 682 | GYTFTS | 788 | DPSESN | 894 | GGYDV | 1000 | RSSQSL | 1106 | GISNRF | 1212 | LQGTH |
| 46 | | YWMH | | | | WDYAI | | AKSYG | | S | | QPYT |
| | | | | | | DY | | NTYLS | | | | |
| Antibody | 683 | GYTFTS | 789 | DPSESN | 895 | FGYDG | 1001 | RSSQSL | 1107 | GISNRF | 1213 | LQGTH |
| 47 | | YWMH | | | | WDYAI | | AKSYG | | S | | QPYT |
| | | | | | | DY | | NTYLS | | | | |
| Antibody | 684 | GYTFTS | 790 | DPSESN | 896 | YGYDG | 1002 | RSSQSL | 1108 | GISNRF | 1214 | LQGTH |
| 48 | | YWMH | | | | WDYAI | | AKSYG | | S | | QPYT |
| | | | | | | DY | | NTYLS | | | | |
| Antibody | 685 | GYTFTS | 791 | DPSESN | 897 | GGYDG | 1003 | RSSQSL | 1109 | GISNRF | 1215 | LQGTH |
| 49 | | YWMH | | | | WDYAI | | AASYG | | S | | QPYT |
| | | | | | | DY | | NTYLS | | | | |
| Antibody | 686 | GYTFTS | 792 | DPSESN | 898 | GGYDG | 1004 | RSSQSL | 1110 | GISNRF | 1216 | LQGTH |
| 50 | | YWMH | | | | WDYAI | | ADSYG | | S | | QPYT |
| | | | | | | DY | | NTYLS | | | | |
| Antibody | 687 | GYTFTS | 793 | DPSESN | 899 | GGYDG | 1005 | RSSQSL | 1111 | GISNRF | 1217 | LQGTH |
| 51 | | YWMH | | | | WDYAI | | AISYGN | | S | | QPYT |
| | | | | | | DY | | TYLS | | | | |
| Antibody | 688 | GYTFTS | 794 | DPSESN | 900 | GGYDG | 1006 | RSSQSL | 1112 | GISNRF | 1218 | LQGTH |
| 52 | | YWMH | | | | WDYAI | | AIFYGN | | S | | QPYT |
| | | | | | | DY | | TYLS | | | | |
| Antibody | 689 | GYTFTS | 795 | DPSESN | 901 | GGYDG | 1007 | RSSQSL | 1113 | GISNRF | 1219 | LQGTH |
| 53 | | YWMH | | | | WDYAI | | AIFYGIT | | S | | QPYT |
| | | | | | | DY | | YLS | | | | |
| Antibody | 690 | GYTFTS | 796 | DPSESN | 902 | GGYDG | 1008 | RSSQSL | 1114 | GISNRF | 1220 | LQGTH |
| 54 | | YWMH | | | | WDYAI | | AIFYGL | | S | | QPYT |
| | | | | | | DY | | TYLS | | | | |
| Antibody | 691 | GYTFTS | 797 | DPSESN | 903 | GGYDG | 1009 | RSSQSL | 1115 | GISNRF | 1221 | LQGTH |
| 55 | | YWMH | | | | WDYAI | | AILYGIT | | S | | QPYT |
| | | | | | | DY | | YLS | | | | |
| Antibody | 692 | GYTFTS | 798 | DPSESN | 904 | GGYDG | 1010 | RSSQSL | 1116 | GISNRF | 1222 | LQGTH |
| 56 | | YWMH | | | | WDYAI | | AILYGL | | S | | QPYT |
| | | | | | | DY | | TYLS | | | | |
| Antibody | 693 | GYTFTS | 799 | DPSESN | 905 | GGYDG | 1011 | RSSQSL | 1117 | GISNRF | 1223 | LOGTH |
| 57 | | YWMH | | | | WDYAI | | ALSYGN | | S | | QPYT |
| | | | | | | DY | | TYLS | | | | |
| Antibody | 694 | GYTFTS | 800 | DPSESN | 906 | GGYDG | 1012 | RSSQSL | 1118 | GISNRF | 1224 | LQGTH |
| 58 | | YWMH | | | | WDYAI | | AMSYG | | S | | QPYT |
| | | | | | | DY | | NTYLS | | | | |
| Antibody | 695 | GYTFTS | 801 | DPSESN | 907 | GGYDG | 1013 | RSSQSL | 1119 | GISNRF | 1225 | LQGTH |
| 59 | | YWMH | | | | WDYAI | | ANSYG | | S | | QPYT |
| | | | | | | DY | | NTYLS | | | | |
| Antibody | 696 | GYTFTS | 802 | DPSESN | 908 | GGYDG | 1014 | RSSQSL | 1120 | GISNRF | 1226 | LQGTH |
| 60 | | YWMH | | | | WDYAI | | APSYGN | | S | | QPYT |
| | | | | | | DY | | TYLS | | | | |
| Antibody | 697 | GYTFTS | 803 | DPSESN | 909 | GGYDG | 1015 | RSSQSL | 1121 | GISNRF | 1227 | LQGTH |
| 61 | | YWMH | | | | WDYAI | | AQSYG | | S | | QPYT |
| | | | | | | DY | | NTYLS | | | | |
| Antibody | 698 | GYTFTS | 804 | DPSESN | 910 | GGYDG | 1016 | RSSQSL | 1122 | GISNRF | 1228 | LQGTH |
| 62 | | YWMH | | | | WDYAI | | ARSYG | | S | | QPYT |
| | | | | | | DY | | NTYLS | | | | |
| Antibody | 699 | GYTFTS | 805 | DPSESN | 911 | GGYDG | 1017 | RSSQSL | 1123 | GISNRF | 1229 | LQGTH |
| 63 | | YWMH | | | | WDYAI | | ARFYGL | | S | | QPYT |
| | | | | | | DY | | TYLS | | | | |
| Antibody | 700 | GYTFTS | 806 | DPSESN | 912 | GGYDG | 1018 | RSSQSL | 1124 | GISNRF | 1230 | LQGTH |
| 64 | | YWMH | | | | WDYAI | | ASSYGN | | S | | QPYT |
| | | | | | | DY | | TYLS | | | | |
| Antibody | 701 | GYTFTS | 807 | DPSESN | 913 | GGYDG | 1019 | RSSQSL | 1125 | GISNRF | 1231 | LQGTH |
| 65 | | YWMH | | | | WDYAI | | ATSYGN | | S | | QPYT |
| | | | | | | DY | | TYLS | | | | |
| Antibody | 702 | GYTFTS | 808 | DPSESN | 914 | GGYDG | 1020 | RSSQSL | 1126 | GISNRF | 1232 | LQGTH |
| 66 | | YWMH | | | | WDYAI | | ATYYGI | | S | | QPYT |
| | | | | | | DY | | TYLS | | | | |
| Antibody | 703 | GYTFTS | 809 | DPSESN | 915 | GGYDG | 1021 | RSSQSL | 1127 | GISNRF | 1233 | LQGTH |
| 67 | | YWMH | | | | WDYAI | | ATYYG | | S | | QPYT |
| | | | | | | DY | | LTYLS | | | | |
| Antibody | 704 | GYTFTS | 810 | DPSESN | 916 | GGYDG | 1022 | RSSQSL | 1128 | GISNRF | 1234 | LQGTH |
| 68 | | YWMH | | | | WDYAI | | AVSYG | | S | | QPYT |
| | | | | | | DY | | NTYLS | | | | |
| Antibody | 705 | GYTFTS | 811 | DPSESN | 917 | GGYDG | 1023 | RSSQSL | 1129 | GISNRF | 1235 | LOGTH |
| 69 | | YWMH | | | | WDYAI | | AYSYG | | S | | QPYT |
| | | | | | | DY | | NTYLS | | | | |
| Antibody | 706 | GYTFTS | 812 | DPSESN | 918 | GGYDG | 1024 | RSSQSL | 1130 | GISNRF | 1236 | LQGTH |
| 70 | | YWMH | | | | WDYAI | | AYFYGI | | S | | QPYT |
| | | | | | | DY | | TYLS | | | | |
| Antibody | 707 | GYTFTS | 813 | DPSESN | 919 | GGYDG | 1025 | RSSQSL | 1131 | GISNRF | 1237 | LQGTH |
| 71 | | YWMH | | | | WDYAI | | AYLYGI | | S | | QPYT |
| | | | | | | DY | | TYLS | | | | |
| Antibody | 708 | GYTFTS | 814 | DPSESN | 920 | GGYDG | 1026 | RSSQSL | 1132 | GISNRF | 1238 | LQGTH |
| 72 | | YWMH | | | | WDYAI | | AKSYGI | | S | | QPYT |
| | | | | | | DY | | TYLS | | | | |
| Antibody | 709 | GYTFTS | 815 | DPSESN | 921 | GGYDG | 1027 | RSSQSL | 1133 | GISNRF | 1239 | LOGTH |
| 73 | | YWMH | | | | WDYAI | | AKSYGL | | S | | QPYT |
| | | | | | | DY | | TYLS | | | | |
| Antibody | 710 | GYTFTS | 816 | DPSESN | 922 | GGYDG | 1028 | RSSQSL | 1134 | GISNRF | 1240 | LQGTH |
| 74 | | YWMH | | | | WDYAI | | AKFYG | | S | | QPYT |
| | | | | | | DY | | NTYLS | | | | |
| Antibody | 711 | GYTFTS | 817 | DPSESN | 923 | GGYDG | 1029 | RSSQSL | 1135 | GISNRF | 1241 | LQGTH |
| 75 | | YWMH | | | | WDYAI | | AKIYGN | | S | | QPYT |
| | | | | | | DY | | TYLS | | | | |
| Antibody | 712 | GYTFTS | 818 | DPSESN | 924 | GGYDG | 1030 | RSSQSL | 1136 | GISNRF | 1242 | LQGTH |
| 76 | | YWMH | | | | WDYAI | | AKLYG | | S | | QPYT |
| | | | | | | DY | | NTYLS | | | | |
| Antibody | 713 | GYTFTS | 819 | DPSESN | 925 | GGYDG | 1031 | RSSQSL | 1137 | GISNRF | 1243 | LQGTH |
| 77 | | YWMH | | | | WDYAI | | AKMYG | | S | | QPYT |
| | | | | | | DY | | NTYLS | | | | |
| Antibody | 714 | GYTFTS | 820 | DPSESN | 926 | GGYDG | 1032 | RSSQSL | 1138 | GISNRF | 1244 | LQGTH |
| 78 | | YWMH | | | | WDYAI | | AKQYG | | S | | QPYT |
| | | | | | | DY | | NTYLS | | | | |
| Antibody | 715 | GYTFTS | 821 | DPSESN | 927 | GGYDG | 1033 | RSSQSL | 1139 | GISNRF | 1245 | LQGTH |
| 79 | | YWMH | | | | WDYAI | | AKYYG | | S | | QPYT |
| | | | | | | DY | | NTYLS | | | | |
| Antibody | 716 | GYTFTS | 822 | DPSESN | 928 | GGYDG | 1034 | RSSQSL | 1140 | GISNRF | 1246 | LQGTH |
| 80 | | YWMH | | | | WDYAI | | AKSHG | | S | | QPYT |
| | | | | | | DY | | NTYLS | | | | |
| Antibody | 717 | GYTFTS | 823 | DPSESN | 929 | GGYDG | 1035 | RSSQSL | 1141 | FISNRFS | 1247 | LQGTH |
| 81 | | YWMH | | | | WDYAI | | AKSYG | | | | QPYT |
| | | | | | | DY | | NTYLS | | | | |
| Antibody | 718 | GYTFTS | 824 | DPSESN | 930 | GGYDG | 1036 | RSSQSL | 1142 | PISNRFS | 1248 | LOGTH |
| 82 | | YWMH | | | | WDYAI | | AKSYG | | | | QPYT |
| | | | | | | DY | | NTYLS | | | | |
| Antibody | 719 | GYTFTS | 825 | DPSESN | 931 | GGYDG | 1037 | RSSQSL | 1143 | YISNRF | 1249 | LQGTH |
| 83 | | YWMH | | | | WDYAI | | AKSYG | | S | | QPYT |
| | | | | | | DY | | NTYLS | | | | |
| Antibody | 720 | GYTFTS | 826 | DPSESN | 932 | GGYDG | 1038 | RSSQSL | 1144 | GISNRF | 1250 | LQFTHQ |
| 84 | | YWMH | | | | WDYAI | | AKSYG | | S | | PYT |
| | | | | | | DY | | NTYLS | | | | |
| Antibody | 721 | GYTFTS | 827 | DPSESN | 933 | GGYDG | 1039 | RSSQSL | 1145 | GISNRF | 1251 | LQFTIQ |
| 85 | | YWMH | | | | WDYAI | | AKSYG | | S | | FYT |
| | | | | | | DY | | NTYLS | | | | |
| Antibody | 722 | GYTFTS | 828 | DPSESN | 934 | GGYDG | 1040 | RSSQSL | 1146 | GISNRF | 1252 | LQFTIQ |
| 86 | | YWMH | | | | WDYAI | | AKSYG | | S | | VYT |
| | | | | | | DY | | NTYLS | | | | |
| Antibody | 723 | GYTFTS | 829 | DPSESN | 935 | GGYDG | 1041 | RSSQSL | 1147 | GISNRF | 1253 | LQFTIQ |
| 87 | | YWMH | | | | WDYAI | | AKSYG | | S | | PYI |
| | | | | | | DY | | NTYLS | | | | |
| Antibody | 724 | GYTFTS | 830 | DPSESN | 936 | GGYDG | 1042 | RSSQSL | 1148 | GISNRF | 1254 | LQFTHQ |
| 88 | | YWMH | | | | WDYAI | | AKSYG | | S | | FYT |
| | | | | | | DY | | NTYLS | | | | |
| Antibody | 725 | GYTFTS | 831 | DPSESN | 937 | GGYDG | 1043 | RSSQSL | 1149 | GISNRF | 1255 | LQFTHQ |
| 89 | | YWMH | | | | WDYAI | | AKSYG | | S | | IYI |
| | | | | | | DY | | NTYLS | | | | |
| Antibody | 726 | GYTFTS | 832 | DPSESN | 938 | GGYDG | 1044 | RSSQSL | 1150 | GISNRF | 1256 | LQFTHQ |
| 90 | | YWMH | | | | WDYAI | | AKSYG | | S | | PYI |
| | | | | | | DY | | NTYLS | | | | |
| Antibody | 727 | GYTFTS | 833 | DPSESN | 939 | GGYDG | 1045 | RSSQSL | 1151 | GISNRF | 1257 | LQRTHQ |
| 91 | | YWMH | | | | WDYAI | | AKSYG | | S | | PYT |
| | | | | | | DY | | NTYLS | | | | |
| Antibody | 728 | GYTFTS | 834 | DPSESN | 940 | GGYDG | 1046 | RSSQSL | 1152 | GISNRF | 1258 | LQRTIQ |
| 92 | | YWMH | | | | WDYAI | | AKSYG | | S | | VYI |
| | | | | | | DY | | NTYLS | | | | |
| Antibody | 729 | GYTFTS | 835 | DPSESN | 941 | GGYDG | 1047 | RSSQSL | 1153 | GISNRF | 1259 | LQRTIQ |
| 93 | | YWMH | | | | WDYAI | | AKSYG | | S | | YYT |
| | | | | | | DY | | NTYLS | | | | |
| Antibody | 730 | GYTFTS | 836 | DPSESN | 942 | GGYDG | 1048 | RSSQSL | 1154 | GISNRF | 1260 | LQVTH |
| 94 | | YWMH | | | | WDYAI | | AKSYG | | S | | QPYT |
| | | | | | | DY | | NTYLS | | | | |
| Antibody | 731 | GYTFTS | 837 | DPSESN | 943 | GGYDG | 1049 | RSSQSL | 1155 | GISNRF | 1261 | LQWTH |
| 95 | | YWMH | | | | WDYAI | | AKSYG | | S | | QPYT |
| | | | | | | DY | | NTYLS | | | | |
| Antibody | 732 | GYTFTS | 838 | DPSESN | 944 | GGYDG | 1050 | RSSQSL | 1156 | GISNRF | 1262 | LQYTH |
| 96 | | YWMH | | | | WDYAI | | AKSYG | | S | | QPYT |
| | | | | | | DY | | NTYLS | | | | |
| Antibody | 733 | GYTFTS | 839 | DPSESN | 945 | GGYDG | 1051 | RSSQSL | 1157 | GISNRF | 1263 | LQYTIQ |
| 97 | | YWMH | | | | WDYAI | | AKSYG | | S | | FYI |
| | | | | | | DY | | NTYLS | | | | |
| Antibody | 734 | GYTFTS | 840 | DPSESN | 946 | GGYDG | 1052 | RSSQSL | 1158 | GISNRF | 1264 | LQYTH |
| 98 | | YWMH | | | | WDYAI | | AKSYG | | S | | QFYI |
| | | | | | | DY | | NTYLS | | | | |
| Antibody | 735 | GYTFTS | 841 | DPSESN | 947 | GGYDG | 1053 | RSSQSL | 1159 | GISNRF | 1265 | LQGTIQ |
| 99 | | YWMH | | | | WDYAI | | AKSYG | | S | | PYT |
| | | | | | | DY | | NTYLS | | | | |
| Antibody | 736 | GYTFTS | 842 | DPSESN | 948 | GGYDG | 1054 | RSSQSL | 1160 | GISNRF | 1266 | LQGTW |
| 100 | | YWMH | | | | WDYAI | | AKSYG | | S | | QPYT |
| | | | | | | DY | | NTYLS | | | | |
| Antibody | 737 | GYTFTS | 843 | DPSESN | 949 | GGYDG | 1055 | RSSQSL | 1161 | GISNRF | 1267 | LQGTH |
| 101 | | YWMH | | | | WDYAI | | AKSYG | | S | | QFYT |
| | | | | | | DY | | NTYLS | | | | |
| Antibody | 738 | GYTFTS | 844 | DPSESN | 950 | GGYDG | 1056 | RSSQSL | 1162 | GISNRF | 1268 | LQGTH |
| 102 | | YWMH | | | | WDYAI | | AKSYG | | S | | QIYT |
| | | | | | | DY | | NTYLS | | | | |
| Antibody | 739 | GYTFTS | 845 | DPSESN | 951 | GGYDG | 1057 | RSSQSL | 1163 | GISNRF | 1269 | LQGTH |
| 103 | | YWMH | | | | WDYAI | | AKSYG | | S | | QVYT |
| | | | | | | DY | | NTYLS | | | | |
| Antibody | 740 | GYTFTS | 846 | DPSESN | 952 | GGYDG | 1058 | RSSQSL | 1164 | GISNRF | 1270 | LQGTH |
| 104 | | YWMH | | | | WDYAI | | AKSYG | | S | | QYYT |
| | | | | | | DY | | NTYLS | | | | |
| Antibody | 741 | GYTFTS | 847 | DPSESN | 953 | GGYDG | 1059 | RSSQSL | 1165 | GISNRF | 1271 | LQGTH |
| 105 | | YWMH | | | | WDYAI | | AKSYG | | S | | QPYI |
| | | | | | | DY | | NTYLS | | | | |
| Antibody | 742 | GYTFTS | 848 | DPSESN | 954 | GGYDG | 1060 | RSSQSL | 1166 | GISNRF | 1272 | LQGTH |
| 106 | | YWMH | | | | WDYAI | | AKSYG | | S | | QPYR |
| | | | | | | DY | | NTYLS | | | | |

IMGT Numbering

| | | | | | | | | | | | |
|---|---|---|---|---|---|---|---|---|---|---|---|
| Antibody 1 | 1273 | GGTFTS | 1379 | IDPSESN | 1485 | ARGGY | 1591 | QSLAKS | GIS | 1803 | LQGTH |
| | | YW | | T | | DGWDY | | YGNTY | | | QPYT |
| | | | | | | AIDY | | | | | |
| Antibody 2 | 1274 | GYTFTS | 1380 | IDPSESN | 1486 | ARGGY | 1592 | QSLAKS | GIS | 1804 | LQGTH |
| | | | | | | DGWDY | | | | | |
| | | YW | | T | | AIDY | | YGNTY | | | QPYT |
| Antibody 3 | 1275 | GYTFTI | 1381 | IDPSESN | 1487 | ARGGY | 1593 | QSLAKS | GIS | 1805 | LQGTH |
| | | YW | | T | | DGWDY | | YGNTY | | | QPYT |
| | | | | | | AIDY | | | | | |
| Antibody 4 | 1276 | GYTFTV | 1382 | IDPSESN | 1488 | ARGGY | 1594 | QSLAKS | GIS | 1806 | LQGTH |
| | | YW | | T | | DGWDY | | YGNTY | | | QPYT |
| | | | | | | AIDY | | | | | |
| Antibody 5 | 1277 | GYTFTS | 1383 | IDPSESN | 1489 | ARGGY | 1595 | QSLAKS | GIS | 1807 | LQGTH |
| | | MW | | T | | DGWDY | | YGNTY | | | QPYT |
| | | | | | | AIDY | | | | | |
| Antibody 6 | 1278 | GYTFTS | 1384 | IDPSESN | 1490 | ARGGY | 1596 | QSLAKS | GIS | 1808 | LQGTH |
| | | QW | | T | | DGWDY | | YGNTY | | | QPYT |
| | | | | | | AIDY | | | | | |
| Antibody 7 | 1279 | GYTFTS | 1385 | IIPSESN | 1491 | ARGGY | 1597 | QSLAKS | GIS | 1809 | LQGTH |
| | | YW | | T | | DGWDY | | YGNTY | | | QPYT |
| | | | | | | AIDY | | | | | |
| Antibody 8 | 1280 | GYTFTS | 1386 | IIPLESN | 1492 | ARGGY | 1598 | QSLAKS | GIS | 1810 | LQGTH |
| | | YW | | T | | DGWDY | | YGNTY | | | QPYT |
| | | | | | | AIDY | | | | | |
| Antibody 9 | 1281 | GYTFTS | 1387 | IIPRESN | 1493 | ARGGY | 1599 | QSLAKS | GIS | 1811 | LQGTH |
| | | YW | | T | | DGWDY | | YGNTY | | | QPYT |
| | | | | | | AIDY | | | | | |
| Antibody | 1282 | GYTFTS | 1388 | IIPVESN | 1494 | ARGGY | 1600 | QSLAKS | GIS | 1812 | LQGTH |
| 10 | | YW | | T | | DGWDY | | YGNTY | | | QPYT |
| | | | | | | AIDY | | | | | |
| Antibody | 1283 | GYTFTS | 1389 | IRPSESN | 1495 | ARGGY | 1601 | QSLAKS | GIS | 1813 | LQGTH |
| 11 | | YW | | T | | DGWDY | | YGNTY | | | QPYT |
| | | | | | | AIDY | | | | | |
| Antibody | 1284 | GYTFTS | 1390 | IRPLESN | 1496 | ARGGY | 1602 | QSLAKS | GIS | 1814 | LQGTH |
| 12 | | YW | | T | | DGWDY | | YGNTY | | | QPYT |
| | | | | | | AIDY | | | | | |
| Antibody | 1285 | GYTFTS | 1391 | IRPRES | 1497 | ARGGY | 1603 | QSLAKS | GIS | 1815 | LQGTH |
| 13 | | YW | | NT | | DGWDY | | YGNTY | | | QPYT |
| | | | | | | AIDY | | | | | |
| Antibody | 1286 | GYTFTS | 1392 | IRPVES | 1498 | ARGGY | 1604 | QSLAKS | GIS | 1816 | LQGTH |
| 14 | | YW | | NT | | DGWDY | | YGNTY | | | QPYT |
| | | | | | | AIDY | | | | | |
| Antibody | 1287 | GYTFTS | 1393 | IDPLES | 1499 | ARGGY | 1605 | QSLAKS | GIS | 1817 | LQGTH |
| 15 | | YW | | NT | | DGWDY | | YGNTY | | | QPYT |
| | | | | | | AIDY | | | | | |
| Antibody | 1288 | GYTFTS | 1394 | IDPMES | 1500 | ARGGY | 1606 | QSLAKS | GIS | 1818 | LQGTH |
| 16 | | YW | | NT | | DGWDY | | YGNTY | | | QPYT |
| | | | | | | AIDY | | | | | |
| Antibody | 1289 | GYTFTS | 1395 | IDPRES | 1501 | ARGGY | 1607 | QSLAKS | GIS | 1819 | LQGTH |
| 17 | | YW | | NT | | DGWDY | | YGNTY | | | QPYT |
| | | | | | | AIDY | | | | | |
| Antibody | 1290 | GYTFTS | 1396 | IDPVES | 1502 | ARGGY | 1608 | QSLAKS | GIS | 1820 | LQGTH |
| 18 | | YW | | NT | | DGWDY | | YGNTY | | | QPYT |
| | | | | | | AIDY | | | | | |
| Antibody | 1291 | GYTFTS | 1397 | IDPSESN | 1503 | ARGGY | 1609 | QSLAKS | GIS | 1821 | LQGTH |
| 19 | | YW | | T | | DGWDY | | YGNTY | | | QPYT |
| | | | | | | FIDY | | | | | |
| Antibody | 1292 | GYTFTS | 1398 | IDPSESN | 1504 | ARGGY | 1610 | QSLAKS | GIS | 1822 | LQGTH |
| 20 | | YW | | T | | DGWDY | | YGNTY | | | QPYT |
| | | | | | | FIYY | | | | | |
| Antibody | 1293 | GYTFTS | 1399 | IDPSESN | 1505 | ARGGY | 1611 | QSLAKS | GIS | 1823 | LQGTH |
| 21 | | YW | | T | | DGWDY | | YGNTY | | | QPYT |
| | | | | | | LIDY | | | | | |
| Antibody | 1294 | GYTFTS | 1400 | IDPSESN | 1506 | ARGGY | 1612 | QSLAKS | GIS | 1824 | LQGTH |
| 22 | | YW | | T | | DGWDY | | YGNTY | | | QPYT |
| | | | | | | VIDY | | | | | |
| Antibody | 1295 | GYTFTS | 1401 | IDPSESN | 1507 | ARGGY | 1613 | QSLAKS | GIS | 1825 | LOGTH |
| 23 | | YW | | T | | DGWDY | | YGNTY | | | QPYT |
| | | | | | | VIYY | | | | | |
| Antibody | 1296 | GYTFTS | 1402 | IDPSESN | 1508 | ARGGY | 1614 | QSLAKS | GIS | 1826 | LQGTH |
| 24 | | YW | | T | | DGWDY | | YGNTY | | | QPYT |
| | | | | | | WIDY | | | | | |
| Antibody | 1297 | GYTFTS | 1403 | IDPSESN | 1509 | ARGGY | 1615 | QSLAKS | GIS | 1827 | LQGTH |
| 25 | | YW | | T | | DGWDY | | YGNTY | | | QPYT |
| | | | | | | YIDY | | | | | |
| Antibody | 1298 | GYTFTS | 1404 | IDPSESN | 1510 | ARGGY | 1616 | QSLAKS | GIS | 1828 | LQGTH |
| 26 | | YW | | T | | DGWDY | | YGNTY | | | QPYT |
| | | | | | | YIYY | | | | | |
| Antibody | 1299 | GYTFTS | 1405 | IDPSESN | 1511 | IRGGYD | 1617 | QSLAKS | GIS | 1829 | LQGTH |
| 27 | | YW | | T | | GWDYA | | YGNTY | | | QPYT |
| | | | | | | IDY | | | | | |
| Antibody | 1300 | GYTFTS | 1406 | IDPSESN | 1512 | LRGGY | 1618 | QSLAKS | GIS | 1830 | LQGTH |
| 28 | | YW | | T | | DGWDY | | YGNTY | | | QPYT |
| | | | | | | AIDY | | | | | |
| Antibody | 1301 | GYTFTS | 1407 | IDPSESN | 1513 | MRGGY | 1619 | QSLAKS | GIS | 1831 | LOGTH |
| 29 | | YW | | T | | DGWDY | | YGNTY | | | QPYT |
| | | | | | | AIDY | | | | | |
| Antibody | 1302 | GYTFTS | 1408 | IDPSESN | 1514 | RRGGY | 1620 | QSLAKS | GIS | 1832 | LQGTH |
| 30 | | YW | | T | | DGWDY | | YGNTY | | | QPYT |
| | | | | | | AIDY | | | | | |
| Antibody | 1303 | GYTFTS | 1409 | IDPSESN | 1515 | VRGGY | 1621 | QSLAKS | GIS | 1833 | LQGTH |
| 31 | | YW | | T | | DGWDY | | YGNTY | | | QPYT |
| | | | | | | AIDY | | | | | |
| Antibody | 1304 | GYTFTS | 1410 | IDPSESN | 1516 | ARGGY | 1622 | QSLAKS | GIS | 1834 | LQGTH |
| 32 | | YW | | T | | WGWDY | | YGNTY | | | QPYT |
| | | | | | | AIDY | | | | | |
| Antibody | 1305 | GYTFTS | 1411 | IDPSESN | 1517 | ARGGY | 1623 | QSLAKS | GIS | 1835 | LQGTH |
| 33 | | YW | | T | | YGWDY | | YGNTY | | | QPYT |
| | | | | | | AIDY | | | | | |
| Antibody | 1306 | GYTFTS | 1412 | IDPSESN | 1518 | ARGGY | 1624 | QSLAKS | GIS | 1836 | LQGTH |
| 34 | | YW | | T | | DGWDY | | YGNTY | | | QPYT |
| | | | | | | AIFY | | | | | |
| Antibody | 1307 | GYTFTS | 1413 | IDPSESN | 1519 | ARGGY | 1625 | QSLAKS | GIS | 1837 | LQGTH |
| 35 | | YW | | T | | DGWDY | | YGNTY | | | QPYT |
| | | | | | | AIMY | | | | | |
| Antibody | 1308 | GYTFTS | 1414 | IDPSESN | 1520 | ARGGY | 1626 | QSLAKS | GIS | 1838 | LOGTH |
| 36 | | YW | | T | | DGWDY | | YGNTY | | | QPYT |
| | | | | | | AIWY | | | | | |
| Antibody | 1309 | GYTFTS | 1415 | IDPSESN | 1521 | ARGGY | 1627 | QSLAKS | GIS | 1839 | LQGTH |
| 37 | | YW | | T | | DGWDY | | YGNTY | | | QPYT |
| | | | | | | AIYY | | | | | |
| Antibody | 1310 | GYTFTS | 1416 | IDPSESN | 1522 | ARGFY | 1628 | QSLAKS | GIS | 1840 | LQGTH |
| 38 | | YW | | T | | DGWDY | | YGNTY | | | QPYT |
| | | | | | | AIDY | | | | | |
| Antibody | 1311 | GYTFTS | 1417 | IDPSESN | 1523 | ARGFY | 1629 | QSLAKS | GIS | 1841 | LQGTH |
| 39 | | YW | | T | | DGWDY | | YGNTY | | | QPYT |
| | | | | | | LIDY | | | | | |
| Antibody | 1312 | GYTFTS | 1418 | IDPSESN | 1524 | ARGFY | 1630 | QSLAKS | GIS | 1842 | LQGTH |
| 40 | | YW | | T | | DGWDY | | YGNTY | | | QPYT |
| | | | | | | AIYY | | | | | |
| Antibody | 1313 | GYTFTS | 1419 | IDPSESN | 1525 | ARGMY | 1631 | QSLAKS | GIS | 1843 | LQGTH |
| 41 | | YW | | T | | DGWDY | | YGNTY | | | QPYT |
| | | | | | | AIDY | | | | | |
| Antibody | 1314 | GYTFTS | 1420 | IDPSESN | 1526 | ARGRY | 1632 | QSLAKS | GIS | 1844 | LQGTH |
| 42 | | YW | | T | | DGWDY | | YGNTY | | | QPYT |
| | | | | | | AIDY | | | | | |
| Antibody | 1315 | GYTFTS | 1421 | IDPSESN | 1527 | ARGVY | 1633 | QSLAKS | GIS | 1845 | LQGTH |
| 43 | | YW | | T | | DGWDY | | YGNTY | | | QPYT |
| | | | | | | AIDY | | | | | |
| Antibody | 1316 | GYTFTS | 1422 | IDPSESN | 1528 | ARGGY | 1634 | QSLAKS | GIS | 1846 | LQGTH |
| 44 | | YW | | T | | DIWDY | | YGNTY | | | QPYT |
| | | | | | | AIDY | | | | | |
| Antibody | 1317 | GYTFTS | 1423 | IDPSESN | 1529 | ARGGY | 1635 | QSLAKS | GIS | 1847 | LQGTH |
| 45 | | YW | | T | | DLWDY | | YGNTY | | | QPYT |
| | | | | | | AIDY | | | | | |
| Antibody | 1318 | GYTFTS | 1424 | IDPSESN | 1530 | ARGGY | 1636 | QSLAKS | GIS | 1848 | LQGTH |
| 46 | | YW | | T | | DVWDY | | YGNTY | | | QPYT |
| | | | | | | AIDY | | | | | |
| Antibody | 1319 | GYTFTS | 1425 | IDPSESN | 1531 | ARFGY | 1637 | QSLAKS | GIS | 1849 | LQGTH |
| 47 | | YW | | T | | DGWDY | | YGNTY | | | QPYT |
| | | | | | | AIDY | | | | | |
| Antibody | 1320 | GYTFTS | 1426 | IDPSESN | 1532 | ARYGY | 1638 | QSLAKS | GIS | 1850 | LQGTH |
| 48 | | YW | | T | | DGWDY | | YGNTY | | | QPYT |
| | | | | | | AIDY | | | | | |
| Antibody | 1321 | GYTFTS | 1427 | IDPSESN | 1533 | ARGGY | 1639 | QSLAAS | GIS | 1851 | LQGTH |
| 49 | | YW | | T | | DGWDY | | YGNTY | | | QPYT |
| | | | | | | AIDY | | | | | |
| Antibody | 1322 | GYTFTS | 1428 | IDPSESN | 1534 | ARGGY | 1640 | QSLADS | GIS | 1852 | LQGTH |
| 50 | | YW | | T | | DGWDY | | YGNTY | | | QPYT |
| | | | | | | AIDY | | | | | |
| Antibody | 1323 | GYTFTS | 1429 | IDPSESN | 1535 | ARGGY | 1641 | QSLAIS | GIS | 1853 | LQGTH |
| 51 | | YW | | T | | DGWDY | | YGNTY | | | QPYT |
| | | | | | | AIDY | | | | | |
| Antibody | 1324 | GYTFTS | 1430 | IDPSESN | 1536 | ARGGY | 1642 | QSLAIF | GIS | 1854 | LQGTH |
| 52 | | YW | | T | | DGWDY | | YGNTY | | | QPYT |
| | | | | | | AIDY | | | | | |
| Antibody | 1325 | GYTFTS | 1431 | IDPSESN | 1537 | ARGGY | 1643 | QSLAIF | GIS | 1855 | LQGTH |
| 53 | | YW | | T | | DGWDY | | YGITY | | | QPYT |
| | | | | | | AIDY | | | | | |
| Antibody | 1326 | GYTFTS | 1432 | IDPSESN | 1538 | ARGGY | 1644 | QSLAIF | GIS | 1856 | LQGTH |
| 54 | | YW | | T | | DGWDY | | YGLTY | | | QPYT |
| | | | | | | AIDY | | | | | |
| Antibody | 1327 | GYTFTS | 1433 | IDPSESN | 1539 | ARGGY | 1645 | QSLAIL | GIS | 1857 | LQGTH |
| 55 | | YW | | T | | DGWDY | | YGITY | | | QPYT |
| | | | | | | AIDY | | | | | |
| Antibody | 1328 | GYTFTS | 1434 | IDPSESN | 1540 | ARGGY | 1646 | QSLAIL | GIS | 1858 | LQGTH |
| 56 | | YW | | T | | DGWDY | | YGLTY | | | QPYT |
| | | | | | | AIDY | | | | | |
| Antibody | 1329 | GYTFTS | 1435 | IDPSESN | 1541 | ARGGY | 1647 | QSLALS | GIS | 1859 | LQGTH |
| 57 | | YW | | T | | DGWDY | | YGNTY | | | QPYT |
| | | | | | | AIDY | | | | | |
| Antibody | 1330 | GYTFTS | 1436 | IDPSESN | 1542 | ARGGY | 1648 | QSLAMS | GIS | 1860 | LQGTH |
| 58 | | YW | | T | | DGWDY | | YGNTY | | | QPYT |
| | | | | | | AIDY | | | | | |
| Antibody | 1331 | GYTFTS | 1437 | IDPSESN | 1543 | ARGGY | 1649 | QSLANS | GIS | 1861 | LQGTH |
| 59 | | YW | | T | | DGWDY | | YGNTY | | | QPYT |
| | | | | | | AIDY | | | | | |
| Antibody | 1332 | GYTFTS | 1438 | IDPSESN | 1544 | ARGGY | 1650 | QSLAPS | GIS | 1862 | LQGTH |
| 60 | | YW | | T | | DGWDY | | YGNTY | | | QPYT |
| | | | | | | AIDY | | | | | |
| Antibody | 1333 | GYTFTS | 1439 | IDPSESN | 1545 | ARGGY | 1651 | QSLAQS | GIS | 1863 | LQGTH |
| 61 | | YW | | T | | DGWDY | | YGNTY | | | QPYT |
| | | | | | | AIDY | | | | | |
| Antibody | 1334 | GYTFTS | 1440 | IDPSESN | 1546 | ARGGY | 1652 | QSLARS | GIS | 1864 | LQGTH |
| 62 | | YW | | T | | DGWDY | | YGNTY | | | QPYT |
| | | | | | | AIDY | | | | | |
| Antibody | 1335 | GYTFTS | 1441 | IDPSESN | 1547 | ARGGY | 1653 | QSLARF | GIS | 1865 | LQGTH |
| 63 | | YW | | T | | DGWDY | | YGLTY | | | QPYT |
| | | | | | | AIDY | | | | | |
| Antibody | 1336 | GYTFTS | 1442 | IDPSESN | 1548 | ARGGY | 1654 | QSLASS | GIS | 1866 | LQGTH |
| 64 | | YW | | T | | DGWDY | | YGNTY | | | QPYT |
| | | | | | | AIDY | | | | | |
| Antibody | 1337 | GYTFTS | 1443 | IDPSESN | 1549 | ARGGY | 1655 | QSLATS | GIS | 1867 | LQGTH |
| 65 | | YW | | T | | DGWDY | | YGNTY | | | QPYT |
| | | | | | | AIDY | | | | | |
| Antibody | 1338 | GYTFTS | 1444 | IDPSESN | 1550 | ARGGY | 1656 | QSLATY | GIS | 1868 | LQGTH |
| 66 | | YW | | T | | DGWDY | | YGITY | | | QPYT |
| | | | | | | AIDY | | | | | |
| Antibody | 1339 | GYTFTS | 1445 | IDPSESN | 1551 | ARGGY | 1657 | QSLATY | GIS | 1869 | LQGTH |
| 67 | | YW | | T | | DGWDY | | YGLTY | | | QPYT |
| | | | | | | AIDY | | | | | |
| Antibody | 1340 | GYTFTS | 1446 | IDPSESN | 1552 | ARGGY | 1658 | QSLAVS | GIS | 1870 | LQGTH |
| 68 | | YW | | T | | DGWDY | | YGNTY | | | QPYT |
| | | | | | | AIDY | | | | | |
| Antibody | 1341 | GYTFTS | 1447 | IDPSESN | 1553 | ARGGY | 1659 | QSLAYS | GIS | 1871 | LQGTH |
| 69 | | YW | | T | | DGWDY | | YGNTY | | | QPYT |
| | | | | | | AIDY | | | | | |
| Antibody | 1342 | GYTFTS | 1448 | IDPSESN | 1554 | ARGGY | 1660 | QSLAYF | GIS | 1872 | LQGTH |
| 70 | | YW | | T | | DGWDY | | YGITY | | | QPYT |
| | | | | | | AIDY | | | | | |
| Antibody | 1343 | GYTFTS | 1449 | IDPSESN | 1555 | ARGGY | 1661 | QSLAYL | GIS | 1873 | LQGTH |
| 71 | | YW | | T | | DGWDY | | YGITY | | | QPYT |
| | | | | | | AIDY | | | | | |
| Antibody | 1344 | GYTFTS | 1450 | IDPSESN | 1556 | ARGGY | 1662 | QSLAKS | GIS | 1874 | LQGTH |
| 72 | | YW | | T | | DGWDY | | YGITY | | | QPYT |
| | | | | | | AIDY | | | | | |
| Antibody | 1345 | GYTFTS | 1451 | IDPSESN | 1557 | ARGGY | 1663 | QSLAKS | GIS | 1875 | LQGTH |
| 73 | | YW | | T | | DGWDY | | YGLTY | | | QPYT |
| | | | | | | AIDY | | | | | |
| Antibody | 1346 | GYTFTS | 1452 | IDPSESN | 1558 | ARGGY | 1664 | QSLAKF | GIS | 1876 | LQGTH |
| 74 | | YW | | T | | DGWDY | | YGNTY | | | QPYT |
| | | | | | | AIDY | | | | | |
| Antibody | 1347 | GYTFTS | 1453 | IDPSESN | 1559 | ARGGY | 1665 | QSLAKI | GIS | 1877 | LQGTH |
| 75 | | YW | | T | | DGWDY | | YGNTY | | | QPYT |
| | | | | | | AIDY | | | | | |
| Antibody | 1348 | GYTFTS | 1454 | IDPSESN | 1560 | ARGGY | 1666 | QSLAKL | GIS | 1878 | LQGTH |
| 76 | | YW | | T | | DGWDY | | YGNTY | | | QPYT |
| | | | | | | AIDY | | | | | |
| Antibody | 1349 | GYTFTS | 1455 | IDPSESN | 1561 | ARGGY | 1667 | QSLAK | GIS | 1879 | LQGTH |
| 77 | | YW | | T | | DGWDY | | MYGNT | | | QPYT |
| | | | | | | AIDY | | Y | | | |
| Antibody | 1350 | GYTFTS | 1456 | IDPSESN | 1562 | ARGGY | 1668 | QSLAKQ | GIS | 1880 | LQGTH |
| 78 | | YW | | T | | DGWDY | | YGNTY | | | QPYT |
| | | | | | | AIDY | | | | | |
| Antibody | 1351 | GYTFTS | 1457 | IDPSESN | 1563 | ARGGY | 1669 | QSLAKY | GIS | 1881 | LQGTH |
| 79 | | YW | | T | | DGWDY | | YGNTY | | | QPYT |
| | | | | | | AIDY | | | | | |
| Antibody | 1352 | GYTFTS | 1458 | IDPSESN | 1564 | ARGGY | 1670 | QSLAKS | GIS | 1882 | LQGTH |
| 80 | | YW | | T | | DGWDY | | HGNTY | | | QPYT |
| | | | | | | AIDY | | | | | |
| Antibody | 1353 | GYTFTS | 1459 | IDPSESN | 1565 | ARGGY | 1671 | QSLAKS | FIS | 1883 | LQGTH |
| 81 | | YW | | T | | DGWDY | | YGNTY | | | QPYT |
| | | | | | | AIDY | | | | | |
| Antibody | 1354 | GYTFTS | 1460 | IDPSESN | 1566 | ARGGY | 1672 | QSLAKS | PIS | 1884 | LQGTH |
| 82 | | YW | | T | | DGWDY | | YGNTY | | | QPYT |
| | | | | | | AIDY | | | | | |
| Antibody | 1355 | GYTFTS | 1461 | IDPSESN | 1567 | ARGGY | 1673 | QSLAKS | YIS | 1885 | LQGTH |
| 83 | | YW | | T | | DGWDY | | YGNTY | | | QPYT |
| | | | | | | AIDY | | | | | |
| Antibody | 1356 | GYTFTS | 1462 | IDPSESN | 1568 | ARGGY | 1674 | QSLAKS | GIS | 1886 | LQFTHQ |
| 84 | | YW | | T | | DGWDY | | YGNTY | | | PYT |
| | | | | | | AIDY | | | | | |
| Antibody | 1357 | GYTFTS | 1463 | IDPSESN | 1569 | ARGGY | 1675 | QSLAKS | GIS | 1887 | LQFTIQ |
| 85 | | YW | | T | | DGWDY | | YGNTY | | | FYT |
| | | | | | | AIDY | | | | | |
| Antibody | 1358 | GYTFTS | 1464 | IDPSESN | 1570 | ARGGY | 1676 | QSLAKS | GIS | 1888 | LQFTIQ |
| 86 | | YW | | T | | DGWDY | | YGNTY | | | VYT |
| | | | | | | AIDY | | | | | |
| Antibody | 1359 | GYTFTS | 1465 | IDPSESN | 1571 | ARGGY | 1677 | QSLAKS | GIS | 1889 | LQFTIQ |
| 87 | | YW | | T | | DGWDY | | YGNTY | | | PYI |
| | | | | | | AIDY | | | | | |
| Antibody | 1360 | GYTFTS | 1466 | IDPSESN | 1572 | ARGGY | 1678 | QSLAKS | GIS | 1890 | LQFTHQ |
| 88 | | YW | | T | | DGWDY | | YGNTY | | | FYT |
| | | | | | | AIDY | | | | | |
| Antibody | 1361 | GYTFTS | 1467 | IDPSESN | 1573 | ARGGY | 1679 | QSLAKS | GIS | 1891 | LQFTHQ |
| 89 | | YW | | T | | DGWDY | | YGNTY | | | IYI |
| | | | | | | AIDY | | | | | |
| Antibody | 1362 | GYTFTS | 1468 | IDPSESN | 1574 | ARGGY | 1680 | QSLAKS | GIS | 1892 | LQFTHQ |
| 90 | | YW | | T | | DGWDY | | YGNTY | | | PYI |
| | | | | | | AIDY | | | | | |
| Antibody | 1363 | GYTFTS | 1469 | IDPSESN | 1575 | ARGGY | 1681 | QSLAKS | GIS | 1893 | LQRTHQ |
| 91 | | YW | | T | | DGWDY | | YGNTY | | | PYT |
| | | | | | | AIDY | | | | | |
| Antibody | 1364 | GYTFTS | 1470 | IDPSESN | 1576 | ARGGY | 1682 | QSLAKS | GIS | 1894 | LQRTIQ |
| 92 | | YW | | T | | DGWDY | | YGNTY | | | VYI |
| | | | | | | AIDY | | | | | |
| Antibody | 1365 | GYTFTS | 1471 | IDPSESN | 1577 | ARGGY | 1683 | QSLAKS | GIS | 1895 | LQRTIQ |
| 93 | | YW | | T | | DGWDY | | YGNTY | | | YYT |
| | | | | | | AIDY | | | | | |
| Antibody | 1366 | GYTFTS | 1472 | IDPSESN | 1578 | ARGGY | 1684 | QSLAKS | GIS | 1896 | LQVTH |
| 94 | | YW | | T | | DGWDY | | YGNTY | | | QPYT |
| | | | | | | AIDY | | | | | |
| Antibody | 1367 | GYTFTS | 1473 | IDPSESN | 1579 | ARGGY | 1685 | QSLAKS | GIS | 1897 | LQWTH |
| 95 | | YW | | T | | DGWDY | | YGNTY | | | QPYT |
| | | | | | | AIDY | | | | | |
| Antibody | 1368 | GYTFTS | 1474 | IDPSESN | 1580 | ARGGY | 1686 | QSLAKS | GIS | 1898 | LQYTH |
| 96 | | YW | | T | | DGWDY | | YGNTY | | | QPYT |
| | | | | | | AIDY | | | | | |
| Antibody | 1369 | GYTFTS | 1475 | IDPSESN | 1581 | ARGGY | 1687 | QSLAKS | GIS | 1899 | LQYTIQ |
| 97 | | YW | | T | | DGWDY | | YGNTY | | | FYI |
| | | | | | | AIDY | | | | | |
| Antibody | 1370 | GYTFTS | 1476 | IDPSESN | 1582 | ARGGY | 1688 | QSLAKS | GIS | 1900 | LQYTH |
| 98 | | YW | | T | | DGWDY | | YGNTY | | | QFYI |
| | | | | | | AIDY | | | | | |
| Antibody | 1371 | GYTFTS | 1477 | IDPSESN | 1583 | ARGGY | 1689 | QSLAKS | GIS | 1901 | LQGTIQ |
| 99 | | YW | | T | | DGWDY | | YGNTY | | | PYT |
| | | | | | | AIDY | | | | | |
| Antibody | 1372 | GYTFTS | 1478 | IDPSESN | 1584 | ARGGY | 1690 | QSLAKS | GIS | 1902 | LQGTW |
| 100 | | YW | | T | | DGWDY | | YGNTY | | | QPYT |
| | | | | | | AIDY | | | | | |
| Antibody | 1373 | GYTFTS | 1479 | IDPSESN | 1585 | ARGGY | 1691 | QSLAKS | GIS | 1903 | LQGTH |
| 101 | | YW | | T | | DGWDY | | YGNTY | | | QFYT |
| | | | | | | AIDY | | | | | |
| Antibody | 1374 | GYTFTS | 1480 | IDPSESN | 1586 | ARGGY | 1692 | QSLAKS | GIS | 1904 | LQGTH |
| 102 | | YW | | T | | DGWDY | | YGNTY | | | QIYT |
| | | | | | | AIDY | | | | | |
| Antibody | 1375 | GYTFTS | 1481 | IDPSESN | 1587 | ARGGY | 1693 | QSLAKS | GIS | 1905 | LQGTH |
| 103 | | YW | | T | | DGWDY | | YGNTY | | | QVYT |
| | | | | | | AIDY | | | | | |
| Antibody | 1376 | GYTFTS | 1482 | IDPSESN | 1588 | ARGGY | 1694 | QSLAKS | GIS | 1906 | LQGTH |
| 104 | | YW | | T | | DGWDY | | YGNTY | | | QYYT |
| | | | | | | AIDY | | | | | |
| Antibody | 1377 | GYTFTS | 1483 | IDPSESN | 1589 | ARGGY | 1695 | QSLAKS | GIS | 1907 | LQGTH |
| 105 | | YW | | T | | DGWDY | | YGNTY | | | QPYI |
| | | | | | | AIDY | | | | | |
| Antibody | 1378 | GYTFTS | 1484 | IDPSESN | 1590 | ARGGY | 1696 | QSLAKS | GIS | 1908 | LQGTH |
| 106 | | YW | | T | | DGWDY | | YGNTY | | | QPYR |
| | | | | | | AIDY | | | | | |

In some embodiments, the α4β7 integrin binding protein comprises a heavy chain variable region comprising a CDR1, CDR2, and CDR3 as listed in Table 1.

In some embodiments, the α4β7 integrin binding protein comprises a light chain variable region comprising a CDR1, CDR2, and CDR3 as listed in Table 1.

In some embodiments, the α4β7 integrin binding protein comprises a heavy chain variable region comprising (a) a CDR1 having an amino acid sequence according to any one of SEQ ID NOs: 1-106, (b) a CDR2 having an amino acid sequence according to any one of SEQ ID NOs: 107-212, and (c) a CDR3 having an amino acid sequence according to any one of SEQ ID NOs: 213-318. In some embodiments, the α4β7 integrin binding protein comprises a light chain variable region comprising (a) a CDR1 having an amino acid sequence according to any one of SEQ ID NOs: 319-424, (b) a CDR2 having an amino acid sequence according to any one of SEQ ID NOs: 425-530, and (c) a CDR3 having an amino acid sequence according to any one of SEQ ID NOs: 531-536.

In some embodiments, the α4β7 integrin binding protein comprises a heavy chain variable region comprising (a) a CDR1 having an amino acid sequence according to any one of SEQ ID NOs: 637-742, (b) a CDR2 having an amino acid sequence according to any one of SEQ ID NOs: 743-848, and (c) a CDR3 having an amino acid sequence according to any one of SEQ ID NOs: 849-954. In some embodiments, the α4β7 integrin binding protein comprises a light chain variable region comprising (a) a CDR1 having an amino acid sequence according to any one of SEQ ID NOs: 955-1060, (b) a CDR2 having an amino acid sequence according to any one of SEQ ID NOs: 1061-1166, and (c) a CDR3 having an amino acid sequence according to any one of SEQ ID NOs: 1167-1272.

In some embodiments, the α4β7 integrin binding protein comprises a heavy chain In some embodiments, the α4β7 integrin binding protein comprises a heavy chain variable region comprising (a) a CDR1 having an amino acid sequence according to any one of SEQ ID NOs: 1273-1378, (b) a CDR2 having an amino acid sequence according to any one of SEQ ID NOs: 1378-1484, and (c) a CDR3 having an amino acid sequence according to any one of SEQ ID NOs: 1485-1590. In some embodiments, the α4β7 integrin binding protein comprises a light chain variable region comprising (a) a CDR1 having an amino acid sequence according to any one of SEQ ID NOs: 1591-1696, (b) a CDR2 having an amino acid sequence according to any one of GIS, YIS, FIS, and PIS, and (c) a CDR3 having an amino acid sequence according to any one of SEQ ID NOs: 1803-1908.

Amino acid sequences of exemplary heavy chain variable regions (VH) and light chain variable regions (VL) of α4β7 integrin binding proteins are provided in Table 2.

TABLE 2
Sequences of heavy chain variable regions (VH) and light chain variable regions
(VL) of α4β7 integrin binding proteins

| | SEQ ID | | SEQ ID | |
|---|---|---|---|---|
| Antibody | NO | VH Amino Acid Sequence | NO | VL Amino Acid Sequence |
| Antibody 1 | 1909 | EVQLVQSGAEVKKPGSSV | 2015 | DVVMTQTPLSLPVTPGQPASI |
| | | KVSCKASGGTFTSYWMH | | SCRSSQSLAKSYGNTYLSWY |
| | | WVRQAPGQGLEWMGEID | | LQKPGQSPQLLIYGISNRFSG |
| | | PSESNTNYNQKFKGRATI | | VPDRFSGSGSGTDFTLKISRV |
| | | TADISTSTAYMELSSLRSE | | EAEDVGVYYCLQGTHQPYTF |
| | | DTAVYYCARGGYDGWD | | GQGTKLEIK |
| | | YAIDYWGQGTLVTVSS | | |
| Antibody 2 | 1910 | EVQLVESGGGVVQPGRSL | 2016 | DVVMTQTPLSLPVTPGQPASI |
| | | RLSCAASGYTFTSYWMH | | SCRSSQSLAKSYGNTYLSWY |
| | | WVRQAPGKGLEWIGEIDP | | LQKPGQSPQLLIYGISNRFSG |
| | | SESNTNYNQKFKGRATIS | | VPDRFSGSGSGTDFTLKISRV |
| | | VDNSKNTAYLQMSSLRA | | EAEDVGVYYCLQGTHQPYTF |
| | | EDTAVYYCARGGYDGW | | GQGTKLEIK |
| | | DYAIDYWGQGTLVTVSS | | |
| Antibody 3 | 1911 | QVQLVQSGAEVKKPGAS | 2017 | DVVMTQSPLSLPVTPGEPASI |
| | | VKVSCKGSGYTFTIYWM | | SCRSSQSLAKSYGNTYLSWY |
| | | HWVRQAPGQRLEWIGEID | | LQKPGQSPQLLIYGISNRFSG |
| | | PSESNTNYNQKFKGRVTL | | VPDRFSGSGSGTDFTLKISRV |
| | | TVDISASTAYMELSSLRSE | | EAEDVGVYYCLQGTHQPYTF |
| | | DTAVYYCARGGYDGWD | | GQGTKVEIK |
| | | YAIDYWGQGTLVTVSS | | |
| Antibody 4 | 1912 | QVQLVQSGAEVKKPGAS | 2018 | DVVMTQSPLSLPVTPGEPASI |
| | | VKVSCKGSGYTFTVYWM | | SCRSSQSLAKSYGNTYLSWY |
| | | HWVRQAPGQRLEWIGEID | | LQKPGQSPQLLIYGISNRFSG |
| | | PSESNTNYNQKFKGRVTL | | VPDRFSGSGSGTDFTLKISRV |
| | | TVDISASTAYMELSSLRSE | | EAEDVGVYYCLQGTHQPYTF |
| | | DTAVYYCARGGYDGWD | | GQGTKVEIK |
| | | YAIDYWGQGTLVTVSS | | |
| Antibody 5 | 1913 | QVQLVQSGAEVKKPGAS | 2019 | DVVMTQSPLSLPVTPGEPASI |
| | | VKVSCKGSGYTFTSMWM | | SCRSSQSLAKSYGNTYLSWY |
| | | HWVRQAPGQRLEWIGEID | | LQKPGQSPQLLIYGISNRFSG |
| | | PSESNTNYNQKFKGRVTL | | VPDRFSGSGSGTDFTLKISRV |
| | | TVDISASTAYMELSSLRSE | | EAEDVGVYYCLQGTHQPYTF |
| | | DTAVYYCARGGYDGWD | | GQGTKVEIK |
| | | YAIDYWGQGTLVTVSS | | |
| Antibody 6 | 1914 | QVQLVQSGAEVKKPGAS | 2020 | DVVMTQSPLSLPVTPGEPASI |
| | | VKVSCKGSGYTFTSQWM | | SCRSSQSLAKSYGNTYLSWY |
| | | HWVRQAPGQRLEWIGEID | | LQKPGQSPQLLIYGISNRFSG |
| | | PSESNTNYNQKFKGRVTL | | VPDRFSGSGSGTDFTLKISRV |
| | | TVDISASTAYMELSSLRSE | | EAEDVGVYYCLQGTHQPYTF |
| | | DTAVYYCARGGYDGWD | | GQGTKVEIK |
| | | YAIDYWGQGTLVTVSS | | |
| Antibody 7 | 1915 | QVQLVQSGAEVKKPGAS | 2021 | DVVMTQSPLSLPVTPGEPASI |
| | | VKVSCKGSGYTFTSYWM | | SCRSSQSLAKSYGNTYLSWY |
| | | HWVRQAPGQRLEWIGEII | | LQKPGQSPQLLIYGISNRFSG |
| | | PSESNTNYNQKFKGRVTL | | VPDRFSGSGSGTDFTLKISRV |
| | | TVDISASTAYMELSSLRSE | | EAEDVGVYYCLQGTHQPYTF |
| | | DTAVYYCARGGYDGWD | | GQGTKVEIK |
| | | YAIDYWGQGTLVTVSS | | |
| Antibody 8 | 1916 | QVQLVQSGAEVKKPGAS | 2022 | DVVMTQSPLSLPVTPGEPASI |
| | | VKVSCKGSGYTFTSYWM | | SCRSSQSLAKSYGNTYLSWY |
| | | HWVRQAPGQRLEWIGEII | | LQKPGQSPQLLIYGISNRFSG |
| | | PLESNTNYNQKFKGRVTL | | VPDRFSGSGSGTDFTLKISRV |
| | | TVDISASTAYMELSSLRSE | | EAEDVGVYYCLQGTHQPYTF |
| | | DTAVYYCARGGYDGWD | | GQGTKVEIK |
| | | YAIDYWGQGTLVTVSS | | |
| Antibody 9 | 1917 | QVQLVQSGAEVKKPGAS | 2023 | DVVMTQSPLSLPVTPGEPASI |
| | | VKVSCKGSGYTFTSYWM | | SCRSSQSLAKSYGNTYLSWY |
| | | HWVRQAPGQRLEWIGEII | | LQKPGQSPQLLIYGISNRFSG |
| | | PRESNTNYNQKFKGRVTL | | VPDRFSGSGSGTDFTLKISRV |
| | | TVDISASTAYMELSSLRSE | | EAEDVGVYYCLQGTHQPYTF |
| | | DTAVYYCARGGYDGWD | | GQGTKVEIK |
| | | YAIDYWGQGTLVTVSS | | |
| Antibody | 1918 | QVQLVQSGAEVKKPGAS | 2024 | DVVMTQSPLSLPVTPGEPASI |
| 10 | | VKVSCKGSGYTFTSYWM | | SCRSSQSLAKSYGNTYLSWY |
| | | HWVRQAPGQRLEWIGEII | | LQKPGQSPQLLIYGISNRFSG |
| | | PVESNTNYNQKFKGRVTL | | VPDRFSGSGSGTDFTLKISRV |
| | | TVDISASTAYMELSSLRSE | | EAEDVGVYYCLQGTHQPYTF |
| | | DTAVYYCARGGYDGWD | | GQGTKVEIK |
| | | YAIDYWGQGTLVTVSS | | |
| Antibody | 1919 | QVQLVQSGAEVKKPGAS | 2025 | DVVMTQSPLSLPVTPGEPASI |
| 11 | | VKVSCKGSGYTFTSYWM | | SCRSSQSLAKSYGNTYLSWY |
| | | HWVRQAPGQRLEWIGEIR | | LQKPGQSPQLLIYGISNRFSG |
| | | PSESNTNYNQKFKGRVTL | | VPDRFSGSGSGTDFTLKISRV |
| | | TVDISASTAYMELSSLRSE | | EAEDVGVYYCLQGTHQPYTF |
| | | DTAVYYCARGGYDGWD | | GQGTKVEIK |
| | | YAIDYWGQGTLVTVSS | | |
| Antibody | 1920 | QVQLVQSGAEVKKPGAS | 2026 | DVVMTQSPLSLPVTPGEPASI |
| 12 | | VKVSCKGSGYTFTSYWM | | SCRSSQSLAKSYGNTYLSWY |
| | | HWVRQAPGQRLEWIGEIR | | LQKPGQSPQLLIYGISNRFSG |
| | | PLESNTNYNQKFKGRVTL | | VPDRFSGSGSGTDFTLKISRV |
| | | TVDISASTAYMELSSLRSE | | EAEDVGVYYCLQGTHQPYTF |
| | | DTAVYYCARGGYDGWD | | GQGTKVEIK |
| | | YAIDYWGQGTLVTVSS | | |
| Antibody | 1921 | QVQLVQSGAEVKKPGAS | 2027 | DVVMTQSPLSLPVTPGEPASI |
| 13 | | VKVSCKGSGYTFTSYWM | | SCRSSQSLAKSYGNTYLSWY |
| | | HWVRQAPGQRLEWIGEIR | | LQKPGQSPQLLIYGISNRFSG |
| | | PRESNTNYNQKFKGRVTL | | VPDRFSGSGSGTDFTLKISRV |
| | | TVDISASTAYMELSSLRSE | | EAEDVGVYYCLQGTHQPYTF |
| | | DTAVYYCARGGYDGWD | | GQGTKVEIK |
| | | YAIDYWGQGTLVTVSS | | |
| Antibody | 1922 | QVQLVQSGAEVKKPGAS | 2028 | DVVMTQSPLSLPVTPGEPASI |
| 14 | | VKVSCKGSGYTFTSYWM | | SCRSSQSLAKSYGNTYLSWY |
| | | HWVRQAPGQRLEWIGEIR | | LQKPGQSPQLLIYGISNRFSG |
| | | PVESNTNYNQKFKGRVTL | | VPDRFSGSGSGTDFTLKISRV |
| | | TVDISASTAYMELSSLRSE | | EAEDVGVYYCLQGTHQPYTF |
| | | DTAVYYCARGGYDGWD | | GQGTKVEIK |
| | | YAIDYWGQGTLVTVSS | | |
| Antibody | 1923 | QVQLVQSGAEVKKPGAS | 2029 | DVVMTQSPLSLPVTPGEPASI |
| 15 | | VKVSCKGSGYTFTSYWM | | SCRSSQSLAKSYGNTYLSWY |
| | | HWVRQAPGQRLEWIGEID | | LQKPGQSPQLLIYGISNRFSG |
| | | PLESNTNYNQKFKGRVTL | | VPDRFSGSGSGTDFTLKISRV |
| | | TVDISASTAYMELSSLRSE | | EAEDVGVYYCLQGTHQPYTF |
| | | DTAVYYCARGGYDGWD | | GQGTKVEIK |
| | | YAIDYWGQGTLVTVSS | | |
| Antibody | 1924 | QVQLVQSGAEVKKPGAS | 2030 | DVVMTQSPLSLPVTPGEPASI |
| 16 | | VKVSCKGSGYTFTSYWM | | SCRSSQSLAKSYGNTYLSWY |
| | | HWVRQAPGQRLEWIGEID | | LQKPGQSPQLLIYGISNRFSG |
| | | PMESNTNYNQKFKGRVT | | VPDRFSGSGSGTDFTLKISRV |
| | | LTVDISASTAYMELSSLRS | | EAEDVGVYYCLQGTHQPYTF |
| | | EDTAVYYCARGGYDGW | | GQGTKVEIK |
| | | DYAIDYWGQGTLVTVSS | | |
| Antibody | 1925 | QVQLVQSGAEVKKPGAS | 2031 | DVVMTQSPLSLPVTPGEPASI |
| 17 | | VKVSCKGSGYTFTSYWM | | SCRSSQSLAKSYGNTYLSWY |
| | | HWVRQAPGQRLEWIGEID | | LQKPGQSPQLLIYGISNRFSG |
| | | PRESNTNYNQKFKGRVTL | | VPDRFSGSGSGTDFTLKISRV |
| | | TVDISASTAYMELSSLRSE | | EAEDVGVYYCLQGTHQPYTF |
| | | DTAVYYCARGGYDGWD | | GQGTKVEIK |
| | | YAIDYWGQGTLVTVSS | | |
| Antibody | 1926 | QVQLVQSGAEVKKPGAS | 2032 | DVVMTQSPLSLPVTPGEPASI |
| 18 | | VKVSCKGSGYTFTSYWM | | SCRSSQSLAKSYGNTYLSWY |
| | | HWVRQAPGQRLEWIGEID | | LQKPGQSPQLLIYGISNRFSG |
| | | PVESNTNYNQKFKGRVTL | | VPDRFSGSGSGTDFTLKISRV |
| | | TVDISASTAYMELSSLRSE | | EAEDVGVYYCLQGTHQPYTF |
| | | DTAVYYCARGGYDGWD | | GQGTKVEIK |
| | | YAIDYWGQGTLVTVSS | | |
| Antibody | 1927 | QVQLVQSGAEVKKPGAS | 2033 | DVVMTQSPLSLPVTPGEPASI |
| 19 | | VKVSCKGSGYTFTSYWM | | SCRSSQSLAKSYGNTYLSWY |
| | | HWVRQAPGQRLEWIGEID | | LQKPGQSPQLLIYGISNRFSG |
| | | PSESNTNYNQKFKGRVTL | | VPDRFSGSGSGTDFTLKISRV |
| | | TVDISASTAYMELSSLRSE | | EAEDVGVYYCLQGTHQPYTF |
| | | DTAVYYCARGGYDGWD | | GQGTKVEIK |
| | | YFIDYWGQGTLVTVSS | | |
| Antibody | 1928 | QVQLVQSGAEVKKPGAS | 2034 | DVVMTQSPLSLPVTPGEPASI |
| 20 | | VKVSCKGSGYTFTSYWM | | SCRSSQSLAKSYGNTYLSWY |
| | | HWVRQAPGQRLEWIGEID | | LQKPGQSPQLLIYGISNRFSG |
| | | PSESNTNYNQKFKGRVTL | | VPDRFSGSGSGTDFTLKISRV |
| | | TVDISASTAYMELSSLRSE | | EAEDVGVYYCLQGTHQPYTF |
| | | DTAVYYCARGGYDGWD | | GQGTKVEIK |
| | | YFIYYWGQGTLVTVSS | | |
| Antibody | 1929 | QVQLVQSGAEVKKPGAS | 2035 | DVVMTQSPLSLPVTPGEPASI |
| 21 | | VKVSCKGSGYTFTSYWM | | SCRSSQSLAKSYGNTYLSWY |
| | | HWVRQAPGQRLEWIGEID | | LQKPGQSPQLLIYGISNRFSG |
| | | PSESNTNYNQKFKGRVTL | | VPDRFSGSGSGTDFTLKISRV |
| | | TVDISASTAYMELSSLRSE | | EAEDVGVYYCLQGTHQPYTF |
| | | DTAVYYCARGGYDGWD | | GQGTKVEIK |
| | | YLIDYWGQGTLVTVSS | | |
| Antibody | 1930 | QVQLVQSGAEVKKPGAS | 2036 | DVVMTQSPLSLPVTPGEPASI |
| 22 | | VKVSCKGSGYTFTSYWM | | SCRSSQSLAKSYGNTYLSWY |
| | | HWVRQAPGQRLEWIGEID | | LQKPGQSPQLLIYGISNRFSG |
| | | PSESNTNYNQKFKGRVTL | | VPDRFSGSGSGTDFTLKISRV |
| | | TVDISASTAYMELSSLRSE | | EAEDVGVYYCLQGTHQPYTF |
| | | DTAVYYCARGGYDGWD | | GQGTKVEIK |
| | | YVIDYWGQGTLVTVSS | | |
| Antibody | 1931 | QVQLVQSGAEVKKPGAS | 2037 | DVVMTQSPLSLPVTPGEPASI |
| 23 | | VKVSCKGSGYTFTSYWM | | SCRSSQSLAKSYGNTYLSWY |
| | | HWVRQAPGQRLEWIGEID | | LQKPGQSPQLLIYGISNRFSG |
| | | PSESNTNYNQKFKGRVTL | | VPDRFSGSGSGTDFTLKISRV |
| | | TVDISASTAYMELSSLRSE | | EAEDVGVYYCLQGTHQPYTF |
| | | DTAVYYCARGGYDGWD | | GQGTKVEIK |
| | | YVIYYWGQGTLVTVSS | | |
| Antibody | 1932 | QVQLVQSGAEVKKPGAS | 2038 | DVVMTQSPLSLPVTPGEPASI |
| 24 | | VKVSCKGSGYTFTSYWM | | SCRSSQSLAKSYGNTYLSWY |
| | | HWVRQAPGQRLEWIGEID | | LQKPGQSPQLLIYGISNRFSG |
| | | PSESNTNYNQKFKGRVTL | | VPDRFSGSGSGTDFTLKISRV |
| | | TVDISASTAYMELSSLRSE | | EAEDVGVYYCLQGTHQPYTF |
| | | DTAVYYCARGGYDGWD | | GQGTKVEIK |
| | | YWIDYWGQGTLVTVSS | | |
| Antibody | 1933 | QVQLVQSGAEVKKPGAS | 2039 | DVVMTQSPLSLPVTPGEPASI |
| 25 | | VKVSCKGSGYTFTSYWM | | SCRSSQSLAKSYGNTYLSWY |
| | | HWVRQAPGQRLEWIGEID | | LQKPGQSPQLLIYGISNRFSG |
| | | PSESNTNYNQKFKGRVTL | | VPDRFSGSGSGTDFTLKISRV |
| | | TVDISASTAYMELSSLRSE | | EAEDVGVYYCLQGTHQPYTF |
| | | DTAVYYCARGGYDGWD | | GQGTKVEIK |
| | | YYIDYWGQGTLVTVSS | | |
| Antibody | 1934 | QVQLVQSGAEVKKPGAS | 2040 | DVVMTQSPLSLPVTPGEPASI |
| 26 | | VKVSCKGSGYTFTSYWM | | SCRSSQSLAKSYGNTYLSWY |
| | | HWVRQAPGQRLEWIGEID | | LQKPGQSPQLLIYGISNRFSG |
| | | PSESNTNYNQKFKGRVTL | | VPDRFSGSGSGTDFTLKISRV |
| | | TVDISASTAYMELSSLRSE | | EAEDVGVYYCLQGTHQPYTF |
| | | DTAVYYCARGGYDGWD | | GQGTKVEIK |
| | | YYIYYWGQGTLVTVSS | | |
| Antibody | 1935 | QVQLVQSGAEVKKPGAS | 2041 | DVVMTQSPLSLPVTPGEPASI |
| 27 | | VKVSCKGSGYTFTSYWM | | SCRSSQSLAKSYGNTYLSWY |
| | | HWVRQAPGQRLEWIGEID | | LQKPGQSPQLLIYGISNRFSG |
| | | PSESNTNYNQKFKGRVTL | | VPDRFSGSGSGTDFTLKISRV |
| | | TVDISASTAYMELSSLRSE | | EAEDVGVYYCLQGTHQPYTF |
| | | DTAVYYCIRGGYDGWDY | | GQGTKVEIK |
| | | AIDYWGQGTLVTVSS | | |
| Antibody | 1936 | QVQLVQSGAEVKKPGAS | 2042 | DVVMTQSPLSLPVTPGEPASI |
| 28 | | VKVSCKGSGYTFTSYWM | | SCRSSQSLAKSYGNTYLSWY |
| | | HWVRQAPGQRLEWIGEID | | LQKPGQSPQLLIYGISNRFSG |
| | | PSESNTNYNQKFKGRVTL | | VPDRFSGSGSGTDFTLKISRV |
| | | TVDISASTAYMELSSLRSE | | EAEDVGVYYCLQGTHQPYTF |
| | | DTAVYYCLRGGYDGWD | | GQGTKVEIK |
| | | YAIDYWGQGTLVTVSS | | |
| Antibody | 1937 | QVQLVQSGAEVKKPGAS | 2043 | DVVMTQSPLSLPVTPGEPASI |
| 29 | | VKVSCKGSGYTFTSYWM | | SCRSSQSLAKSYGNTYLSWY |
| | | HWVRQAPGQRLEWIGEID | | LQKPGQSPQLLIYGISNRFSG |
| | | PSESNTNYNQKFKGRVTL | | VPDRFSGSGSGTDFTLKISRV |
| | | TVDISASTAYMELSSLRSE | | EAEDVGVYYCLQGTHQPYTF |
| | | DTAVYYCMRGGYDGWD | | GQGTKVEIK |
| | | YAIDYWGQGTLVTVSS | | |
| Antibody | 1938 | QVQLVQSGAEVKKPGAS | 2044 | DVVMTQSPLSLPVTPGEPASI |
| 30 | | VKVSCKGSGYTFTSYWM | | SCRSSQSLAKSYGNTYLSWY |
| | | HWVRQAPGQRLEWIGEID | | LQKPGQSPQLLIYGISNRFSG |
| | | PSESNTNYNQKFKGRVTL | | VPDRFSGSGSGTDFTLKISRV |
| | | TVDISASTAYMELSSLRSE | | EAEDVGVYYCLQGTHQPYTF |
| | | DTAVYYCRRGGYDGWD | | GQGTKVEIK |
| | | YAIDYWGQGTLVTVSS | | |
| Antibody | 1939 | QVQLVQSGAEVKKPGAS | 2045 | DVVMTQSPLSLPVTPGEPASI |
| 31 | | VKVSCKGSGYTFTSYWM | | SCRSSQSLAKSYGNTYLSWY |
| | | HWVRQAPGQRLEWIGEID | | LQKPGQSPQLLIYGISNRFSG |
| | | PSESNTNYNQKFKGRVTL | | VPDRFSGSGSGTDFTLKISRV |
| | | TVDISASTAYMELSSLRSE | | EAEDVGVYYCLQGTHQPYTF |
| | | DTAVYYCVRGGYDGWD | | GQGTKVEIK |
| | | YAIDYWGQGTLVTVSS | | |
| Antibody | 1940 | QVQLVQSGAEVKKPGAS | 2046 | DVVMTQSPLSLPVTPGEPASI |
| 32 | | VKVSCKGSGYTFTSYWM | | SCRSSQSLAKSYGNTYLSWY |
| | | HWVRQAPGQRLEWIGEID | | LQKPGQSPQLLIYGISNRFSG |
| | | PSESNTNYNQKFKGRVTL | | VPDRFSGSGSGTDFTLKISRV |
| | | TVDISASTAYMELSSLRSE | | EAEDVGVYYCLQGTHQPYTF |
| | | DTAVYYCARGGYWGWD | | GQGTKVEIK |
| | | YAIDYWGQGTLVTVSS | | |
| Antibody | 1941 | QVQLVQSGAEVKKPGAS | 2047 | DVVMTQSPLSLPVTPGEPASI |
| 33 | | VKVSCKGSGYTFTSYWM | | SCRSSQSLAKSYGNTYLSWY |
| | | HWVRQAPGQRLEWIGEID | | LQKPGQSPQLLIYGISNRFSG |
| | | PSESNTNYNQKFKGRVTL | | VPDRFSGSGSGTDFTLKISRV |
| | | TVDISASTAYMELSSLRSE | | EAEDVGVYYCLQGTHQPYTF |
| | | DTAVYYCARGGYYGWD | | GQGTKVEIK |
| | | YAIDYWGQGTLVTVSS | | |
| Antibody | 1942 | QVQLVQSGAEVKKPGAS | 2048 | DVVMTQSPLSLPVTPGEPASI |
| 34 | | VKVSCKGSGYTFTSYWM | | SCRSSQSLAKSYGNTYLSWY |
| | | HWVRQAPGQRLEWIGEID | | LQKPGQSPQLLIYGISNRFSG |
| | | PSESNTNYNQKFKGRVTL | | VPDRFSGSGSGTDFTLKISRV |
| | | TVDISASTAYMELSSLRSE | | EAEDVGVYYCLQGTHQPYTF |
| | | DTAVYYCARGGYDGWD | | GQGTKVEIK |
| | | YAIFYWGQGTLVTVSS | | |
| Antibody | 1943 | QVQLVQSGAEVKKPGAS | 2049 | DVVMTQSPLSLPVTPGEPASI |
| 35 | | VKVSCKGSGYTFTSYWM | | SCRSSQSLAKSYGNTYLSWY |
| | | HWVRQAPGQRLEWIGEID | | LQKPGQSPQLLIYGISNRFSG |
| | | PSESNTNYNQKFKGRVTL | | VPDRFSGSGSGTDFTLKISRV |
| | | TVDISASTAYMELSSLRSE | | EAEDVGVYYCLQGTHQPYTF |
| | | DTAVYYCARGGYDGWD | | GQGTKVEIK |
| | | YAIMYWGQGTLVTVSS | | |
| Antibody | 1944 | QVQLVQSGAEVKKPGAS | 2050 | DVVMTQSPLSLPVTPGEPASI |
| 36 | | VKVSCKGSGYTFTSYWM | | SCRSSQSLAKSYGNTYLSWY |
| | | HWVRQAPGQRLEWIGEID | | LQKPGQSPQLLIYGISNRFSG |
| | | PSESNTNYNQKFKGRVTL | | VPDRFSGSGSGTDFTLKISRV |
| | | TVDISASTAYMELSSLRSE | | EAEDVGVYYCLQGTHQPYTF |
| | | DTAVYYCARGGYDGWD | | GQGTKVEIK |
| | | YAIWYWGQGTLVTVSS | | |
| Antibody | 1945 | QVQLVQSGAEVKKPGAS | 2051 | DVVMTQSPLSLPVTPGEPASI |
| 37 | | VKVSCKGSGYTFTSYWM | | SCRSSQSLAKSYGNTYLSWY |
| | | HWVRQAPGQRLEWIGEID | | LQKPGQSPQLLIYGISNRFSG |
| | | PSESNTNYNQKFKGRVTL | | VPDRFSGSGSGTDFTLKISRV |
| | | TVDISASTAYMELSSLRSE | | EAEDVGVYYCLQGTHQPYTF |
| | | DTAVYYCARGGYDGWD | | GQGTKVEIK |
| | | YAIYYWGQGTLVTVSS | | |
| Antibody | 1946 | QVQLVQSGAEVKKPGAS | 2052 | DVVMTQSPLSLPVTPGEPASI |
| 38 | | VKVSCKGSGYTFTSYWM | | SCRSSQSLAKSYGNTYLSWY |
| | | HWVRQAPGQRLEWIGEID | | LQKPGQSPQLLIYGISNRFSG |
| | | PSESNTNYNQKFKGRVTL | | VPDRFSGSGSGTDFTLKISRV |
| | | TVDISASTAYMELSSLRSE | | EAEDVGVYYCLQGTHQPYTF |
| | | DTAVYYCARGFYDGWD | | GQGTKVEIK |
| | | YAIDYWGQGTLVTVSS | | |
| Antibody | 1947 | QVQLVQSGAEVKKPGAS | 2053 | DVVMTQSPLSLPVTPGEPASI |
| 39 | | VKVSCKGSGYTFTSYWM | | SCRSSQSLAKSYGNTYLSWY |
| | | HWVRQAPGQRLEWIGEID | | LQKPGQSPQLLIYGISNRFSG |
| | | PSESNTNYNQKFKGRVTL | | VPDRFSGSGSGTDFTLKISRV |
| | | TVDISASTAYMELSSLRSE | | EAEDVGVYYCLQGTHQPYTF |
| | | DTAVYYCARGFYDGWD | | GQGTKVEIK |
| | | YLIDYWGQGTLVTVSS | | |
| Antibody | 1948 | QVQLVQSGAEVKKPGAS | 2054 | DVVMTQSPLSLPVTPGEPASI |
| 40 | | VKVSCKGSGYTFTSYWM | | SCRSSQSLAKSYGNTYLSWY |
| | | HWVRQAPGQRLEWIGEID | | LQKPGQSPQLLIYGISNRFSG |
| | | PSESNTNYNQKFKGRVTL | | VPDRFSGSGSGTDFTLKISRV |
| | | TVDISASTAYMELSSLRSE | | EAEDVGVYYCLQGTHQPYTF |
| | | DTAVYYCARGFYDGWD | | GQGTKVEIK |
| | | YAIYYWGQGTLVTVSS | | |
| Antibody | 1949 | QVQLVQSGAEVKKPGAS | 2055 | DVVMTQSPLSLPVTPGEPASI |
| 41 | | VKVSCKGSGYTFTSYWM | | SCRSSQSLAKSYGNTYLSWY |
| | | HWVRQAPGQRLEWIGEID | | LQKPGQSPQLLIYGISNRFSG |
| | | PSESNTNYNQKFKGRVTL | | VPDRFSGSGSGTDFTLKISRV |
| | | TVDISASTAYMELSSLRSE | | EAEDVGVYYCLQGTHQPYTF |
| | | DTAVYYCARGMYDGWD | | GQGTKVEIK |
| | | YAIDYWGQGTLVTVSS | | |
| Antibody | 1950 | QVQLVQSGAEVKKPGAS | 2056 | DVVMTQSPLSLPVTPGEPASI |
| 42 | | VKVSCKGSGYTFTSYWM | | SCRSSQSLAKSYGNTYLSWY |
| | | HWVRQAPGQRLEWIGEID | | LQKPGQSPQLLIYGISNRFSG |
| | | PSESNTNYNQKFKGRVTL | | VPDRFSGSGSGTDFTLKISRV |
| | | TVDISASTAYMELSSLRSE | | EAEDVGVYYCLQGTHQPYTF |
| | | DTAVYYCARGRYDGWD | | GQGTKVEIK |
| | | YAIDYWGQGTLVTVSS | | |
| Antibody | 1951 | QVQLVQSGAEVKKPGAS | 2057 | DVVMTQSPLSLPVTPGEPASI |
| 43 | | VKVSCKGSGYTFTSYWM | | SCRSSQSLAKSYGNTYLSWY |
| | | HWVRQAPGQRLEWIGEID | | LQKPGQSPQLLIYGISNRFSG |
| | | PSESNTNYNQKFKGRVTL | | VPDRFSGSGSGTDFTLKISRV |
| | | TVDISASTAYMELSSLRSE | | EAEDVGVYYCLQGTHQPYTF |
| | | DTAVYYCARGVYDGWD | | GQGTKVEIK |
| | | YAIDYWGQGTLVTVSS | | |
| Antibody | 1952 | QVQLVQSGAEVKKPGAS | 2058 | DVVMTQSPLSLPVTPGEPASI |
| 44 | | VKVSCKGSGYTFTSYWM | | SCRSSQSLAKSYGNTYLSWY |
| | | HWVRQAPGQRLEWIGEID | | LQKPGQSPQLLIYGISNRFSG |
| | | PSESNTNYNQKFKGRVTL | | VPDRFSGSGSGTDFTLKISRV |
| | | TVDISASTAYMELSSLRSE | | EAEDVGVYYCLQGTHQPYTF |
| | | DTAVYYCARGGYDIWDY | | GQGTKVEIK |
| | | AIDYWGQGTLVTVSS | | |
| Antibody | 1953 | QVQLVQSGAEVKKPGAS | 2059 | DVVMTQSPLSLPVTPGEPASI |
| 45 | | VKVSCKGSGYTFTSYWM | | SCRSSQSLAKSYGNTYLSWY |
| | | HWVRQAPGQRLEWIGEID | | LQKPGQSPQLLIYGISNRFSG |
| | | PSESNTNYNQKFKGRVTL | | VPDRFSGSGSGTDFTLKISRV |
| | | TVDISASTAYMELSSLRSE | | EAEDVGVYYCLQGTHQPYTF |
| | | DTAVYYCARGGYDLWD | | GQGTKVEIK |
| | | YAIDYWGQGTLVTVSS | | |
| Antibody | 1954 | QVQLVQSGAEVKKPGAS | 2060 | DVVMTQSPLSLPVTPGEPASI |
| 46 | | VKVSCKGSGYTFTSYWM | | SCRSSQSLAKSYGNTYLSWY |
| | | HWVRQAPGQRLEWIGEID | | LQKPGQSPQLLIYGISNRFSG |
| | | PSESNTNYNQKFKGRVTL | | VPDRFSGSGSGTDFTLKISRV |
| | | TVDISASTAYMELSSLRSE | | EAEDVGVYYCLQGTHQPYTF |
| | | DTAVYYCARGGYDVWD | | GQGTKVEIK |
| | | YAIDYWGQGTLVTVSS | | |
| Antibody | 1955 | QVQLVQSGAEVKKPGAS | 2061 | DVVMTQSPLSLPVTPGEPASI |
| 47 | | VKVSCKGSGYTFTSYWM | | SCRSSQSLAKSYGNTYLSWY |
| | | HWVRQAPGQRLEWIGEID | | LQKPGQSPQLLIYGISNRFSG |
| | | PSESNTNYNQKFKGRVTL | | VPDRFSGSGSGTDFTLKISRV |
| | | TVDISASTAYMELSSLRSE | | EAEDVGVYYCLQGTHQPYTF |
| | | DTAVYYCARFGYDGWD | | GQGTKVEIK |
| | | YAIDYWGQGTLVTVSS | | |
| Antibody | 1956 | QVQLVQSGAEVKKPGAS | 2062 | DVVMTQSPLSLPVTPGEPASI |
| 48 | | VKVSCKGSGYTFTSYWM | | SCRSSQSLAKSYGNTYLSWY |
| | | HWVRQAPGQRLEWIGEID | | LQKPGQSPQLLIYGISNRFSG |
| | | PSESNTNYNQKFKGRVTL | | VPDRFSGSGSGTDFTLKISRV |
| | | TVDISASTAYMELSSLRSE | | EAEDVGVYYCLQGTHQPYTF |
| | | DTAVYYCARYGYDGWD | | GQGTKVEIK |
| | | YAIDYWGQGTLVTVSS | | |
| Antibody | 1957 | QVQLVQSGAEVKKPGAS | 2063 | DVVMTQSPLSLPVTPGEPASI |
| 49 | | VKVSCKGSGYTFTSYWM | | SCRSSQSLAASYGNTYLSWY |
| | | HWVRQAPGQRLEWIGEID | | LQKPGQSPQLLIYGISNRFSG |
| | | PSESNTNYNQKFKGRVTL | | VPDRFSGSGSGTDFTLKISRV |
| | | TVDISASTAYMELSSLRSE | | EAEDVGVYYCLQGTHQPYTF |
| | | DTAVYYCARGGYDGWD | | GQGTKVEIK |
| | | YAIDYWGQGTLVTVSS | | |
| Antibody | 1958 | QVQLVQSGAEVKKPGAS | 2064 | DVVMTQSPLSLPVTPGEPASI |
| 50 | | VKVSCKGSGYTFTSYWM | | SCRSSQSLADSYGNTYLSWY |
| | | HWVRQAPGQRLEWIGEID | | LQKPGQSPQLLIYGISNRFSG |
| | | PSESNTNYNQKFKGRVTL | | VPDRFSGSGSGTDFTLKISRV |
| | | TVDISASTAYMELSSLRSE | | EAEDVGVYYCLQGTHQPYTF |
| | | DTAVYYCARGGYDGWD | | GQGTKVEIK |
| | | YAIDYWGQGTLVTVSS | | |
| Antibody | 1959 | QVQLVQSGAEVKKPGAS | 2065 | DVVMTQSPLSLPVTPGEPASI |
| 51 | | VKVSCKGSGYTFTSYWM | | SCRSSQSLAISYGNTYLSWYL |
| | | HWVRQAPGQRLEWIGEID | | QKPGQSPQLLIYGISNRFSGVP |
| | | PSESNTNYNQKFKGRVTL | | DRFSGSGSGTDFTLKISRVEA |
| | | TVDISASTAYMELSSLRSE | | EDVGVYYCLQGTHQPYTFGQ |
| | | DTAVYYCARGGYDGWD | | GTKVEIK |
| | | YAIDYWGQGTLVTVSS | | |
| Antibody | 1960 | QVQLVQSGAEVKKPGAS | 2066 | DVVMTQSPLSLPVTPGEPASI |
| 52 | | VKVSCKGSGYTFTSYWM | | SCRSSQSLAIFYGNTYLSWYL |
| | | HWVRQAPGQRLEWIGEID | | QKPGQSPQLLIYGISNRFSGVP |
| | | PSESNTNYNQKFKGRVTL | | DRFSGSGSGTDFTLKISRVEA |
| | | TVDISASTAYMELSSLRSE | | EDVGVYYCLQGTHQPYTFGQ |
| | | DTAVYYCARGGYDGWD | | GTKVEIK |
| | | YAIDYWGQGTLVTVSS | | |
| Antibody | 1961 | QVQLVQSGAEVKKPGAS | 2067 | DVVMTQSPLSLPVTPGEPASI |
| 53 | | VKVSCKGSGYTFTSYWM | | SCRSSQSLAIFYGITYLSWYL |
| | | HWVRQAPGQRLEWIGEID | | QKPGQSPQLLIYGISNRFSGVP |
| | | PSESNTNYNQKFKGRVTL | | DRFSGSGSGTDFTLKISRVEA |
| | | TVDISASTAYMELSSLRSE | | EDVGVYYCLQGTHQPYTFGQ |
| | | DTAVYYCARGGYDGWD | | GTKVEIK |
| | | YAIDYWGQGTLVTVSS | | |
| Antibody | 1962 | QVQLVQSGAEVKKPGAS | 2068 | DVVMTQSPLSLPVTPGEPASI |
| 54 | | VKVSCKGSGYTFTSYWM | | SCRSSQSLAIFYGLTYLSWYL |
| | | HWVRQAPGQRLEWIGEID | | QKPGQSPQLLIYGISNRFSGVP |
| | | PSESNTNYNQKFKGRVTL | | DRFSGSGSGTDFTLKISRVEA |
| | | TVDISASTAYMELSSLRSE | | EDVGVYYCLQGTHQPYTFGQ |
| | | DTAVYYCARGGYDGWD | | GTKVEIK |
| | | YAIDYWGQGTLVTVSS | | |
| Antibody | 1963 | QVQLVQSGAEVKKPGAS | 2069 | DVVMTQSPLSLPVTPGEPASI |
| 55 | | VKVSCKGSGYTFTSYWM | | SCRSSQSLAILYGITYLSWYL |
| | | HWVRQAPGQRLEWIGEID | | QKPGQSPQLLIYGISNRFSGVP |
| | | PSESNTNYNQKFKGRVTL | | DRFSGSGSGTDFTLKISRVEA |
| | | TVDISASTAYMELSSLRSE | | EDVGVYYCLQGTHQPYTFGQ |
| | | DTAVYYCARGGYDGWD | | GTKVEIK |
| | | YAIDYWGQGTLVTVSS | | |
| Antibody | 1964 | QVQLVQSGAEVKKPGAS | 2070 | DVVMTQSPLSLPVTPGEPASI |
| 56 | | VKVSCKGSGYTFTSYWM | | SCRSSQSLAILYGLTYLSWYL |
| | | HWVRQAPGQRLEWIGEID | | QKPGQSPQLLIYGISNRFSGVP |
| | | PSESNTNYNQKFKGRVTL | | DRFSGSGSGTDFTLKISRVEA |
| | | TVDISASTAYMELSSLRSE | | EDVGVYYCLQGTHQPYTFGQ |
| | | DTAVYYCARGGYDGWD | | GTKVEIK |
| | | YAIDYWGQGTLVTVSS | | |
| Antibody | 1965 | QVQLVQSGAEVKKPGAS | 2071 | DVVMTQSPLSLPVTPGEPASI |
| 57 | | VKVSCKGSGYTFTSYWM | | SCRSSQSLALSYGNTYLSWY |
| | | HWVRQAPGQRLEWIGEID | | LQKPGQSPQLLIYGISNRFSG |
| | | PSESNTNYNQKFKGRVTL | | VPDRFSGSGSGTDFTLKISRV |
| | | TVDISASTAYMELSSLRSE | | EAEDVGVYYCLQGTHQPYTF |
| | | DTAVYYCARGGYDGWD | | GQGTKVEIK |
| | | YAIDYWGQGTLVTVSS | | |
| Antibody | 1966 | QVQLVQSGAEVKKPGAS | 2072 | DVVMTQSPLSLPVTPGEPASI |
| 58 | | VKVSCKGSGYTFTSYWM | | SCRSSQSLAMSYGNTYLSWY |
| | | HWVRQAPGQRLEWIGEID | | LQKPGQSPQLLIYGISNRFSG |
| | | PSESNTNYNQKFKGRVTL | | VPDRFSGSGSGTDFTLKISRV |
| | | TVDISASTAYMELSSLRSE | | EAEDVGVYYCLQGTHQPYTF |
| | | DTAVYYCARGGYDGWD | | GQGTKVEIK |
| | | YAIDYWGQGTLVTVSS | | |
| Antibody | 1967 | QVQLVQSGAEVKKPGAS | 2073 | DVVMTQSPLSLPVTPGEPASI |
| 59 | | VKVSCKGSGYTFTSYWM | | SCRSSQSLANSYGNTYLSWY |
| | | HWVRQAPGQRLEWIGEID | | LQKPGQSPQLLIYGISNRFSG |
| | | PSESNTNYNQKFKGRVTL | | VPDRFSGSGSGTDFTLKISRV |
| | | TVDISASTAYMELSSLRSE | | EAEDVGVYYCLQGTHQPYTF |
| | | DTAVYYCARGGYDGWD | | GQGTKVEIK |
| | | YAIDYWGQGTLVTVSS | | |
| Antibody | 1968 | QVQLVQSGAEVKKPGAS | 2074 | DVVMTQSPLSLPVTPGEPASI |
| 60 | | VKVSCKGSGYTFTSYWM | | SCRSSQSLAPSYGNTYLSWYL |
| | | HWVRQAPGQRLEWIGEID | | QKPGQSPQLLIYGISNRFSGVP |
| | | PSESNTNYNQKFKGRVTL | | DRFSGSGSGTDFTLKISRVEA |
| | | TVDISASTAYMELSSLRSE | | EDVGVYYCLQGTHQPYTFGQ |
| | | DTAVYYCARGGYDGWD | | GTKVEIK |
| | | YAIDYWGQGTLVTVSS | | |
| Antibody | 1969 | QVQLVQSGAEVKKPGAS | 2075 | DVVMTQSPLSLPVTPGEPASI |
| 61 | | VKVSCKGSGYTFTSYWM | | SCRSSQSLAQSYGNTYLSWY |
| | | HWVRQAPGQRLEWIGEID | | LQKPGQSPQLLIYGISNRFSG |
| | | PSESNTNYNQKFKGRVTL | | VPDRFSGSGSGTDFTLKISRV |
| | | TVDISASTAYMELSSLRSE | | EAEDVGVYYCLQGTHQPYTF |
| | | DTAVYYCARGGYDGWD | | GQGTKVEIK |
| | | YAIDYWGQGTLVTVSS | | |
| Antibody | 1970 | QVQLVQSGAEVKKPGAS | 2076 | DVVMTQSPLSLPVTPGEPASI |
| 62 | | VKVSCKGSGYTFTSYWM | | SCRSSQSLARSYGNTYLSWY |
| | | HWVRQAPGQRLEWIGEID | | LQKPGQSPQLLIYGISNRFSG |
| | | PSESNTNYNQKFKGRVTL | | VPDRFSGSGSGTDFTLKISRV |
| | | TVDISASTAYMELSSLRSE | | EAEDVGVYYCLQGTHQPYTF |
| | | DTAVYYCARGGYDGWD | | GQGTKVEIK |
| | | YAIDYWGQGTLVTVSS | | |
| Antibody | 1971 | QVQLVQSGAEVKKPGAS | 2077 | DVVMTQSPLSLPVTPGEPASI |
| 63 | | VKVSCKGSGYTFTSYWM | | SCRSSQSLARFYGLTYLSWYL |
| | | HWVRQAPGQRLEWIGEID | | QKPGQSPQLLIYGISNRFSGVP |
| | | PSESNTNYNQKFKGRVTL | | DRFSGSGSGTDFTLKISRVEA |
| | | TVDISASTAYMELSSLRSE | | EDVGVYYCLQGTHQPYTFGQ |
| | | DTAVYYCARGGYDGWD | | GTKVEIK |
| | | YAIDYWGQGTLVTVSS | | |
| Antibody | 1972 | QVQLVQSGAEVKKPGAS | 2078 | DVVMTQSPLSLPVTPGEPASI |
| 64 | | VKVSCKGSGYTFTSYWM | | SCRSSQSLASSYGNTYLSWYL |
| | | HWVRQAPGQRLEWIGEID | | QKPGQSPQLLIYGISNRFSGVP |
| | | PSESNTNYNQKFKGRVTL | | DRFSGSGSGTDFTLKISRVEA |
| | | TVDISASTAYMELSSLRSE | | EDVGVYYCLQGTHQPYTFGQ |
| | | DTAVYYCARGGYDGWD | | GTKVEIK |
| | | YAIDYWGQGTLVTVSS | | |
| Antibody | 1973 | QVQLVQSGAEVKKPGAS | 2079 | DVVMTQSPLSLPVTPGEPASI |
| 65 | | VKVSCKGSGYTFTSYWM | | SCRSSQSLATSYGNTYLSWY |
| | | HWVRQAPGQRLEWIGEID | | LQKPGQSPQLLIYGISNRFSG |
| | | PSESNTNYNQKFKGRVTL | | VPDRFSGSGSGTDFTLKISRV |
| | | TVDISASTAYMELSSLRSE | | EAEDVGVYYCLQGTHQPYTF |
| | | DTAVYYCARGGYDGWD | | GQGTKVEIK |
| | | YAIDYWGQGTLVTVSS | | |
| Antibody | 1974 | QVQLVQSGAEVKKPGAS | 2080 | DVVMTQSPLSLPVTPGEPASI |
| 66 | | VKVSCKGSGYTFTSYWM | | SCRSSQSLATYYGITYLSWYL |
| | | HWVRQAPGQRLEWIGEID | | QKPGQSPQLLIYGISNRFSGVP |
| | | PSESNTNYNQKFKGRVTL | | DRFSGSGSGTDFTLKISRVEA |
| | | TVDISASTAYMELSSLRSE | | EDVGVYYCLQGTHQPYTFGQ |
| | | DTAVYYCARGGYDGWD | | GTKVEIK |
| | | YAIDYWGQGTLVTVSS | | |
| Antibody | 1975 | QVQLVQSGAEVKKPGAS | 2081 | DVVMTQSPLSLPVTPGEPASI |
| 67 | | VKVSCKGSGYTFTSYWM | | SCRSSQSLATYYGLTYLSWY |
| | | HWVRQAPGQRLEWIGEID | | LQKPGQSPQLLIYGISNRFSG |
| | | PSESNTNYNQKFKGRVTL | | VPDRFSGSGSGTDFTLKISRV |
| | | TVDISASTAYMELSSLRSE | | EAEDVGVYYCLQGTHQPYTF |
| | | DTAVYYCARGGYDGWD | | GQGTKVEIK |
| | | YAIDYWGQGTLVTVSS | | |
| Antibody | 1976 | QVQLVQSGAEVKKPGAS | 2082 | DVVMTQSPLSLPVTPGEPASI |
| 68 | | VKVSCKGSGYTFTSYWM | | SCRSSQSLAVSYGNTYLSWY |
| | | HWVRQAPGQRLEWIGEID | | LQKPGQSPQLLIYGISNRFSG |
| | | PSESNTNYNQKFKGRVTL | | VPDRFSGSGSGTDFTLKISRV |
| | | TVDISASTAYMELSSLRSE | | EAEDVGVYYCLQGTHQPYTF |
| | | DTAVYYCARGGYDGWD | | GQGTKVEIK |
| | | YAIDYWGQGTLVTVSS | | |
| Antibody | 1977 | QVQLVQSGAEVKKPGAS | 2083 | DVVMTQSPLSLPVTPGEPASI |
| 69 | | VKVSCKGSGYTFTSYWM | | SCRSSQSLAYSYGNTYLSWY |
| | | HWVRQAPGQRLEWIGEID | | LQKPGQSPQLLIYGISNRFSG |
| | | PSESNTNYNQKFKGRVTL | | VPDRFSGSGSGTDFTLKISRV |
| | | TVDISASTAYMELSSLRSE | | EAEDVGVYYCLQGTHQPYTF |
| | | DTAVYYCARGGYDGWD | | GQGTKVEIK |
| | | YAIDYWGQGTLVTVSS | | |
| Antibody | 1978 | QVQLVQSGAEVKKPGAS | 2084 | DVVMTQSPLSLPVTPGEPASI |
| 70 | | VKVSCKGSGYTFTSYWM | | SCRSSQSLAYFYGITYLSWYL |
| | | HWVRQAPGQRLEWIGEID | | QKPGQSPQLLIYGISNRFSGVP |
| | | PSESNTNYNQKFKGRVTL | | DRFSGSGSGTDFTLKISRVEA |
| | | TVDISASTAYMELSSLRSE | | EDVGVYYCLQGTHQPYTFGQ |
| | | DTAVYYCARGGYDGWD | | GTKVEIK |
| | | YAIDYWGQGTLVTVSS | | |
| Antibody | 1979 | QVQLVQSGAEVKKPGAS | 2085 | DVVMTQSPLSLPVTPGEPASI |
| 71 | | VKVSCKGSGYTFTSYWM | | SCRSSQSLAYLYGITYLSWYL |
| | | HWVRQAPGQRLEWIGEID | | QKPGQSPQLLIYGISNRFSGVP |
| | | PSESNTNYNQKFKGRVTL | | DRFSGSGSGTDFTLKISRVEA |
| | | TVDISASTAYMELSSLRSE | | EDVGVYYCLQGTHQPYTFGQ |
| | | DTAVYYCARGGYDGWD | | GTKVEIK |
| | | YAIDYWGQGTLVTVSS | | |
| Antibody | 1980 | QVQLVQSGAEVKKPGAS | 2086 | DVVMTQSPLSLPVTPGEPASI |
| 72 | | VKVSCKGSGYTFTSYWM | | SCRSSQSLAKSYGITYLSWYL |
| | | HWVRQAPGQRLEWIGEID | | QKPGQSPQLLIYGISNRFSGVP |
| | | PSESNTNYNQKFKGRVTL | | DRFSGSGSGTDFTLKISRVEA |
| | | TVDISASTAYMELSSLRSE | | EDVGVYYCLQGTHQPYTFGQ |
| | | DTAVYYCARGGYDGWD | | GTKVEIK |
| | | YAIDYWGQGTLVTVSS | | |
| Antibody | 1981 | QVQLVQSGAEVKKPGAS | 2087 | DVVMTQSPLSLPVTPGEPASI |
| 73 | | VKVSCKGSGYTFTSYWM | | SCRSSQSLAKSYGLTYLSWY |
| | | HWVRQAPGQRLEWIGEID | | LQKPGQSPQLLIYGISNRFSG |
| | | PSESNTNYNQKFKGRVTL | | VPDRFSGSGSGTDFTLKISRV |
| | | TVDISASTAYMELSSLRSE | | EAEDVGVYYCLQGTHQPYTF |
| | | DTAVYYCARGGYDGWD | | GQGTKVEIK |
| | | YAIDYWGQGTLVTVSS | | |
| Antibody | 1982 | QVQLVQSGAEVKKPGAS | 2088 | DVVMTQSPLSLPVTPGEPASI |
| 74 | | VKVSCKGSGYTFTSYWM | | SCRSSQSLAKFYGNTYLSWY |
| | | HWVRQAPGQRLEWIGEID | | LQKPGQSPQLLIYGISNRFSG |
| | | PSESNTNYNQKFKGRVTL | | VPDRFSGSGSGTDFTLKISRV |
| | | TVDISASTAYMELSSLRSE | | EAEDVGVYYCLQGTHQPYTF |
| | | DTAVYYCARGGYDGWD | | GQGTKVEIK |
| | | YAIDYWGQGTLVTVSS | | |
| Antibody | 1983 | QVQLVQSGAEVKKPGAS | 2089 | DVVMTQSPLSLPVTPGEPASI |
| 75 | | VKVSCKGSGYTFTSYWM | | SCRSSQSLAKIYGNTYLSWYL |
| | | HWVRQAPGQRLEWIGEID | | QKPGQSPQLLIYGISNRFSGVP |
| | | PSESNTNYNQKFKGRVTL | | DRFSGSGSGTDFTLKISRVEA |
| | | TVDISASTAYMELSSLRSE | | EDVGVYYCLQGTHQPYTFGQ |
| | | DTAVYYCARGGYDGWD | | GTKVEIK |
| | | YAIDYWGQGTLVTVSS | | |
| Antibody | 1984 | QVQLVQSGAEVKKPGAS | 2090 | DVVMTQSPLSLPVTPGEPASI |
| 76 | | VKVSCKGSGYTFTSYWM | | SCRSSQSLAKLYGNTYLSWY |
| | | HWVRQAPGQRLEWIGEID | | LQKPGQSPQLLIYGISNRFSG |
| | | PSESNTNYNQKFKGRVTL | | VPDRFSGSGSGTDFTLKISRV |
| | | TVDISASTAYMELSSLRSE | | EAEDVGVYYCLQGTHQPYTF |
| | | DTAVYYCARGGYDGWD | | GQGTKVEIK |
| | | YAIDYWGQGTLVTVSS | | |
| Antibody | 1985 | QVQLVQSGAEVKKPGAS | 2091 | DVVMTQSPLSLPVTPGEPASI |
| 77 | | VKVSCKGSGYTFTSYWM | | SCRSSQSLAKMYGNTYLSWY |
| | | HWVRQAPGQRLEWIGEID | | LQKPGQSPQLLIYGISNRFSG |
| | | PSESNTNYNQKFKGRVTL | | VPDRFSGSGSGTDFTLKISRV |
| | | TVDISASTAYMELSSLRSE | | EAEDVGVYYCLQGTHQPYTF |
| | | DTAVYYCARGGYDGWD | | GQGTKVEIK |
| | | YAIDYWGQGTLVTVSS | | |
| Antibody | 1986 | QVQLVQSGAEVKKPGAS | 2092 | DVVMTQSPLSLPVTPGEPASI |
| 78 | | VKVSCKGSGYTFTSYWM | | SCRSSQSLAKQYGNTYLSWY |
| | | HWVRQAPGQRLEWIGEID | | LQKPGQSPQLLIYGISNRFSG |
| | | PSESNTNYNQKFKGRVTL | | VPDRFSGSGSGTDFTLKISRV |
| | | TVDISASTAYMELSSLRSE | | EAEDVGVYYCLQGTHQPYTF |
| | | DTAVYYCARGGYDGWD | | GQGTKVEIK |
| | | YAIDYWGQGTLVTVSS | | |
| Antibody | 1987 | QVQLVQSGAEVKKPGAS | 2093 | DVVMTQSPLSLPVTPGEPASI |
| 79 | | VKVSCKGSGYTFTSYWM | | SCRSSQSLAKYYGNTYLSWY |
| | | HWVRQAPGQRLEWIGEID | | LQKPGQSPQLLIYGISNRFSG |
| | | PSESNTNYNQKFKGRVTL | | VPDRFSGSGSGTDFTLKISRV |
| | | TVDISASTAYMELSSLRSE | | EAEDVGVYYCLQGTHQPYTF |
| | | DTAVYYCARGGYDGWD | | GQGTKVEIK |
| | | YAIDYWGQGTLVTVSS | | |
| Antibody | 1988 | QVQLVQSGAEVKKPGAS | 2094 | DVVMTQSPLSLPVTPGEPASI |
| 80 | | VKVSCKGSGYTFTSYWM | | SCRSSQSLAKSHGNTYLSWY |
| | | HWVRQAPGQRLEWIGEID | | LQKPGQSPQLLIYGISNRFSG |
| | | PSESNTNYNQKFKGRVTL | | VPDRFSGSGSGTDFTLKISRV |
| | | TVDISASTAYMELSSLRSE | | EAEDVGVYYCLQGTHQPYTF |
| | | DTAVYYCARGGYDGWD | | GQGTKVEIK |
| | | YAIDYWGQGTLVTVSS | | |
| Antibody | 1989 | QVQLVQSGAEVKKPGAS | 2095 | DVVMTQSPLSLPVTPGEPASI |
| 81 | | VKVSCKGSGYTFTSYWM | | SCRSSQSLAKSYGNTYLSWY |
| | | HWVRQAPGQRLEWIGEID | | LQKPGQSPQLLIYFISNRFSGV |
| | | PSESNTNYNQKFKGRVTL | | PDRFSGSGSGTDFTLKISRVE |
| | | TVDISASTAYMELSSLRSE | | AEDVGVYYCLQGTHQPYTFG |
| | | DTAVYYCARGGYDGWD | | QGTKVEIK |
| | | YAIDYWGQGTLVTVSS | | |
| Antibody | 1990 | QVQLVQSGAEVKKPGAS | 2096 | DVVMTQSPLSLPVTPGEPASI |
| 82 | | VKVSCKGSGYTFTSYWM | | SCRSSQSLAKSYGNTYLSWY |
| | | HWVRQAPGQRLEWIGEID | | LQKPGQSPQLLIYPISNRFSGV |
| | | PSESNTNYNQKFKGRVTL | | PDRFSGSGSGTDFTLKISRVE |
| | | TVDISASTAYMELSSLRSE | | AEDVGVYYCLQGTHQPYTFG |
| | | DTAVYYCARGGYDGWD | | QGTKVEIK |
| | | YAIDYWGQGTLVTVSS | | |
| Antibody | 1991 | QVQLVQSGAEVKKPGAS | 2097 | DVVMTQSPLSLPVTPGEPASI |
| 83 | | VKVSCKGSGYTFTSYWM | | SCRSSQSLAKSYGNTYLSWY |
| | | HWVRQAPGQRLEWIGEID | | LQKPGQSPQLLIYYISNRFSG |
| | | PSESNTNYNQKFKGRVTL | | VPDRFSGSGSGTDFTLKISRV |
| | | TVDISASTAYMELSSLRSE | | EAEDVGVYYCLQGTHQPYTF |
| | | DTAVYYCARGGYDGWD | | GQGTKVEIK |
| | | YAIDYWGQGTLVTVSS | | |
| Antibody | 1992 | QVQLVQSGAEVKKPGAS | 2098 | DVVMTQSPLSLPVTPGEPASI |
| 84 | | VKVSCKGSGYTFTSYWM | | SCRSSQSLAKSYGNTYLSWY |
| | | HWVRQAPGQRLEWIGEID | | LQKPGQSPQLLIYGISNRFSG |
| | | PSESNTNYNQKFKGRVTL | | VPDRFSGSGSGTDFTLKISRV |
| | | TVDISASTAYMELSSLRSE | | EAEDVGVYYCLQFTHQPYTF |
| | | DTAVYYCARGGYDGWD | | GQGTKVEIK |
| | | YAIDYWGQGTLVTVSS | | |
| Antibody | 1993 | QVQLVQSGAEVKKPGAS | 2099 | DVVMTQSPLSLPVTPGEPASI |
| 85 | | VKVSCKGSGYTFTSYWM | | SCRSSQSLAKSYGNTYLSWY |
| | | HWVRQAPGQRLEWIGEID | | LQKPGQSPQLLIYGISNRFSG |
| | | PSESNTNYNQKFKGRVTL | | VPDRFSGSGSGTDFTLKISRV |
| | | TVDISASTAYMELSSLRSE | | EAEDVGVYYCLQFTIQFYTFG |
| | | DTAVYYCARGGYDGWD | | QGTKVEIK |
| | | YAIDYWGQGTLVTVSS | | |
| Antibody | 1994 | QVQLVQSGAEVKKPGAS | 2100 | DVVMTQSPLSLPVTPGEPASI |
| 86 | | VKVSCKGSGYTFTSYWM | | SCRSSQSLAKSYGNTYLSWY |
| | | HWVRQAPGQRLEWIGEID | | LQKPGQSPQLLIYGISNRFSG |
| | | PSESNTNYNQKFKGRVTL | | VPDRFSGSGSGTDFTLKISRV |
| | | TVDISASTAYMELSSLRSE | | EAEDVGVYYCLQFTIQVYTF |
| | | DTAVYYCARGGYDGWD | | GQGTKVEIK |
| | | YAIDYWGQGTLVTVSS | | |
| Antibody | 1995 | QVQLVQSGAEVKKPGAS | 2101 | DVVMTQSPLSLPVTPGEPASI |
| 87 | | VKVSCKGSGYTFTSYWM | | SCRSSQSLAKSYGNTYLSWY |
| | | HWVRQAPGQRLEWIGEID | | LQKPGQSPQLLIYGISNRFSG |
| | | PSESNTNYNQKFKGRVTL | | VPDRFSGSGSGTDFTLKISRV |
| | | TVDISASTAYMELSSLRSE | | EAEDVGVYYCLQFTIQPYIFG |
| | | DTAVYYCARGGYDGWD | | QGTKVEIK |
| | | YAIDYWGQGTLVTVSS | | |
| Antibody | 1996 | QVQLVQSGAEVKKPGAS | 2102 | DVVMTQSPLSLPVTPGEPASI |
| 88 | | VKVSCKGSGYTFTSYWM | | SCRSSQSLAKSYGNTYLSWY |
| | | HWVRQAPGQRLEWIGEID | | LQKPGQSPQLLIYGISNRFSG |
| | | PSESNTNYNQKFKGRVTL | | VPDRFSGSGSGTDFTLKISRV |
| | | TVDISASTAYMELSSLRSE | | EAEDVGVYYCLQFTHQFYTF |
| | | DTAVYYCARGGYDGWD | | GQGTKVEIK |
| | | YAIDYWGQGTLVTVSS | | |
| Antibody | 1997 | QVQLVQSGAEVKKPGAS | 2103 | DVVMTQSPLSLPVTPGEPASI |
| 89 | | VKVSCKGSGYTFTSYWM | | SCRSSQSLAKSYGNTYLSWY |
| | | HWVRQAPGQRLEWIGEID | | LQKPGQSPQLLIYGISNRFSG |
| | | PSESNTNYNQKFKGRVTL | | VPDRFSGSGSGTDFTLKISRV |
| | | TVDISASTAYMELSSLRSE | | EAEDVGVYYCLQFTHQIYIFG |
| | | DTAVYYCARGGYDGWD | | QGTKVEIK |
| | | YAIDYWGQGTLVTVSS | | |
| Antibody | 1998 | QVQLVQSGAEVKKPGAS | 2104 | DVVMTQSPLSLPVTPGEPASI |
| 90 | | VKVSCKGSGYTFTSYWM | | SCRSSQSLAKSYGNTYLSWY |
| | | HWVRQAPGQRLEWIGEID | | LQKPGQSPQLLIYGISNRFSG |
| | | PSESNTNYNQKFKGRVTL | | VPDRFSGSGSGTDFTLKISRV |
| | | TVDISASTAYMELSSLRSE | | EAEDVGVYYCLQFTHQPYIF |
| | | DTAVYYCARGGYDGWD | | GQGTKVEIK |
| | | YAIDYWGQGTLVTVSS | | |
| Antibody | 1999 | QVQLVQSGAEVKKPGAS | 2105 | DVVMTQSPLSLPVTPGEPASI |
| 91 | | VKVSCKGSGYTFTSYWM | | SCRSSQSLAKSYGNTYLSWY |
| | | HWVRQAPGQRLEWIGEID | | LQKPGQSPQLLIYGISNRFSG |
| | | PSESNTNYNQKFKGRVTL | | VPDRFSGSGSGTDFTLKISRV |
| | | TVDISASTAYMELSSLRSE | | EAEDVGVYYCLQRTHQPYTF |
| | | DTAVYYCARGGYDGWD | | GQGTKVEIK |
| | | YAIDYWGQGTLVTVSS | | |
| Antibody | 2000 | QVQLVQSGAEVKKPGAS | 2106 | DVVMTQSPLSLPVTPGEPASI |
| 92 | | VKVSCKGSGYTFTSYWM | | SCRSSQSLAKSYGNTYLSWY |
| | | HWVRQAPGQRLEWIGEID | | LQKPGQSPQLLIYGISNRFSG |
| | | PSESNTNYNQKFKGRVTL | | VPDRFSGSGSGTDFTLKISRV |
| | | TVDISASTAYMELSSLRSE | | EAEDVGVYYCLQRTIQVYIFG |
| | | DTAVYYCARGGYDGWD | | QGTKVEIK |
| | | YAIDYWGQGTLVTVSS | | |
| Antibody | 2001 | QVQLVQSGAEVKKPGAS | 2107 | DVVMTQSPLSLPVTPGEPASI |
| 93 | | VKVSCKGSGYTFTSYWM | | SCRSSQSLAKSYGNTYLSWY |
| | | HWVRQAPGQRLEWIGEID | | LQKPGQSPQLLIYGISNRFSG |
| | | PSESNTNYNQKFKGRVTL | | VPDRFSGSGSGTDFTLKISRV |
| | | TVDISASTAYMELSSLRSE | | EAEDVGVYYCLQRTIQYYTF |
| | | DTAVYYCARGGYDGWD | | GQGTKVEIK |
| | | YAIDYWGQGTLVTVSS | | |
| Antibody | 2002 | QVQLVQSGAEVKKPGAS | 2108 | DVVMTQSPLSLPVTPGEPASI |
| 94 | | VKVSCKGSGYTFTSYWM | | SCRSSQSLAKSYGNTYLSWY |
| | | HWVRQAPGQRLEWIGEID | | LQKPGQSPQLLIYGISNRFSG |
| | | PSESNTNYNQKFKGRVTL | | VPDRFSGSGSGTDFTLKISRV |
| | | TVDISASTAYMELSSLRSE | | EAEDVGVYYCLQVTHQPYTF |
| | | DTAVYYCARGGYDGWD | | GQGTKVEIK |
| | | YAIDYWGQGTLVTVSS | | |
| Antibody | 2003 | QVQLVQSGAEVKKPGAS | 2109 | DVVMTQSPLSLPVTPGEPASI |
| 95 | | VKVSCKGSGYTFTSYWM | | SCRSSQSLAKSYGNTYLSWY |
| | | HWVRQAPGQRLEWIGEID | | LQKPGQSPQLLIYGISNRFSG |
| | | PSESNTNYNQKFKGRVTL | | VPDRFSGSGSGTDFTLKISRV |
| | | TVDISASTAYMELSSLRSE | | EAEDVGVYYCLQWTHQPYT |
| | | DTAVYYCARGGYDGWD | | FGQGTKVEIK |
| | | YAIDYWGQGTLVTVSS | | |
| Antibody | 2004 | QVQLVQSGAEVKKPGAS | 2110 | DVVMTQSPLSLPVTPGEPASI |
| 96 | | VKVSCKGSGYTFTSYWM | | SCRSSQSLAKSYGNTYLSWY |
| | | HWVRQAPGQRLEWIGEID | | LQKPGQSPQLLIYGISNRFSG |
| | | PSESNTNYNQKFKGRVTL | | VPDRFSGSGSGTDFTLKISRV |
| | | TVDISASTAYMELSSLRSE | | EAEDVGVYYCLQYTHQPYTF |
| | | DTAVYYCARGGYDGWD | | GQGTKVEIK |
| | | YAIDYWGQGTLVTVSS | | |
| Antibody | 2005 | QVQLVQSGAEVKKPGAS | 2111 | DVVMTQSPLSLPVTPGEPASI |
| 97 | | VKVSCKGSGYTFTSYWM | | SCRSSQSLAKSYGNTYLSWY |
| | | HWVRQAPGQRLEWIGEID | | LQKPGQSPQLLIYGISNRFSG |
| | | PSESNTNYNQKFKGRVTL | | VPDRFSGSGSGTDFTLKISRV |
| | | TVDISASTAYMELSSLRSE | | EAEDVGVYYCLQYTIQFYIFG |
| | | DTAVYYCARGGYDGWD | | QGTKVEIK |
| | | YAIDYWGQGTLVTVSS | | |
| Antibody | 2006 | QVQLVQSGAEVKKPGAS | 2112 | DVVMTQSPLSLPVTPGEPASI |
| 98 | | VKVSCKGSGYTFTSYWM | | SCRSSQSLAKSYGNTYLSWY |
| | | HWVRQAPGQRLEWIGEID | | LQKPGQSPQLLIYGISNRFSG |
| | | PSESNTNYNQKFKGRVTL | | VPDRFSGSGSGTDFTLKISRV |
| | | TVDISASTAYMELSSLRSE | | EAEDVGVYYCLQYTHQFYIF |
| | | DTAVYYCARGGYDGWD | | GQGTKVEIK |
| | | YAIDYWGQGTLVTVSS | | |
| Antibody | 2007 | QVQLVQSGAEVKKPGAS | 2113 | DVVMTQSPLSLPVTPGEPASI |
| 99 | | VKVSCKGSGYTFTSYWM | | SCRSSQSLAKSYGNTYLSWY |
| | | HWVRQAPGQRLEWIGEID | | LQKPGQSPQLLIYGISNRFSG |
| | | PSESNTNYNQKFKGRVTL | | VPDRFSGSGSGTDFTLKISRV |
| | | TVDISASTAYMELSSLRSE | | EAEDVGVYYCLQGTIQPYTF |
| | | DTAVYYCARGGYDGWD | | GQGTKVEIK |
| | | YAIDYWGQGTLVTVSS | | |
| Antibody | 2008 | QVQLVQSGAEVKKPGAS | 2114 | DVVMTQSPLSLPVTPGEPASI |
| 100 | | VKVSCKGSGYTFTSYWM | | SCRSSQSLAKSYGNTYLSWY |
| | | HWVRQAPGQRLEWIGEID | | LQKPGQSPQLLIYGISNRFSG |
| | | PSESNTNYNQKFKGRVTL | | VPDRFSGSGSGTDFTLKISRV |
| | | TVDISASTAYMELSSLRSE | | EAEDVGVYYCLQGTWQPYT |
| | | DTAVYYCARGGYDGWD | | FGQGTKVEIK |
| | | YAIDYWGQGTLVTVSS | | |
| Antibody | 2009 | QVQLVQSGAEVKKPGAS | 2115 | DVVMTQSPLSLPVTPGEPASI |
| 101 | | VKVSCKGSGYTFTSYWM | | SCRSSQSLAKSYGNTYLSWY |
| | | HWVRQAPGQRLEWIGEID | | LQKPGQSPQLLIYGISNRFSG |
| | | PSESNTNYNQKFKGRVTL | | VPDRFSGSGSGTDFTLKISRV |
| | | TVDISASTAYMELSSLRSE | | EAEDVGVYYCLQGTHQFYTF |
| | | DTAVYYCARGGYDGWD | | GQGTKVEIK |
| | | YAIDYWGQGTLVTVSS | | |
| Antibody | 2010 | QVQLVQSGAEVKKPGAS | 2116 | DVVMTQSPLSLPVTPGEPASI |
| 102 | | VKVSCKGSGYTFTSYWM | | SCRSSQSLAKSYGNTYLSWY |
| | | HWVRQAPGQRLEWIGEID | | LQKPGQSPQLLIYGISNRFSG |
| | | PSESNTNYNQKFKGRVTL | | VPDRFSGSGSGTDFTLKISRV |
| | | TVDISASTAYMELSSLRSE | | EAEDVGVYYCLQGTHQIYTF |
| | | DTAVYYCARGGYDGWD | | GQGTKVEIK |
| | | YAIDYWGQGTLVTVSS | | |
| Antibody | 2011 | QVQLVQSGAEVKKPGAS | 2117 | DVVMTQSPLSLPVTPGEPASI |
| 103 | | VKVSCKGSGYTFTSYWM | | SCRSSQSLAKSYGNTYLSWY |
| | | HWVRQAPGQRLEWIGEID | | LQKPGQSPQLLIYGISNRFSG |
| | | PSESNTNYNQKFKGRVTL | | VPDRFSGSGSGTDFTLKISRV |
| | | TVDISASTAYMELSSLRSE | | EAEDVGVYYCLQGTHQVYTF |
| | | DTAVYYCARGGYDGWD | | GQGTKVEIK |
| | | YAIDYWGQGTLVTVSS | | |
| Antibody | 2012 | QVQLVQSGAEVKKPGAS | 2118 | DVVMTQSPLSLPVTPGEPASI |
| 104 | | VKVSCKGSGYTFTSYWM | | SCRSSQSLAKSYGNTYLSWY |
| | | HWVRQAPGQRLEWIGEID | | LQKPGQSPQLLIYGISNRFSG |
| | | PSESNTNYNQKFKGRVTL | | VPDRFSGSGSGTDFTLKISRV |
| | | TVDISASTAYMELSSLRSE | | EAEDVGVYYCLQGTHQYYTF |
| | | DTAVYYCARGGYDGWD | | GQGTKVEIK |
| | | YAIDYWGQGTLVTVSS | | |
| Antibody | 2013 | QVQLVQSGAEVKKPGAS | 2119 | DVVMTQSPLSLPVTPGEPASI |
| 105 | | VKVSCKGSGYTFTSYWM | | SCRSSQSLAKSYGNTYLSWY |
| | | HWVRQAPGQRLEWIGEID | | LQKPGQSPQLLIYGISNRFSG |
| | | PSESNTNYNQKFKGRVTL | | VPDRFSGSGSGTDFTLKISRV |
| | | TVDISASTAYMELSSLRSE | | EAEDVGVYYCLQGTHQPYIF |
| | | DTAVYYCARGGYDGWD | | GQGTKVEIK |
| | | YAIDYWGQGTLVTVSS | | |
| Antibody | 2014 | QVQLVQSGAEVKKPGAS | 2120 | DVVMTQSPLSLPVTPGEPASI |
| 106 | | VKVSCKGSGYTFTSYWM | | SCRSSQSLAKSYGNTYLSWY |
| | | HWVRQAPGQRLEWIGEID | | LQKPGQSPQLLIYGISNRFSG |
| | | PSESNTNYNQKFKGRVTL | | VPDRFSGSGSGTDFTLKISRV |
| | | TVDISASTAYMELSSLRSE | | EAEDVGVYYCLQGTHQPYRF |
| | | DTAVYYCARGGYDGWD | | GQGTKVEIK |
| | | YAIDYWGQGTLVTVSS | | |

In some embodiments, the α4β7 integrin binding protein comprises a heavy chain variable region (VH) comprising an amino acid sequence having at least 80% sequence identity, at least 85% identity, at least 90% identity, at least 95% identity, at least 96% identity, at least 97% identity, at least 98% identity, at least 99% identity or 100% with an amino acid sequence set out in Table 2.

In some embodiments, the α4β7 integrin binding protein comprises a heavy chain variable region (VH) that comprises an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identical to the heavy chain variable region (VH) of an α4β7 integrin binding protein disclosed in Table 2, and a light chain variable region (VL) that comprises an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identical to the light chain variable region (VL) of the same α4β7 integrin binding protein disclosed in Table 2.

### Fc Modifications

Described herein are α4β7 integrin binding proteins comprising modified Fc regions. Unless otherwise specified herein, numbering of amino acid residues in the Fc region or constant region is according to the EU numbering system, also called the EU index, as described in Kabat et al, Sequences of Proteins of Immunological Interest, 5th Ed. Public Health Service, National Institutes of Health, Bethesda, MD, 1991.

In some embodiments, the α4β7 integrin binding proteins comprise a modified Fc comprising one or more modifications. In some embodiments, the one or more modifications are located in a Fc from IgG1 (e.g., human IgG1 (hIgG1). In some embodiments, the one or more modifications are located in a Fc from IgG4 (e.g., human IgG4 (hIgG4). In some embodiments, the one or more modifications are located in a Fc from IgG2. In some embodiments, the one or more modifications promote selective binding of Fc-gamma receptors.

Amino acid sequences of exemplary Fc sequences are provided in Table 3.

TABLE 3
Fc Sequences

| | SEQ | |
|---|---|---|
| | ID | |
| Name | NO | Fc Sequence |
| hIgG1 | 2121 | ASTKGPSVFPLAPSSKSTSGGTAALGCLVKDYFPEPVTVSWNSGALT |
| | | SGVHTFPAVLQSSGLYSLSSVVTVPSSSLGTQTYICNVNHKPSNTKV |
| | | DKKVEPKSCDKTHTCPPCPAPELLGGPSVFLFPPKPKDTLMISRTPEV |
| | | TCVVVDVSHEDPEVKFNWYVDGVEVHNAKTKPREEQYNSTYRVVS |
| | | VLTVLHQDWLNGKEYKCKVSNKALPAPIEKTISKAKGQPREPQVYT |
| | | LPPSRDELTKNQVSLTCLVKGFYPSDIAVEWESNGQPENNYKTTPPV |
| | | LDSDGSFFLYSKLTVDKSRWQQGNVFSCSVMHEALHNHYTQKSLSL |
| | | SPG |
| hIgG4 | 2122 | ASTKGPSVFPLAPCSRSTSESTAALGCLVKDYFPEPVTVSWNSGALT |
| | | SGVHTFPAVLQSSGLYSLSSVVTVPSSSLGTKTYTCNVDHKPSNTKV |
| | | DKRVESKYGPPCPSCPAPEFLGGPSVFLFPPKPKDTLMISRTPEVTCV |
| | | VVDVSQEDPEVQFNWYVDGVEVHNAKTKPREEQFNSTYRVVSVLT |
| | | VLHQDWLNGKEYKCKVSNKGLPSSIEKTISKAKGQPREPQVYTLPPS |
| | | QEEMTKNQVSLTCLVKGFYPSDIAVEWESNGQPENNYKTTPPVLDS |
| | | DGSFFLYSRLTVDKSRWQEGNVFSCSVMHEALHNHYTQKSLSLSLG |
| | | K |
| IgG2 | 2123 | ASTKGPSVFPLAPCSRSTSESTAALGCLVKDYFPEPVTVSWNSGALT |
| | | SGVHTFPAVLQSSGLYSLSSVVTVPSSNFGTQTYTCNVDHKPSNTKV |
| | | DKTVERKCCVECPPCPAPPVAGPSVFLFPPKPKDTLMISRTPEVTCVV |
| | | VDVSHEDPEVQFNWYVDGVEVHNAKTKPREEQFNSTFRVVSVLTV |
| | | VHQDWLNGKEYKCKVSNKGLPAPIEKTISKTKGQPREPQVYTLPPSR |
| | | EEMTKNQVSLTCLVKGFYPSDIAVEWESNGQPENNYKTTPPMLDSD |
| | | GSFFLYSKLTVDKSRWQQGNVFSCSVMHEALHNHYTQKSLSLSP |
| IgG4-SP | 2124 | ASTKGPSVFPLAPCSRSTSESTAALGCLVKDYFPEPVTVSWNSGALT |
| | | SGVHTFPAVLQSSGLYSLSSVVTVPSSSLGTKTYTCNVDHKPSNTKV |
| | | DKRVESKYGPPCPPCPAPEFLGGPSVFLFPPKPKDTLMISRTPEVTCV |
| | | VVDVSQEDPEVQFNWYVDGVEVHNAKTKPREEQFNSTYRVVSVLT |
| | | VLHQDWLNGKEYKCKVSNKGLPSSIEKTISKAKGQPREPQVYTLPPS |
| | | QEEMTKNQVSLTCLVKGFYPSDIAVEWESNGQPENNYKTTPPVLDS |
| | | DGSFFLYSRLTVDKSRWQEGNVFSCSVMHEALHNHYTQKSLSLSLG |
| | | K |
| IgG4-SPLE | 2125 | ASTKGPSVFPLAPCSRSTSESTAALGCLVKDYFPEPVTVSWNSGALT |
| | | SGVHTFPAVLQSSGLYSLSSVVTVPSSSLGTKTYTCNVDHKPSNTKV |
| | | DKRVESKYGPPCPPCPAPEELGGPSVFLFPPKPKDTLMISRTPEVTCV |
| | | VVDVSQEDPEVQFNWYVDGVEVHNAKTKPREEQFNSTYRVVSVLT |
| | | VLHQDWLNGKEYKCKVSNKGLPSSIEKTISKAKGQPREPQVYTLPPS |
| | | QEEMTKNQVSLTCLVKGFYPSDIAVEWESNGQPENNYKTTPPVLDS |
| | | DGSFFLYSRLTVDKSRWQEGNVFSCSVMHEALHNHYTQKSLSLSLG |
| | | K |
| hIgG1-N297A | 2126 | ASTKGPSVFPLAPSSKSTSGGTAALGCLVKDYFPEPVTVSWNSGALT |
| | | SGVHTFPAVLQSSGLYSLSSVVTVPSSSLGTQTYICNVNHKPSNTKV |
| | | DKKVEPKSCDKTHTCPPCPAPELLGGPSVFLFPPKPKDTLMISRTPEV |
| | | TCVVVDVSHEDPEVKFNWYVDGVEVHNAKTKPREEQYASTYRVVS |
| | | VLTVLHQDWLNGKEYKCKVSNKALPAPIEKTISKAKGQPREPQVYT |
| | | LPPSRDELTKNQVSLTCLVKGFYPSDIAVEWESNGQPENNYKTTPPV |
| | | LDSDGSFFLYSKLTVDKSRWQQGNVFSCSVMHEALHNHYTQKSLSL |
| | | SPG |
| hIgG1-D265A | 2127 | ASTKGPSVFPLAPSSKSTSGGTAALGCLVKDYFPEPVTVSWNSGALT |
| | | SGVHTFPAVLQSSGLYSLSSVVTVPSSSLGTQTYICNVNHKPSNTKV |
| | | DKKVEPKSCDKTHTCPPCPAPELLGGPSVFLFPPKPKDTLMISRTPEV |
| | | TCVVVAVSHEDPEVKFNWYVDGVEVHNAKTKPREEQYNSTYRVVS |
| | | VLTVLHQDWLNGKEYKCKVSNKALPAPIEKTISKAKGQPREPQVYT |
| | | LPPSRDELTKNQVSLTCLVKGFYPSDIAVEWESNGQPENNYKTTPPV |
| | | LDSDGSFFLYSKLTVDKSRWQQGNVFSCSVMHEALHNHYTQKSLSL |
| | | SPG |
| hIgG1-LALA | 2128 | ASTKGPSVFPLAPSSKSTSGGTAALGCLVKDYFPEPVTVSWNSGALT |
| | | SGVHTFPAVLQSSGLYSLSSVVTVPSSSLGTQTYICNVNHKPSNTKV |
| | | DKKVEPKSCDKTHTCPPCPAPEAAGGPSVFLFPPKPKDTLMISRTPEV |
| | | TCVVVDVSHEDPEVKFNWYVDGVEVHNAKTKPREEQYNSTYRVVS |
| | | VLTVLHQDWLNGKEYKCKVSNKALPAPIEKTISKAKGQPREPQVYT |
| | | LPPSRDELTKNQVSLTCLVKGFYPSDIAVEWESNGQPENNYKTTPPV |
| | | LDSDGSFFLYSKLTVDKSRWQQGNVFSCSVMHEALHNHYTQKSLSL |
| | | SPG |
| hIgG1-LAGA | 2129 | ASTKGPSVFPLAPSSKSTSGGTAALGCLVKDYFPEPVTVSWNSGALT |
| | | SGVHTFPAVLQSSGLYSLSSVVTVPSSSLGTQTYICNVNHKPSNTKV |
| | | DKKVEPKSCDKTHTCPPCPAPELAGAPSVFLFPPKPKDTLMISRTPEV |
| | | TCVVVDVSHEDPEVKFNWYVDGVEVHNAKTKPREEQYNSTYRVVS |
| | | VLTVLHQDWLNGKEYKCKVSNKALPAPIEKTISKAKGQPREPQVYT |
| | | LPPSRDELTKNQVSLTCLVKGFYPSDIAVEWESNGQPENNYKTTPPV |
| | | LDSDGSFFLYSKLTVDKSRWQQGNVFSCSVMHEALHNHYTQKSLSL |
| | | SPG |
| hIgG1- | 2130 | ASTKGPSVFPLAPSSKSTSGGTAALGCLVKDYFPEPVTVSWNSGALT |
| LALAGA | | SGVHTFPAVLQSSGLYSLSSVVTVPSSSLGTQTYICNVNHKPSNTKV |
| | | DKKVEPKSCDKTHTCPPCPAPEAAGAPSVFLFPPKPKDTLMISRTPEV |
| | | TCVVVDVSHEDPEVKFNWYVDGVEVHNAKTKPREEQYNSTYRVVS |
| | | VLTVLHQDWLNGKEYKCKVSNKALPAPIEKTISKAKGQPREPQVYT |
| | | LPPSRDELTKNQVSLTCLVKGFYPSDIAVEWESNGQPENNYKTTPPV |
| | | LDSDGSFFLYSKLTVDKSRWQQGNVFSCSVMHEALHNHYTQKSLSL |
| | | SPG |
| hIgG1- | 2131 | ASTKGPSVFPLAPSSKSTSGGTAALGCLVKDYFPEPVTVSWNSGALT |
| LALAPG | | SGVHTFPAVLQSSGLYSLSSVVTVPSSSLGTQTYICNVNHKPSNTKV |
| | | DKKVEPKSCDKTHTCPPCPAPEAAGGPSVFLFPPKPKDTLMISRTPEV |
| | | TCVVVDVSHEDPEVKFNWYVDGVEVHNAKTKPREEQYNSTYRVVS |
| | | VLTVLHQDWLNGKEYKCKVSNKALGAPIEKTISKAKGQPREPQVYT |
| | | LPPSRDELTKNQVSLTCLVKGFYPSDIAVEWESNGQPENNYKTTPPV |
| | | LDSDGSFFLYSKLTVDKSRWQQGNVFSCSVMHEALHNHYTQKSLSL |
| | | SPG |
| hIgG1-YTE | 2132 | ASTKGPSVFPLAPSSKSTSGGTAALGCLVKDYFPEPVTVSWNSGALT |
| | | SGVHTFPAVLQSSGLYSLSSVVTVPSSSLGTQTYICNVNHKPSNTKV |
| | | DKKVEPKSCDKTHTCPPCPAPELLGGPSVFLFPPKPKDTLYITREPEV |
| | | TCVVVDVSHEDPEVKFNWYVDGVEVHNAKTKPREEQYNSTYRVVS |
| | | VLTVLHQDWLNGKEYKCKVSNKALPAPIEKTISKAKGQPREPQVYT |
| | | LPPSRDELTKNQVSLTCLVKGFYPSDIAVEWESNGQPENNYKTTPPV |
| | | LDSDGSFFLYSKLTVDKSRWQQGNVFSCSVMHEALHNHYTQKSLSL |
| | | SPG |
| hIgG1- | 2133 | ASTKGPSVFPLAPSSKSTSGGTAALGCLVKDYFPEPVTVSWNSGALT |
| N297A/YTE | | SGVHTFPAVLQSSGLYSLSSVVTVPSSSLGTQTYICNVNHKPSNTKV |
| | | DKKVEPKSCDKTHTCPPCPAPELLGGPSVFLFPPKPKDTLYITREPEV |
| | | TCVVVDVSHEDPEVKFNWYVDGVEVHNAKTKPREEQYASTYRVVS |
| | | VLTVLHQDWLNGKEYKCKVSNKALPAPIEKTISKAKGQPREPQVYT |
| | | LPPSRDELTKNQVSLTCLVKGFYPSDIAVEWESNGQPENNYKTTPPV |
| | | LDSDGSFFLYSKLTVDKSRWQQGNVFSCSVMHEALHNHYTQKSLSL |
| | | SPG |
| hIgG1- | 2134 | ASTKGPSVFPLAPSSKSTSGGTAALGCLVKDYFPEPVTVSWNSGALT |
| D265A/YTE | | SGVHTFPAVLQSSGLYSLSSVVTVPSSSLGTQTYICNVNHKPSNTKV |
| | | DKKVEPKSCDKTHTCPPCPAPELLGGPSVFLFPPKPKDTLYITREPEV |
| | | TCVVVAVSHEDPEVKFNWYVDGVEVHNAKTKPREEQYNSTYRVVS |
| | | VLTVLHQDWLNGKEYKCKVSNKALPAPIEKTISKAKGQPREPQVYT |
| | | LPPSRDELTKNQVSLTCLVKGFYPSDIAVEWESNGQPENNYKTTPPV |
| | | LDSDGSFFLYSKLTVDKSRWQQGNVFSCSVMHEALHNHYTQKSLSL |
| | | SPG |
| hIgG1- | 2135 | ASTKGPSVFPLAPSSKSTSGGTAALGCLVKDYFPEPVTVSWNSGALT |
| LALA/YTE | | SGVHTFPAVLQSSGLYSLSSVVTVPSSSLGTQTYICNVNHKPSNTKV |
| | | DKKVEPKSCDKTHTCPPCPAPEAAGGPSVFLFPPKPKDTLYITREPEV |
| | | TCVVVDVSHEDPEVKFNWYVDGVEVHNAKTKPREEQYNSTYRVVS |
| | | VLTVLHQDWLNGKEYKCKVSNKALPAPIEKTISKAKGQPREPQVYT |
| | | LPPSRDELTKNQVSLTCLVKGFYPSDIAVEWESNGQPENNYKTTPPV |
| | | LDSDGSFFLYSKLTVDKSRWQQGNVFSCSVMHEALHNHYTQKSLSL |
| | | SPG |
| hIgG1- | 2136 | ASTKGPSVFPLAPSSKSTSGGTAALGCLVKDYFPEPVTVSWNSGALT |
| LAGA/YTE | | SGVHTFPAVLQSSGLYSLSSVVTVPSSSLGTQTYICNVNHKPSNTKV |
| | | DKKVEPKSCDKTHTCPPCPAPELAGAPSVFLFPPKPKDTLYITREPEV |
| | | TCVVVDVSHEDPEVKFNWYVDGVEVHNAKTKPREEQYNSTYRVVS |
| | | VLTVLHQDWLNGKEYKCKVSNKALPAPIEKTISKAKGQPREPQVYT |
| | | LPPSRDELTKNQVSLTCLVKGFYPSDIAVEWESNGQPENNYKTTPPV |
| | | LDSDGSFFLYSKLTVDKSRWQQGNVFSCSVMHEALHNHYTQKSLSL |
| | | SPG |
| hIgG1- | 2137 | ASTKGPSVFPLAPSSKSTSGGTAALGCLVKDYFPEPVTVSWNSGALT |
| LALAGA/YTE | | SGVHTFPAVLQSSGLYSLSSVVTVPSSSLGTQTYICNVNHKPSNTKV |
| | | DKKVEPKSCDKTHTCPPCPAPEAAGAPSVFLFPPKPKDTLYITREPEV |
| | | TCVVVDVSHEDPEVKFNWYVDGVEVHNAKTKPREEQYNSTYRVVS |
| | | VLTVLHQDWLNGKEYKCKVSNKALPAPIEKTISKAKGQPREPQVYT |
| | | LPPSRDELTKNQVSLTCLVKGFYPSDIAVEWESNGQPENNYKTTPPV |
| | | LDSDGSFFLYSKLTVDKSRWQQGNVFSCSVMHEALHNHYTQKSLSL |
| | | SPG |
| hIgG1- | 2138 | ASTKGPSVFPLAPSSKSTSGGTAALGCLVKDYFPEPVTVSWNSGALT |
| LALAPG/YTE | | SGVHTFPAVLQSSGLYSLSSVVTVPSSSLGTQTYICNVNHKPSNTKV |
| | | DKKVEPKSCDKTHTCPPCPAPEAAGGPSVFLFPPKPKDTLYITREPEV |
| | | TCVVVDVSHEDPEVKFNWYVDGVEVHNAKTKPREEQYNSTYRVVS |
| | | VLTVLHQDWLNGKEYKCKVSNKALGAPIEKTISKAKGQPREPQVYT |
| | | LPPSRDELTKNQVSLTCLVKGFYPSDIAVEWESNGQPENNYKTTPPV |
| | | LDSDGSFFLYSKLTVDKSRWQQGNVFSCSVMHEALHNHYTQKSLSL |
| | | SPG |
| hIgG1-LS | 2139 | ASTKGPSVFPLAPSSKSTSGGTAALGCLVKDYFPEPVTVSWNSGALT |
| | | SGVHTFPAVLQSSGLYSLSSVVTVPSSSLGTQTYICNVNHKPSNTKV |
| | | DKKVEPKSCDKTHTCPPCPAPELLGGPSVFLFPPKPKDTLMISRTPEV |
| | | TCVVVDVSHEDPEVKFNWYVDGVEVHNAKTKPREEQYNSTYRVVS |
| | | VLTVLHQDWLNGKEYKCKVSNKALPAPIEKTISKAKGQPREPQVYT |
| | | LPPSRDELTKNQVSLTCLVKGFYPSDIAVEWESNGQPENNYKTTPPV |
| | | LDSDGSFFLYSKLTVDKSRWQQGNVFSCSVLHEALHSHYTQKSLSLS |
| | | PG |
| hIgG1- | 2140 | ASTKGPSVFPLAPSSKSTSGGTAALGCLVKDYFPEPVTVSWNSGALT |
| N297A/LS | | SGVHTFPAVLQSSGLYSLSSVVTVPSSSLGTQTYICNVNHKPSNTKV |
| | | DKKVEPKSCDKTHTCPPCPAPELLGGPSVFLFPPKPKDTLMISRTPEV |
| | | TCVVVDVSHEDPEVKFNWYVDGVEVHNAKTKPREEQYASTYRVVS |
| | | VLTVLHQDWLNGKEYKCKVSNKALPAPIEKTISKAKGQPREPQVYT |
| | | LPPSRDELTKNQVSLTCLVKGFYPSDIAVEWESNGQPENNYKTTPPV |
| | | LDSDGSFFLYSKLTVDKSRWQQGNVFSCSVLHEALHSHYTQKSLSLS |
| | | PG |
| hIgG1- | 2141 | ASTKGPSVFPLAPSSKSTSGGTAALGCLVKDYFPEPVTVSWNSGALT |
| D265A/LS | | SGVHTFPAVLQSSGLYSLSSVVTVPSSSLGTQTYICNVNHKPSNTKV |
| | | DKKVEPKSCDKTHTCPPCPAPELLGGPSVFLFPPKPKDTLMISRTPEV |
| | | TCVVVAVSHEDPEVKFNWYVDGVEVHNAKTKPREEQYNSTYRVVS |
| | | VLTVLHQDWLNGKEYKCKVSNKALPAPIEKTISKAKGQPREPQVYT |
| | | LPPSRDELTKNQVSLTCLVKGFYPSDIAVEWESNGQPENNYKTTPPV |
| | | LDSDGSFFLYSKLTVDKSRWQQGNVFSCSVLHEALHSHYTQKSLSLS |
| | | PG |
| hIgG1- | 2142 | ASTKGPSVFPLAPSSKSTSGGTAALGCLVKDYFPEPVTVSWNSGALT |
| LALA/LS | | SGVHTFPAVLQSSGLYSLSSVVTVPSSSLGTQTYICNVNHKPSNTKV |
| | | DKKVEPKSCDKTHTCPPCPAPEAAGGPSVFLFPPKPKDTLMISRTPEV |
| | | TCVVVDVSHEDPEVKFNWYVDGVEVHNAKTKPREEQYNSTYRVVS |
| | | VLTVLHQDWLNGKEYKCKVSNKALPAPIEKTISKAKGQPREPQVYT |
| | | LPPSRDELTKNQVSLTCLVKGFYPSDIAVEWESNGQPENNYKTTPPV |
| | | LDSDGSFFLYSKLTVDKSRWQQGNVFSCSVLHEALHSHYTQKSLSLS |
| | | PG |
| hIgG1- | 2143 | ASTKGPSVFPLAPSSKSTSGGTAALGCLVKDYFPEPVTVSWNSGALT |
| LAGA/LS | | SGVHTFPAVLQSSGLYSLSSVVTVPSSSLGTQTYICNVNHKPSNTKV |
| | | DKKVEPKSCDKTHTCPPCPAPELAGAPSVFLFPPKPKDTLMISRTPEV |
| | | TCVVVDVSHEDPEVKFNWYVDGVEVHNAKTKPREEQYNSTYRVVS |
| | | VLTVLHQDWLNGKEYKCKVSNKALPAPIEKTISKAKGQPREPQVYT |
| | | LPPSRDELTKNQVSLTCLVKGFYPSDIAVEWESNGQPENNYKTTPPV |
| | | LDSDGSFFLYSKLTVDKSRWQQGNVFSCSVLHEALHSHYTQKSLSLS |
| | | PG |
| hIgG1- | 2144 | ASTKGPSVFPLAPSSKSTSGGTAALGCLVKDYFPEPVTVSWNSGALT |
| LALAGA/LS | | SGVHTFPAVLQSSGLYSLSSVVTVPSSSLGTQTYICNVNHKPSNTKV |
| | | DKKVEPKSCDKTHTCPPCPAPEAAGAPSVFLFPPKPKDTLMISRTPEV |
| | | TCVVVDVSHEDPEVKFNWYVDGVEVHNAKTKPREEQYNSTYRVVS |
| | | VLTVLHQDWLNGKEYKCKVSNKALPAPIEKTISKAKGQPREPQVYT |
| | | LPPSRDELTKNQVSLTCLVKGFYPSDIAVEWESNGQPENNYKTTPPV |
| | | LDSDGSFFLYSKLTVDKSRWQQGNVFSCSVLHEALHSHYTQKSLSLS |
| | | PG |
| hIgG1- | 2145 | ASTKGPSVFPLAPSSKSTSGGTAALGCLVKDYFPEPVTVSWNSGALT |
| LALAPG/LS | | SGVHTFPAVLQSSGLYSLSSVVTVPSSSLGTQTYICNVNHKPSNTKV |
| | | DKKVEPKSCDKTHTCPPCPAPEAAGGPSVFLFPPKPKDTLMISRTPEV |
| | | TCVVVDVSHEDPEVKFNWYVDGVEVHNAKTKPREEQYNSTYRVVS |
| | | VLTVLHQDWLNGKEYKCKVSNKALGAPIEKTISKAKGQPREPQVYT |
| | | LPPSRDELTKNQVSLTCLVKGFYPSDIAVEWESNGQPENNYKTTPPV |
| | | LDSDGSFFLYSKLTVDKSRWQQGNVFSCSVLHEALHSHYTQKSLSLS |
| | | PG |
| hIgG1-DHS | 2146 | ASTKGPSVFPLAPSSKSTSGGTAALGCLVKDYFPEPVTVSWNSGALT |
| | | SGVHTFPAVLQSSGLYSLSSVVTVPSSSLGTQTYICNVNHKPSNTKV |
| | | DKKVEPKSCDKTHTCPPCPAPELLGGPSVFLFPPKPKDTLMISRTPEV |
| | | TCVVVDVSHEDPEVKFNWYVDGVEVHNAKTKPREEQYNSTYRVVS |
| | | VLTVDHHDWLNGKEYKCKVSNKALPAPIEKTISKAKGQPREPQVYT |
| | | LPPSRDELTKNQVSLTCLVKGFYPSDIAVEWESNGQPENNYKTTPPV |
| | | LDSDGSFFLYSKLTVDKSRWQQGNVFSCSVMHEALHSHYTQKSLSL |
| | | SPG |
| hIgG1- | 2147 | ASTKGPSVFPLAPSSKSTSGGTAALGCLVKDYFPEPVTVSWNSGALT |
| N297A/DHS | | SGVHTFPAVLQSSGLYSLSSVVTVPSSSLGTQTYICNVNHKPSNTKV |
| | | DKKVEPKSCDKTHTCPPCPAPELLGGPSVFLFPPKPKDTLMISRTPEV |
| | | TCVVVDVSHEDPEVKFNWYVDGVEVHNAKTKPREEQYASTYRVVS |
| | | VLTVDHHDWLNGKEYKCKVSNKALPAPIEKTISKAKGQPREPQVYT |
| | | LPPSRDELTKNQVSLTCLVKGFYPSDIAVEWESNGQPENNYKTTPPV |
| | | LDSDGSFFLYSKLTVDKSRWQQGNVFSCSVMHEALHSHYTQKSLSL |
| | | SPG |
| hIgG1- | 2148 | ASTKGPSVFPLAPSSKSTSGGTAALGCLVKDYFPEPVTVSWNSGALT |
| D265A/DHS | | SGVHTFPAVLQSSGLYSLSSVVTVPSSSLGTQTYICNVNHKPSNTKV |
| | | DKKVEPKSCDKTHTCPPCPAPELLGGPSVFLFPPKPKDTLMISRTPEV |
| | | TCVVVAVSHEDPEVKFNWYVDGVEVHNAKTKPREEQYNSTYRVVS |
| | | VLTVDHHDWLNGKEYKCKVSNKALPAPIEKTISKAKGQPREPQVYT |
| | | LPPSRDELTKNQVSLTCLVKGFYPSDIAVEWESNGQPENNYKTTPPV |
| | | LDSDGSFFLYSKLTVDKSRWQQGNVFSCSVMHEALHSHYTQKSLSL |
| | | SPG |
| hIgG1- | 2149 | ASTKGPSVFPLAPSSKSTSGGTAALGCLVKDYFPEPVTVSWNSGALT |
| LALA/DHS | | SGVHTFPAVLQSSGLYSLSSVVTVPSSSLGTQTYICNVNHKPSNTKV |
| | | DKKVEPKSCDKTHTCPPCPAPEAAGGPSVFLFPPKPKDTLMISRTPEV |
| | | TCVVVDVSHEDPEVKFNWYVDGVEVHNAKTKPREEQYNSTYRVVS |
| | | VLTVDHHDWLNGKEYKCKVSNKALPAPIEKTISKAKGQPREPQVYT |
| | | LPPSRDELTKNQVSLTCLVKGFYPSDIAVEWESNGQPENNYKTTPPV |
| | | LDSDGSFFLYSKLTVDKSRWQQGNVFSCSVMHEALHSHYTQKSLSL |
| | | SPG |
| hIgG1- | 2150 | ASTKGPSVFPLAPSSKSTSGGTAALGCLVKDYFPEPVTVSWNSGALT |
| LAGA/DHS | | SGVHTFPAVLQSSGLYSLSSVVTVPSSSLGTQTYICNVNHKPSNTKV |
| | | DKKVEPKSCDKTHTCPPCPAPELAGAPSVFLFPPKPKDTLMISRTPEV |
| | | TCVVVDVSHEDPEVKFNWYVDGVEVHNAKTKPREEQYNSTYRVVS |
| | | VLTVDHHDWLNGKEYKCKVSNKALPAPIEKTISKAKGQPREPQVYT |
| | | LPPSRDELTKNQVSLTCLVKGFYPSDIAVEWESNGQPENNYKTTPPV |
| | | LDSDGSFFLYSKLTVDKSRWQQGNVFSCSVMHEALHSHYTQKSLSL |
| | | SPG |
| hIgG1- | 2151 | ASTKGPSVFPLAPSSKSTSGGTAALGCLVKDYFPEPVTVSWNSGALT |
| LALAGA/DHS | | SGVHTFPAVLQSSGLYSLSSVVTVPSSSLGTQTYICNVNHKPSNTKV |
| | | DKKVEPKSCDKTHTCPPCPAPEAAGAPSVFLFPPKPKDTLMISRTPEV |
| | | TCVVVDVSHEDPEVKFNWYVDGVEVHNAKTKPREEQYNSTYRVVS |
| | | VLTVDHHDWLNGKEYKCKVSNKALPAPIEKTISKAKGQPREPQVYT |
| | | LPPSRDELTKNQVSLTCLVKGFYPSDIAVEWESNGQPENNYKTTPPV |
| | | LDSDGSFFLYSKLTVDKSRWQQGNVFSCSVMHEALHSHYTQKSLSL |
| | | SPG |
| hIgG1- | 2152 | ASTKGPSVFPLAPSSKSTSGGTAALGCLVKDYFPEPVTVSWNSGALT |
| LALAPG/DHS | | SGVHTFPAVLQSSGLYSLSSVVTVPSSSLGTQTYICNVNHKPSNTKV |
| | | DKKVEPKSCDKTHTCPPCPAPEAAGGPSVFLFPPKPKDTLMISRTPEV |
| | | TCVVVDVSHEDPEVKFNWYVDGVEVHNAKTKPREEQYNSTYRVVS |
| | | VLTVDHHDWLNGKEYKCKVSNKALGAPIEKTISKAKGQPREPQVYT |
| | | LPPSRDELTKNQVSLTCLVKGFYPSDIAVEWESNGQPENNYKTTPPV |
| | | LDSDGSFFLYSKLTVDKSRWQQGNVFSCSVMHEALHSHYTQKSLSL |
| | | SPG |
| hIgG4-YTE | 2153 | ASTKGPSVFPLAPCSRSTSESTAALGCLVKDYFPEPVTVSWNSGALT |
| | | SGVHTFPAVLQSSGLYSLSSVVTVPSSSLGTKTYTCNVDHKPSNTKV |
| | | DKRVESKYGPPCPSCPAPEFLGGPSVFLFPPKPKDTLYITREPEVTCV |
| | | VVDVSQEDPEVQFNWYVDGVEVHNAKTKPREEQFNSTYRVVSVLT |
| | | VLHQDWLNGKEYKCKVSNKGLPSSIEKTISKAKGQPREPQVYTLPPS |
| | | QEEMTKNQVSLTCLVKGFYPSDIAVEWESNGQPENNYKTTPPVLDS |
| | | DGSFFLYSRLTVDKSRWQEGNVFSCSVMHEALHNHYTQKSLSLSLG |
| | | K |
| hIgG4- | 2154 | ASTKGPSVFPLAPCSRSTSESTAALGCLVKDYFPEPVTVSWNSGALT |
| SP/YTE | | SGVHTFPAVLQSSGLYSLSSVVTVPSSSLGTKTYTCNVDHKPSNTKV |
| | | DKRVESKYGPPCPPCPAPEFLGGPSVFLFPPKPKDTLYITREPEVTCV |
| | | VVDVSQEDPEVQFNWYVDGVEVHNAKTKPREEQFNSTYRVVSVLT |
| | | VLHQDWLNGKEYKCKVSNKGLPSSIEKTISKAKGQPREPQVYTLPPS |
| | | QEEMTKNQVSLTCLVKGFYPSDIAVEWESNGQPENNYKTTPPVLDS |
| | | DGSFFLYSRLTVDKSRWQEGNVFSCSVMHEALHNHYTQKSLSLSLG |
| | | K |
| hIgG4- | 2155 | ASTKGPSVFPLAPCSRSTSESTAALGCLVKDYFPEPVTVSWNSGALT |
| SPLE/YTE | | SGVHTFPAVLQSSGLYSLSSVVTVPSSSLGTKTYTCNVDHKPSNTKV |
| | | DKRVESKYGPPCPPCPAPEELGGPSVFLFPPKPKDTLYITREPEVTCV |
| | | VVDVSQEDPEVQFNWYVDGVEVHNAKTKPREEQFNSTYRVVSVLT |
| | | VLHQDWLNGKEYKCKVSNKGLPSSIEKTISKAKGQPREPQVYTLPPS |
| | | QEEMTKNQVSLTCLVKGFYPSDIAVEWESNGQPENNYKTTPPVLDS |
| | | DGSFFLYSRLTVDKSRWQEGNVFSCSVMHEALHNHYTQKSLSLSLG |
| | | K |
| hIgG4-LS | 2156 | ASTKGPSVFPLAPCSRSTSESTAALGCLVKDYFPEPVTVSWNSGALT |
| | | SGVHTFPAVLQSSGLYSLSSVVTVPSSSLGTKTYTCNVDHKPSNTKV |
| | | DKRVESKYGPPCPSCPAPEFLGGPSVFLFPPKPKDTLMISRTPEVTCV |
| | | VVDVSQEDPEVQFNWYVDGVEVHNAKTKPREEQFNSTYRVVSVLT |
| | | VLHQDWLNGKEYKCKVSNKGLPSSIEKTISKAKGQPREPQVYTLPPS |
| | | QEEMTKNQVSLTCLVKGFYPSDIAVEWESNGQPENNYKTTPPVLDS |
| | | DGSFFLYSRLTVDKSRWQEGNVFSCSVLHEALHSYTQKSLSLSLGK |
| hIgG4-SP/LS | 2157 | ASTKGPSVFPLAPCSRSTSESTAALGCLVKDYFPEPVTVSWNSGALT |
| | | SGVHTFPAVLQSSGLYSLSSVVTVPSSSLGTKTYTCNVDHKPSNTKV |
| | | DKRVESKYGPPCPPCPAPEFLGGPSVFLFPPKPKDTLMISRTPEVTCV |
| | | VVDVSQEDPEVQFNWYVDGVEVHNAKTKPREEQFNSTYRVVSVLT |
| | | VLHQDWLNGKEYKCKVSNKGLPSSIEKTISKAKGQPREPQVYTLPPS |
| | | QEEMTKNQVSLTCLVKGFYPSDIAVEWESNGQPENNYKTTPPVLDS |
| | | DGSFFLYSRLTVDKSRWQEGNVFSCSVLHEALHSHYTQKSLSLSLGK |
| hIgG4- | 2158 | ASTKGPSVFPLAPCSRSTSESTAALGCLVKDYFPEPVTVSWNSGALT |
| SPLE/LS | | SGVHTFPAVLQSSGLYSLSSVVTVPSSSLGTKTYTCNVDHKPSNTKV |
| | | DKRVESKYGPPCPPCPAPEELGGPSVFLFPPKPKDTLMISRTPEVTCV |
| | | VVDVSQEDPEVQFNWYVDGVEVHNAKTKPREEQFNSTYRVVSVLT |
| | | VLHQDWLNGKEYKCKVSNKGLPSSIEKTISKAKGQPREPQVYTLPPS |
| | | QEEMTKNQVSLTCLVKGFYPSDIAVEWESNGQPENNYKTTPPVLDS |
| | | DGSFFLYSRLTVDKSRWQEGNVFSCSVLHEALHSHYTQKSLSLSLGK |
| hIgG4-DHS | 2159 | ASTKGPSVFPLAPCSRSTSESTAALGCLVKDYFPEPVTVSWNSGALT |
| | | SGVHTFPAVLQSSGLYSLSSVVTVPSSSLGTKTYTCNVDHKPSNTKV |
| | | DKRVESKYGPPCPSCPAPEFLGGPSVFLFPPKPKDTLMISRTPEVTCV |
| | | VVDVSQEDPEVQFNWYVDGVEVHNAKTKPREEQFNSTYRVVSVLT |
| | | VDHHDWLNGKEYKCKVSNKGLPSSIEKTISKAKGQPREPQVYTLPPS |
| | | QEEMTKNQVSLTCLVKGFYPSDIAVEWESNGQPENNYKTTPPVLDS |
| | | DGSFFLYSRLTVDKSRWQEGNVFSCSVMHEALHSHYTQKSLSLSLG |
| | | K |
| hIgG4- | 2160 | ASTKGPSVFPLAPCSRSTSESTAALGCLVKDYFPEPVTVSWNSGALT |
| SP/DHS | | SGVHTFPAVLQSSGLYSLSSVVTVPSSSLGTKTYTCNVDHKPSNTKV |
| | | DKRVESKYGPPCPPCPAPEFLGGPSVFLFPPKPKDTLMISRTPEVTCV |
| | | VVDVSQEDPEVQFNWYVDGVEVHNAKTKPREEQFNSTYRVVSVLT |
| | | VDHHDWLNGKEYKCKVSNKGLPSSIEKTISKAKGQPREPQVYTLPPS |
| | | QEEMTKNQVSLTCLVKGFYPSDIAVEWESNGQPENNYKTTPPVLDS |
| | | DGSFFLYSRLTVDKSRWQEGNVFSCSVMHEALHSHYTQKSLSLSLG |
| | | K |
| hIgG4- | 2161 | ASTKGPSVFPLAPCSRSTSESTAALGCLVKDYFPEPVTVSWNSGALT |
| SPLE/DHS | | SGVHTFPAVLQSSGLYSLSSVVTVPSSSLGTKTYTCNVDHKPSNTKV |
| | | DKRVESKYGPPCPPCPAPEELGGPSVFLFPPKPKDTLMISRTPEVTCV |
| | | VVDVSQEDPEVQFNWYVDGVEVHNAKTKPREEQFNSTYRVVSVLT |
| | | VDHHDWLNGKEYKCKVSNKGLPSSIEKTISKAKGQPREPQVYTLPPS |
| | | QEEMTKNQVSLTCLVKGFYPSDIAVEWESNGQPENNYKTTPPVLDS |
| | | DGSFFLYSRLTVDKSRWQEGNVFSCSVMHEALHSHYTQKSLSLSLG |
| | | K |
| hIgG2-YTE | 2162 | ASTKGPSVFPLAPCSRSTSESTAALGCLVKDYFPEPVTVSWNSGALT |
| | | SGVHTFPAVLQSSGLYSLSSVVTVPSSNFGTQTYTCNVDHKPSNTKV |
| | | DKTVERKCCVECPPCPAPPVAGPSVFLFPPKPKDTLYITREPEVTCVV |
| | | VDVSHEDPEVQFNWYVDGVEVHNAKTKPREEQFNSTFRVVSVLTV |
| | | VHQDWLNGKEYKCKVSNKGLPAPIEKTISKTKGQPREPQVYTLPPSR |
| | | EEMTKNQVSLTCLVKGFYPSDIAVEWESNGQPENNYKTTPPMLDSD |
| | | GSFFLYSKLTVDKSRWQQGNVFSCSVLHEALHSHYTQKSLSLSP |
| hIgG2-LS | 2163 | ASTKGPSVFPLAPCSRSTSESTAALGCLVKDYFPEPVTVSWNSGALT |
| | | SGVHTFPAVLQSSGLYSLSSVVTVPSSNFGTQTYTCNVDHKPSNTKV |
| | | DKTVERKCCVECPPCPAPPVAGPSVFLFPPKPKDTLMISRTPEVTCVV |
| | | VDVSHEDPEVQFNWYVDGVEVHNAKTKPREEQFNSTFRVVSVLTV |
| | | VHQDWLNGKEYKCKVSNKGLPAPIEKTISKTKGQPREPQVYTLPPSR |
| | | EEMTKNQVSLTCLVKGFYPSDIAVEWESNGQPENNYKTTPPMLDSD |
| | | GSFFLYSKLTVDKSRWQQGNVFSCSVMHEALHNHYTQKSLSLSP |
| hIgG2-DHS | 2164 | ASTKGPSVFPLAPCSRSTSESTAALGCLVKDYFPEPVTVSWNSGALT |
| | | SGVHTFPAVLQSSGLYSLSSVVTVPSSNFGTQTYTCNVDHKPSNTKV |
| | | DKTVERKCCVECPPCPAPPVAGPSVFLFPPKPKDTLMISRTPEVTCVV |
| | | VDVSHEDPEVQFNWYVDGVEVHNAKTKPREEQFNSTFRVVSVLTV |
| | | DHHDWLNGKEYKCKVSNKGLPAPIEKTISKTKGQPREPQVYTLPPSR |
| | | EEMTKNQVSLTCLVKGFYPSDIAVEWESNGQPENNYKTTPPMLDSD |
| | | GSFFLYSKLTVDKSRWQQGNVFSCSVMHEALHSHYTQKSLSLSP |
| IgG4-SP | 2165 | ASTKGPSVFPLAPCSRSTSESTAALGCLVKDYFPEPVTVSWNSGALT |
| | | SGVHTFPAVLQSSGLYSLSSVVTVPSSSLGTKTYTCNVDHKPSNTKV |
| | | DKRVESKYGPPCPPCPAPEFLGGPSVFLFPPKPKDTLMISRTPEVTCV |
| | | VVDVSQEDPEVQFNWYVDGVEVHNAKTKPREEQFNSTYRVVSVLT |
| | | VLHQDWLNGKEYKCKVSNKGLPSSIEKTISKAKGQPREPQVYTLPPS |
| | | QEEMTKNQVSLTCLVKGFYPSDIAVEWESNGQPENNYKTTPPVLDS |
| | | DGSFFLYSRLTVDKSRWQEGNVFSCSVMHEALHNHYTQKSLSLSLG |
| | | K |
| hIgG1-LA | 2166 | ASTKGPSVFPLAPSSKSTSGGTAALGCLVKDYFPEPVTVSWNSGALT |
| | | SGVHTFPAVLQSSGLYSLSSVVTVPSSSLGTQTYICNVNHKPSNTKV |
| | | DKKVEPKSCDKTHTCPPCPAPELLGGPSVFLFPPKPKDTLMISRTPEV |
| | | TCVVVDVSHEDPEVKFNWYVDGVEVHNAKTKPREEQYNSTYRVVS |
| | | VLTVLHQDWLNGKEYKCKVSNKALPAPIEKTISKAKGQPREPQVYT |
| | | LPPSRDELTKNQVSLTCLVKGFYPSDIAVEWESNGQPENNYKTTPPV |
| | | LDSDGSFFLYSKLTVDKSRWQQGNVFSCSVLHEALHAHYTQKSLSL |
| | | SPG |
| hIgG1- | 2167 | ASTKGPSVFPLAPSSKSTSGGTAALGCLVKDYFPEPVTVSWNSGALT |
| N297A/LA | | SGVHTFPAVLQSSGLYSLSSVVTVPSSSLGTQTYICNVNHKPSNTKV |
| | | DKKVEPKSCDKTHTCPPCPAPELLGGPSVFLFPPKPKDTLMISRTPEV |
| | | TCVVVDVSHEDPEVKFNWYVDGVEVHNAKTKPREEQYASTYRVVS |
| | | VLTVLHQDWLNGKEYKCKVSNKALPAPIEKTISKAKGQPREPQVYT |
| | | LPPSRDELTKNQVSLTCLVKGFYPSDIAVEWESNGQPENNYKTTPPV |
| | | LDSDGSFFLYSKLTVDKSRWQQGNVFSCSVLHEALHAHYTQKSLSL |
| | | SPG |
| hIgG1- | 2168 | ASTKGPSVFPLAPSSKSTSGGTAALGCLVKDYFPEPVTVSWNSGALT |
| D265A/LA | | SGVHTFPAVLQSSGLYSLSSVVTVPSSSLGTQTYICNVNHKPSNTKV |
| | | DKKVEPKSCDKTHTCPPCPAPELLGGPSVFLFPPKPKDTLMISRTPEV |
| | | TCVVVAVSHEDPEVKFNWYVDGVEVHNAKTKPREEQYNSTYRVVS |
| | | VLTVLHQDWLNGKEYKCKVSNKALPAPIEKTISKAKGQPREPQVYT |
| | | LPPSRDELTKNQVSLTCLVKGFYPSDIAVEWESNGQPENNYKTTPPV |
| | | LDSDGSFFLYSKLTVDKSRWQQGNVFSCSVLHEALHAHYTQKSLSL |
| | | SPG |
| hIgG1- | 2169 | ASTKGPSVFPLAPSSKSTSGGTAALGCLVKDYFPEPVTVSWNSGALT |
| LALA/LA | | SGVHTFPAVLQSSGLYSLSSVVTVPSSSLGTQTYICNVNHKPSNTKV |
| | | DKKVEPKSCDKTHTCPPCPAPEAAGGPSVFLFPPKPKDTLMISRTPEV |
| | | TCVVVDVSHEDPEVKFNWYVDGVEVHNAKTKPREEQYNSTYRVVS |
| | | VLTVLHQDWLNGKEYKCKVSNKALPAPIEKTISKAKGQPREPQVYT |
| | | LPPSRDELTKNQVSLTCLVKGFYPSDIAVEWESNGQPENNYKTTPPV |
| | | LDSDGSFFLYSKLTVDKSRWQQGNVFSCSVLHEALHAHYTQKSLSL |
| | | SPG |
| hIgG1- | 2170 | ASTKGPSVFPLAPSSKSTSGGTAALGCLVKDYFPEPVTVSWNSGALT |
| LAGA/LA | | SGVHTFPAVLQSSGLYSLSSVVTVPSSSLGTQTYICNVNHKPSNTKV |
| | | DKKVEPKSCDKTHTCPPCPAPELAGAPSVFLFPPKPKDTLMISRTPEV |
| | | TCVVVDVSHEDPEVKFNWYVDGVEVHNAKTKPREEQYNSTYRVVS |
| | | VLTVLHQDWLNGKEYKCKVSNKALPAPIEKTISKAKGQPREPQVYT |
| | | LPPSRDELTKNQVSLTCLVKGFYPSDIAVEWESNGQPENNYKTTPPV |
| | | LDSDGSFFLYSKLTVDKSRWQQGNVFSCSVLHEALHAHYTQKSLSL |
| | | SPG |
| hIgG1- | 2171 | ASTKGPSVFPLAPSSKSTSGGTAALGCLVKDYFPEPVTVSWNSGALT |
| LALAGA/LA | | SGVHTFPAVLQSSGLYSLSSVVTVPSSSLGTQTYICNVNHKPSNTKV |
| | | DKKVEPKSCDKTHTCPPCPAPEAAGAPSVFLFPPKPKDTLMISRTPEV |
| | | TCVVVDVSHEDPEVKFNWYVDGVEVHNAKTKPREEQYNSTYRVVS |
| | | VLTVLHQDWLNGKEYKCKVSNKALPAPIEKTISKAKGQPREPQVYT |
| | | LPPSRDELTKNQVSLTCLVKGFYPSDIAVEWESNGQPENNYKTTPPV |
| | | LDSDGSFFLYSKLTVDKSRWQQGNVFSCSVLHEALHAHYTQKSLSL |
| | | SPG |
| hIgG1- | 2172 | ASTKGPSVFPLAPSSKSTSGGTAALGCLVKDYFPEPVTVSWNSGALT |
| LALAPG/LA | | SGVHTFPAVLQSSGLYSLSSVVTVPSSSLGTQTYICNVNHKPSNTKV |
| | | DKKVEPKSCDKTHTCPPCPAPEAAGGPSVFLFPPKPKDTLMISRTPEV |
| | | TCVVVDVSHEDPEVKFNWYVDGVEVHNAKTKPREEQYNSTYRVVS |
| | | VLTVLHQDWLNGKEYKCKVSNKALGAPIEKTISKAKGQPREPQVYT |
| | | LPPSRDELTKNQVSLTCLVKGFYPSDIAVEWESNGQPENNYKTTPPV |
| | | LDSDGSFFLYSKLTVDKSRWQQGNVFSCSVLHEALHAHYTQKSLSL |
| | | SPG |
| hIgG1-N434A | 2173 | ASTKGPSVFPLAPSSKSTSGGTAALGCLVKDYFPEPVTVSWNSGALT |
| | | SGVHTFPAVLQSSGLYSLSSVVTVPSSSLGTQTYICNVNHKPSNTKV |
| | | DKKVEPKSCDKTHTCPPCPAPELLGGPSVFLFPPKPKDTLMISRTPEV |
| | | TCVVVDVSHEDPEVKFNWYVDGVEVHNAKTKPREEQYNSTYRVVS |
| | | VLTVLHQDWLNGKEYKCKVSNKALPAPIEKTISKAKGQPREPQVYT |
| | | LPPSRDELTKNQVSLTCLVKGFYPSDIAVEWESNGQPENNYKTTPPV |
| | | LDSDGSFFLYSKLTVDKSRWQQGNVFSCSVMHEALHAHYTQKSLSL |
| | | SPG |
| hIgG1- | 2174 | ASTKGPSVFPLAPSSKSTSGGTAALGCLVKDYFPEPVTVSWNSGALT |
| N297A/ | | SGVHTFPAVLQSSGLYSLSSVVTVPSSSLGTQTYICNVNHKPSNTKV |
| N434A | | DKKVEPKSCDKTHTCPPCPAPELLGGPSVFLFPPKPKDTLMISRTPEV |
| | | TCVVVDVSHEDPEVKFNWYVDGVEVHNAKTKPREEQYASTYRVVS |
| | | VLTVLHQDWLNGKEYKCKVSNKALPAPIEKTISKAKGQPREPQVYT |
| | | LPPSRDELTKNQVSLTCLVKGFYPSDIAVEWESNGQPENNYKTTPPV |
| | | LDSDGSFFLYSKLTVDKSRWQQGNVFSCSVMHEALHAHYTQKSLSL |
| | | SPG |
| hIgG1- | 2175 | ASTKGPSVFPLAPSSKSTSGGTAALGCLVKDYFPEPVTVSWNSGALT |
| D265A/ | | SGVHTFPAVLQSSGLYSLSSVVTVPSSSLGTQTYICNVNHKPSNTKV |
| N434A | | DKKVEPKSCDKTHTCPPCPAPELLGGPSVFLFPPKPKDTLMISRTPEV |
| | | TCVVVAVSHEDPEVKFNWYVDGVEVHNAKTKPREEQYNSTYRVVS |
| | | VLTVLHQDWLNGKEYKCKVSNKALPAPIEKTISKAKGQPREPQVYT |
| | | LPPSRDELTKNQVSLTCLVKGFYPSDIAVEWESNGQPENNYKTTPPV |
| | | LDSDGSFFLYSKLTVDKSRWQQGNVFSCSVMHEALHAHYTQKSLSL |
| | | SPG |
| hIgG1-LALA/ | 2176 | ASTKGPSVFPLAPSSKSTSGGTAALGCLVKDYFPEPVTVSWNSGALT |
| N434A | | SGVHTFPAVLQSSGLYSLSSVVTVPSSSLGTQTYICNVNHKPSNTKV |
| | | DKKVEPKSCDKTHTCPPCPAPEAAGGPSVFLFPPKPKDTLMISRTPEV |
| | | TCVVVDVSHEDPEVKFNWYVDGVEVHNAKTKPREEQYNSTYRVVS |
| | | VLTVLHQDWLNGKEYKCKVSNKALPAPIEKTISKAKGQPREPQVYT |
| | | LPPSRDELTKNQVSLTCLVKGFYPSDIAVEWESNGQPENNYKTTPPV |
| | | LDSDGSFFLYSKLTVDKSRWQQGNVFSCSVMHEALHAHYTQKSLSL |
| | | SPG |
| hIgG1-LAGA/ | 2177 | ASTKGPSVFPLAPSSKSTSGGTAALGCLVKDYFPEPVTVSWNSGALT |
| N434A | | SGVHTFPAVLQSSGLYSLSSVVTVPSSSLGTQTYICNVNHKPSNTKV |
| | | DKKVEPKSCDKTHTCPPCPAPELAGAPSVFLFPPKPKDTLMISRTPEV |
| | | TCVVVDVSHEDPEVKFNWYVDGVEVHNAKTKPREEQYNSTYRVVS |
| | | VLTVLHQDWLNGKEYKCKVSNKALPAPIEKTISKAKGQPREPQVYT |
| | | LPPSRDELTKNQVSLTCLVKGFYPSDIAVEWESNGQPENNYKTTPPV |
| | | LDSDGSFFLYSKLTVDKSRWQQGNVFSCSVMHEALHAHYTQKSLSL |
| | | SPG |
| hIgG1- | 2178 | ASTKGPSVFPLAPSSKSTSGGTAALGCLVKDYFPEPVTVSWNSGALT |
| LALAGA/ | | SGVHTFPAVLQSSGLYSLSSVVTVPSSSLGTQTYICNVNHKPSNTKV |
| N434A | | DKKVEPKSCDKTHTCPPCPAPEAAGAPSVFLFPPKPKDTLMISRTPEV |
| | | TCVVVDVSHEDPEVKFNWYVDGVEVHNAKTKPREEQYNSTYRVVS |
| | | VLTVLHQDWLNGKEYKCKVSNKALPAPIEKTISKAKGQPREPQVYT |
| | | LPPSRDELTKNQVSLTCLVKGFYPSDIAVEWESNGQPENNYKTTPPV |
| | | LDSDGSFFLYSKLTVDKSRWQQGNVFSCSVMHEALHAHYTQKSLSL |
| | | SPG |
| hIgG1- | 2179 | ASTKGPSVFPLAPSSKSTSGGTAALGCLVKDYFPEPVTVSWNSGALT |
| LALAPG/ | | SGVHTFPAVLQSSGLYSLSSVVTVPSSSLGTQTYICNVNHKPSNTKV |
| N434A | | DKKVEPKSCDKTHTCPPCPAPEAAGGPSVFLFPPKPKDTLMISRTPEV |
| | | TCVVVDVSHEDPEVKFNWYVDGVEVHNAKTKPREEQYNSTYRVVS |
| | | VLTVLHQDWLNGKEYKCKVSNKALGAPIEKTISKAKGQPREPQVYT |
| | | LPPSRDELTKNQVSLTCLVKGFYPSDIAVEWESNGQPENNYKTTPPV |
| | | LDSDGSFFLYSKLTVDKSRWQQGNVFSCSVMHEALHAHYTQKSLSL |
| | | SPG |
| hIgG1- | 2180 | ASTKGPSVFPLAPSSKSTSGGTAALGCLVKDYFPEPVTVSWNSGALT |
| N434W | | SGVHTFPAVLQSSGLYSLSSVVTVPSSSLGTQTYICNVNHKPSNTKV |
| | | DKKVEPKSCDKTHTCPPCPAPELLGGPSVFLFPPKPKDTLMISRTPEV |
| | | TCVVVDVSHEDPEVKFNWYVDGVEVHNAKTKPREEQYNSTYRVVS |
| | | VLTVLHQDWLNGKEYKCKVSNKALPAPIEKTISKAKGQPREPQVYT |
| | | LPPSRDELTKNQVSLTCLVKGFYPSDIAVEWESNGQPENNYKTTPPV |
| | | LDSDGSFFLYSKLTVDKSRWQQGNVFSCSVMHEALHWHYTQKSLS |
| | | LSPG |
| hIgG1- | 2181 | ASTKGPSVFPLAPSSKSTSGGTAALGCLVKDYFPEPVTVSWNSGALT |
| N297A/ | | SGVHTFPAVLQSSGLYSLSSVVTVPSSSLGTQTYICNVNHKPSNTKV |
| N434W | | DKKVEPKSCDKTHTCPPCPAPELLGGPSVFLFPPKPKDTLMISRTPEV |
| | | TCVVVDVSHEDPEVKFNWYVDGVEVHNAKTKPREEQYASTYRVVS |
| | | VLTVLHQDWLNGKEYKCKVSNKALPAPIEKTISKAKGQPREPQVYT |
| | | LPPSRDELTKNQVSLTCLVKGFYPSDIAVEWESNGQPENNYKTTPPV |
| | | LDSDGSFFLYSKLTVDKSRWQQGNVFSCSVMHEALHWHYTQKSLS |
| | | LSPG |
| hIgG1- | 2182 | ASTKGPSVFPLAPSSKSTSGGTAALGCLVKDYFPEPVTVSWNSGALT |
| D265A/ | | SGVHTFPAVLQSSGLYSLSSVVTVPSSSLGTQTYICNVNHKPSNTKV |
| N434W | | DKKVEPKSCDKTHTCPPCPAPELLGGPSVFLFPPKPKDTLMISRTPEV |
| | | TCVVVAVSHEDPEVKFNWYVDGVEVHNAKTKPREEQYNSTYRVVS |
| | | VLTVLHQDWLNGKEYKCKVSNKALPAPIEKTISKAKGQPREPQVYT |
| | | LPPSRDELTKNQVSLTCLVKGFYPSDIAVEWESNGQPENNYKTTPPV |
| | | LDSDGSFFLYSKLTVDKSRWQQGNVFSCSVMHEALHWHYTQKSLS |
| | | LSPG |
| hIgG1-LALA/ | 2183 | ASTKGPSVFPLAPSSKSTSGGTAALGCLVKDYFPEPVTVSWNSGALT |
| N434W | | SGVHTFPAVLQSSGLYSLSSVVTVPSSSLGTQTYICNVNHKPSNTKV |
| | | DKKVEPKSCDKTHTCPPCPAPEAAGGPSVFLFPPKPKDTLMISRTPEV |
| | | TCVVVDVSHEDPEVKFNWYVDGVEVHNAKTKPREEQYNSTYRVVS |
| | | VLTVLHQDWLNGKEYKCKVSNKALPAPIEKTISKAKGQPREPQVYT |
| | | LPPSRDELTKNQVSLTCLVKGFYPSDIAVEWESNGQPENNYKTTPPV |
| | | LDSDGSFFLYSKLTVDKSRWQQGNVFSCSVMHEALHWHYTQKSLS |
| | | LSPG |
| hIgG1-LAGA/ | 2184 | ASTKGPSVFPLAPSSKSTSGGTAALGCLVKDYFPEPVTVSWNSGALT |
| N434W | | SGVHTFPAVLQSSGLYSLSSVVTVPSSSLGTQTYICNVNHKPSNTKV |
| | | DKKVEPKSCDKTHTCPPCPAPELAGAPSVFLFPPKPKDTLMISRTPEV |
| | | TCVVVDVSHEDPEVKFNWYVDGVEVHNAKTKPREEQYNSTYRVVS |
| | | VLTVLHQDWLNGKEYKCKVSNKALPAPIEKTISKAKGQPREPQVYT |
| | | LPPSRDELTKNQVSLTCLVKGFYPSDIAVEWESNGQPENNYKTTPPV |
| | | LDSDGSFFLYSKLTVDKSRWQQGNVFSCSVMHEALHWHYTQKSLS |
| | | LSPG |
| hIgG1- | 2185 | ASTKGPSVFPLAPSSKSTSGGTAALGCLVKDYFPEPVTVSWNSGALT |
| LALAGA/ | | SGVHTFPAVLQSSGLYSLSSVVTVPSSSLGTQTYICNVNHKPSNTKV |
| N434W | | DKKVEPKSCDKTHTCPPCPAPEAAGAPSVFLFPPKPKDTLMISRTPEV |
| | | TCVVVDVSHEDPEVKFNWYVDGVEVHNAKTKPREEQYNSTYRVVS |
| | | VLTVLHQDWLNGKEYKCKVSNKALPAPIEKTISKAKGQPREPQVYT |
| | | LPPSRDELTKNQVSLTCLVKGFYPSDIAVEWESNGQPENNYKTTPPV |
| | | LDSDGSFFLYSKLTVDKSRWQQGNVFSCSVMHEALHWHYTQKSLS |
| | | LSPG |
| hIgG1- | 2186 | ASTKGPSVFPLAPSSKSTSGGTAALGCLVKDYFPEPVTVSWNSGALT |
| LALAPG/ | | SGVHTFPAVLQSSGLYSLSSVVTVPSSSLGTQTYICNVNHKPSNTKV |
| N434W | | DKKVEPKSCDKTHTCPPCPAPEAAGGPSVFLFPPKPKDTLMISRTPEV |
| | | TCVVVDVSHEDPEVKFNWYVDGVEVHNAKTKPREEQYNSTYRVVS |
| | | VLTVLHQDWLNGKEYKCKVSNKALGAPIEKTISKAKGQPREPQVYT |
| | | LPPSRDELTKNQVSLTCLVKGFYPSDIAVEWESNGQPENNYKTTPPV |
| | | LDSDGSFFLYSKLTVDKSRWQQGNVFSCSVMHEALHWHYTQKSLS |
| | | LSPG |
| hIgG1/DQ | 2187 | ASTKGPSVFPLAPSSKSTSGGTAALGCLVKDYFPEPVTVSWNSGALT |
| | | SGVHTFPAVLQSSGLYSLSSVVTVPSSSLGTQTYICNVNHKPSNTKV |
| | | DKKVEPKSCDKTHTCPPCPAPELLGGPSVFLFPPKPKDTLMISRDPEV |
| | | TCVVVDVSHEDPEVKFNWYVDGVEVHNAKTKPREEQYNSTYRVVS |
| | | VLQVLHQDWLNGKEYKCKVSNKALPAPIEKTISKAKGQPREPQVYT |
| | | LPPSRDELTKNQVSLTCLVKGFYPSDIAVEWESNGQPENNYKTTPPV |
| | | LDSDGSFFLYSKLTVDKSRWQQGNVFSCSVMHEALHNHYTQKSLSL |
| | | SPG |
| hIgG1- | 2188 | ASTKGPSVFPLAPSSKSTSGGTAALGCLVKDYFPEPVTVSWNSGALT |
| N297A/DQ | | SGVHTFPAVLQSSGLYSLSSVVTVPSSSLGTQTYICNVNHKPSNTKV |
| | | DKKVEPKSCDKTHTCPPCPAPELLGGPSVFLFPPKPKDTLMISRDPEV |
| | | TCVVVDVSHEDPEVKFNWYVDGVEVHNAKTKPREEQYASTYRVVS |
| | | VLQVLHQDWLNGKEYKCKVSNKALPAPIEKTISKAKGQPREPQVYT |
| | | LPPSRDELTKNQVSLTCLVKGFYPSDIAVEWESNGQPENNYKTTPPV |
| | | LDSDGSFFLYSKLTVDKSRWQQGNVFSCSVMHEALHNHYTQKSLSL |
| | | SPG |
| hIgG1- | 2189 | ASTKGPSVFPLAPSSKSTSGGTAALGCLVKDYFPEPVTVSWNSGALT |
| D265A/DQ | | SGVHTFPAVLQSSGLYSLSSVVTVPSSSLGTQTYICNVNHKPSNTKV |
| | | DKKVEPKSCDKTHTCPPCPAPELLGGPSVFLFPPKPKDTLMISRDPEV |
| | | TCVVVAVSHEDPEVKFNWYVDGVEVHNAKTKPREEQYNSTYRVVS |
| | | VLQVLHQDWLNGKEYKCKVSNKALPAPIEKTISKAKGQPREPQVYT |
| | | LPPSRDELTKNQVSLTCLVKGFYPSDIAVEWESNGQPENNYKTTPPV |
| | | LDSDGSFFLYSKLTVDKSRWQQGNVFSCSVMHEALHNHYTQKSLSL |
| | | SPG |
| hIgG1- | 2190 | ASTKGPSVFPLAPSSKSTSGGTAALGCLVKDYFPEPVTVSWNSGALT |
| LALA/DQ | | SGVHTFPAVLQSSGLYSLSSVVTVPSSSLGTQTYICNVNHKPSNTKV |
| | | DKKVEPKSCDKTHTCPPCPAPEAAGGPSVFLFPPKPKDTLMISRDPE |
| | | VTCVVVDVSHEDPEVKFNWYVDGVEVHNAKTKPREEQYNSTYRV |
| | | VSVLQVLHQDWLNGKEYKCKVSNKALPAPIEKTISKAKGQPREPQV |
| | | YTLPPSRDELTKNQVSLTCLVKGFYPSDIAVEWESNGQPENNYKTTP |
| | | PVLDSDGSFFLYSKLTVDKSRWQQGNVFSCSVMHEALHNHYTQKSL |
| | | SLSPG |
| hIgG1- | 2191 | ASTKGPSVFPLAPSSKSTSGGTAALGCLVKDYFPEPVTVSWNSGALT |
| LAGA/DQ | | SGVHTFPAVLQSSGLYSLSSVVTVPSSSLGTQTYICNVNHKPSNTKV |
| | | DKKVEPKSCDKTHTCPPCPAPELAGAPSVFLFPPKPKDTLMISRDPEV |
| | | TCVVVDVSHEDPEVKFNWYVDGVEVHNAKTKPREEQYNSTYRVVS |
| | | VLQVLHQDWLNGKEYKCKVSNKALPAPIEKTISKAKGQPREPQVYT |
| | | LPPSRDELTKNQVSLTCLVKGFYPSDIAVEWESNGQPENNYKTTPPV |
| | | LDSDGSFFLYSKLTVDKSRWQQGNVFSCSVMHEALHNHYTQKSLSL |
| | | SPG |
| hIgG1- | 2192 | ASTKGPSVFPLAPSSKSTSGGTAALGCLVKDYFPEPVTVSWNSGALT |
| LALAGA/DQ | | SGVHTFPAVLQSSGLYSLSSVVTVPSSSLGTQTYICNVNHKPSNTKV |
| | | DKKVEPKSCDKTHTCPPCPAPEAAGAPSVFLFPPKPKDTLMISRDPE |
| | | VTCVVVDVSHEDPEVKFNWYVDGVEVHNAKTKPREEQYNSTYRV |
| | | VSVLQVLHQDWLNGKEYKCKVSNKALPAPIEKTISKAKGQPREPQV |
| | | YTLPPSRDELTKNQVSLTCLVKGFYPSDIAVEWESNGQPENNYKTTP |
| | | PVLDSDGSFFLYSKLTVDKSRWQQGNVFSCSVMHEALHNHYTQKSL |
| | | SLSPG |
| hIgG1- | 2193 | ASTKGPSVFPLAPSSKSTSGGTAALGCLVKDYFPEPVTVSWNSGALT |
| LALAPG/DQ | | SGVHTFPAVLQSSGLYSLSSVVTVPSSSLGTQTYICNVNHKPSNTKV |
| | | DKKVEPKSCDKTHTCPPCPAPEAAGGPSVFLFPPKPKDTLMISRDPE |
| | | VTCVVVDVSHEDPEVKFNWYVDGVEVHNAKTKPREEQYNSTYRV |
| | | VSVLQVLHQDWLNGKEYKCKVSNKALGAPIEKTISKAKGQPREPQV |
| | | YTLPPSRDELTKNQVSLTCLVKGFYPSDIAVEWESNGQPENNYKTTP |
| | | PVLDSDGSFFLYSKLTVDKSRWQQGNVFSCSVMHEALHNHYTQKSL |
| | | SLSPG |
| hIgG1/DW | 2194 | ASTKGPSVFPLAPSSKSTSGGTAALGCLVKDYFPEPVTVSWNSGALT |
| | | SGVHTFPAVLQSSGLYSLSSVVTVPSSSLGTQTYICNVNHKPSNTKV |
| | | DKKVEPKSCDKTHTCPPCPAPELLGGPSVFLFPPKPKDTLMISRDPEV |
| | | TCVVVDVSHEDPEVKFNWYVDGVEVHNAKTKPREEQYNSTYRVVS |
| | | VLWVLHQDWLNGKEYKCKVSNKALPAPIEKTISKAKGQPREPQVYT |
| | | LPPSRDELTKNQVSLTCLVKGFYPSDIAVEWESNGQPENNYKTTPPV |
| | | LDSDGSFFLYSKLTVDKSRWQQGNVFSCSVMHEALHNHYTQKSLSL |
| | | SPG |
| hIgG1- | 2195 | ASTKGPSVFPLAPSSKSTSGGTAALGCLVKDYFPEPVTVSWNSGALT |
| N297A/DW | | SGVHTFPAVLQSSGLYSLSSVVTVPSSSLGTQTYICNVNHKPSNTKV |
| | | DKKVEPKSCDKTHTCPPCPAPELLGGPSVFLFPPKPKDTLMISRDPEV |
| | | TCVVVDVSHEDPEVKFNWYVDGVEVHNAKTKPREEQYASTYRVVS |
| | | VLWVLHQDWLNGKEYKCKVSNKALPAPIEKTISKAKGQPREPQVYT |
| | | LPPSRDELTKNQVSLTCLVKGFYPSDIAVEWESNGQPENNYKTTPPV |
| | | LDSDGSFFLYSKLTVDKSRWQQGNVFSCSVMHEALHNHYTQKSLSL |
| | | SPG |
| hIgG1- | 2196 | ASTKGPSVFPLAPSSKSTSGGTAALGCLVKDYFPEPVTVSWNSGALT |
| D265A/DW | | SGVHTFPAVLQSSGLYSLSSVVTVPSSSLGTQTYICNVNHKPSNTKV |
| | | DKKVEPKSCDKTHTCPPCPAPELLGGPSVFLFPPKPKDTLMISRDPEV |
| | | TCVVVAVSHEDPEVKFNWYVDGVEVHNAKTKPREEQYNSTYRVVS |
| | | VLWVLHQDWLNGKEYKCKVSNKALPAPIEKTISKAKGQPREPQVYT |
| | | LPPSRDELTKNQVSLTCLVKGFYPSDIAVEWESNGQPENNYKTTPPV |
| | | LDSDGSFFLYSKLTVDKSRWQQGNVFSCSVMHEALHNHYTQKSLSL |
| | | SPG |
| hIgG1- | 2197 | ASTKGPSVFPLAPSSKSTSGGTAALGCLVKDYFPEPVTVSWNSGALT |
| LALA/DW | | SGVHTFPAVLQSSGLYSLSSVVTVPSSSLGTQTYICNVNHKPSNTKV |
| | | DKKVEPKSCDKTHTCPPCPAPEAAGGPSVFLFPPKPKDTLMISRDPE |
| | | VTCVVVDVSHEDPEVKFNWYVDGVEVHNAKTKPREEQYNSTYRV |
| | | VSVLWVLHQDWLNGKEYKCKVSNKALPAPIEKTISKAKGQPREPQV |
| | | YTLPPSRDELTKNQVSLTCLVKGFYPSDIAVEWESNGQPENNYKTTP |
| | | PVLDSDGSFFLYSKLTVDKSRWQQGNVFSCSVMHEALHNHYTQKSL |
| | | SLSPG |
| hIgG1- | 2198 | ASTKGPSVFPLAPSSKSTSGGTAALGCLVKDYFPEPVTVSWNSGALT |
| LAGA/DW | | SGVHTFPAVLQSSGLYSLSSVVTVPSSSLGTQTYICNVNHKPSNTKV |
| | | DKKVEPKSCDKTHTCPPCPAPELAGAPSVFLFPPKPKDTLMISRDPEV |
| | | TCVVVDVSHEDPEVKFNWYVDGVEVHNAKTKPREEQYNSTYRVVS |
| | | VLWVLHQDWLNGKEYKCKVSNKALPAPIEKTISKAKGQPREPQVYT |
| | | LPPSRDELTKNQVSLTCLVKGFYPSDIAVEWESNGQPENNYKTTPPV |
| | | LDSDGSFFLYSKLTVDKSRWQQGNVFSCSVMHEALHNHYTQKSLSL |
| | | SPG |
| hIgG1- | 2199 | ASTKGPSVFPLAPSSKSTSGGTAALGCLVKDYFPEPVTVSWNSGALT |
| LALAGA/DW | | SGVHTFPAVLQSSGLYSLSSVVTVPSSSLGTQTYICNVNHKPSNTKV |
| | | DKKVEPKSCDKTHTCPPCPAPEAAGAPSVFLFPPKPKDTLMISRDPE |
| | | VTCVVVDVSHEDPEVKFNWYVDGVEVHNAKTKPREEQYNSTYRV |
| | | VSVLWVLHQDWLNGKEYKCKVSNKALPAPIEKTISKAKGQPREPQV |
| | | YTLPPSRDELTKNQVSLTCLVKGFYPSDIAVEWESNGQPENNYKTTP |
| | | PVLDSDGSFFLYSKLTVDKSRWQQGNVFSCSVMHEALHNHYTQKSL |
| | | SLSPG |
| hIgG1- | 2200 | ASTKGPSVFPLAPSSKSTSGGTAALGCLVKDYFPEPVTVSWNSGALT |
| LALAPG/DW | | SGVHTFPAVLQSSGLYSLSSVVTVPSSSLGTQTYICNVNHKPSNTKV |
| | | DKKVEPKSCDKTHTCPPCPAPEAAGGPSVFLFPPKPKDTLMISRDPE |
| | | VTCVVVDVSHEDPEVKFNWYVDGVEVHNAKTKPREEQYNSTYRV |
| | | VSVLWVLHQDWLNGKEYKCKVSNKALGAPIEKTISKAKGQPREPQ |
| | | VYTLPPSRDELTKNQVSLTCLVKGFYPSDIAVEWESNGQPENNYKTT |
| | | PPVLDSDGSFFLYSKLTVDKSRWQQGNVFSCSVMHEALHNHYTQKS |
| | | LSLSPG |
| hIgG1/YD | 2201 | ASTKGPSVFPLAPSSKSTSGGTAALGCLVKDYFPEPVTVSWNSGALT |
| | | SGVHTFPAVLQSSGLYSLSSVVTVPSSSLGTQTYICNVNHKPSNTKV |
| | | DKKVEPKSCDKTHTCPPCPAPELLGGPSVFLFPPKPKDTLYISRDPEV |
| | | TCVVVDVSHEDPEVKFNWYVDGVEVHNAKTKPREEQYNSTYRVVS |
| | | VLTVLHQDWLNGKEYKCKVSNKALPAPIEKTISKAKGQPREPQVYT |
| | | LPPSRDELTKNQVSLTCLVKGFYPSDIAVEWESNGQPENNYKTTPPV |
| | | LDSDGSFFLYSKLTVDKSRWQQGNVFSCSVMHEALHNHYTQKSLSL |
| | | SPG |
| hIgG1- | 2202 | ASTKGPSVFPLAPSSKSTSGGTAALGCLVKDYFPEPVTVSWNSGALT |
| N297A/YD | | SGVHTFPAVLQSSGLYSLSSVVTVPSSSLGTQTYICNVNHKPSNTKV |
| | | DKKVEPKSCDKTHTCPPCPAPELLGGPSVFLFPPKPKDTLYISRDPEV |
| | | TCVVVDVSHEDPEVKFNWYVDGVEVHNAKTKPREEQYASTYRVVS |
| | | VLTVLHQDWLNGKEYKCKVSNKALPAPIEKTISKAKGQPREPQVYT |
| | | LPPSRDELTKNQVSLTCLVKGFYPSDIAVEWESNGQPENNYKTTPPV |
| | | LDSDGSFFLYSKLTVDKSRWQQGNVFSCSVMHEALHNHYTQKSLSL |
| | | SPG |
| hIgG1- | 2203 | ASTKGPSVFPLAPSSKSTSGGTAALGCLVKDYFPEPVTVSWNSGALT |
| D265A/YD | | SGVHTFPAVLQSSGLYSLSSVVTVPSSSLGTQTYICNVNHKPSNTKV |
| | | DKKVEPKSCDKTHTCPPCPAPELLGGPSVFLFPPKPKDTLYISRDPEV |
| | | TCVVVAVSHEDPEVKFNWYVDGVEVHNAKTKPREEQYNSTYRVVS |
| | | VLTVLHQDWLNGKEYKCKVSNKALPAPIEKTISKAKGQPREPQVYT |
| | | LPPSRDELTKNQVSLTCLVKGFYPSDIAVEWESNGQPENNYKTTPPV |
| | | LDSDGSFFLYSKLTVDKSRWQQGNVFSCSVMHEALHNHYTQKSLSL |
| | | SPG |
| hIgG1- | 2204 | ASTKGPSVFPLAPSSKSTSGGTAALGCLVKDYFPEPVTVSWNSGALT |
| LALA/YD | | SGVHTFPAVLQSSGLYSLSSVVTVPSSSLGTQTYICNVNHKPSNTKV |
| | | DKKVEPKSCDKTHTCPPCPAPEAAGGPSVFLFPPKPKDTLYISRDPEV |
| | | TCVVVDVSHEDPEVKFNWYVDGVEVHNAKTKPREEQYNSTYRVVS |
| | | VLTVLHQDWLNGKEYKCKVSNKALPAPIEKTISKAKGQPREPQVYT |
| | | LPPSRDELTKNQVSLTCLVKGFYPSDIAVEWESNGQPENNYKTTPPV |
| | | LDSDGSFFLYSKLTVDKSRWQQGNVFSCSVMHEALHNHYTQKSLSL |
| | | SPG |
| hIgG1- | 2205 | ASTKGPSVFPLAPSSKSTSGGTAALGCLVKDYFPEPVTVSWNSGALT |
| LAGA/YD | | SGVHTFPAVLQSSGLYSLSSVVTVPSSSLGTQTYICNVNHKPSNTKV |
| | | DKKVEPKSCDKTHTCPPCPAPELAGAPSVFLFPPKPKDTLYISRDPEV |
| | | TCVVVDVSHEDPEVKFNWYVDGVEVHNAKTKPREEQYNSTYRVVS |
| | | VLTVLHQDWLNGKEYKCKVSNKALPAPIEKTISKAKGQPREPQVYT |
| | | LPPSRDELTKNQVSLTCLVKGFYPSDIAVEWESNGQPENNYKTTPPV |
| | | LDSDGSFFLYSKLTVDKSRWQQGNVFSCSVMHEALHNHYTQKSLSL |
| | | SPG |
| hIgG1- | 2206 | ASTKGPSVFPLAPSSKSTSGGTAALGCLVKDYFPEPVTVSWNSGALT |
| LALAGA/YD | | SGVHTFPAVLQSSGLYSLSSVVTVPSSSLGTQTYICNVNHKPSNTKV |
| | | DKKVEPKSCDKTHTCPPCPAPEAAGAPSVFLFPPKPKDTLYISRDPEV |
| | | TCVVVDVSHEDPEVKFNWYVDGVEVHNAKTKPREEQYNSTYRVVS |
| | | VLTVLHQDWLNGKEYKCKVSNKALPAPIEKTISKAKGQPREPQVYT |
| | | LPPSRDELTKNQVSLTCLVKGFYPSDIAVEWESNGQPENNYKTTPPV |
| | | LDSDGSFFLYSKLTVDKSRWQQGNVFSCSVMHEALHNHYTQKSLSL |
| | | SPG |
| hIgG1- | 2207 | ASTKGPSVFPLAPSSKSTSGGTAALGCLVKDYFPEPVTVSWNSGALT |
| LALAPG/YD | | SGVHTFPAVLQSSGLYSLSSVVTVPSSSLGTQTYICNVNHKPSNTKV |
| | | DKKVEPKSCDKTHTCPPCPAPEAAGGPSVFLFPPKPKDTLYISRDPEV |
| | | TCVVVDVSHEDPEVKFNWYVDGVEVHNAKTKPREEQYNSTYRVVS |
| | | VLTVLHQDWLNGKEYKCKVSNKALGAPIEKTISKAKGQPREPQVYT |
| | | LPPSRDELTKNQVSLTCLVKGFYPSDIAVEWESNGQPENNYKTTPPV |
| | | LDSDGSFFLYSKLTVDKSRWQQGNVFSCSVMHEALHNHYTQKSLSL |
| | | SPG |
| hIgG1/QVV | 2208 | ASTKGPSVFPLAPSSKSTSGGTAALGCLVKDYFPEPVTVSWNSGALT |
| | | SGVHTFPAVLQSSGLYSLSSVVTVPSSSLGTQTYICNVNHKPSNTKV |
| | | DKKVEPKSCDKTHTCPPCPAPELLGGPSVFLFPPKPKDTLMISRTPEV |
| | | TCVVVDVSHEDPEVKFNWYVDGVEVHNAKTKPREEQYNSTYRVVS |
| | | VLQVLHVDWLNGKEYKCKVSNKALPAPIEKTISKAKGQPREPQVYT |
| | | LPPSRDELTKNQVSLTCLVKGFYPSDIVVEWESNGQPENNYKTTPPV |
| | | LDSDGSFFLYSKLTVDKSRWQQGNVFSCSVMHEALHNHYTQKSLSL |
| | | SPG |
| hIgG1- | 2209 | ASTKGPSVFPLAPSSKSTSGGTAALGCLVKDYFPEPVTVSWNSGALT |
| N297A/QVV | | SGVHTFPAVLQSSGLYSLSSVVTVPSSSLGTQTYICNVNHKPSNTKV |
| | | DKKVEPKSCDKTHTCPPCPAPELLGGPSVFLFPPKPKDTLMISRTPEV |
| | | TCVVVDVSHEDPEVKFNWYVDGVEVHNAKTKPREEQYASTYRVVS |
| | | VLQVLHVDWLNGKEYKCKVSNKALPAPIEKTISKAKGQPREPQVYT |
| | | LPPSRDELTKNQVSLTCLVKGFYPSDIVVEWESNGQPENNYKTTPPV |
| | | LDSDGSFFLYSKLTVDKSRWQQGNVFSCSVMHEALHNHYTQKSLSL |
| | | SPG |
| hIgG1- | 2210 | ASTKGPSVFPLAPSSKSTSGGTAALGCLVKDYFPEPVTVSWNSGALT |
| D265A/QVV | | SGVHTFPAVLQSSGLYSLSSVVTVPSSSLGTQTYICNVNHKPSNTKV |
| | | DKKVEPKSCDKTHTCPPCPAPELLGGPSVFLFPPKPKDTLMISRTPEV |
| | | TCVVVAVSHEDPEVKFNWYVDGVEVHNAKTKPREEQYNSTYRVVS |
| | | VLQVLHVDWLNGKEYKCKVSNKALPAPIEKTISKAKGQPREPQVYT |
| | | LPPSRDELTKNQVSLTCLVKGFYPSDIVVEWESNGQPENNYKTTPPV |
| | | LDSDGSFFLYSKLTVDKSRWQQGNVFSCSVMHEALHNHYTQKSLSL |
| | | SPG |
| hIgG1- | 2211 | ASTKGPSVFPLAPSSKSTSGGTAALGCLVKDYFPEPVTVSWNSGALT |
| LALA/QVV | | SGVHTFPAVLQSSGLYSLSSVVTVPSSSLGTQTYICNVNHKPSNTKV |
| | | DKKVEPKSCDKTHTCPPCPAPEAAGGPSVFLFPPKPKDTLMISRTPEV |
| | | TCVVVDVSHEDPEVKFNWYVDGVEVHNAKTKPREEQYNSTYRVVS |
| | | VLQVLHVDWLNGKEYKCKVSNKALPAPIEKTISKAKGQPREPQVYT |
| | | LPPSRDELTKNQVSLTCLVKGFYPSDIVVEWESNGQPENNYKTTPPV |
| | | LDSDGSFFLYSKLTVDKSRWQQGNVFSCSVMHEALHNHYTQKSLSL |
| | | SPG |
| hIgG1- | 2212 | ASTKGPSVFPLAPSSKSTSGGTAALGCLVKDYFPEPVTVSWNSGALT |
| LAGA/QVV | | SGVHTFPAVLQSSGLYSLSSVVTVPSSSLGTQTYICNVNHKPSNTKV |
| | | DKKVEPKSCDKTHTCPPCPAPELAGAPSVFLFPPKPKDTLMISRTPEV |
| | | TCVVVDVSHEDPEVKFNWYVDGVEVHNAKTKPREEQYNSTYRVVS |
| | | VLQVLHVDWLNGKEYKCKVSNKALPAPIEKTISKAKGQPREPQVYT |
| | | LPPSRDELTKNQVSLTCLVKGFYPSDIVVEWESNGQPENNYKTTPPV |
| | | LDSDGSFFLYSKLTVDKSRWQQGNVFSCSVMHEALHNHYTQKSLSL |
| | | SPG |
| hIgG1- | 2213 | ASTKGPSVFPLAPSSKSTSGGTAALGCLVKDYFPEPVTVSWNSGALT |
| LALAGA/QVV | | SGVHTFPAVLQSSGLYSLSSVVTVPSSSLGTQTYICNVNHKPSNTKV |
| | | DKKVEPKSCDKTHTCPPCPAPEAAGAPSVFLFPPKPKDTLMISRTPEV |
| | | TCVVVDVSHEDPEVKFNWYVDGVEVHNAKTKPREEQYNSTYRVVS |
| | | VLQVLHVDWLNGKEYKCKVSNKALPAPIEKTISKAKGQPREPQVYT |
| | | LPPSRDELTKNQVSLTCLVKGFYPSDIVVEWESNGQPENNYKTTPPV |
| | | LDSDGSFFLYSKLTVDKSRWQQGNVFSCSVMHEALHNHYTQKSLSL |
| | | SPG |
| hIgG1- | 2214 | ASTKGPSVFPLAPSSKSTSGGTAALGCLVKDYFPEPVTVSWNSGALT |
| LALAPG/QVV | | SGVHTFPAVLQSSGLYSLSSVVTVPSSSLGTQTYICNVNHKPSNTKV |
| | | DKKVEPKSCDKTHTCPPCPAPEAAGGPSVFLFPPKPKDTLMISRTPEV |
| | | TCVVVDVSHEDPEVKFNWYVDGVEVHNAKTKPREEQYNSTYRVVS |
| | | VLQVLHVDWLNGKEYKCKVSNKALGAPIEKTISKAKGQPREPQVYT |
| | | LPPSRDELTKNQVSLTCLVKGFYPSDIVVEWESNGQPENNYKTTPPV |
| | | LDSDGSFFLYSKLTVDKSRWQQGNVFSCSVMHEALHNHYTQKSLSL |
| | | SPG |
| hIgG1/DDRVV | 2215 | ASTKGPSVFPLAPSSKSTSGGTAALGCLVKDYFPEPVTVSWNSGALT |
| | | SGVHTFPAVLQSSGLYSLSSVVTVPSSSLGTQTYICNVNHKPSNTKV |
| | | DKKVEPKSCDKTHTCPPCPAPELLGGPSVFLFPPKPKDTLMISRDPEV |
| | | TCVVVDVSHEDPEVKFNWYVDGVEVDNAKTKPREEQYNSTYRVVS |
| | | VLRVLHVDWLNGKEYKCKVSNKALPAPIEKTISKAKGQPREPQVYT |
| | | LPPSRDELTKNQVSLTCLVKGFYPSDIVVEWESNGQPENNYKTTPPV |
| | | LDSDGSFFLYSKLTVDKSRWQQGNVFSCSVMHEALHNHYTQKSLSL |
| | | SPG |
| hIgG1- | 2216 | ASTKGPSVFPLAPSSKSTSGGTAALGCLVKDYFPEPVTVSWNSGALT |
| N297A/DDRVV | | SGVHTFPAVLQSSGLYSLSSVVTVPSSSLGTQTYICNVNHKPSNTKV |
| | | DKKVEPKSCDKTHTCPPCPAPELLGGPSVFLFPPKPKDTLMISRDPEV |
| | | TCVVVDVSHEDPEVKFNWYVDGVEVDNAKTKPREEQYASTYRVVS |
| | | VLRVLHVDWLNGKEYKCKVSNKALPAPIEKTISKAKGQPREPQVYT |
| | | LPPSRDELTKNQVSLTCLVKGFYPSDIVVEWESNGQPENNYKTTPPV |
| | | LDSDGSFFLYSKLTVDKSRWQQGNVFSCSVMHEALHNHYTQKSLSL |
| | | SPG |
| hIgG1- | 2217 | ASTKGPSVFPLAPSSKSTSGGTAALGCLVKDYFPEPVTVSWNSGALT |
| D265A/DDRVV | | SGVHTFPAVLQSSGLYSLSSVVTVPSSSLGTQTYICNVNHKPSNTKV |
| | | DKKVEPKSCDKTHTCPPCPAPELLGGPSVFLFPPKPKDTLMISRDPEV |
| | | TCVVVAVSHEDPEVKFNWYVDGVEVDNAKTKPREEQYNSTYRVVS |
| | | VLRVLHVDWLNGKEYKCKVSNKALPAPIEKTISKAKGQPREPQVYT |
| | | LPPSRDELTKNQVSLTCLVKGFYPSDIVVEWESNGQPENNYKTTPPV |
| | | LDSDGSFFLYSKLTVDKSRWQQGNVFSCSVMHEALHNHYTQKSLSL |
| | | SPG |
| hIgG1- | 2218 | ASTKGPSVFPLAPSSKSTSGGTAALGCLVKDYFPEPVTVSWNSGALT |
| LALA/DDRVV | | SGVHTFPAVLQSSGLYSLSSVVTVPSSSLGTQTYICNVNHKPSNTKV |
| | | DKKVEPKSCDKTHTCPPCPAPEAAGGPSVFLFPPKPKDTLMISRDPE |
| | | VTCVVVDVSHEDPEVKFNWYVDGVEVDNAKTKPREEQYNSTYRV |
| | | VSVLRVLHVDWLNGKEYKCKVSNKALPAPIEKTISKAKGQPREPQV |
| | | YTLPPSRDELTKNQVSLTCLVKGFYPSDIVVEWESNGQPENNYKTTP |
| | | PVLDSDGSFFLYSKLTVDKSRWQQGNVFSCSVMHEALHNHYTQKSL |
| | | SLSPG |
| hIgG1- | 2219 | ASTKGPSVFPLAPSSKSTSGGTAALGCLVKDYFPEPVTVSWNSGALT |
| LAGA/DDRVV | | SGVHTFPAVLQSSGLYSLSSVVTVPSSSLGTQTYICNVNHKPSNTKV |
| | | DKKVEPKSCDKTHTCPPCPAPELAGAPSVFLFPPKPKDTLMISRDPEV |
| | | TCVVVDVSHEDPEVKFNWYVDGVEVDNAKTKPREEQYNSTYRVVS |
| | | VLRVLHVDWLNGKEYKCKVSNKALPAPIEKTISKAKGQPREPQVYT |
| | | LPPSRDELTKNQVSLTCLVKGFYPSDIVVEWESNGQPENNYKTTPPV |
| | | LDSDGSFFLYSKLTVDKSRWQQGNVFSCSVMHEALHNHYTQKSLSL |
| | | SPG |
| hIgG1- | 2220 | ASTKGPSVFPLAPSSKSTSGGTAALGCLVKDYFPEPVTVSWNSGALT |
| LALAGA/DDRVV | | SGVHTFPAVLQSSGLYSLSSVVTVPSSSLGTQTYICNVNHKPSNTKV |
| | | DKKVEPKSCDKTHTCPPCPAPEAAGAPSVFLFPPKPKDTLMISRDPE |
| | | VTCVVVDVSHEDPEVKFNWYVDGVEVDNAKTKPREEQYNSTYRV |
| | | VSVLRVLHVDWLNGKEYKCKVSNKALPAPIEKTISKAKGQPREPQV |
| | | YTLPPSRDELTKNQVSLTCLVKGFYPSDIVVEWESNGQPENNYKTTP |
| | | PVLDSDGSFFLYSKLTVDKSRWQQGNVFSCSVMHEALHNHYTQKSL |
| | | SLSPG |
| hIgG1- | 2221 | ASTKGPSVFPLAPSSKSTSGGTAALGCLVKDYFPEPVTVSWNSGALT |
| LALAPG/DDRVV | | SGVHTFPAVLQSSGLYSLSSVVTVPSSSLGTQTYICNVNHKPSNTKV |
| | | DKKVEPKSCDKTHTCPPCPAPEAAGGPSVFLFPPKPKDTLMISRDPE |
| | | VTCVVVDVSHEDPEVKFNWYVDGVEVDNAKTKPREEQYNSTYRV |
| | | VSVLRVLHVDWLNGKEYKCKVSNKALGAPIEKTISKAKGQPREPQV |
| | | YTLPPSRDELTKNQVSLTCLVKGFYPSDIVVEWESNGQPENNYKTTP |
| | | PVLDSDGSFFLYSKLTVDKSRWQQGNVFSCSVMHEALHNHYTQKSL |
| | | SLSPG |
| hIgG1- | 2222 | ASTKGPSVFPLAPSSKSTSGGTAALGCLVKDYFPEPVTVSWNSGALT |
| Q311R/M428L | | SGVHTFPAVLQSSGLYSLSSVVTVPSSSLGTQTYICNVNHKPSNTKV |
| | | DKKVEPKSCDKTHTCPPCPAPELLGGPSVFLFPPKPKDTLMISRTPEV |
| | | TCVVVDVSHEDPEVKFNWYVDGVEVHNAKTKPREEQYNSTYRVVS |
| | | VLTVLHRDWLNGKEYKCKVSNKALPAPIEKTISKAKGQPREPQVYT |
| | | LPPSRDELTKNQVSLTCLVKGFYPSDIAVEWESNGQPENNYKTTPPV |
| | | LDSDGSFFLYSKLTVDKSRWQQGNVFSCSVLHEALHNHYTQKSLSL |
| | | SPG |
| hIgG4- | 2223 | ASTKGPSVFPLAPCSRSTSESTAALGCLVKDYFPEPVTVSWNSGALT |
| Q311R/M428L | | SGVHTFPAVLQSSGLYSLSSVVTVPSSSLGTKTYTCNVDHKPSNTKV |
| | | DKRVESKYGPPCPSCPAPEFLGGPSVFLFPPKPKDTLMISRTPEVTCV |
| | | VVDVSQEDPEVQFNWYVDGVEVHNAKTKPREEQFNSTYRVVSVLT |
| | | VLHRDWLNGKEYKCKVSNKGLPSSIEKTISKAKGQPREPQVYTLPPS |
| | | QEEMTKNQVSLTCLVKGFYPSDIAVEWESNGQPENNYKTTPPVLDS |
| | | DGSFFLYSRLTVDKSRWQEGNVFSCSVLHEALHNHYTQKSLSLSLG |
| | | K |
| IgG4- | 2224 | ASTKGPSVFPLAPCSRSTSESTAALGCLVKDYFPEPVTVSWNSGALT |
| SP/Q311R/ | | SGVHTFPAVLQSSGLYSLSSVVTVPSSSLGTKTYTCNVDHKPSNTKV |
| M428L | | DKRVESKYGPPCPPCPAPEFLGGPSVFLFPPKPKDTLMISRTPEVTCV |
| | | VVDVSQEDPEVQFNWYVDGVEVHNAKTKPREEQFNSTYRVVSVLT |
| | | VLHRDWLNGKEYKCKVSNKGLPSSIEKTISKAKGQPREPQVYTLPPS |
| | | QEEMTKNQVSLTCLVKGFYPSDIAVEWESNGQPENNYKTTPPVLDS |
| | | DGSFFLYSRLTVDKSRWQEGNVFSCSVLHEALHNHYTQKSLSLSLG |
| | | K |
| IgG4- | 2225 | ASTKGPSVFPLAPCSRSTSESTAALGCLVKDYFPEPVTVSWNSGALT |
| SPLE/Q311R/ | | SGVHTFPAVLQSSGLYSLSSVVTVPSSSLGTKTYTCNVDHKPSNTKV |
| M428L | | DKRVESKYGPPCPPCPAPEELGGPSVFLFPPKPKDTLMISRTPEVTCV |
| | | VVDVSQEDPEVQFNWYVDGVEVHNAKTKPREEQFNSTYRVVSVLT |
| | | VLHRDWLNGKEYKCKVSNKGLPSSIEKTISKAKGQPREPQVYTLPPS |
| | | QEEMTKNQVSLTCLVKGFYPSDIAVEWESNGQPENNYKTTPPVLDS |
| | | DGSFFLYSRLTVDKSRWQEGNVFSCSVLHEALHNHYTQKSLSLSLG |
| | | K |
| IgG2- | 2226 | ASTKGPSVFPLAPCSRSTSESTAALGCLVKDYFPEPVTVSWNSGALT |
| Q311R/M428L | | SGVHTFPAVLQSSGLYSLSSVVTVPSSNFGTQTYTCNVDHKPSNTKV |
| | | DKTVERKCCVECPPCPAPPVAGPSVFLFPPKPKDTLMISRTPEVTCVV |
| | | VDVSHEDPEVQFNWYVDGVEVHNAKTKPREEQFNSTFRVVSVLTV |
| | | VHRDWLNGKEYKCKVSNKGLPAPIEKTISKTKGQPREPQVYTLPPSR |
| | | EEMTKNQVSLTCLVKGFYPSDIAVEWESNGQPENNYKTTPPMLDSD |
| | | GSFFLYSKLTVDKSRWQQGNVFSCSVLHEALHNHYTQKSLSLSP |
| hIgG1- | 2227 | ASTKGPSVFPLAPSSKSTSGGTAALGCLVKDYFPEPVTVSWNSGALT |
| N297A/Q311R/ | | SGVHTFPAVLQSSGLYSLSSVVTVPSSSLGTQTYICNVNHKPSNTKV |
| M428L | | DKKVEPKSCDKTHTCPPCPAPELLGGPSVFLFPPKPKDTLMISRTPEV |
| | | TCVVVDVSHEDPEVKFNWYVDGVEVHNAKTKPREEQYASTYRVVS |
| | | VLTVLHRDWLNGKEYKCKVSNKALPAPIEKTISKAKGQPREPQVYT |
| | | LPPSRDELTKNQVSLTCLVKGFYPSDIAVEWESNGQPENNYKTTPPV |
| | | LDSDGSFFLYSKLTVDKSRWQQGNVFSCSVLHEALHNHYTQKSLSL |
| | | SPG |
| hIgG1- | 2228 | ASTKGPSVFPLAPSSKSTSGGTAALGCLVKDYFPEPVTVSWNSGALT |
| D265A/Q311R/ | | SGVHTFPAVLQSSGLYSLSSVVTVPSSSLGTQTYICNVNHKPSNTKV |
| M428L | | DKKVEPKSCDKTHTCPPCPAPELLGGPSVFLFPPKPKDTLMISRTPEV |
| | | TCVVVAVSHEDPEVKFNWYVDGVEVHNAKTKPREEQYNSTYRVVS |
| | | VLTVLHRDWLNGKEYKCKVSNKALPAPIEKTISKAKGQPREPQVYT |
| | | LPPSRDELTKNQVSLTCLVKGFYPSDIAVEWESNGQPENNYKTTPPV |
| | | LDSDGSFFLYSKLTVDKSRWQQGNVFSCSVLHEALHNHYTQKSLSL |
| | | SPG |
| hIgG1- | 2229 | ASTKGPSVFPLAPSSKSTSGGTAALGCLVKDYFPEPVTVSWNSGALT |
| LALA/Q311R/ | | SGVHTFPAVLQSSGLYSLSSVVTVPSSSLGTQTYICNVNHKPSNTKV |
| M428L | | DKKVEPKSCDKTHTCPPCPAPEAAGGPSVFLFPPKPKDTLMISRTPEV |
| | | TCVVVDVSHEDPEVKFNWYVDGVEVHNAKTKPREEQYNSTYRVVS |
| | | VLTVLHRDWLNGKEYKCKVSNKALPAPIEKTISKAKGQPREPQVYT |
| | | LPPSRDELTKNQVSLTCLVKGFYPSDIAVEWESNGQPENNYKTTPPV |
| | | LDSDGSFFLYSKLTVDKSRWQQGNVFSCSVLHEALHNHYTQKSLSL |
| | | SPG |
| hIgG1- | 2230 | ASTKGPSVFPLAPSSKSTSGGTAALGCLVKDYFPEPVTVSWNSGALT |
| LAGA/Q311R/ | | SGVHTFPAVLQSSGLYSLSSVVTVPSSSLGTQTYICNVNHKPSNTKV |
| M428L | | DKKVEPKSCDKTHTCPPCPAPELAGAPSVFLFPPKPKDTLMISRTPEV |
| | | TCVVVDVSHEDPEVKFNWYVDGVEVHNAKTKPREEQYNSTYRVVS |
| | | VLTVLHRDWLNGKEYKCKVSNKALPAPIEKTISKAKGQPREPQVYT |
| | | LPPSRDELTKNQVSLTCLVKGFYPSDIAVEWESNGQPENNYKTTPPV |
| | | LDSDGSFFLYSKLTVDKSRWQQGNVFSCSVLHEALHNHYTQKSLSL |
| | | SPG |
| hIgG1- | 2231 | ASTKGPSVFPLAPSSKSTSGGTAALGCLVKDYFPEPVTVSWNSGALT |
| LALAGA/Q311R/ | | SGVHTFPAVLQSSGLYSLSSVVTVPSSSLGTQTYICNVNHKPSNTKV |
| M428L | | DKKVEPKSCDKTHTCPPCPAPEAAGAPSVFLFPPKPKDTLMISRTPEV |
| | | TCVVVDVSHEDPEVKFNWYVDGVEVHNAKTKPREEQYNSTYRVVS |
| | | VLTVLHRDWLNGKEYKCKVSNKALPAPIEKTISKAKGQPREPQVYT |
| | | LPPSRDELTKNQVSLTCLVKGFYPSDIAVEWESNGQPENNYKTTPPV |
| | | LDSDGSFFLYSKLTVDKSRWQQGNVFSCSVLHEALHNHYTQKSLSL |
| | | SPG |
| hIgG1- | 2232 | ASTKGPSVFPLAPSSKSTSGGTAALGCLVKDYFPEPVTVSWNSGALT |
| LALAPG/Q311R/ | | SGVHTFPAVLQSSGLYSLSSVVTVPSSSLGTQTYICNVNHKPSNTKV |
| M428L | | DKKVEPKSCDKTHTCPPCPAPEAAGGPSVFLFPPKPKDTLMISRTPEV |
| | | TCVVVDVSHEDPEVKFNWYVDGVEVHNAKTKPREEQYNSTYRVVS |
| | | VLTVLHRDWLNGKEYKCKVSNKALGAPIEKTISKAKGQPREPQVYT |
| | | LPPSRDELTKNQVSLTCLVKGFYPSDIAVEWESNGQPENNYKTTPPV |
| | | LDSDGSFFLYSKLTVDKSRWQQGNVFSCSVLHEALHNHYTQKSLSL |
| | | SPG |

In some embodiments, the α4β7 integrin binding protein comprises a Fc, wherein the Fc has an amino acid sequence with a terminal Lysine as compared to any one of SEQ ID NO: 2121, 2123, 2126-2152, 2162-2164, 2166-2222, 2226-2232. In some embodiments, the α4β7 integrin binding protein comprises a Fc, wherein the Fc has an amino acid sequence that lacks a terminal Lysine as compared to any one of SEQ ID NO: 2122, 2124, 2125, 2153-2161, 2165, 2223-2225.

In some embodiments, the α4β7 integrin binding protein comprises a Fc comprising one or more modifications in SEQ ID NO: 2121. In some embodiments, the α4β7 integrin binding protein comprises a Fc comprising one or more modifications in SEQ ID NO: 2122. In some embodiments, the α4β7 integrin binding protein comprises a Fc comprising one or more modifications in SEQ ID NO: 2123. In some embodiments, the Fc comprises an amino acid sequence having at least 80% sequence identity with the amino acid sequence according to any one of SEQ ID NOs: 2121-2123. In some embodiments, the Fc comprises an amino acid sequence having at least 85% sequence identity with the amino acid sequence according to any one of SEQ ID NOs: 2121-2123. In some embodiments, the Fc comprises an amino acid sequence having at least 90% sequence identity with the amino acid sequence according to any one of SEQ ID NOs: 2121-2123. In some embodiments, the Fc comprises an amino acid sequence having at least 95% sequence identity with the amino acid sequence according to any one of SEQ ID NOs: 2121-2123. In some embodiments, the Fc comprises an amino acid sequence having at least 96% sequence identity with the amino acid sequence according to any one of SEQ ID NOs: 2121-2123. In some embodiments, the Fc comprises an amino acid sequence having at least 97% sequence identity with the amino acid sequence according to any one of SEQ ID NOs: 2121-2123. In some embodiments, the Fc comprises an amino acid sequence having at least 98% sequence identity with the amino acid sequence according to any one of SEQ ID NOs: 2121-2123. In some embodiments, the Fc comprises an amino acid sequence having at least 99% sequence identity with the amino acid sequence according to any one of SEQ ID NOs: 2121-2123. In some embodiments, the Fc comprises the amino acid sequence according to any one of SEQ ID NOs: 2121-2123.

In some embodiments, one or more modifications in the modified Fc is selected from the group consisting of: S298A, E333A, K334A, K326A, F243L, R292P, Y300L, V305I, P396L, F243L, R292P, Y300L, L235V, P396L, F243L, S239D, 1332E, A330L, S267E, L328F, D265S, S239E, K326A, A327H, G237F, K326E, G236A, D270L, H268D, S324T, L234F, N325L, V266L, and S267D. In some embodiments, one or more modifications in the modified Fc is selected from the group consisting of S228P, M252Y, S254T, T256E, T256D, T250Q, H285D, T307A, T307Q, T307R, T307W, L309D, Q411H, Q311V, A378V, E380A, M428L, N434A, N434S, N297A, D265A, L234A, L235A, and N434W.

In some embodiments, the modified Fc comprises a specific combination of amino acid substitutions selected from the group consisting of: L234A/L235A; V234A/G237A; L235A/G237A/E318A; S228P/L236E; H268QN309L/A330S/A331S; C220S/C226S/C229S/P238S; C226S/C229S/E3233P/L235V/L235A; L234F/L235E/P331S; C226S/P230S; L234A/G237A; L234A/L235A/G237A; Q311R/M428L; and L234A/L235A/P329G.

In some embodiments, the modified Fc comprises a specific combination of amino acid substitutions selected from the group consisting of M428L/N434S (LS); M252Y/S254T/T256E (YTE); T250Q/M428L; T307A/E380A/N434A; T256D/T307Q (DQ); T256D/T307W (DW); M252Y/T256D (YD); T307Q/Q311V/A378V (QVV); T256D/H285D/T307R/Q311V/A378V (DDRVV); L309D/Q311H/N434S (DHS); S228P/L235E (SPLE); L234A/L235A (LALA); M428L/N434A (LA); L234A/G237A (LAGA); L234A/L235A/G237A (LALAGA); L234A/L235A/P329G (LALAPG); N297A/YTE; D265A/YTE; LALA/YTE; LAGA/YTE; LALAGA/YTE; LALAPG/YTE; N297A/LS; D265A/LS; LALA/LS; LAGA/LS; LALAGA/LS; LALAPG/LS; N297A/DHS; D265A/DHS; LALA/DHS; LAGA/DHS; LALAGA/DHS; LALAPG/DHS; SP/YTE; SPLE/YTE; SP/LS; SPLE/LS; SP/DHS; SPLE/DHS; N297A/LA; D265A/LA; LALA/LA; LAGA/LA; LALAGA/LA; LALAPG/LA; N297A/N434A; D265A/N434A; LALA/N434A; LAGA/N434A; LALAGA/N434A; LALAPG/N434A; N297A/N434W; D265A/N434W; LALA/N434W; LAGA/N434W; LALAGA/N434W; LALAPG/N434W; N297A/DQ; D265A/DQ; LALA/DQ; LAGA/DQ; LALAGA/DQ; LALAPG/DQ; N297A/DW; D265A/DW; LALA/DW; LAGA/DW; LALAGA/DW; LALAPG/DW; N297A/YD; D265A/YD; LALA/YD; LAGA/YD; LALAGA/YD; LALAPG/YD; N297A/QVV; D265A/QVV; LALA/QVV; LAGA/QVV, LALAGA/QVV; LALAPG/QVV; N297A/DDRVV; D265A/DDRVV; LALA/DDRVV; LAGA/DDRVV; LALAGA/DDRVV; LALAPG/DDRVV; SP/Q311R/M428L; SPLE/Q311R/M428L; N297A/Q311R/M428L; D265A/Q311R/M428L; LALA/Q311R/M428L; LAGA/Q311R/M428L; LALAGA/Q311R/M428L; and LALAPG/Q311R/M428L. In some embodiments, the modified Fc comprises a specific combination of amino acid substitutions selected from the group consisting of M428L/N434S (LS) and M252Y/S254T/T256E (YTE). In some embodiments, the modified Fc comprises M428L/N434S (LS) (e.g., SEQ ID NO: 2139, SEQ ID NO: 2156, SEQ ID NO: 2163) modifications. In some embodiments, the modified Fc comprises M252Y/S254T/T256E (YTE) (e.g., SEQ ID NO: 2132, SEQ ID NO: 2153, SEQ ID NO: 2162) modifications.

In some embodiments, the α4β7 integrin binding proteins described herein include modifications to improve its ability to mediate effector function. Such modifications are known in the art and include afucosylation, or engineering of the affinity of the Fc towards an activating receptor, mainly FCGR3a for antibody-dependent cellular cytotoxicity (ADCC), and towards C1q for complement-dependent cytotoxicity (CDC).

In some aspects, an antibody provided herein comprises a Fc domain (e.g., IgG1) with reduced fucose content at position Asn 297 (EU numbering) compared to a naturally occurring Fc domain. Such Fc domains are known to have improved ADCC. In some aspects, such antibodies do not comprise any fucose at position Asn 297.

In some embodiments, the α4β7 integrin binding proteins described herein comprise an Fc region with one or more amino acid substitutions which improve ADCC, such as a substitution at one or more of positions 298, 333, and 334 of the Fc region. In some embodiments, an antibody provided herein comprises an Fc region with one or more amino acid substitutions at positions 239, 332, and 330.

In some embodiments, the Fc comprises an amino acid sequence having at least 80% sequence identity with the amino acid sequence according to any one of SEQ ID NOs: 2124-2232. In some embodiments, the Fc comprises an amino acid sequence having at least 85% sequence identity with the amino acid sequence according to any one of SEQ ID NOs: 2124-2232. In some embodiments, the Fc comprises an amino acid sequence having at least 90% sequence identity with the amino acid sequence according to any one of SEQ ID NOs: 2124-2232. In some embodiments, the Fc comprises an amino acid sequence having at least 95% sequence identity with the amino acid sequence according to any one of SEQ ID NOs: 2124-2232. In some embodiments, the Fc comprises an amino acid sequence having at least 96% sequence identity with the amino acid sequence according to any one of SEQ ID NOs: 2124-2232. In some embodiments, the Fc comprises an amino acid sequence having at least 97% sequence identity with the amino acid sequence according to any one of SEQ ID NOs: 2124-2232. In some embodiments, the Fc comprises an amino acid sequence having at least 98% sequence identity with the amino acid sequence according to any one of SEQ ID NOs: 2124-2232. In some embodiments, the Fc comprises an amino acid sequence having at least 99% sequence identity with the amino acid sequence according to any one of SEQ ID NOs: 2124-2232. In some embodiments, the Fc comprises the amino acid sequence according to any one of SEQ ID NOs: 2124-2232.

In some embodiments, the α4β7 integrin binding proteins described herein comprise an Fc region with at least one galactose residue in the oligosaccharide attached to the Fc region. Such antibody variants may have improved CDC function.

In some embodiments, the α4β7 integrin binding proteins described herein comprise one or more alterations that improve or diminish C1q binding and/or CDC.

In certain embodiments, the Fc region comprises one or more amino acid substitutions, wherein the one or more substitutions result in an increase in one or more of antibody half-life, ADCC activity, ADCP activity, or CDC activity compared with the Fc without the one or more substitutions. In certain embodiments, the one or more amino acid substitutions results in increased antibody half-life at pH 6.0 compared to an antibody comprising a wild-type Fc region. In certain embodiments, the antibody has an increased half-life that is about 10,000-fold, 1,000-fold, 500-fold, 100-fold, 50-fold, 20-fold, 10-fold, 9-fold, 8-fold, 7-fold, 6-fold, 5-fold, 4.5-fold, 4-fold, 3.5-fold, 3-fold, 2.5-fold, 2-fold, 1.95-fold, 1.9-fold, 1.85-fold, 1.8-fold, 1.75-fold, 1.7-fold, 1.65-fold, 1.6-fold, 1.55-fold, 1.50-fold, 1.45-fold, 1.4-fold, 1.35-fold, 1.3-fold, 1.25-fold, 1.2-fold, 1.15-fold, 1.1-fold, or 1.05-fold longer compared to an antibody comprising a wild-type Fc region.

In certain embodiments, the Fc region comprises one or more amino acid substitutions, wherein the one or more substitutions result in a decrease in one or more of ADCC activity, ADCP activity, or CDC activity compared with the Fc without the one or more substitutions.

In certain embodiments, the Fc region binds an Fcγ Receptor selected from the group consisting of: FcγRI, FcγRIIa, FcγRIIb, FcγRIIc, FcγRIIIa, and FcγRIIIb. In certain embodiments, the Fc region binds an Fcγ Receptor with higher affinity at pH 6.0 compared to an antibody comprising a wild-type Fc region.

In some embodiments, the α4β7 integrin binding proteins described herein comprise an extended half-life (i.e., serum half-life). In some embodiments, the α4β7 integrin binding proteins described herein comprise a half-life of at least about 14, 28, 42, 56, 70, 84, 96, or more than 96 weeks. In some embodiments, the α4β7 integrin binding proteins described herein comprise a half-life in a range of about 14 days to about 96 days, about 14 days to about 84 days, about 14 days to about 70 days, about 14 days to about 56 days, about 14 days to about 42 days, about 14 days to about 28 days, of about 28 days to about 96 days, about 28 days to about 84 days, about 28 days to about 70 days, about 28 days to about 56 days, about 28 days to about 42 days, of about 42 days to about 96 days, about 42 days to about 84 days, about 42 days to about 70 days, about 42 days to about 56 days, of about 56 days to about 96 days, about 56 days to about 84 days, about 56 days to about 70 days, of about 70 days to about 96 days, about 70 days to about 84 days, or of about 84 days to about 96 days. In some embodiments, the α4β7 integrin binding proteins described herein comprise a half-life in a range of about 42 days to about 56 days. In some embodiments, the α4β7 integrin binding proteins described herein comprise a half-life of at least about 50 days. In some embodiments, the α4β7 integrin binding proteins described herein comprise a half-life of about 50 days. Methods of measuring half-life are known in the art. In some embodiments, the half-life is measured in a non-human primate. In some embodiments, the half-life is measured in a human. In some embodiments, the half-life is measured following intravenous administration. In some embodiments, the half-life is measured following subcutaneous administration.

In some embodiments, the α4β7 integrin binding proteins described herein have a half-life that is at least 20% longer than a comparator antibody. In some embodiments, the comparator antibody comprises the same complementarity determining regions and variable regions but different Fc regions. In some embodiments, the half-life of the α4β7 integrin binding proteins described herein is at least 30%, at least 40%, at least 50%, at least 60%, at least 70%, at least 80%, or at least 90% longer than the half-life of the comparator antibody. In some embodiments, the half-life of the α4β7 integrin binding proteins described herein is longer than the half-life of the comparator antibody by at least 2 fold, at least 3 fold, at least 4 fold, at least 5 fold, at least 6 fold, at least 7 fold, at least 8 fold, at least 9 fold, or at least 10 fold.

### Methods of Treatment

Described herein, in certain embodiments, are methods of treating an inflammatory bowel disease in a patient in need thereof, the method comprising subcutaneously or intravenously administering to the patient an effective amount of an α4β7 integrin binding protein comprising: a) a heavy chain variable region (VH) comprising (i) a CDR1 having an amino acid sequence according to SEQ ID NO: 3, (ii) a CDR2 having an amino acid sequence according to SEQ ID NO: 109, and (iii) a CDR3 having an amino acid sequence according to SEQ ID NO: 215; and a light chain variable region (VL) comprising (i) a CDR1 having an amino acid sequence according to SEQ ID NO: 321, (ii) a CDR2 having an amino acid sequence according to SEQ ID NO: 427, and (iii) a CDR3 having an amino acid sequence according to SEQ ID NO: 533; b) a heavy chain variable region (VH) comprising (i) a CDR1 having an amino acid sequence according to SEQ ID NO: 4, (ii) a CDR2 having an amino acid sequence according to SEQ ID NO: 110, and (iii) a CDR3 having an amino acid sequence according to SEQ ID NO: 216; and a light chain variable region (VL) comprising (i) a CDR1 having an amino acid sequence according to SEQ ID NO: 322, (ii) a CDR2 having an amino acid sequence according to SEQ ID NO: 428, and (iii) a CDR3 having an amino acid sequence according to SEQ ID NO: 534; c) a heavy chain variable region (VH) comprising (i) a CDR1 having an amino acid sequence according to SEQ ID NO: 5, (ii) a CDR2 having an amino acid sequence according to SEQ ID NO: 111, and (iii) a CDR3 having an amino acid sequence according to SEQ ID NO: 217; and a light chain variable region (VL) comprising (i) a CDR1 having an amino acid sequence according to SEQ ID NO: 323, (ii) a CDR2 having an amino acid sequence according to SEQ ID NO: 429, and (iii) a CDR3 having an amino acid sequence according to SEQ ID NO: 535; d) a heavy chain variable region (VH) comprising (i) a CDR1 having an amino acid sequence according to SEQ ID NO: 6, (ii) a CDR2 having an amino acid sequence according to SEQ ID NO: 112, and (iii) a CDR3 having an amino acid sequence according to SEQ ID NO: 218; and a light chain variable region (VL) comprising (i) a CDR1 having an amino acid sequence according to SEQ ID NO: 324, (ii) a CDR2 having an amino acid sequence according to SEQ ID NO: 430, and (iii) a CDR3 having an amino acid sequence according to SEQ ID NO: 536; e) a heavy chain variable region (VH) comprising (i) a CDR1 having an amino acid sequence according to SEQ ID NO: 81, (ii) a CDR2 having an amino acid sequence according to SEQ ID NO: 187, and (iii) a CDR3 having an amino acid sequence according to SEQ ID NO: 293; and a light chain variable region (VL) comprising (i) a CDR1 having an amino acid sequence according to SEQ ID NO: 399, (ii) a CDR2 having an amino acid sequence according to SEQ ID NO: 505, and (iii) a CDR3 having an amino acid sequence according to SEQ ID NO: 611; f) a heavy chain variable region (VH) comprising (i) a CDR1 having an amino acid sequence according to SEQ ID NO: 82, (ii) a CDR2 having an amino acid sequence according to SEQ ID NO: 188, and (iii) a CDR3 having an amino acid sequence according to SEQ ID NO: 294; and a light chain variable region (VL) comprising (i) a CDR1 having an amino acid sequence according to SEQ ID NO: 400, (ii) a CDR2 having an amino acid sequence according to SEQ ID NO: 506, and (iii) a CDR3 having an amino acid sequence according to SEQ ID NO: 612; or g) a heavy chain variable region (VH) comprising (i) a CDR1 having an amino acid sequence according to SEQ ID NO: 83, (ii) a CDR2 having an amino acid sequence according to SEQ ID NO: 189, and (iii) a CDR3 having an amino acid sequence according to SEQ ID NO: 295; and a light chain variable region (VL) comprising (i) a CDR1 having an amino acid sequence according to SEQ ID NO: 401, (ii) a CDR2 having an amino acid sequence according to SEQ ID NO: 507, and (iii) a CDR3 having an amino acid sequence according to SEQ ID NO: 613.

Further described herein, in certain embodiments, are methods of treating an inflammatory bowel disease in a patient in need thereof, the method comprising subcutaneously or intravenously administering to the patient an effective amount of an α4β7 integrin binding protein comprising: a) a heavy chain variable region (VH) comprising (i) a CDR1 having an amino acid sequence according to any one of SEQ ID NOs: 7-26, 32-58, 60-80, and 84-106; (ii) a CDR2 having an amino acid sequence according to any one of SEQ ID NOs: 113-132, 138-164, 166-186, and 190-212; and (iii) a CDR3 having an amino acid sequence according to any one of SEQ ID NOs: 219-238, 244-270, 272-292, and 296-318; and b) a light chain variable region (VL) comprising (i) a CDR1 having an amino acid sequence according to any one of SEQ ID NOs: 325-344, 350-376, 378-398, and 402-424; (ii) a CDR2 having an amino acid sequence according to any one of SEQ ID NOs: 431-450, 456-482, 484-504, and 508-530; and (iii) a CDR3 having an amino acid sequence according to any one of SEQ ID NOs: 537-556, 562-588, 590-610, and 614-636.

Further described herein, in certain embodiments, are methods of treating an inflammatory bowel disease in a patient in need thereof, the method comprising subcutaneously or intravenously administering to the patient an effective amount of an α4β7 integrin binding protein comprising: a) a heavy chain variable region (VH) comprising the amino acid sequence according to any one of SEQ ID NOs: 1909, 1910, 1935-1939, 1967; and b) a light chain variable region (VL) comprising the amino acid sequence according to any one of SEQ ID NOs: 2015, 2016, 2041-2045, 2073.

Further described herein, in certain embodiments, are methods of treating an inflammatory bowel disease in a patient in need thereof, the method comprising subcutaneously or intravenously administering to the patient an effective amount of an α4β7 integrin binding protein as described above. comprising: a) a heavy chain variable region (VH) comprising (i) a CDR1 having an amino acid sequence according to any one of SEQ ID NOs: 1-106, (ii) a CDR2 having an amino acid sequence according to any one of SEQ ID NOs: 107-212, and (iii) a CDR3 having an amino acid sequence according to any one of SEQ ID NOs: 213-318; b) a light chain variable region (VL) comprising (i) a CDR1 having an amino acid sequence according to any one of SEQ ID NOs: 319-424, (ii) a CDR2 having an amino acid sequence according to any one of SEQ ID NOs: 425-530, and (iii) a CDR3 having an amino acid sequence according to any one of SEQ ID NOs: 531-636; and c) a modified Fc that comprises amino acid modifications M252Y, S254T, and T256E (YTE) and/or L234A/G237A (LAGA).

Further described herein, in certain embodiments, are methods of treating an inflammatory bowel disease in a patient in need thereof, the method comprising subcutaneously or intravenously administering to the patient an effective amount of an α4β7 integrin binding protein comprising a modified Fc that extends half-life of the α4β7 binding protein as compared to an α4β7 binding protein that does not comprise the modified Fc.

Further described herein, in certain embodiments, are methods of treating an inflammatory bowel disease in a patient in need thereof, the method comprising subcutaneously or intravenously administering to the patient an effective amount of an α4β7 integrin binding protein, wherein the α4β7 binding protein specifically binds to an epitope of α4β7 and comprises a Fc domain comprising amino acid modifications M252Y, S254T, and T256E (YTE) and/or L234A/G237A (LAGA).

In some embodiments, the inflammatory bowel disease is Crohn's disease or ulcerative colitis. In some embodiments, the inflammatory bowel disease is ulcerative colitis. In some embodiments, the inflammatory bowel disease is Crohn's disease.

In some embodiments, administration of the α4β7 integrin binding protein is intravenous, intratumoral, intramuscular, subcutaneous, intralesional, intraintestinal, intracolonic, intrarectal, intrapouch, or intraperitoneal. In some embodiments, administration of the α4β7 integrin binding protein is through a parenteral route such as intravenous, intramuscular, subcutaneous, intraarterial, or intraperitoneal administration. In some embodiments, administration of the α4β7 integrin binding protein is intravenous or subcutaneous. In some embodiments, administration of the α4β7 integrin binding protein is intravenous. In some embodiments, administration of the α4β7 integrin binding protein is subcutaneous.

Administration of the α4β7 integrin binding protein can occur at various intervals and various doses.

### Pharmaceutical Compositions

The present disclosure also features pharmaceutical compositions that contain a therapeutically effective amount of the α4β7 integrin binding proteins described herein. The composition can be formulated for use in a variety of drug delivery systems. One or more physiologically acceptable excipients or carriers can also be included in the composition for proper formulation. Suitable formulations for use in the present disclosure are found in Remington's Pharmaceutical Sciences, Mack Publishing Company, Philadelphia, Pa., 17th ed., 1985. For a brief review of methods for drug delivery, see, e.g., Langer (Science 249:1527-1533, 1990).

In some embodiments, a pharmaceutical composition may contain formulation materials for modifying, maintaining or preserving, for example, the pH, osmolarity, viscosity, clarity, color, isotonicity, odor, sterility, stability, rate of dissolution or release, adsorption or penetration of the composition. In such embodiments, suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides; disaccharides; and other carbohydrates (such as glucose, mannose or dextrins); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate, triton, tromethamine, lecithin, cholesterol, tyloxapal); stability enhancing agents (such as sucrose or sorbitol); tonicity enhancing agents (such as alkali metal halides, preferably sodium or potassium chloride, mannitol sorbitol); delivery vehicles; diluents; excipients and/or pharmaceutical adjuvants (see, Remington's Pharmaceutical Sciences, 18th ed. (Mack Publishing Company, 1990)).

In some embodiments, a pharmaceutical composition is citrate-free.

In some embodiments, a pharmaceutical composition may contain nanoparticles, e.g., polymeric nanoparticles, liposomes, or micelles.

In some embodiments, a pharmaceutical composition may contain a sustained- or controlled-delivery formulation. Techniques for formulating sustained- or controlled-delivery means, such as liposome carriers, bio-erodible microparticles or porous beads and depot injections, are also known to those skilled in the art. Sustained-release preparations may include, e.g., porous polymeric microparticles or semipermeable polymer matrices in the form of shaped articles, e.g., films, or microcapsules. Sustained release matrices may include polyesters, hydrogels, polylactides, copolymers of L-glutamic acid and gamma ethyl-L-glutamate, poly (2-hydroxyethyl-inethacrylate), ethylene vinyl acetate, or poly-D(−)-3-hydroxybutyric acid. Sustained release compositions may also include liposomes that can be prepared by any of several methods known in the art.

Pharmaceutical compositions containing an α4β7 integrin binding protein disclosed herein can be presented in a dosage unit form and can be prepared by any suitable method. A pharmaceutical composition should be formulated to be compatible with its intended route of administration. Examples of routes of administration are intravenous (IV), intradermal, inhalation, transdermal, topical, transmucosal, intrathecal and rectal administration. In some embodiments, the α4β7 integrin binding protein disclosed herein is administered intravenously or subcutaneously. In some embodiments, the α4β7 integrin binding protein disclosed herein is administered intravenously. In some embodiments, the α4β7 integrin binding protein disclosed herein is administered subcutaneously.

Useful formulations can be prepared by methods known in the pharmaceutical art. For example, see Remington's Pharmaceutical Sciences, 18th ed. (Mack Publishing Company, 1990). Formulation components suitable for parenteral administration include a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as EDTA; buffers such as acetates, citrates or phosphates; and agents for the adjustment of tonicity such as sodium chloride or dextrose. In some embodiments, the formulation for parenteral administration is citrate-free.

For intravenous or subcutaneous administration, suitable carriers include physiological saline, bacteriostatic water, Cremophor EL™ (BASF, Parsippany, NJ) or phosphate buffered saline (PBS). The carrier should be stable under the conditions of manufacture and storage, and should be preserved against microorganisms. The carrier can be a solvent or dispersion medium containing, for example, water, ethanol, polyol (for example, glycerol, propylene glycol, and liquid polyetheylene glycol), and suitable mixtures thereof.

An intravenous or subcutaneous drug delivery formulation may be contained in a syringe, pen, or bag. In some embodiments, the bag is connected to a channel comprising a tube and/or a needle. In some embodiments, the formulation is a lyophilized formulation or a liquid formulation. In some embodiments, the formulation is an injectable liquid formulation. Various devices can be used to deliver liquid formulations by subcutaneous route of administration, including on-body infusion devices, autoinjector devices, prefilled syringes, and syringes. Generally, administration time depends on volume and device, and can range from seconds to minutes.

These compositions may be sterilized by conventional sterilization techniques, or may be sterile filtered. The resulting aqueous solutions may be packaged for use as-is, or lyophilized, the lyophilized preparation being combined with a sterile aqueous carrier prior to administration.

A polyol, which acts as a tonicifier and may stabilize the α4β7 integrin binding protein, may also be included in the formulation. The polyol is added to the formulation in an amount which may vary with respect to the desired isotonicity of the formulation. In some embodiments, the aqueous formulation is isotonic. The amount of polyol added may also be altered with respect to the molecular weight of the polyol. For example, a lower amount of a monosaccharide (e.g., mannitol) is added, compared to a disaccharide (such as trehalose). In some embodiments, the polyol which is used in the formulation as a tonicity agent is mannitol.

A detergent or surfactant may also be added to the formulation. Exemplary detergents include nonionic detergents such as polysorbates (e.g., polysorbates 20, 80 etc.) or poloxamers (e.g., poloxamer 188). The amount of detergent added is such that it reduces aggregation of the formulated antibody and/or minimizes the formation of particulates in the formulation and/or reduces adsorption. In some embodiments, the formulation may include a surfactant which is a polysorbate. In some embodiments, the formulation may contain the detergent polysorbate 80 or Tween 80. Tween 80 is a term used to describe polyoxyethylene (20) sorbitanmonooleate (see Fiedler, Lexikon der Hifsstoffe, Editio Cantor Verlag Aulendorf, 4th edi., 1996).

In embodiments, the protein product of the present disclosure is formulated as a liquid formulation. In some embodiments, the liquid formulation is prepared in combination with a sugar at stabilizing levels. In some embodiments, the liquid formulation is prepared in an aqueous carrier. In some embodiments, a stabilizer is added in an amount no greater than that which may result in a viscosity undesirable or unsuitable for intravenous administration. In some embodiments, the sugar is disaccharides, e.g., sucrose. In some embodiments, the liquid formulation may also include one or more of a buffering agent, a surfactant, and a preservative.

In some embodiments, the pH of the liquid formulation is set by addition of a pharmaceutically acceptable acid and/or base. In some embodiments, the pharmaceutically acceptable acid is hydrochloric acid. In some embodiments, the base is sodium hydroxide.

The aqueous carrier of interest herein is one which is pharmaceutically acceptable (safe and non-toxic for administration to a human) and is useful for the preparation of a liquid formulation. Illustrative carriers include sterile water for injection (SWFI), bacteriostatic water for injection (BWFI), a pH buffered solution (e.g., phosphate-buffered saline), sterile saline solution, Ringer's solution or dextrose solution.

A preservative may be optionally added to the formulations herein to reduce bacterial action. The addition of a preservative may, for example, facilitate the production of a multi-use (multiple-dose) formulation.

The α4β7 integrin binding protein may be lyophilized to produce a lyophilized formulation including the proteins and a lyoprotectant. The lyoprotectant may be sugar, e.g., disaccharides. In some embodiments, the lyoprotectant is sucrose or maltose. The lyophilized formulation may also include one or more of a buffering agent, a surfactant, a bulking agent, and/or a preservative.

The amount of sucrose or maltose useful for stabilization of the lyophilized drug product may be in a weight ratio of at least 1:2 protein to sucrose or maltose. In some embodiments, the protein to sucrose or maltose weight ratio is of from 1:2 to 1:5. In some embodiments, the pH of the formulation, prior to lyophilization, is set by addition of a pharmaceutically acceptable acid and/or base. In some embodiments, the pharmaceutically acceptable acid is hydrochloric acid. In some embodiments, the pharmaceutically acceptable base is sodium hydroxide.

In some embodiments, the α4β7 integrin binding protein is administered at an uniform dose. Alternatively, a patient's dose can be tailored to the approximate body weight or surface area of the patient. Other factors in determining the appropriate dosage can include the disease or condition to be treated or prevented, the severity of the disease, the route of administration, and the age, sex, and medical condition of the patient. Further refinement of the calculations necessary to determine the appropriate dosage for treatment is routinely made by those skilled in the art, especially in light of the dosage information and assays disclosed herein. The dosage can also be determined through the use of known assays for determining dosages used in conjunction with appropriate dose-response data. An individual patient's dosage can be adjusted as the progress of the disease is monitored. Blood levels of the targetable construct or complex in a patient can be measured to see if the dosage needs to be adjusted to reach or maintain an effective concentration. Pharmacogenomics may be used to determine which targetable constructs and/or complexes, and dosages thereof, are most likely to be effective for a given individual (Schmitz et al., Clinica Chimica Acta 308: 43-53, 2001; Steimer et al., Clinica Chimica Acta 308: 33-41, 2001).

### Methods of Preparation

The α4β7 integrin binding proteins described above can be made using recombinant DNA technology well known to a skilled person in the art. For example, one or more isolated polynucleotides encoding the α4β7 integrin binding protein can be ligated to other appropriate nucleotide sequences, including, for example, constant region coding sequences, and expression control sequences, to produce conventional gene expression constructs (i.e., expression vectors) encoding the desired α4β7 integrin binding proteins. Production of defined gene constructs is within routine skill in the art.

Nucleic acids encoding desired α4β7 integrin binding proteins can be incorporated (ligated) into expression vectors, which can be introduced into host cells through conventional transfection or transformation techniques. Exemplary host cells are E. coli cells, Chinese hamster ovary (CHO) cells, human embryonic kidney 293 (HEK 293) cells, HeLa cells, baby hamster kidney (BHK) cells, monkey kidney cells (COS), human hepatocellular carcinoma cells (e.g., Hep G2), and myeloma cells that do not otherwise produce IgG protein. Transformed host cells can be grown under conditions that permit the host cells to express the genes that encode α4β7 integrin binding proteins.

Specific expression and purification conditions will vary depending upon the expression system employed. For example, if a gene is to be expressed in E. coli, it is first cloned into an expression vector by positioning the engineered gene downstream from a suitable bacterial promoter, e.g., Trp or Tac, and a prokaryotic signal sequence. The expressed protein may be secreted. The expressed protein may accumulate in refractile or inclusion bodies, which can be harvested after disruption of the cells by French press or sonication. The refractile bodies then are solubilized, and the protein may be refolded and/or cleaved by methods known in the art.

If the engineered gene is to be expressed in eukaryotic host cells, e.g., CHO cells, it is first inserted into an expression vector containing a suitable eukaryotic promoter, a secretion signal, a poly A sequence, and a stop codon. Optionally, the vector or gene construct may contain enhancers and introns. In embodiments involving fusion proteins comprising an α4β7 integrin binding protein or portion thereof, the expression vector optionally contains sequences encoding all or part of a constant region, enabling an entire, or a part of, a heavy or light chain to be expressed. The gene construct can be introduced into eukaryotic host cells using conventional techniques.

In some embodiments, in order to express an α4β7 integrin binding protein, an N-terminal signal sequence is included in the protein construct. Exemplary N-terminal signal sequences include signal sequences from interleukin-2, CD-5, IgG kappa light chain, trypsinogen, serum albumin, and prolactin.

After transfection, single clones can be isolated for cell bank generation using methods known in the art, such as limited dilution, ELISA, FACS, microscopy, or Clonepix. Clones can be cultured under conditions suitable for bio-reactor scale-up and maintained expression of the α4β7 integrin binding proteins.

The α4β7 integrin binding proteins can be isolated and purified using methods known in the art including centrifugation, depth filtration, cell lysis, homogenization, freeze-thawing, affinity purification, gel filtration, ion exchange chromatography, hydrophobic interaction exchange chromatography, and mixed-mode chromatography.

### Specific Embodiments

Non-limiting specific embodiments are described below, each of which is considered to be within the present disclosure.

Embodiment 1. An α4β7 binding protein comprising:
a) a heavy chain variable region (VH) comprising (i) a CDR1 having an amino acid sequence according to SEQ ID NO: 3, (ii) a CDR2 having an amino acid sequence according to SEQ ID NO: 109, and (iii) a CDR3 having an amino acid sequence according to SEQ ID NO: 215; and a light chain variable region (VL) comprising (i) a CDR1 having an amino acid sequence according to SEQ ID NO: 321, (ii) a CDR2 having an amino acid sequence according to SEQ ID NO: 427, and (iii) a CDR3 having an amino acid sequence according to SEQ ID NO: 533;
b) a heavy chain variable region (VH) comprising (i) a CDR1 having an amino acid sequence according to SEQ ID NO: 4, (ii) a CDR2 having an amino acid sequence according to SEQ ID NO: 110, and (iii) a CDR3 having an amino acid sequence according to SEQ ID NO: 216; and a light chain variable region (VL) comprising (i) a CDR1 having an amino acid sequence according to SEQ ID NO: 322, (ii) a CDR2 having an amino acid sequence according to SEQ ID NO: 428, and (iii) a CDR3 having an amino acid sequence according to SEQ ID NO: 534;
c) a heavy chain variable region (VH) comprising (i) a CDR1 having an amino acid sequence according to SEQ ID NO: 5, (ii) a CDR2 having an amino acid sequence according to SEQ ID NO: 111, and (iii) a CDR3 having an amino acid sequence according to SEQ ID NO: 217; and a light chain variable region (VL) comprising (i) a CDR1 having an amino acid sequence according to SEQ ID NO: 323, (ii) a CDR2 having an amino acid sequence according to SEQ ID NO: 429, and (iii) a CDR3 having an amino acid sequence according to SEQ ID NO: 535;
d) a heavy chain variable region (VH) comprising (i) a CDR1 having an amino acid sequence according to SEQ ID NO: 6, (ii) a CDR2 having an amino acid sequence according to SEQ ID NO: 112, and (iii) a CDR3 having an amino acid sequence according to SEQ ID NO: 218; and a light chain variable region (VL) comprising (i) a CDR1 having an amino acid sequence according to SEQ ID NO: 324, (ii) a CDR2 having an amino acid sequence according to SEQ ID NO: 430, and (iii) a CDR3 having an amino acid sequence according to SEQ ID NO: 536;
e) a heavy chain variable region (VH) comprising (i) a CDR1 having an amino acid sequence according to SEQ ID NO: 81, (ii) a CDR2 having an amino acid sequence according to SEQ ID NO: 187, and (iii) a CDR3 having an amino acid sequence according to SEQ ID NO: 293; and a light chain variable region (VL) comprising (i) a CDR1 having an amino acid sequence according to SEQ ID NO: 399, (ii) a CDR2 having an amino acid sequence according to SEQ ID NO: 505, and (iii) a CDR3 having an amino acid sequence according to SEQ ID NO: 611;
f) a heavy chain variable region (VH) comprising (i) a CDR1 having an amino acid sequence according to SEQ ID NO: 82, (ii) a CDR2 having an amino acid sequence according to SEQ ID NO: 188, and (iii) a CDR3 having an amino acid sequence according to SEQ ID NO: 294; and a light chain variable region (VL) comprising (i) a CDR1 having an amino acid sequence according to SEQ ID NO: 400, (ii) a CDR2 having an amino acid sequence according to SEQ ID NO: 506, and (iii) a CDR3 having an amino acid sequence according to SEQ ID NO: 612; or
g) a heavy chain variable region (VH) comprising (i) a CDR1 having an amino acid sequence according to SEQ ID NO: 83, (ii) a CDR2 having an amino acid sequence according to SEQ ID NO: 189, and (iii) a CDR3 having an amino acid sequence according to SEQ ID NO: 295; and a light chain variable region (VL) comprising (i) a CDR1 having an amino acid sequence according to SEQ ID NO: 401, (ii) a CDR2 having an amino acid sequence according to SEQ ID NO: 507, and (iii) a CDR3 having an amino acid sequence according to SEQ ID NO: 613.

Embodiment 2. The α4β7 binding protein of embodiment 1, wherein the VH comprises a sequence having at least 80% sequence identity to the amino acid sequence of any one of SEQ ID NOs: 1911-1914 and 1925-1927; and the VL comprises a sequence having at least 80% sequence identity to the amino acid sequence of any one of SEQ ID NOs: 2017-2020 and 2031-2033.

Embodiment 3. The α4β7 binding protein of embodiment 2, wherein the VH comprises a sequence having at least 80% sequence identity to the amino acid sequence of SEQ ID NO: 1911 and the VL comprises a sequence having at least 80% sequence identity to the amino acid sequence of SEQ ID NO: 2017.

Embodiment 4. The α4β7 binding protein of embodiment 2, wherein the VH comprises a sequence having at least 80% sequence identity to the amino acid sequence of SEQ ID NO: 1912 and the VL comprises a sequence having at least 80% sequence identity to the amino acid sequence of SEQ ID NO: 2018.

Embodiment 5. The α4β7 binding protein of embodiment 2, wherein the VH comprises a sequence having at least 80% sequence identity to the amino acid sequence of SEQ ID NO: 1913 and the VL comprises a sequence having at least 80% sequence identity to the amino acid sequence of SEQ ID NO: 2019.

Embodiment 6. The α4β7 binding protein of embodiment 2, wherein the VH comprises a sequence having at least 80% sequence identity to the amino acid sequence of SEQ ID NO: 1914 and the VL comprises a sequence having at least 80% sequence identity to the amino acid sequence of SEQ ID NO: 2020.

Embodiment 7. The α4β7 binding protein of embodiment 2, wherein the VH comprises a sequence having at least 80% sequence identity to the amino acid sequence of SEQ ID NO: 1925 and the VL comprises a sequence having at least 80% sequence identity to the amino acid sequence of SEQ ID NO: 2031.

Embodiment 8. The α4β7 binding protein of embodiment 2, wherein the VH comprises a sequence having at least 80% sequence identity to the amino acid sequence of SEQ ID NO: 1926 and the VL comprises a sequence having at least 80% sequence identity to the amino acid sequence of SEQ ID NO: 2032.

Embodiment 9. The α4β7 binding protein of embodiment 2, wherein the VH comprises a sequence having at least 80% sequence identity to the amino acid sequence of SEQ ID NO: 1927 and the VL comprises a sequence having at least 80% sequence identity to the amino acid sequence of SEQ ID NO: 2033.

Embodiment 10. An α4β7 binding protein comprising:
a) a heavy chain variable region (VH) comprising (i) a CDR1 having an amino acid sequence according to any one of SEQ ID NOs: 7-26, 32-58, 60-80, and 84-106; (ii) a CDR2 having an amino acid sequence according to any one of SEQ ID NOs: 113-132, 138-164, 166-186, and 190-212; and (iii) a CDR3 having an amino acid sequence according to any one of SEQ ID NOs: 219-238, 244-270, 272-292, and 296-318; and
b) a light chain variable region (VL) comprising (i) a CDR1 having an amino acid sequence according to any one of SEQ ID NOs: 325-344, 350-376, 378-398, and 402-424; (ii) a CDR2 having an amino acid sequence according to any one of SEQ ID NOs: 431-450, 456-482, 484-504, and 508-530; and (iii) a CDR3 having an amino acid sequence according to any one of SEQ ID NOs: 537-556, 562-588, 590-610, and 614-636.

Embodiment 11. The α4β7 binding protein of embodiment 10, wherein the VH comprises (i) a CDR1 having an amino acid sequence according to SEQ ID NO: 7; (ii) a CDR2 having an amino acid sequence according to SEQ ID NO: 113; and (iii) a CDR3 having an amino acid sequence according to SEQ ID NO: 219; and the VL comprises (i) a CDR1 having an amino acid sequence according to SEQ ID NO: 325; (ii) a CDR2 having an amino acid sequence according to SEQ ID NO: 431; and (iii) a CDR3 having an amino acid sequence according to SEQ ID NO: 537.

Embodiment 12. The α4β7 binding protein of embodiment 10, wherein the VH comprises (i) a CDR1 having an amino acid sequence according to SEQ ID NO: 8; (ii) a CDR2 having an amino acid sequence according to SEQ ID NO: 114; and (iii) a CDR3 having an amino acid sequence according to SEQ ID NO: 220; and the VL comprises (i) a CDR1 having an amino acid sequence according to SEQ ID NO: 326; (ii) a CDR2 having an amino acid sequence according to SEQ ID NO: 432; and (iii) a CDR3 having an amino acid sequence according to SEQ ID NO: 538.

Embodiment 13. The α4β7 binding protein of embodiment 10, wherein the VH comprises (i) a CDR1 having an amino acid sequence according to SEQ ID NO: 9; (ii) a CDR2 having an amino acid sequence according to SEQ ID NO: 115; and (iii) a CDR3 having an amino acid sequence according to SEQ ID NO: 221; and the VL comprises (i) a CDR1 having an amino acid sequence according to SEQ ID NO: 327; (ii) a CDR2 having an amino acid sequence according to SEQ ID NO: 433; and (iii) a CDR3 having an amino acid sequence according to SEQ ID NO: 539.

Embodiment 14. The α4β7 binding protein of embodiment 10, wherein the VH comprises (i) a CDR1 having an amino acid sequence according to SEQ ID NO: 10; (ii) a CDR2 having an amino acid sequence according to SEQ ID NO: 116; and (iii) a CDR3 having an amino acid sequence according to SEQ ID NO: 222; and the VL comprises (i) a CDR1 having an amino acid sequence according to SEQ ID NO: 328; (ii) a CDR2 having an amino acid sequence according to SEQ ID NO: 434; and (iii) a CDR3 having an amino acid sequence according to SEQ ID NO: 540.

Embodiment 15. The α4β7 binding protein of embodiment 10, wherein the VH comprises (i) a CDR1 having an amino acid sequence according to SEQ ID NO: 11; (ii) a CDR2 having an amino acid sequence according to SEQ ID NO: 117; and (iii) a CDR3 having an amino acid sequence according to SEQ ID NO: 223; and the VL comprises (i) a CDR1 having an amino acid sequence according to SEQ ID NO: 329; (ii) a CDR2 having an amino acid sequence according to SEQ ID NO: 435; and (iii) a CDR3 having an amino acid sequence according to SEQ ID NO: 541.

Embodiment 16. The α4β7 binding protein of embodiment 10, wherein the VH comprises (i) a CDR1 having an amino acid sequence according to SEQ ID NO: 12; (ii) a CDR2 having an amino acid sequence according to SEQ ID NO: 118; and (iii) a CDR3 having an amino acid sequence according to SEQ ID NO: 224; and the VL comprises (i) a CDR1 having an amino acid sequence according to SEQ ID NO: 330; (ii) a CDR2 having an amino acid sequence according to SEQ ID NO: 436; and (iii) a CDR3 having an amino acid sequence according to SEQ ID NO: 542.

Embodiment 17. The α4β7 binding protein of embodiment 10, wherein the VH comprises (i) a CDR1 having an amino acid sequence according to SEQ ID NO: 13; (ii) a CDR2 having an amino acid sequence according to SEQ ID NO: 119; and (iii) a CDR3 having an amino acid sequence according to SEQ ID NO: 225; and the VL comprises (i) a CDR1 having an amino acid sequence according to SEQ ID NO: 331; (ii) a CDR2 having an amino acid sequence according to SEQ ID NO: 437; and (iii) a CDR3 having an amino acid sequence according to SEQ ID NO: 543.

Embodiment 18. The α4β7 binding protein of embodiment 10, wherein the VH comprises (i) a CDR1 having an amino acid sequence according to SEQ ID NO: 14; (ii) a CDR2 having an amino acid sequence according to SEQ ID NO: 120; and (iii) a CDR3 having an amino acid sequence according to SEQ ID NO: 226; and the VL comprises (i) a CDR1 having an amino acid sequence according to SEQ ID NO: 332; (ii) a CDR2 having an amino acid sequence according to SEQ ID NO: 438; and (iii) a CDR3 having an amino acid sequence according to SEQ ID NO: 544.

Embodiment 19. The α4β7 binding protein of embodiment 10, wherein the VH comprises (i) a CDR1 having an amino acid sequence according to SEQ ID NO: 15; (ii) a CDR2 having an amino acid sequence according to SEQ ID NO: 121; and (iii) a CDR3 having an amino acid sequence according to SEQ ID NO: 227; and the VL comprises (i) a CDR1 having an amino acid sequence according to SEQ ID NO: 333; (ii) a CDR2 having an amino acid sequence according to SEQ ID NO: 439; and (iii) a CDR3 having an amino acid sequence according to SEQ ID NO: 545.

Embodiment 20. The α4β7 binding protein of embodiment 10, wherein the VH comprises (i) a CDR1 having an amino acid sequence according to SEQ ID NO: 16; (ii) a CDR2 having an amino acid sequence according to SEQ ID NO: 122; and (iii) a CDR3 having an amino acid sequence according to SEQ ID NO: 228; and the VL comprises (i) a CDR1 having an amino acid sequence according to SEQ ID NO: 334; (ii) a CDR2 having an amino acid sequence according to SEQ ID NO: 440; and (iii) a CDR3 having an amino acid sequence according to SEQ ID NO: 546.

Embodiment 21. The α4β7 binding protein of embodiment 10, wherein the VH comprises (i) a CDR1 having an amino acid sequence according to SEQ ID NO: 17; (ii) a CDR2 having an amino acid sequence according to SEQ ID NO: 123; and (iii) a CDR3 having an amino acid sequence according to SEQ ID NO: 229; and the VL comprises (i) a CDR1 having an amino acid sequence according to SEQ ID NO: 335; (ii) a CDR2 having an amino acid sequence according to SEQ ID NO: 441; and (iii) a CDR3 having an amino acid sequence according to SEQ ID NO: 547.

Embodiment 22. The α4β7 binding protein of embodiment 10, wherein the VH comprises (i) a CDR1 having an amino acid sequence according to SEQ ID NO: 18; (ii) a CDR2 having an amino acid sequence according to SEQ ID NO: 124; and (iii) a CDR3 having an amino acid sequence according to SEQ ID NO: 230; and the VL comprises (i) a CDR1 having an amino acid sequence according to SEQ ID NO: 336; (ii) a CDR2 having an amino acid sequence according to SEQ ID NO: 442; and (iii) a CDR3 having an amino acid sequence according to SEQ ID NO: 548.

Embodiment 23. The α4β7 binding protein of embodiment 10, wherein the VH comprises (i) a CDR1 having an amino acid sequence according to SEQ ID NO: 19; (ii) a CDR2 having an amino acid sequence according to SEQ ID NO: 125; and (iii) a CDR3 having an amino acid sequence according to SEQ ID NO: 231; and the VL comprises (i) a CDR1 having an amino acid sequence according to SEQ ID NO: 337; (ii) a CDR2 having an amino acid sequence according to SEQ ID NO: 443; and (iii) a CDR3 having an amino acid sequence according to SEQ ID NO: 549.

Embodiment 24. The α4β7 binding protein of embodiment 10, wherein the VH comprises (i) a CDR1 having an amino acid sequence according to SEQ ID NO: 20; (ii) a CDR2 having an amino acid sequence according to SEQ ID NO: 126; and (iii) a CDR3 having an amino acid sequence according to SEQ ID NO: 232; and the VL comprises (i) a CDR1 having an amino acid sequence according to SEQ ID NO: 338; (ii) a CDR2 having an amino acid sequence according to SEQ ID NO: 444; and (iii) a CDR3 having an amino acid sequence according to SEQ ID NO: 550.

Embodiment 25. The α4β7 binding protein of embodiment 10, wherein the VH comprises (i) a CDR1 having an amino acid sequence according to SEQ ID NO: 21; (ii) a CDR2 having an amino acid sequence according to SEQ ID NO: 127; and (iii) a CDR3 having an amino acid sequence according to SEQ ID NO: 233; and the VL comprises (i) a CDR1 having an amino acid sequence according to SEQ ID NO: 339; (ii) a CDR2 having an amino acid sequence according to SEQ ID NO: 445; and (iii) a CDR3 having an amino acid sequence according to SEQ ID NO: 551.

Embodiment 26. The α4β7 binding protein of embodiment 10, wherein the VH comprises (i) a CDR1 having an amino acid sequence according to SEQ ID NO: 22; (ii) a CDR2 having an amino acid sequence according to SEQ ID NO: 128; and (iii) a CDR3 having an amino acid sequence according to SEQ ID NO: 234; and the VL comprises (i) a CDR1 having an amino acid sequence according to SEQ ID NO: 340; (ii) a CDR2 having an amino acid sequence according to SEQ ID NO: 446; and (iii) a CDR3 having an amino acid sequence according to SEQ ID NO: 552.

Embodiment 27. The α4β7 binding protein of embodiment 10, wherein the VH comprises (i) a CDR1 having an amino acid sequence according to SEQ ID NO: 23; (ii) a CDR2 having an amino acid sequence according to SEQ ID NO: 129; and (iii) a CDR3 having an amino acid sequence according to SEQ ID NO: 235; and the VL comprises (i) a CDR1 having an amino acid sequence according to SEQ ID NO: 341; (ii) a CDR2 having an amino acid sequence according to SEQ ID NO: 447; and (iii) a CDR3 having an amino acid sequence according to SEQ ID NO: 553.

Embodiment 28. The α4β7 binding protein of embodiment 10, wherein the VH comprises (i) a CDR1 having an amino acid sequence according to SEQ ID NO: 24; (ii) a CDR2 having an amino acid sequence according to SEQ ID NO: 130; and (iii) a CDR3 having an amino acid sequence according to SEQ ID NO: 236; and the VL comprises (i) a CDR1 having an amino acid sequence according to SEQ ID NO: 342; (ii) a CDR2 having an amino acid sequence according to SEQ ID NO: 448; and (iii) a CDR3 having an amino acid sequence according to SEQ ID NO: 554.

Embodiment 29. The α4β7 binding protein of embodiment 10, wherein the VH comprises (i) a CDR1 having an amino acid sequence according to SEQ ID NO: 25; (ii) a CDR2 having an amino acid sequence according to SEQ ID NO: 131; and (iii) a CDR3 having an amino acid sequence according to SEQ ID NO: 237; and the VL comprises (i) a CDR1 having an amino acid sequence according to SEQ ID NO: 343; (ii) a CDR2 having an amino acid sequence according to SEQ ID NO: 449; and (iii) a CDR3 having an amino acid sequence according to SEQ ID NO: 555.

Embodiment 30. The α4β7 binding protein of embodiment 10, wherein the VH comprises (i) a CDR1 having an amino acid sequence according to SEQ ID NO: 26; (ii) a CDR2 having an amino acid sequence according to SEQ ID NO: 132; and (iii) a CDR3 having an amino acid sequence according to SEQ ID NO: 238; and the VL comprises (i) a CDR1 having an amino acid sequence according to SEQ ID NO: 344; (ii) a CDR2 having an amino acid sequence according to SEQ ID NO: 450; and (iii) a CDR3 having an amino acid sequence according to SEQ ID NO: 556.

Embodiment 31. The α4β7 binding protein of embodiment 10, wherein the VH comprises (i) a CDR1 having an amino acid sequence according to SEQ ID NO: 32; (ii) a CDR2 having an amino acid sequence according to SEQ ID NO: 138; and (iii) a CDR3 having an amino acid sequence according to SEQ ID NO: 244; and the VL comprises (i) a CDR1 having an amino acid sequence according to SEQ ID NO: 350; (ii) a CDR2 having an amino acid sequence according to SEQ ID NO: 456; and (iii) a CDR3 having an amino acid sequence according to SEQ ID NO: 562.

Embodiment 32. The α4β7 binding protein of embodiment 10, wherein the VH comprises (i) a CDR1 having an amino acid sequence according to SEQ ID NO: 33; (ii) a CDR2 having an amino acid sequence according to SEQ ID NO: 139; and (iii) a CDR3 having an amino acid sequence according to SEQ ID NO: 245; and the VL comprises (i) a CDR1 having an amino acid sequence according to SEQ ID NO: 351; (ii) a CDR2 having an amino acid sequence according to SEQ ID NO: 457; and (iii) a CDR3 having an amino acid sequence according to SEQ ID NO: 563.

Embodiment 33. The α4β7 binding protein of embodiment 10, wherein the VH comprises (i) a CDR1 having an amino acid sequence according to SEQ ID NO: 34; (ii) a CDR2 having an amino acid sequence according to SEQ ID NO: 140; and (iii) a CDR3 having an amino acid sequence according to SEQ ID NO: 246; and the VL comprises (i) a CDR1 having an amino acid sequence according to SEQ ID NO: 352; (ii) a CDR2 having an amino acid sequence according to SEQ ID NO: 458; and (iii) a CDR3 having an amino acid sequence according to SEQ ID NO: 564.

Embodiment 34. The α4β7 binding protein of embodiment 10, wherein the VH comprises (i) a CDR1 having an amino acid sequence according to SEQ ID NO: 35; (ii) a CDR2 having an amino acid sequence according to SEQ ID NO: 141; and (iii) a CDR3 having an amino acid sequence according to SEQ ID NO: 247; and the VL comprises (i) a CDR1 having an amino acid sequence according to SEQ ID NO: 353; (ii) a CDR2 having an amino acid sequence according to SEQ ID NO: 459; and (iii) a CDR3 having an amino acid sequence according to SEQ ID NO: 565.

Embodiment 35. The α4β7 binding protein of embodiment 10, wherein the VH comprises (i) a CDR1 having an amino acid sequence according to SEQ ID NO: 36; (ii) a CDR2 having an amino acid sequence according to SEQ ID NO: 142; and (iii) a CDR3 having an amino acid sequence according to SEQ ID NO: 248; and the VL comprises (i) a CDR1 having an amino acid sequence according to SEQ ID NO: 354; (ii) a CDR2 having an amino acid sequence according to SEQ ID NO: 460; and (iii) a CDR3 having an amino acid sequence according to SEQ ID NO: 566.

Embodiment 36. The α4β7 binding protein of embodiment 10, wherein the VH comprises (i) a CDR1 having an amino acid sequence according to SEQ ID NO: 37; (ii) a CDR2 having an amino acid sequence according to SEQ ID NO: 143; and (iii) a CDR3 having an amino acid sequence according to SEQ ID NO: 249; and the VL comprises (i) a CDR1 having an amino acid sequence according to SEQ ID NO: 355; (ii) a CDR2 having an amino acid sequence according to SEQ ID NO: 461; and (iii) a CDR3 having an amino acid sequence according to SEQ ID NO: 567.

Embodiment 37. The α4β7 binding protein of embodiment 10, wherein the VH comprises (i) a CDR1 having an amino acid sequence according to SEQ ID NO: 38; (ii) a CDR2 having an amino acid sequence according to SEQ ID NO: 144; and (iii) a CDR3 having an amino acid sequence according to SEQ ID NO: 250; and the VL comprises (i) a CDR1 having an amino acid sequence according to SEQ ID NO: 356; (ii) a CDR2 having an amino acid sequence according to SEQ ID NO: 462; and (iii) a CDR3 having an amino acid sequence according to SEQ ID NO: 568.

Embodiment 38. The α4β7 binding protein of embodiment 10, wherein the VH comprises (i) a CDR1 having an amino acid sequence according to SEQ ID NO: 39; (ii) a CDR2 having an amino acid sequence according to SEQ ID NO: 145; and (iii) a CDR3 having an amino acid sequence according to SEQ ID NO: 251; and the VL comprises (i) a CDR1 having an amino acid sequence according to SEQ ID NO: 357; (ii) a CDR2 having an amino acid sequence according to SEQ ID NO: 463; and (iii) a CDR3 having an amino acid sequence according to SEQ ID NO: 569.

Embodiment 39. The α4β7 binding protein of embodiment 10, wherein the VH comprises (i) a CDR1 having an amino acid sequence according to SEQ ID NO: 40; (ii) a CDR2 having an amino acid sequence according to SEQ ID NO: 146; and (iii) a CDR3 having an amino acid sequence according to SEQ ID NO: 252; and the VL comprises (i) a CDR1 having an amino acid sequence according to SEQ ID NO: 358; (ii) a CDR2 having an amino acid sequence according to SEQ ID NO: 464; and (iii) a CDR3 having an amino acid sequence according to SEQ ID NO: 570.

Embodiment 40. The α4β7 binding protein of embodiment 10, wherein the VH comprises (i) a CDR1 having an amino acid sequence according to SEQ ID NO: 41; (ii) a CDR2 having an amino acid sequence according to SEQ ID NO: 147; and (iii) a CDR3 having an amino acid sequence according to SEQ ID NO: 253; and the VL comprises (i) a CDR1 having an amino acid sequence according to SEQ ID NO: 359; (ii) a CDR2 having an amino acid sequence according to SEQ ID NO: 465; and (iii) a CDR3 having an amino acid sequence according to SEQ ID NO: 571.

Embodiment 41. The α4β7 binding protein of embodiment 10, wherein the VH comprises (i) a CDR1 having an amino acid sequence according to SEQ ID NO: 42; (ii) a CDR2 having an amino acid sequence according to SEQ ID NO: 148; and (iii) a CDR3 having an amino acid sequence according to SEQ ID NO: 254; and the VL comprises (i) a CDR1 having an amino acid sequence according to SEQ ID NO: 360; (ii) a CDR2 having an amino acid sequence according to SEQ ID NO: 466; and (iii) a CDR3 having an amino acid sequence according to SEQ ID NO: 572.

Embodiment 42. The α4β7 binding protein of embodiment 10, wherein the VH comprises (i) a CDR1 having an amino acid sequence according to SEQ ID NO: 43; (ii) a CDR2 having an amino acid sequence according to SEQ ID NO: 149; and (iii) a CDR3 having an amino acid sequence according to SEQ ID NO: 255; and the VL comprises (i) a CDR1 having an amino acid sequence according to SEQ ID NO: 361; (ii) a CDR2 having an amino acid sequence according to SEQ ID NO: 467; and (iii) a CDR3 having an amino acid sequence according to SEQ ID NO: 573.

Embodiment 43. The α4β7 binding protein of embodiment 10, wherein the VH comprises (i) a CDR1 having an amino acid sequence according to SEQ ID NO: 44; (ii) a CDR2 having an amino acid sequence according to SEQ ID NO: 150; and (iii) a CDR3 having an amino acid sequence according to SEQ ID NO: 256; and the VL comprises (i) a CDR1 having an amino acid sequence according to SEQ ID NO: 362; (ii) a CDR2 having an amino acid sequence according to SEQ ID NO: 468; and (iii) a CDR3 having an amino acid sequence according to SEQ ID NO: 574.

Embodiment 44. The α4β7 binding protein of embodiment 10, wherein the VH comprises (i) a CDR1 having an amino acid sequence according to SEQ ID NO: 45; (ii) a CDR2 having an amino acid sequence according to SEQ ID NO: 151; and (iii) a CDR3 having an amino acid sequence according to SEQ ID NO: 257; and the VL comprises (i) a CDR1 having an amino acid sequence according to SEQ ID NO: 363; (ii) a CDR2 having an amino acid sequence according to SEQ ID NO: 469; and (iii) a CDR3 having an amino acid sequence according to SEQ ID NO: 575.

Embodiment 45. The α4β7 binding protein of embodiment 10, wherein the VH comprises (i) a CDR1 having an amino acid sequence according to SEQ ID NO: 46; (ii) a CDR2 having an amino acid sequence according to SEQ ID NO: 152; and (iii) a CDR3 having an amino acid sequence according to SEQ ID NO: 258; and the VL comprises (i) a CDR1 having an amino acid sequence according to SEQ ID NO: 364; (ii) a CDR2 having an amino acid sequence according to SEQ ID NO: 470; and (iii) a CDR3 having an amino acid sequence according to SEQ ID NO: 576.

Embodiment 46. The α4β7 binding protein of embodiment 10, wherein the VH comprises (i) a CDR1 having an amino acid sequence according to SEQ ID NO: 47; (ii) a CDR2 having an amino acid sequence according to SEQ ID NO: 153; and (iii) a CDR3 having an amino acid sequence according to SEQ ID NO: 259; and the VL comprises (i) a CDR1 having an amino acid sequence according to SEQ ID NO: 365; (ii) a CDR2 having an amino acid sequence according to SEQ ID NO: 471; and (iii) a CDR3 having an amino acid sequence according to SEQ ID NO: 577.

Embodiment 47. The α4β7 binding protein of embodiment 10, wherein the VH comprises (i) a CDR1 having an amino acid sequence according to SEQ ID NO: 48; (ii) a CDR2 having an amino acid sequence according to SEQ ID NO: 154; and (iii) a CDR3 having an amino acid sequence according to SEQ ID NO: 260; and the VL comprises (i) a CDR1 having an amino acid sequence according to SEQ ID NO: 366; (ii) a CDR2 having an amino acid sequence according to SEQ ID NO: 472; and (iii) a CDR3 having an amino acid sequence according to SEQ ID NO: 578.

Embodiment 48. The α4β7 binding protein of embodiment 10, wherein the VH comprises (i) a CDR1 having an amino acid sequence according to SEQ ID NO: 49; (ii) a CDR2 having an amino acid sequence according to SEQ ID NO: 155; and (iii) a CDR3 having an amino acid sequence according to SEQ ID NO: 261; and the VL comprises (i) a CDR1 having an amino acid sequence according to SEQ ID NO: 367; (ii) a CDR2 having an amino acid sequence according to SEQ ID NO: 473; and (iii) a CDR3 having an amino acid sequence according to SEQ ID NO: 579.

Embodiment 49. The α4β7 binding protein of embodiment 10, wherein the VH comprises (i) a CDR1 having an amino acid sequence according to SEQ ID NO: 50; (ii) a CDR2 having an amino acid sequence according to SEQ ID NO: 156; and (iii) a CDR3 having an amino acid sequence according to SEQ ID NO: 262; and the VL comprises (i) a CDR1 having an amino acid sequence according to SEQ ID NO: 368; (ii) a CDR2 having an amino acid sequence according to SEQ ID NO: 474; and (iii) a CDR3 having an amino acid sequence according to SEQ ID NO: 580.

Embodiment 50. The α4β7 binding protein of embodiment 10, wherein the VH comprises (i) a CDR1 having an amino acid sequence according to SEQ ID NO: 51; (ii) a CDR2 having an amino acid sequence according to SEQ ID NO: 157; and (iii) a CDR3 having an amino acid sequence according to SEQ ID NO: 263; and the VL comprises (i) a CDR1 having an amino acid sequence according to SEQ ID NO: 369; (ii) a CDR2 having an amino acid sequence according to SEQ ID NO: 475; and (iii) a CDR3 having an amino acid sequence according to SEQ ID NO: 581.

Embodiment 51. The α4β7 binding protein of embodiment 10, wherein the VH comprises (i) a CDR1 having an amino acid sequence according to SEQ ID NO: 52; (ii) a CDR2 having an amino acid sequence according to SEQ ID NO: 158; and (iii) a CDR3 having an amino acid sequence according to SEQ ID NO: 264; and the VL comprises (i) a CDR1 having an amino acid sequence according to SEQ ID NO: 370; (ii) a CDR2 having an amino acid sequence according to SEQ ID NO: 476; and (iii) a CDR3 having an amino acid sequence according to SEQ ID NO: 582.

Embodiment 52. The α4β7 binding protein of embodiment 10, wherein the VH comprises (i) a CDR1 having an amino acid sequence according to SEQ ID NO: 53; (ii) a CDR2 having an amino acid sequence according to SEQ ID NO: 159; and (iii) a CDR3 having an amino acid sequence according to SEQ ID NO: 265; and the VL comprises (i) a CDR1 having an amino acid sequence according to SEQ ID NO: 371; (ii) a CDR2 having an amino acid sequence according to SEQ ID NO: 477; and (iii) a CDR3 having an amino acid sequence according to SEQ ID NO: 583.

Embodiment 53. The α4β7 binding protein of embodiment 10, wherein the VH comprises (i) a CDR1 having an amino acid sequence according to SEQ ID NO: 54; (ii) a CDR2 having an amino acid sequence according to SEQ ID NO: 160; and (iii) a CDR3 having an amino acid sequence according to SEQ ID NO: 266; and the VL comprises (i) a CDR1 having an amino acid sequence according to SEQ ID NO: 372; (ii) a CDR2 having an amino acid sequence according to SEQ ID NO: 478; and (iii) a CDR3 having an amino acid sequence according to SEQ ID NO: 584.

Embodiment 54. The α4β7 binding protein of embodiment 10, wherein the VH comprises (i) a CDR1 having an amino acid sequence according to SEQ ID NO: 55; (ii) a CDR2 having an amino acid sequence according to SEQ ID NO: 161; and (iii) a CDR3 having an amino acid sequence according to SEQ ID NO: 267; and the VL comprises (i) a CDR1 having an amino acid sequence according to SEQ ID NO: 373; (ii) a CDR2 having an amino acid sequence according to SEQ ID NO: 479; and (iii) a CDR3 having an amino acid sequence according to SEQ ID NO: 585.

Embodiment 55. The α4β7 binding protein of embodiment 10, wherein the VH comprises (i) a CDR1 having an amino acid sequence according to SEQ ID NO: 56; (ii) a CDR2 having an amino acid sequence according to SEQ ID NO: 162; and (iii) a CDR3 having an amino acid sequence according to SEQ ID NO: 268; and the VL comprises (i) a CDR1 having an amino acid sequence according to SEQ ID NO: 374; (ii) a CDR2 having an amino acid sequence according to SEQ ID NO: 480; and (iii) a CDR3 having an amino acid sequence according to SEQ ID NO: 586.

Embodiment 56. The α4β7 binding protein of embodiment 10, wherein the VH comprises (i) a CDR1 having an amino acid sequence according to SEQ ID NO: 57; (ii) a CDR2 having an amino acid sequence according to SEQ ID NO: 163; and (iii) a CDR3 having an amino acid sequence according to SEQ ID NO: 269; and the VL comprises (i) a CDR1 having an amino acid sequence according to SEQ ID NO: 375; (ii) a CDR2 having an amino acid sequence according to SEQ ID NO: 481; and (iii) a CDR3 having an amino acid sequence according to SEQ ID NO: 587.

Embodiment 57. The α4β7 binding protein of embodiment 10, wherein the VH comprises (i) a CDR1 having an amino acid sequence according to SEQ ID NO: 58; (ii) a CDR2 having an amino acid sequence according to SEQ ID NO: 164; and (iii) a CDR3 having an amino acid sequence according to SEQ ID NO: 270; and the VL comprises (i) a CDR1 having an amino acid sequence according to SEQ ID NO: 376; (ii) a CDR2 having an amino acid sequence according to SEQ ID NO: 482; and (iii) a CDR3 having an amino acid sequence according to SEQ ID NO: 588.

Embodiment 58. The α4β7 binding protein of embodiment 10, wherein the VH comprises (i) a CDR1 having an amino acid sequence according to SEQ ID NO: 60; (ii) a CDR2 having an amino acid sequence according to SEQ ID NO: 166; and (iii) a CDR3 having an amino acid sequence according to SEQ ID NO: 272; and the VL comprises (i) a CDR1 having an amino acid sequence according to SEQ ID NO: 378; (ii) a CDR2 having an amino acid sequence according to SEQ ID NO: 484; and (iii) a CDR3 having an amino acid sequence according to SEQ ID NO: 590.

Embodiment 59. The α4β7 binding protein of embodiment 10, wherein the VH comprises (i) a CDR1 having an amino acid sequence according to SEQ ID NO: 61; (ii) a CDR2 having an amino acid sequence according to SEQ ID NO: 167; and (iii) a CDR3 having an amino acid sequence according to SEQ ID NO: 273; and the VL comprises (i) a CDR1 having an amino acid sequence according to SEQ ID NO: 379; (ii) a CDR2 having an amino acid sequence according to SEQ ID NO: 485; and (iii) a CDR3 having an amino acid sequence according to SEQ ID NO: 591.

Embodiment 60. The α4β7 binding protein of embodiment 10, wherein the VH comprises (i) a CDR1 having an amino acid sequence according to SEQ ID NO: 62; (ii) a CDR2 having an amino acid sequence according to SEQ ID NO: 168; and (iii) a CDR3 having an amino acid sequence according to SEQ ID NO: 274; and the VL comprises (i) a CDR1 having an amino acid sequence according to SEQ ID NO: 380; (ii) a CDR2 having an amino acid sequence according to SEQ ID NO: 486; and (iii) a CDR3 having an amino acid sequence according to SEQ ID NO: 592.

Embodiment 61. The α4β7 binding protein of embodiment 10, wherein the VH comprises (i) a CDR1 having an amino acid sequence according to SEQ ID NO: 63; (ii) a CDR2 having an amino acid sequence according to SEQ ID NO: 169; and (iii) a CDR3 having an amino acid sequence according to SEQ ID NO: 275; and the VL comprises (i) a CDR1 having an amino acid sequence according to SEQ ID NO: 381; (ii) a CDR2 having an amino acid sequence according to SEQ ID NO: 487; and (iii) a CDR3 having an amino acid sequence according to SEQ ID NO: 593.

Embodiment 62. The α4β7 binding protein of embodiment 10, wherein the VH comprises (i) a CDR1 having an amino acid sequence according to SEQ ID NO: 64; (ii) a CDR2 having an amino acid sequence according to SEQ ID NO: 170; and (iii) a CDR3 having an amino acid sequence according to SEQ ID NO: 276; and the VL comprises (i) a CDR1 having an amino acid sequence according to SEQ ID NO: 382; (ii) a CDR2 having an amino acid sequence according to SEQ ID NO: 488; and (iii) a CDR3 having an amino acid sequence according to SEQ ID NO: 594.

Embodiment 63. The α4β7 binding protein of embodiment 10, wherein the VH comprises (i) a CDR1 having an amino acid sequence according to SEQ ID NO: 65; (ii) a CDR2 having an amino acid sequence according to SEQ ID NO: 171; and (iii) a CDR3 having an amino acid sequence according to SEQ ID NO: 277; and the VL comprises (i) a CDR1 having an amino acid sequence according to SEQ ID NO: 383; (ii) a CDR2 having an amino acid sequence according to SEQ ID NO: 489; and (iii) a CDR3 having an amino acid sequence according to SEQ ID NO: 595.

Embodiment 64. The α4β7 binding protein of embodiment 10, wherein the VH comprises (i) a CDR1 having an amino acid sequence according to SEQ ID NO: 66; (ii) a CDR2 having an amino acid sequence according to SEQ ID NO: 172; and (iii) a CDR3 having an amino acid sequence according to SEQ ID NO: 278; and the VL comprises (i) a CDR1 having an amino acid sequence according to SEQ ID NO: 384; (ii) a CDR2 having an amino acid sequence according to SEQ ID NO: 490; and (iii) a CDR3 having an amino acid sequence according to SEQ ID NO: 596.

Embodiment 65. The α4β7 binding protein of embodiment 10, wherein the VH comprises (i) a CDR1 having an amino acid sequence according to SEQ ID NO: 67; (ii) a CDR2 having an amino acid sequence according to SEQ ID NO: 173; and (iii) a CDR3 having an amino acid sequence according to SEQ ID NO: 279; and the VL comprises (i) a CDR1 having an amino acid sequence according to SEQ ID NO: 385; (ii) a CDR2 having an amino acid sequence according to SEQ ID NO: 491; and (iii) a CDR3 having an amino acid sequence according to SEQ ID NO: 597. The α4β7 binding protein of embodiment 10, wherein the VH comprises (i) a CDR1 having an amino acid sequence according to SEQ ID NO: 68; (ii) a CDR2 having an amino acid sequence according to SEQ ID NO: 174; and (iii) a CDR3 having an amino acid sequence according to SEQ ID NO: 280; and the VL comprises (i) a CDR1 having an amino acid sequence according to SEQ ID NO: 386; (ii) a CDR2 having an amino acid sequence according to SEQ ID NO: 492; and (iii) a CDR3 having an amino acid sequence according to SEQ ID NO: 598.

Embodiment 66. The α4β7 binding protein of embodiment 10, wherein the VH comprises (i) a CDR1 having an amino acid sequence according to SEQ ID NO: 69; (ii) a CDR2 having an amino acid sequence according to SEQ ID NO: 175; and (iii) a CDR3 having an amino acid sequence according to SEQ ID NO: 281; and the VL comprises (i) a CDR1 having an amino acid sequence according to SEQ ID NO: 387; (ii) a CDR2 having an amino acid sequence according to SEQ ID NO: 493; and (iii) a CDR3 having an amino acid sequence according to SEQ ID NO: 599.

Embodiment 67. The α4β7 binding protein of embodiment 10, wherein the VH comprises (i) a CDR1 having an amino acid sequence according to SEQ ID NO: 70; (ii) a CDR2 having an amino acid sequence according to SEQ ID NO: 176; and (iii) a CDR3 having an amino acid sequence according to SEQ ID NO: 282; and the VL comprises (i) a CDR1 having an amino acid sequence according to SEQ ID NO: 388; (ii) a CDR2 having an amino acid sequence according to SEQ ID NO: 494; and (iii) a CDR3 having an amino acid sequence according to SEQ ID NO: 600.

Embodiment 68. The α4β7 binding protein of embodiment 10, wherein the VH comprises (i) a CDR1 having an amino acid sequence according to SEQ ID NO: 71; (ii) a CDR2 having an amino acid sequence according to SEQ ID NO: 177; and (iii) a CDR3 having an amino acid sequence according to SEQ ID NO: 283; and the VL comprises (i) a CDR1 having an amino acid sequence according to SEQ ID NO: 389; (ii) a CDR2 having an amino acid sequence according to SEQ ID NO: 495; and (iii) a CDR3 having an amino acid sequence according to SEQ ID NO: 601.

Embodiment 69. The α4β7 binding protein of embodiment 10, wherein the VH comprises (i) a CDR1 having an amino acid sequence according to SEQ ID NO: 72; (ii) a CDR2 having an amino acid sequence according to SEQ ID NO: 178; and (iii) a CDR3 having an amino acid sequence according to SEQ ID NO: 284; and the VL comprises (i) a CDR1 having an amino acid sequence according to SEQ ID NO: 390; (ii) a CDR2 having an amino acid sequence according to SEQ ID NO: 496; and (iii) a CDR3 having an amino acid sequence according to SEQ ID NO: 602.

Embodiment 70. The α4β7 binding protein of embodiment 10, wherein the VH comprises (i) a CDR1 having an amino acid sequence according to SEQ ID NO: 73; (ii) a CDR2 having an amino acid sequence according to SEQ ID NO: 179; and (iii) a CDR3 having an amino acid sequence according to SEQ ID NO: 285; and the VL comprises (i) a CDR1 having an amino acid sequence according to SEQ ID NO: 391; (ii) a CDR2 having an amino acid sequence according to SEQ ID NO: 497; and (iii) a CDR3 having an amino acid sequence according to SEQ ID NO: 603.

Embodiment 71. The α4β7 binding protein of embodiment 10, wherein the VH comprises (i) a CDR1 having an amino acid sequence according to SEQ ID NO: 74; (ii) a CDR2 having an amino acid sequence according to SEQ ID NO: 180; and (iii) a CDR3 having an amino acid sequence according to SEQ ID NO: 286; and the VL comprises (i) a CDR1 having an amino acid sequence according to SEQ ID NO: 392; (ii) a CDR2 having an amino acid sequence according to SEQ ID NO: 498; and (iii) a CDR3 having an amino acid sequence according to SEQ ID NO: 604.

Embodiment 72. The α4β7 binding protein of embodiment 10, wherein the VH comprises (i) a CDR1 having an amino acid sequence according to SEQ ID NO: 75; (ii) a CDR2 having an amino acid sequence according to SEQ ID NO: 181; and (iii) a CDR3 having an amino acid sequence according to SEQ ID NO: 287; and the VL comprises (i) a CDR1 having an amino acid sequence according to SEQ ID NO: 393; (ii) a CDR2 having an amino acid sequence according to SEQ ID NO: 499; and (iii) a CDR3 having an amino acid sequence according to SEQ ID NO: 605.

Embodiment 73. The α4β7 binding protein of embodiment 10, wherein the VH comprises (i) a CDR1 having an amino acid sequence according to SEQ ID NO: 76; (ii) a CDR2 having an amino acid sequence according to SEQ ID NO: 182; and (iii) a CDR3 having an amino acid sequence according to SEQ ID NO: 288; and the VL comprises (i) a CDR1 having an amino acid sequence according to SEQ ID NO: 394; (ii) a CDR2 having an amino acid sequence according to SEQ ID NO: 500; and (iii) a CDR3 having an amino acid sequence according to SEQ ID NO: 606.

Embodiment 74. The α4β7 binding protein of embodiment 10, wherein the VH comprises (i) a CDR1 having an amino acid sequence according to SEQ ID NO: 77; (ii) a CDR2 having an amino acid sequence according to SEQ ID NO: 183; and (iii) a CDR3 having an amino acid sequence according to SEQ ID NO: 289; and the VL comprises (i) a CDR1 having an amino acid sequence according to SEQ ID NO: 395; (ii) a CDR2 having an amino acid sequence according to SEQ ID NO: 501; and (iii) a CDR3 having an amino acid sequence according to SEQ ID NO: 607.

Embodiment 75. The α4β7 binding protein of embodiment 10, wherein the VH comprises (i) a CDR1 having an amino acid sequence according to SEQ ID NO: 78; (ii) a CDR2 having an amino acid sequence according to SEQ ID NO: 184; and (iii) a CDR3 having an amino acid sequence according to SEQ ID NO: 290; and the VL comprises (i) a CDR1 having an amino acid sequence according to SEQ ID NO: 396; (ii) a CDR2 having an amino acid sequence according to SEQ ID NO: 502; and (iii) a CDR3 having an amino acid sequence according to SEQ ID NO: 608.

Embodiment 76. The α4β7 binding protein of embodiment 10, wherein the VH comprises (i) a CDR1 having an amino acid sequence according to SEQ ID NO: 79; (ii) a CDR2 having an amino acid sequence according to SEQ ID NO: 185; and (iii) a CDR3 having an amino acid sequence according to SEQ ID NO: 291; and the VL comprises (i) a CDR1 having an amino acid sequence according to SEQ ID NO: 397; (ii) a CDR2 having an amino acid sequence according to SEQ ID NO: 503; and (iii) a CDR3 having an amino acid sequence according to SEQ ID NO: 609.

Embodiment 77. The α4β7 binding protein of embodiment 10, wherein the VH comprises (i) a CDR1 having an amino acid sequence according to SEQ ID NO: 80; (ii) a CDR2 having an amino acid sequence according to SEQ ID NO: 186; and (iii) a CDR3 having an amino acid sequence according to SEQ ID NO: 292; and the VL comprises (i) a CDR1 having an amino acid sequence according to SEQ ID NO: 398; (ii) a CDR2 having an amino acid sequence according to SEQ ID NO: 504; and (iii) a CDR3 having an amino acid sequence according to SEQ ID NO: 610.

Embodiment 78. The α4β7 binding protein of embodiment 10, wherein the VH comprises (i) a CDR1 having an amino acid sequence according to SEQ ID NO: 84; (ii) a CDR2 having an amino acid sequence according to SEQ ID NO: 190; and (iii) a CDR3 having an amino acid sequence according to SEQ ID NO: 296; and the VL comprises (i) a CDR1 having an amino acid sequence according to SEQ ID NO: 402; (ii) a CDR2 having an amino acid sequence according to SEQ ID NO: 508; and (iii) a CDR3 having an amino acid sequence according to SEQ ID NO: 614.

Embodiment 79. The α4β7 binding protein of embodiment 10, wherein the VH comprises (i) a CDR1 having an amino acid sequence according to SEQ ID NO: 85; (ii) a CDR2 having an amino acid sequence according to SEQ ID NO: 191; and (iii) a CDR3 having an amino acid sequence according to SEQ ID NO: 297; and the VL comprises (i) a CDR1 having an amino acid sequence according to SEQ ID NO: 403; (ii) a CDR2 having an amino acid sequence according to SEQ ID NO: 509; and (iii) a CDR3 having an amino acid sequence according to SEQ ID NO: 615.

Embodiment 80. The α4β7 binding protein of embodiment 10, wherein the VH comprises (i) a CDR1 having an amino acid sequence according to SEQ ID NO: 86; (ii) a CDR2 having an amino acid sequence according to SEQ ID NO: 192; and (iii) a CDR3 having an amino acid sequence according to SEQ ID NO: 298; and the VL comprises (i) a CDR1 having an amino acid sequence according to SEQ ID NO: 404; (ii) a CDR2 having an amino acid sequence according to SEQ ID NO: 510; and (iii) a CDR3 having an amino acid sequence according to SEQ ID NO: 616.

Embodiment 81. The α4β7 binding protein of embodiment 10, wherein the VH comprises (i) a CDR1 having an amino acid sequence according to SEQ ID NO: 87; (ii) a CDR2 having an amino acid sequence according to SEQ ID NO: 193; and (iii) a CDR3 having an amino acid sequence according to SEQ ID NO: 299; and the VL comprises (i) a CDR1 having an amino acid sequence according to SEQ ID NO: 405; (ii) a CDR2 having an amino acid sequence according to SEQ ID NO: 511; and (iii) a CDR3 having an amino acid sequence according to SEQ ID NO: 617.

Embodiment 82. The α4β7 binding protein of embodiment 10, wherein the VH comprises (i) a CDR1 having an amino acid sequence according to SEQ ID NO: 88; (ii) a CDR2 having an amino acid sequence according to SEQ ID NO: 194; and (iii) a CDR3 having an amino acid sequence according to SEQ ID NO: 300; and the VL comprises (i) a CDR1 having an amino acid sequence according to SEQ ID NO: 406; (ii) a CDR2 having an amino acid sequence according to SEQ ID NO: 512; and (iii) a CDR3 having an amino acid sequence according to SEQ ID NO: 618.

Embodiment 83. The α4β7 binding protein of embodiment 10, wherein the VH comprises (i) a CDR1 having an amino acid sequence according to SEQ ID NO: 89; (ii) a CDR2 having an amino acid sequence according to SEQ ID NO: 195; and (iii) a CDR3 having an amino acid sequence according to SEQ ID NO: 301; and the VL comprises (i) a CDR1 having an amino acid sequence according to SEQ ID NO: 407; (ii) a CDR2 having an amino acid sequence according to SEQ ID NO: 513; and (iii) a CDR3 having an amino acid sequence according to SEQ ID NO: 619.

Embodiment 84. The α4β7 binding protein of embodiment 10, wherein the VH comprises (i) a CDR1 having an amino acid sequence according to SEQ ID NO: 90; (ii) a CDR2 having an amino acid sequence according to SEQ ID NO: 196; and (iii) a CDR3 having an amino acid sequence according to SEQ ID NO: 302; and the VL comprises (i) a CDR1 having an amino acid sequence according to SEQ ID NO: 408; (ii) a CDR2 having an amino acid sequence according to SEQ ID NO: 514; and (iii) a CDR3 having an amino acid sequence according to SEQ ID NO: 620.

Embodiment 85. The α4β7 binding protein of embodiment 10, wherein the VH comprises (i) a CDR1 having an amino acid sequence according to SEQ ID NO: 91; (ii) a CDR2 having an amino acid sequence according to SEQ ID NO: 197; and (iii) a CDR3 having an amino acid sequence according to SEQ ID NO: 303; and the VL comprises (i) a CDR1 having an amino acid sequence according to SEQ ID NO: 409; (ii) a CDR2 having an amino acid sequence according to SEQ ID NO: 515; and (iii) a CDR3 having an amino acid sequence according to SEQ ID NO: 621.

Embodiment 86. The α4β7 binding protein of embodiment 10, wherein the VH comprises (i) a CDR1 having an amino acid sequence according to SEQ ID NO: 92; (ii) a CDR2 having an amino acid sequence according to SEQ ID NO: 198; and (iii) a CDR3 having an amino acid sequence according to SEQ ID NO: 304; and the VL comprises (i) a CDR1 having an amino acid sequence according to SEQ ID NO: 410; (ii) a CDR2 having an amino acid sequence according to SEQ ID NO: 516; and (iii) a CDR3 having an amino acid sequence according to SEQ ID NO: 622.

Embodiment 87. The α4β7 binding protein of embodiment 10, wherein the VH comprises (i) a CDR1 having an amino acid sequence according to SEQ ID NO: 93; (ii) a CDR2 having an amino acid sequence according to SEQ ID NO: 199; and (iii) a CDR3 having an amino acid sequence according to SEQ ID NO: 305; and the VL comprises (i) a CDR1 having an amino acid sequence according to SEQ ID NO: 411; (ii) a CDR2 having an amino acid sequence according to SEQ ID NO: 517; and (iii) a CDR3 having an amino acid sequence according to SEQ ID NO: 623.

Embodiment 88. The α4β7 binding protein of embodiment 10, wherein the VH comprises (i) a CDR1 having an amino acid sequence according to SEQ ID NO: 94; (ii) a CDR2 having an amino acid sequence according to SEQ ID NO: 200; and (iii) a CDR3 having an amino acid sequence according to SEQ ID NO: 306; and the VL comprises (i) a CDR1 having an amino acid sequence according to SEQ ID NO: 412; (ii) a CDR2 having an amino acid sequence according to SEQ ID NO: 518; and (iii) a CDR3 having an amino acid sequence according to SEQ ID NO: 624.

Embodiment 89. The α4β7 binding protein of embodiment 10, wherein the VH comprises (i) a CDR1 having an amino acid sequence according to SEQ ID NO: 95; (ii) a CDR2 having an amino acid sequence according to SEQ ID NO: 201; and (iii) a CDR3 having an amino acid sequence according to SEQ ID NO: 307; and the VL comprises (i) a CDR1 having an amino acid sequence according to SEQ ID NO: 413; (ii) a CDR2 having an amino acid sequence according to SEQ ID NO: 519; and (iii) a CDR3 having an amino acid sequence according to SEQ ID NO: 625.

Embodiment 90. The α4β7 binding protein of embodiment 10, wherein the VH comprises (i) a CDR1 having an amino acid sequence according to SEQ ID NO: 96; (ii) a CDR2 having an amino acid sequence according to SEQ ID NO: 202; and (iii) a CDR3 having an amino acid sequence according to SEQ ID NO: 308; and the VL comprises (i) a CDR1 having an amino acid sequence according to SEQ ID NO: 414; (ii) a CDR2 having an amino acid sequence according to SEQ ID NO: 520; and (iii) a CDR3 having an amino acid sequence according to SEQ ID NO: 626.

Embodiment 91. The α4β7 binding protein of embodiment 10, wherein the VH comprises (i) a CDR1 having an amino acid sequence according to SEQ ID NO: 97; (ii) a CDR2 having an amino acid sequence according to SEQ ID NO: 203; and (iii) a CDR3 having an amino acid sequence according to SEQ ID NO: 309; and the VL comprises (i) a CDR1 having an amino acid sequence according to SEQ ID NO: 415; (ii) a CDR2 having an amino acid sequence according to SEQ ID NO: 521; and (iii) a CDR3 having an amino acid sequence according to SEQ ID NO: 627.

Embodiment 92. The α4β7 binding protein of embodiment 10, wherein the VH comprises (i) a CDR1 having an amino acid sequence according to SEQ ID NO: 98; (ii) a CDR2 having an amino acid sequence according to SEQ ID NO: 204; and (iii) a CDR3 having an amino acid sequence according to SEQ ID NO: 310; and the VL comprises (i) a CDR1 having an amino acid sequence according to SEQ ID NO: 416; (ii) a CDR2 having an amino acid sequence according to SEQ ID NO: 522; and (iii) a CDR3 having an amino acid sequence according to SEQ ID NO: 628.

Embodiment 93. The α4β7 binding protein of embodiment 10, wherein the VH comprises (i) a CDR1 having an amino acid sequence according to SEQ ID NO: 99; (ii) a CDR2 having an amino acid sequence according to SEQ ID NO: 205; and (iii) a CDR3 having an amino acid sequence according to SEQ ID NO: 311; and the VL comprises (i) a CDR1 having an amino acid sequence according to SEQ ID NO: 417; (ii) a CDR2 having an amino acid sequence according to SEQ ID NO: 523; and (iii) a CDR3 having an amino acid sequence according to SEQ ID NO: 629.

Embodiment 94. The α4β7 binding protein of embodiment 10, wherein the VH comprises (i) a CDR1 having an amino acid sequence according to SEQ ID NO: 100; (ii) a CDR2 having an amino acid sequence according to SEQ ID NO: 206; and (iii) a CDR3 having an amino acid sequence according to SEQ ID NO: 312; and the VL comprises (i) a CDR1 having an amino acid sequence according to SEQ ID NO: 418; (ii) a CDR2 having an amino acid sequence according to SEQ ID NO: 524; and (iii) a CDR3 having an amino acid sequence according to SEQ ID NO: 630.

Embodiment 95. The α4β7 binding protein of embodiment 10, wherein the VH comprises (i) a CDR1 having an amino acid sequence according to SEQ ID NO: 101; (ii) a CDR2 having an amino acid sequence according to SEQ ID NO: 207; and (iii) a CDR3 having an amino acid sequence according to SEQ ID NO: 313; and the VL comprises (i) a CDR1 having an amino acid sequence according to SEQ ID NO: 419; (ii) a CDR2 having an amino acid sequence according to SEQ ID NO: 525; and (iii) a CDR3 having an amino acid sequence according to SEQ ID NO: 631.

Embodiment 96. The α4β7 binding protein of embodiment 10, wherein the VH comprises (i) a CDR1 having an amino acid sequence according to SEQ ID NO: 102; (ii) a CDR2 having an amino acid sequence according to SEQ ID NO: 208; and (iii) a CDR3 having an amino acid sequence according to SEQ ID NO: 314; and the VL comprises (i) a CDR1 having an amino acid sequence according to SEQ ID NO: 420; (ii) a CDR2 having an amino acid sequence according to SEQ ID NO: 526; and (iii) a CDR3 having an amino acid sequence according to SEQ ID NO: 632.

Embodiment 97. The α4β7 binding protein of embodiment 10, wherein the VH comprises (i) a CDR1 having an amino acid sequence according to SEQ ID NO: 103; (ii) a CDR2 having an amino acid sequence according to SEQ ID NO: 209; and (iii) a CDR3 having an amino acid sequence according to SEQ ID NO: 315; and the VL comprises (i) a CDR1 having an amino acid sequence according to SEQ ID NO: 421; (ii) a CDR2 having an amino acid sequence according to SEQ ID NO: 527; and (iii) a CDR3 having an amino acid sequence according to SEQ ID NO: 633.

Embodiment 98. The α4β7 binding protein of embodiment 10, wherein the VH comprises (i) a CDR1 having an amino acid sequence according to SEQ ID NO: 104; (ii) a CDR2 having an amino acid sequence according to SEQ ID NO: 210; and (iii) a CDR3 having an amino acid sequence according to SEQ ID NO: 316; and the VL comprises (i) a CDR1 having an amino acid sequence according to SEQ ID NO: 422; (ii) a CDR2 having an amino acid sequence according to SEQ ID NO: 528; and (iii) a CDR3 having an amino acid sequence according to SEQ ID NO: 634.

Embodiment 99. The α4β7 binding protein of embodiment 10, wherein the VH comprises (i) a CDR1 having an amino acid sequence according to SEQ ID NO: 105; (ii) a CDR2 having an amino acid sequence according to SEQ ID NO: 211; and (iii) a CDR3 having an amino acid sequence according to SEQ ID NO: 317; and the VL comprises (i) a CDR1 having an amino acid sequence according to SEQ ID NO: 423; (ii) a CDR2 having an amino acid sequence according to SEQ ID NO: 529; and (iii) a CDR3 having an amino acid sequence according to SEQ ID NO: 635.

Embodiment 100. The α4β7 binding protein of embodiment 10, wherein the VH comprises (i) a CDR1 having an amino acid sequence according to SEQ ID NO: 106; (ii) a CDR2 having an amino acid sequence according to SEQ ID NO: 212; and (iii) a CDR3 having an amino acid sequence according to SEQ ID NO: 318; and the VL comprises (i) a CDR1 having an amino acid sequence according to SEQ ID NO: 424; (ii) a CDR2 having an amino acid sequence according to SEQ ID NO: 530; and (iii) a CDR3 having an amino acid sequence according to SEQ ID NO: 636.

Embodiment 101. The α4β7 binding protein of any one of embodiments 10-100, wherein the VH comprises a sequence having at least 80% sequence identity to the amino acid sequence of any one of SEQ ID NOs: 1915-1934, 1940-1966, 1968-1988, and 1992-2014; and the VL comprises a sequence having at least 80% sequence identity to the amino acid sequence of any one of SEQ ID NOs: 2021-2040, 2046-2072, 2074-2094, and 2098-2120.

Embodiment 102. The α4β7 binding protein of embodiment 101, wherein the VH comprises a sequence having at least 80% sequence identity to the amino acid sequence of SEQ ID NO: 127 and the VL comprises a sequence having at least 80% sequence identity to the amino acid sequence of SEQ ID NO: 134.

Embodiment 103. The α4β7 binding protein of embodiment 101, wherein the VH comprises a sequence having at least 80% sequence identity to the amino acid sequence of SEQ ID NO: 1915 and the VL comprises a sequence having at least 80% sequence identity to the amino acid sequence of SEQ ID NO: 2021.

Embodiment 104. The α4β7 binding protein of embodiment 101, wherein the VH comprises a sequence having at least 80% sequence identity to the amino acid sequence of SEQ ID NO: 1916 and the VL comprises a sequence having at least 80% sequence identity to the amino acid sequence of SEQ ID NO: 2022.

Embodiment 105. The α4β7 binding protein of embodiment 101, wherein the VH comprises a sequence having at least 80% sequence identity to the amino acid sequence of SEQ ID NO: 1917 and the VL comprises a sequence having at least 80% sequence identity to the amino acid sequence of SEQ ID NO: 2023.

Embodiment 106. The α4β7 binding protein of embodiment 101, wherein the VH comprises a sequence having at least 80% sequence identity to the amino acid sequence of SEQ ID NO: 1918 and the VL comprises a sequence having at least 80% sequence identity to the amino acid sequence of SEQ ID NO: 2024.

Embodiment 107. The α4β7 binding protein of embodiment 101, wherein the VH comprises a sequence having at least 80% sequence identity to the amino acid sequence of SEQ ID NO: 1919 and the VL comprises a sequence having at least 80% sequence identity to the amino acid sequence of SEQ ID NO: 2025.

Embodiment 108. The α4β7 binding protein of embodiment 101, wherein the VH comprises a sequence having at least 80% sequence identity to the amino acid sequence of SEQ ID NO: 1920 and the VL comprises a sequence having at least 80% sequence identity to the amino acid sequence of SEQ ID NO: 2026.

Embodiment 109. The α4β7 binding protein of embodiment 101, wherein the VH comprises a sequence having at least 80% sequence identity to the amino acid sequence of SEQ ID NO: 1921 and the VL comprises a sequence having at least 80% sequence identity to the amino acid sequence of SEQ ID NO: 2027.

Embodiment 110. The α4β7 binding protein of embodiment 101, wherein the VH comprises a sequence having at least 80% sequence identity to the amino acid sequence of SEQ ID NO: 1922 and the VL comprises a sequence having at least 80% sequence identity to the amino acid sequence of SEQ ID NO: 2028.

Embodiment 111. The α4β7 binding protein of embodiment 101, wherein the VH comprises a sequence having at least 80% sequence identity to the amino acid sequence of SEQ ID NO: 1923 and the VL comprises a sequence having at least 80% sequence identity to the amino acid sequence of SEQ ID NO: 2029.

Embodiment 112. The α4β7 binding protein of embodiment 101, wherein the VH comprises a sequence having at least 80% sequence identity to the amino acid sequence of SEQ ID NO: 1924 and the VL comprises a sequence having at least 80% sequence identity to the amino acid sequence of SEQ ID NO: 2030.

Embodiment 113. The α4β7 binding protein of embodiment 101, wherein the VH comprises a sequence having at least 80% sequence identity to the amino acid sequence of SEQ ID NO: 1925 and the VL comprises a sequence having at least 80% sequence identity to the amino acid sequence of SEQ ID NO: 2031.

Embodiment 114. The α4β7 binding protein of embodiment 101, wherein the VH comprises a sequence having at least 80% sequence identity to the amino acid sequence of SEQ ID NO: 1926 and the VL comprises a sequence having at least 80% sequence identity to the amino acid sequence of SEQ ID NO: 2032.

Embodiment 115. The α4β7 binding protein of embodiment 101, wherein the VH comprises a sequence having at least 80% sequence identity to the amino acid sequence of SEQ ID NO: 1927 and the VL comprises a sequence having at least 80% sequence identity to the amino acid sequence of SEQ ID NO: 2033.

Embodiment 116. The α4β7 binding protein of embodiment 101, wherein the VH comprises a sequence having at least 80% sequence identity to the amino acid sequence of SEQ ID NO: 1928 and the VL comprises a sequence having at least 80% sequence identity to the amino acid sequence of SEQ ID NO: 2034.

Embodiment 117. The α4β7 binding protein of embodiment 101, wherein the VH comprises a sequence having at least 80% sequence identity to the amino acid sequence of SEQ ID NO: 1929 and the VL comprises a sequence having at least 80% sequence identity to the amino acid sequence of SEQ ID NO: 2035.

Embodiment 118. The α4β7 binding protein of embodiment 101, wherein the VH comprises a sequence having at least 80% sequence identity to the amino acid sequence of SEQ ID NO: 1930 and the VL comprises a sequence having at least 80% sequence identity to the amino acid sequence of SEQ ID NO: 2036.

Embodiment 119. The α4β7 binding protein of embodiment 101, wherein the VH comprises a sequence having at least 80% sequence identity to the amino acid sequence of SEQ ID NO: 1931 and the VL comprises a sequence having at least 80% sequence identity to the amino acid sequence of SEQ ID NO: 2037.

Embodiment 120. The α4β7 binding protein of embodiment 101, wherein the VH comprises a sequence having at least 80% sequence identity to the amino acid sequence of SEQ ID NO: 1932 and the VL comprises a sequence having at least 80% sequence identity to the amino acid sequence of SEQ ID NO: 2038.

Embodiment 121. The α4β7 binding protein of embodiment 101, wherein the VH comprises a sequence having at least 80% sequence identity to the amino acid sequence of SEQ ID NO: 1933 and the VL comprises a sequence having at least 80% sequence identity to the amino acid sequence of SEQ ID NO: 2039.

Embodiment 122. The α4β7 binding protein of embodiment 101, wherein the VH comprises a sequence having at least 80% sequence identity to the amino acid sequence of SEQ ID NO: 1934 and the VL comprises a sequence having at least 80% sequence identity to the amino acid sequence of SEQ ID NO: 2040.

Embodiment 123. The α4β7 binding protein of embodiment 101, wherein the VH comprises a sequence having at least 80% sequence identity to the amino acid sequence of SEQ ID NO: 1940 and the VL comprises a sequence having at least 80% sequence identity to the amino acid sequence of SEQ ID NO: 2046.

Embodiment 124. The α4β7 binding protein of embodiment 101, wherein the VH comprises a sequence having at least 80% sequence identity to the amino acid sequence of SEQ ID NO: 1941 and the VL comprises a sequence having at least 80% sequence identity to the amino acid sequence of SEQ ID NO: 2047.

Embodiment 125. The α4β7 binding protein of embodiment 101, wherein the VH comprises a sequence having at least 80% sequence identity to the amino acid sequence of SEQ ID NO: 1942 and the VL comprises a sequence having at least 80% sequence identity to the amino acid sequence of SEQ ID NO: 2048.

Embodiment 126. The α4β7 binding protein of embodiment 101, wherein the VH comprises a sequence having at least 80% sequence identity to the amino acid sequence of SEQ ID NO: 1943 and the VL comprises a sequence having at least 80% sequence identity to the amino acid sequence of SEQ ID NO: 2049.

Embodiment 127. The α4β7 binding protein of embodiment 101, wherein the VH comprises a sequence having at least 80% sequence identity to the amino acid sequence of SEQ ID NO: 1944 and the VL comprises a sequence having at least 80% sequence identity to the amino acid sequence of SEQ ID NO: 2050.

Embodiment 128. The α4β7 binding protein of embodiment 101, wherein the VH comprises a sequence having at least 80% sequence identity to the amino acid sequence of SEQ ID NO: 1945 and the VL comprises a sequence having at least 80% sequence identity to the amino acid sequence of SEQ ID NO: 2051.

Embodiment 129. The α4β7 binding protein of embodiment 101, wherein the VH comprises a sequence having at least 80% sequence identity to the amino acid sequence of SEQ ID NO: 1946 and the VL comprises a sequence having at least 80% sequence identity to the amino acid sequence of SEQ ID NO: 2052.

Embodiment 130. The α4β7 binding protein of embodiment 101, wherein the VH comprises a sequence having at least 80% sequence identity to the amino acid sequence of SEQ ID NO: 1947 and the VL comprises a sequence having at least 80% sequence identity to the amino acid sequence of SEQ ID NO: 2053.

Embodiment 131. The α4β7 binding protein of embodiment 101, wherein the VH comprises a sequence having at least 80% sequence identity to the amino acid sequence of SEQ ID NO: 1948 and the VL comprises a sequence having at least 80% sequence identity to the amino acid sequence of SEQ ID NO: 2054.

Embodiment 132. The α4β7 binding protein of embodiment 101, wherein the VH comprises a sequence having at least 80% sequence identity to the amino acid sequence of SEQ ID NO: 1949 and the VL comprises a sequence having at least 80% sequence identity to the amino acid sequence of SEQ ID NO: 2055.

Embodiment 133. The α4β7 binding protein of embodiment 101, wherein the VH comprises a sequence having at least 80% sequence identity to the amino acid sequence of SEQ ID NO: 1950 and the VL comprises a sequence having at least 80% sequence identity to the amino acid sequence of SEQ ID NO: 2056.

Embodiment 134. The α4β7 binding protein of embodiment 101, wherein the VH comprises a sequence having at least 80% sequence identity to the amino acid sequence of SEQ ID NO: 1951 and the VL comprises a sequence having at least 80% sequence identity to the amino acid sequence of SEQ ID NO: 2057.

Embodiment 135. The α4β7 binding protein of embodiment 101, wherein the VH comprises a sequence having at least 80% sequence identity to the amino acid sequence of SEQ ID NO: 1952 and the VL comprises a sequence having at least 80% sequence identity to the amino acid sequence of SEQ ID NO: 2058.

Embodiment 136. The α4β7 binding protein of embodiment 101, wherein the VH comprises a sequence having at least 80% sequence identity to the amino acid sequence of SEQ ID NO: 1953 and the VL comprises a sequence having at least 80% sequence identity to the amino acid sequence of SEQ ID NO: 2059.

Embodiment 137. The α4β7 binding protein of embodiment 101, wherein the VH comprises a sequence having at least 80% sequence identity to the amino acid sequence of SEQ ID NO: 1954 and the VL comprises a sequence having at least 80% sequence identity to the amino acid sequence of SEQ ID NO: 2060.

Embodiment 138. The α4β7 binding protein of embodiment 101, wherein the VH comprises a sequence having at least 80% sequence identity to the amino acid sequence of SEQ ID NO: 1955 and the VL comprises a sequence having at least 80% sequence identity to the amino acid sequence of SEQ ID NO: 2061.

Embodiment 139. The α4β7 binding protein of embodiment 101, wherein the VH comprises a sequence having at least 80% sequence identity to the amino acid sequence of SEQ ID NO: 1956 and the VL comprises a sequence having at least 80% sequence identity to the amino acid sequence of SEQ ID NO: 2062.

Embodiment 140. The α4β7 binding protein of embodiment 101, wherein the VH comprises a sequence having at least 80% sequence identity to the amino acid sequence of SEQ ID NO: 1957 and the VL comprises a sequence having at least 80% sequence identity to the amino acid sequence of SEQ ID NO: 2063.

Embodiment 141. The α4β7 binding protein of embodiment 101, wherein the VH comprises a sequence having at least 80% sequence identity to the amino acid sequence of SEQ ID NO: 1958 and the VL comprises a sequence having at least 80% sequence identity to the amino acid sequence of SEQ ID NO: 2064.

Embodiment 142. The α4β7 binding protein of embodiment 101, wherein the VH comprises a sequence having at least 80% sequence identity to the amino acid sequence of SEQ ID NO: 1959 and the VL comprises a sequence having at least 80% sequence identity to the amino acid sequence of SEQ ID NO: 2065.

Embodiment 143. The α4β7 binding protein of embodiment 101, wherein the VH comprises a sequence having at least 80% sequence identity to the amino acid sequence of SEQ ID NO: 1960 and the VL comprises a sequence having at least 80% sequence identity to the amino acid sequence of SEQ ID NO: 2066.

Embodiment 144. The α4β7 binding protein of embodiment 101, wherein the VH comprises a sequence having at least 80% sequence identity to the amino acid sequence of SEQ ID NO: 1961 and the VL comprises a sequence having at least 80% sequence identity to the amino acid sequence of SEQ ID NO: 2067.

Embodiment 145. The α4β7 binding protein of embodiment 101, wherein the VH comprises a sequence having at least 80% sequence identity to the amino acid sequence of SEQ ID NO: 1962 and the VL comprises a sequence having at least 80% sequence identity to the amino acid sequence of SEQ ID NO: 2068.

Embodiment 146. The α4β7 binding protein of embodiment 101, wherein the VH comprises a sequence having at least 80% sequence identity to the amino acid sequence of SEQ ID NO: 1963 and the VL comprises a sequence having at least 80% sequence identity to the amino acid sequence of SEQ ID NO: 2069.

Embodiment 147. The α4β7 binding protein of embodiment 101, wherein the VH comprises a sequence having at least 80% sequence identity to the amino acid sequence of SEQ ID NO: 1964 and the VL comprises a sequence having at least 80% sequence identity to the amino acid sequence of SEQ ID NO: 2070.

Embodiment 148. The α4β7 binding protein of embodiment 101, wherein the VH comprises a sequence having at least 80% sequence identity to the amino acid sequence of SEQ ID NO: 1965 and the VL comprises a sequence having at least 80% sequence identity to the amino acid sequence of SEQ ID NO: 2071.

Embodiment 149. The α4β7 binding protein of embodiment 101, wherein the VH comprises a sequence having at least 80% sequence identity to the amino acid sequence of SEQ ID NO: 1966 and the VL comprises a sequence having at least 80% sequence identity to the amino acid sequence of SEQ ID NO: 2072.

Embodiment 150. The α4β7 binding protein of embodiment 101, wherein the VH comprises a sequence having at least 80% sequence identity to the amino acid sequence of SEQ ID NO: 1968 and the VL comprises a sequence having at least 80% sequence identity to the amino acid sequence of SEQ ID NO: 2074.

Embodiment 151. The α4β7 binding protein of embodiment 101, wherein the VH comprises a sequence having at least 80% sequence identity to the amino acid sequence of SEQ ID NO: 1969 and the VL comprises a sequence having at least 80% sequence identity to the amino acid sequence of SEQ ID NO: 2075.

Embodiment 152. The α4β7 binding protein of embodiment 101, wherein the VH comprises a sequence having at least 80% sequence identity to the amino acid sequence of SEQ ID NO: 1970 and the VL comprises a sequence having at least 80% sequence identity to the amino acid sequence of SEQ ID NO: 2076.

Embodiment 153. The α4β7 binding protein of embodiment 101, wherein the VH comprises a sequence having at least 80% sequence identity to the amino acid sequence of SEQ ID NO: 1971 and the VL comprises a sequence having at least 80% sequence identity to the amino acid sequence of SEQ ID NO: 2077.

Embodiment 154. The α4β7 binding protein of embodiment 101, wherein the VH comprises a sequence having at least 80% sequence identity to the amino acid sequence of SEQ ID NO: 1972 and the VL comprises a sequence having at least 80% sequence identity to the amino acid sequence of SEQ ID NO: 2078.

Embodiment 155. The α4β7 binding protein of embodiment 101, wherein the VH comprises a sequence having at least 80% sequence identity to the amino acid sequence of SEQ ID NO: 1973 and the VL comprises a sequence having at least 80% sequence identity to the amino acid sequence of SEQ ID NO: 2079.

Embodiment 156. The α4β7 binding protein of embodiment 101, wherein the VH comprises a sequence having at least 80% sequence identity to the amino acid sequence of SEQ ID NO: 1974 and the VL comprises a sequence having at least 80% sequence identity to the amino acid sequence of SEQ ID NO: 2080.

Embodiment 157. The α4β7 binding protein of embodiment 101, wherein the VH comprises a sequence having at least 80% sequence identity to the amino acid sequence of SEQ ID NO: 1975 and the VL comprises a sequence having at least 80% sequence identity to the amino acid sequence of SEQ ID NO: 2081.

Embodiment 158. The α4β7 binding protein of embodiment 101, wherein the VH comprises a sequence having at least 80% sequence identity to the amino acid sequence of SEQ ID NO: 1976 and the VL comprises a sequence having at least 80% sequence identity to the amino acid sequence of SEQ ID NO: 2082.

Embodiment 159. The α4β7 binding protein of embodiment 101, wherein the VH comprises a sequence having at least 80% sequence identity to the amino acid sequence of SEQ ID NO: 1977 and the VL comprises a sequence having at least 80% sequence identity to the amino acid sequence of SEQ ID NO: 2083.

Embodiment 160. The α4β7 binding protein of embodiment 101, wherein the VH comprises a sequence having at least 80% sequence identity to the amino acid sequence of SEQ ID NO: 1978 and the VL comprises a sequence having at least 80% sequence identity to the amino acid sequence of SEQ ID NO: 2084.

Embodiment 161. The α4β7 binding protein of embodiment 101, wherein the VH comprises a sequence having at least 80% sequence identity to the amino acid sequence of SEQ ID NO: 1979 and the VL comprises a sequence having at least 80% sequence identity to the amino acid sequence of SEQ ID NO: 2085.

Embodiment 162. The α4β7 binding protein of embodiment 101, wherein the VH comprises a sequence having at least 80% sequence identity to the amino acid sequence of SEQ ID NO: 1980 and the VL comprises a sequence having at least 80% sequence identity to the amino acid sequence of SEQ ID NO: 2086.

Embodiment 163. The α4β7 binding protein of embodiment 101, wherein the VH comprises a sequence having at least 80% sequence identity to the amino acid sequence of SEQ ID NO: 1981 and the VL comprises a sequence having at least 80% sequence identity to the amino acid sequence of SEQ ID NO: 2087.

Embodiment 164. The α4β7 binding protein of embodiment 101, wherein the VH comprises a sequence having at least 80% sequence identity to the amino acid sequence of SEQ ID NO: 1982 and the VL comprises a sequence having at least 80% sequence identity to the amino acid sequence of SEQ ID NO: 2088.

Embodiment 165. The α4β7 binding protein of embodiment 101, wherein the VH comprises a sequence having at least 80% sequence identity to the amino acid sequence of SEQ ID NO: 1983 and the VL comprises a sequence having at least 80% sequence identity to the amino acid sequence of SEQ ID NO: 2089.

Embodiment 166. The α4β7 binding protein of embodiment 101, wherein the VH comprises a sequence having at least 80% sequence identity to the amino acid sequence of SEQ ID NO: 1984 and the VL comprises a sequence having at least 80% sequence identity to the amino acid sequence of SEQ ID NO: 2090.

Embodiment 167. The α4β7 binding protein of embodiment 101, wherein the VH comprises a sequence having at least 80% sequence identity to the amino acid sequence of SEQ ID NO: 1985 and the VL comprises a sequence having at least 80% sequence identity to the amino acid sequence of SEQ ID NO: 2091.

Embodiment 168. The α4β7 binding protein of embodiment 101, wherein the VH comprises a sequence having at least 80% sequence identity to the amino acid sequence of SEQ ID NO: 1986 and the VL comprises a sequence having at least 80% sequence identity to the amino acid sequence of SEQ ID NO: 2092.

Embodiment 169. The α4β7 binding protein of embodiment 101, wherein the VH comprises a sequence having at least 80% sequence identity to the amino acid sequence of SEQ ID NO: 1987 and the VL comprises a sequence having at least 80% sequence identity to the amino acid sequence of SEQ ID NO: 2093.

Embodiment 170. The α4β7 binding protein of embodiment 101, wherein the VH comprises a sequence having at least 80% sequence identity to the amino acid sequence of SEQ ID NO: 1992 and the VL comprises a sequence having at least 80% sequence identity to the amino acid sequence of SEQ ID NO: 2098.

Embodiment 171. The α4β7 binding protein of embodiment 101, wherein the VH comprises a sequence having at least 80% sequence identity to the amino acid sequence of SEQ ID NO: 1993 and the VL comprises a sequence having at least 80% sequence identity to the amino acid sequence of SEQ ID NO: 2099.

Embodiment 172. The α4β7 binding protein of embodiment 101, wherein the VH comprises a sequence having at least 80% sequence identity to the amino acid sequence of SEQ ID NO: 1994 and the VL comprises a sequence having at least 80% sequence identity to the amino acid sequence of SEQ ID NO: 2100.

Embodiment 173. The α4β7 binding protein of embodiment 101, wherein the VH comprises a sequence having at least 80% sequence identity to the amino acid sequence of SEQ ID NO: 1995 and the VL comprises a sequence having at least 80% sequence identity to the amino acid sequence of SEQ ID NO: 2101.

Embodiment 174. The α4β7 binding protein of embodiment 101, wherein the VH comprises a sequence having at least 80% sequence identity to the amino acid sequence of SEQ ID NO: 1996 and the VL comprises a sequence having at least 80% sequence identity to the amino acid sequence of SEQ ID NO: 2102.

Embodiment 175. The α4β7 binding protein of embodiment 101, wherein the VH comprises a sequence having at least 80% sequence identity to the amino acid sequence of SEQ ID NO: 1997 and the VL comprises a sequence having at least 80% sequence identity to the amino acid sequence of SEQ ID NO: 2103.

Embodiment 176. The α4β7 binding protein of embodiment 101, wherein the VH comprises a sequence having at least 80% sequence identity to the amino acid sequence of SEQ ID NO: 1998 and the VL comprises a sequence having at least 80% sequence identity to the amino acid sequence of SEQ ID NO: 2104.

Embodiment 177. The α4β7 binding protein of embodiment 101, wherein the VH comprises a sequence having at least 80% sequence identity to the amino acid sequence of SEQ ID NO: 1999 and the VL comprises a sequence having at least 80% sequence identity to the amino acid sequence of SEQ ID NO: 2105.

Embodiment 178. The α4β7 binding protein of embodiment 101, wherein the VH comprises a sequence having at least 80% sequence identity to the amino acid sequence of SEQ ID NO: 2000 and the VL comprises a sequence having at least 80% sequence identity to the amino acid sequence of SEQ ID NO: 2106.

Embodiment 179. The α4β7 binding protein of embodiment 101, wherein the VH comprises a sequence having at least 80% sequence identity to the amino acid sequence of SEQ ID NO: 2001 and the VL comprises a sequence having at least 80% sequence identity to the amino acid sequence of SEQ ID NO: 2107.

Embodiment 180. The α4β7 binding protein of embodiment 101, wherein the VH comprises a sequence having at least 80% sequence identity to the amino acid sequence of SEQ ID NO: 2002 and the VL comprises a sequence having at least 80% sequence identity to the amino acid sequence of SEQ ID NO: 2108.

Embodiment 181. The α4β7 binding protein of embodiment 101, wherein the VH comprises a sequence having at least 80% sequence identity to the amino acid sequence of SEQ ID NO: 2003 and the VL comprises a sequence having at least 80% sequence identity to the amino acid sequence of SEQ ID NO: 2109.

Embodiment 182. The α4β7 binding protein of embodiment 101, wherein the VH comprises a sequence having at least 80% sequence identity to the amino acid sequence of SEQ ID NO: 2004 and the VL comprises a sequence having at least 80% sequence identity to the amino acid sequence of SEQ ID NO: 2110.

Embodiment 183. The α4β7 binding protein of embodiment 101, wherein the VH comprises a sequence having at least 80% sequence identity to the amino acid sequence of SEQ ID NO: 2005 and the VL comprises a sequence having at least 80% sequence identity to the amino acid sequence of SEQ ID NO: 2111.

Embodiment 184. The α4β7 binding protein of embodiment 101, wherein the VH comprises a sequence having at least 80% sequence identity to the amino acid sequence of SEQ ID NO: 2006 and the VL comprises a sequence having at least 80% sequence identity to the amino acid sequence of SEQ ID NO: 2112.

Embodiment 185. The α4β7 binding protein of embodiment 101, wherein the VH comprises a sequence having at least 80% sequence identity to the amino acid sequence of SEQ ID NO: 2007 and the VL comprises a sequence having at least 80% sequence identity to the amino acid sequence of SEQ ID NO: 2113.

Embodiment 186. The α4β7 binding protein of embodiment 101, wherein the VH comprises a sequence having at least 80% sequence identity to the amino acid sequence of SEQ ID NO: 2008 and the VL comprises a sequence having at least 80% sequence identity to the amino acid sequence of SEQ ID NO: 2114.

Embodiment 187. The α4β7 binding protein of embodiment 101, wherein the VH comprises a sequence having at least 80% sequence identity to the amino acid sequence of SEQ ID NO: 2009 and the VL comprises a sequence having at least 80% sequence identity to the amino acid sequence of SEQ ID NO: 2115.

Embodiment 188. The α4β7 binding protein of embodiment 101, wherein the VH comprises a sequence having at least 80% sequence identity to the amino acid sequence of SEQ ID NO: 2010 and the VL comprises a sequence having at least 80% sequence identity to the amino acid sequence of SEQ ID NO: 2116.

Embodiment 189. The α4β7 binding protein of embodiment 101, wherein the VH comprises a sequence having at least 80% sequence identity to the amino acid sequence of SEQ ID NO: 2011 and the VL comprises a sequence having at least 80% sequence identity to the amino acid sequence of SEQ ID NO: 2117.

Embodiment 190. The α4β7 binding protein of embodiment 101, wherein the VH comprises a sequence having at least 80% sequence identity to the amino acid sequence of SEQ ID NO: 2012 and the VL comprises a sequence having at least 80% sequence identity to the amino acid sequence of SEQ ID NO: 2118.

Embodiment 191. The α4β7 binding protein of embodiment 101, wherein the VH comprises a sequence having at least 80% sequence identity to the amino acid sequence of SEQ ID NO: 2013 and the VL comprises a sequence having at least 80% sequence identity to the amino acid sequence of SEQ ID NO: 2119.

Embodiment 192. The α4β7 binding protein of embodiment 101, wherein the VH comprises a sequence having at least 80% sequence identity to the amino acid sequence of SEQ ID NO: 2014 and the VL comprises a sequence having at least 80% sequence identity to the amino acid sequence of SEQ ID NO: 2120.

Embodiment 193. An α4β7 binding protein comprising:
a) a heavy chain variable region (VH) comprising the amino acid sequence according to any one of SEQ ID NOs: 1909, 1910, 1935-1939, 1967; and
b) a light chain variable region (VL) comprising the amino acid sequence according to any one of SEQ ID NOs: 2015, 2016, 2041-2045, 2073.

Embodiment 194. The α4β7 binding protein of embodiment 193, wherein the VH comprises the amino acid sequence according to SEQ ID NO: 1909; and the VL comprises the amino acid sequence according to SEQ ID NO: 2015.

Embodiment 195. The α4β7 binding protein of embodiment 193, wherein the VH comprises the amino acid sequence according to SEQ ID NO: 1910; and the VL comprises the amino acid sequence according to SEQ ID NO: 2016.

Embodiment 196. The α4β7 binding protein of embodiment 193, wherein the VH comprises the amino acid sequence according to SEQ ID NO: 1935; and the VL comprises the amino acid sequence according to SEQ ID NO: 2041.

Embodiment 197. The α4β7 binding protein of embodiment 193, wherein the VH comprises the amino acid sequence according to SEQ ID NO: 1936; and the VL comprises the amino acid sequence according to SEQ ID NO: 2042.

Embodiment 198. The α4β7 binding protein of embodiment 193, wherein the VH comprises the amino acid sequence according to SEQ ID NO: 1937; and the VL comprises the amino acid sequence according to SEQ ID NO: 2043.

Embodiment 199. The α4β7 binding protein of embodiment 193, wherein the VH comprises the amino acid sequence according to SEQ ID NO: 1938; and the VL comprises the amino acid sequence according to SEQ ID NO: 2044.

Embodiment 200. The α4β7 binding protein of embodiment 193, wherein the VH comprises the amino acid sequence according to SEQ ID NO: 1939; and the VL comprises the amino acid sequence according to SEQ ID NO: 2045.

Embodiment 201. The α4β7 binding protein of embodiment 193, wherein the VH comprises the amino acid sequence according to SEQ ID NO: 1967; and the VL comprises the amino acid sequence according to SEQ ID NO: 2073.

Embodiment 202. An α4β7 binding protein comprising:
c) a heavy chain variable region (VH) comprising (i) a CDR1 having an amino acid sequence according to any one of SEQ ID NOs: 1-106, (ii) a CDR2 having an amino acid sequence according to any one of SEQ ID NOs: 107-212, and (iii) a CDR3 having an amino acid sequence according to any one of SEQ ID NOs: 213-318;
d) a light chain variable region (VL) comprising (i) a CDR1 having an amino acid sequence according to any one of SEQ ID NOs: 319-424, (ii) a CDR2 having an amino acid sequence according to any one of SEQ ID NOs: 425-530, and (iii) a CDR3 having an amino acid sequence according to any one of SEQ ID NOs: 531-636; and
e) a modified Fc that comprises amino acid modifications M252Y, S254T, and T256E (YTE) and/or L234A/G237A (LAGA).

Embodiment 203. An α4β7 binding protein comprising:
a) a heavy chain variable region (VH) comprising (i) a CDR1 having an amino acid sequence according to any one of SEQ ID NOs: 1-106, (ii) a CDR2 having an amino acid sequence according to any one of SEQ ID NOs: 107-212, and (iii) a CDR3 having an amino acid sequence according to any one of SEQ ID NOs: 213-318;
b) a light chain variable region (VL) comprising (i) a CDR1 having an amino acid sequence according to any one of SEQ ID NOs: 319-424, (ii) a CDR2 having an amino acid sequence according to any one of SEQ ID NOs: 425-530, and (iii) a CDR3 having an amino acid sequence according to any one of SEQ ID NOs: 531-636; and
c) a modified Fc that extends half-life of the α4β7 binding protein as compared to an α4β7 binding protein that does not comprise the modified Fc.

Embodiment 204. An α4β7 binding protein, wherein the α4β7 binding protein specifically binds to an epitope of α4β7 and comprises a Fc domain comprising amino acid modifications M252Y, S254T, and T256E (YTE) and/or L234A/G237A (LAGA).

Embodiment 205. The α4β7 binding protein of any one of embodiments 202-204, wherein the Fc is an IgG1, IgG2 or IgG4 immunoglobulin Fc domain.

Embodiment 206. The α4β7 binding protein of embodiment 205, wherein the Fc is an IgG1 immunoglobulin domain.

Embodiment 207. The α4β7 binding protein of embodiment 205, wherein the Fc is an IgG2 immunoglobulin domain.

Embodiment 208. The α4β7 binding protein of embodiment 205, wherein the Fc is an IgG4 immunoglobulin domain.

Embodiment 209. A method of treating an inflammatory bowel disease in a patient in need thereof, the method comprising subcutaneously or intravenously administering to the patient an effective amount of an α4β7 binding protein of any one of embodiments 1-208.

Embodiment 210. The method of embodiment 209, wherein the inflammatory bowel disease is Crohn's disease or ulcerative colitis.

Embodiment 211. The method of embodiment 210, wherein the inflammatory bowel disease is ulcerative colitis.

Embodiment 212. The method of embodiment 210, wherein the inflammatory bowel disease is Crohn's disease.

Embodiment 213. The method of any one of embodiments 209-212, wherein administration of the α4β7 binding protein is subcutaneous.

Embodiment 214. The method of any one of embodiments 209-212, wherein administration of the α4β7 binding protein is intravenous.

Embodiment 215. A method of treating an inflammatory disease in a patient in need thereof, the method comprising subcutaneously or intravenously administering to the patient an effective amount of an α4β7 binding protein of any one of embodiments 1-208.

Embodiment 216. The method of embodiment 215, wherein the inflammatory disease is psoriasis.

Embodiment 217. The method of embodiment 215, wherein the inflammatory disease is psoriatic arthritis.

Embodiment 218. The method of embodiment 215, wherein the inflammatory disease is hidradenitis suppurativa.

Embodiment 219. The method of any one of embodiments 215-218, wherein administration of the α4β7 binding protein is subcutaneous.

Embodiment 220. The method of any one of embodiments 215-218, wherein administration of the α4β7 binding protein is intravenous.

Embodiment 221. An isolated nucleic acid encoding the α4β7 binding protein of any one of embodiments 1-208.

Embodiment 222. A recombinant host cell comprising the isolated nucleic acid of embodiment 221.

### EXAMPLES

The disclosure now being generally described, will be more readily understood by reference to the following examples, which are included merely for purposes of illustration of certain aspects and embodiments of the present disclosure, and is not intended to limit the disclosure.

### Example 1. In Silico Affinity Maturation and Liability Removal of an Anti-α4P7 Integrin Comparator Antibody

Residue scanning analysis of an anti-α4β7 integrin comparator antibody, which was humanized from the parental antibody ACT-1, was performed. The three-dimensional structure of ACT-1 in complex with its target antigen, human integrin α4β7 integrin, PDB Code 3V4P, was loaded into the MOE software.

Prior to analysis, the antibody structure was optimized using MOE's automated structural preparation workflow. Structural issues such as missing atoms or geometric outliers were corrected, and any other non-essential crystallographic molecules were removed to prepare the antibody model for the residue scanning analysis. Hydrogen atoms were added to the model using the Protonate 3D module in MOE, maintaining physiological pH conditions. A restrained minimization was then performed to complete the preparation. The six Complementarity Determining Regions (CDRs) were identified using the MOE Antibody module. Each CDR region was thoroughly examined, and the residues involved were noted for further analysis.

MOE's Residue Scanning tool was employed to perform a systematic scan of all the residues in the CDR regions. The residues were mutated one by one into all other 19 natural amino acids excluding cysteine, and the effects on binding affinity, stability, and other protein properties were predicted using the scoring functions in MOE. Based on the results, we identified key residues in the CDR regions that could be mutated without negatively impacting the binding affinity, stability, or developability of the comparator antibody. Combinations of mutations identified within each CDR were then evaluated in a similar fashion. Results are summarized in Table 4.

TABLE 4
Affinity and Stability of α4β7 Antibodies

| Antibody | ΔAffinity | ΔStability |
|---|---|---|
| Comparator | 0 | 0 |
| Antibody 3 | −1.263 | −0.023 |
| Antibody 4 | −1.157 | −0.041 |
| Antibody 5 | −4.978 | −0.410 |
| Antibody 6 | −5.114 | −0.080 |
| Antibody 7 | −0.264 | −0.474 |
| Antibody 8 | −3.208 | −1.516 |
| Antibody 9 | −8.055 | −2.402 |
| Antibody 10 | −2.632 | −1.704 |
| Antibody 11 | −1.522 | −0.148 |
| Antibody 12 | −6.234 | −1.380 |
| Antibody 13 | −11.591 | −1.619 |
| Antibody 14 | −4.707 | −0.900 |
| Antibody 15 | −1.449 | −0.381 |
| Antibody 16 | −1.862 | −0.111 |
| Antibody 17 | −4.178 | −0.489 |
| Antibody 18 | −0.333 | −0.060 |
| Antibody 19 | −0.867 | −1.198 |
| Antibody 20 | −0.474 | −2.921 |
| Antibody 21 | −0.370 | −0.071 |
| Antibody 22 | −0.165 | −0.323 |
| Antibody 23 | −0.995 | −2.163 |
| Antibody 24 | −1.577 | −0.177 |
| Antibody 25 | −1.973 | −0.173 |
| Antibody 26 | −0.576 | −2.986 |
| Antibody 27 | −0.066 | −0.858 |
| Antibody 28 | −0.277 | −0.405 |
| Antibody 29 | −0.035 | −0.306 |
| Antibody 30 | −0.053 | −0.308 |
| Antibody 31 | −0.112 | −0.537 |
| Antibody 32 | −2.617 | −1.049 |
| Antibody 33 | −0.403 | −1.248 |
| Antibody 34 | −0.157 | −1.092 |
| Antibody 35 | −0.045 | −0.483 |
| Antibody 36 | −0.556 | −1.257 |
| Antibody 37 | −0.297 | −0.856 |
| Antibody 38 | −0.083 | −1.714 |
| Antibody 39 | −0.138 | −1.767 |
| Antibody 40 | −0.753 | −3.059 |
| Antibody 41 | −2.172 | −1.166 |
| Antibody 42 | −4.673 | −0.936 |
| Antibody 43 | −0.724 | −1.043 |
| Antibody 44 | −5.345 | −0.448 |
| Antibody 45 | −6.568 | −0.770 |
| Antibody 46 | −3.807 | −0.373 |
| Antibody 47 | −0.131 | −0.128 |
| Antibody 48 | −0.228 | −0.180 |
| Antibody 49 | −3.968 | −0.640 |
| Antibody 50 | −5.855 | −0.407 |
| Antibody 51 | −0.263 | −0.368 |
| Antibody 52 | −0.764 | −2.181 |
| Antibody 53 | −1.711 | −2.767 |
| Antibody 54 | −4.572 | −2.634 |
| Antibody 55 | −1.107 | −2.510 |
| Antibody 56 | −1.401 | −2.407 |
| Antibody 57 | −0.383 | −0.033 |
| Antibody 58 | −3.894 | −1.337 |
| Antibody 59 | −4.109 | −0.556 |
| Antibody 60 | −4.398 | −0.099 |
| Antibody 61 | −4.040 | −1.278 |
| Antibody 62 | −3.698 | −1.531 |
| Antibody 63 | −4.152 | −2.183 |
| Antibody 64 | −5.873 | −0.332 |
| Antibody 65 | −3.976 | −1.208 |
| Antibody 66 | −4.425 | −2.279 |
| Antibody 67 | −5.004 | −2.274 |
| Antibody 68 | −0.500 | −0.102 |
| Antibody 69 | −4.225 | −1.022 |
| Antibody 70 | −9.501 | −2.634 |
| Antibody 71 | −1.102 | −2.164 |
| Antibody 72 | −0.748 | −0.195 |
| Antibody 73 | −6.167 | −0.339 |
| Antibody 74 | −5.074 | −0.440 |
| Antibody 75 | −1.201 | −0.070 |
| Antibody 76 | −1.864 | −0.190 |
| Antibody 77 | −4.980 | −0.660 |
| Antibody 78 | −5.317 | −0.141 |
| Antibody 79 | −4.866 | −0.300 |
| Antibody 80 | −2.600 | −0.143 |
| Antibody 81 | −0.217 | −0.354 |
| Antibody 82 | −0.150 | −0.204 |
| Antibody 83 | −0.297 | −0.217 |
| Antibody 84 | −0.033 | −1.597 |
| Antibody 85 | −0.013 | −3.418 |
| Antibody 86 | −0.006 | −3.183 |
| Antibody 87 | −0.021 | −3.554 |
| Antibody 88 | −0.023 | −2.943 |
| Antibody 89 | −0.021 | −2.415 |
| Antibody 90 | −0.028 | −2.890 |
| Antibody 91 | −0.006 | −1.419 |
| Antibody 92 | −0.148 | −2.796 |
| Antibody 93 | 0.000 | −3.088 |
| Antibody 94 | −0.008 | −1.379 |
| Antibody 95 | −0.138 | −0.800 |
| Antibody 96 | −0.041 | −1.172 |
| Antibody 97 | −0.144 | −3.014 |
| Antibody 98 | −0.221 | −2.542 |
| Antibody 99 | −0.004 | −0.158 |
| Antibody 100 | −0.002 | −0.002 |
| Antibody 101 | −0.005 | −0.664 |
| Antibody 102 | −0.005 | −0.329 |
| Antibody 103 | −0.004 | −0.144 |
| Antibody 104 | −0.003 | −0.060 |
| Antibody 105 | −0.001 | −0.279 |
| Antibody 106 | −0.001 | −0.238 |

ΔAffinity is a measure of predicted change in affinity from the comparator antibody. The absolute value of the number corresponds to the predicted magnitude of improvement. Improvements in affinity are indicated by negative values. Values are unitless predictions.
ΔStability is a measure of predicted change in stability from the comparator antibody. The absolute value of the number corresponds to the predicted magnitude of improvement. Improvements in stability are indicated by negative values. Values are unitless predictions.

### Example 2. Rehumanization of the Anti-α4β7 Integrin Comparator Antibody

Complementarity-determining region (CDR) grafting approach was used to humanize the parental mouse anti-human α4β7 ACT-1 mouse antibody, the parental monoclonal antibody of the comparator antibody. The parental mouse heavy and light sequences were modeled onto a human antibody framework. A set of human heavy and light chains were selected for humanization. The goal was to design pairs of these heavy and light chains that resulted in improved biophysical properties of the parental antibody while retaining α4β7 integrin binding. These humanized molecules were designed for improved developability profile during scale up in bioprocess.

### Example 3. Determination of Antibody Affinity to α4β7 Integrin

A SPR system equipped with a CM5 chip functionalized with an anti-human Fc antibody was used to determine the binding kinetic rates and affinity constants at 25° C. in a running buffer of HBS-P+ with the addition of 1 mM Mn2+, 1 mM Ca2+, and 1 mM Mg2+. Following a stabilization period in running buffer, antibodies previously diluted to 1-1.5 μg/mL were captured on the chip for 30 seconds at a flow rate of 10 μL/min. Subsequently, recombinant human α4β7 integrin was prepared at concentrations of 1.563 nM, 3.125 nM, 6.25 nM, 12.5 nM, 25 nM, 50 nM and 100 nM and injected at a flow rate of 30 μL/min for 180 seconds followed by a dissociation phase with just running buffer at a flow rate of 30 μL/min for 1200 seconds. Samples were injected in a multi-cycle manner over freshly captured mAb, by regenerating the capture surfaces with injection of 10 mM Glycine, pH 1.5. The data was processed and analyzed with SPR analysis software, where sensorgrams were fit to a 1:1 binding model to determine the apparent association (ka) and dissociation rate constants (kd). Their ratio provided the apparent equilibrium dissociation constant or affinity constant (KD=kd/ka). Results are summarized in Table 5.

TABLE 5

| | Association Constant | Dissociation Constant | Apparent |
|---|---|---|---|
| Antibody | (M⁻¹s⁻¹) | (s⁻¹) | K_{D} (nM) |
| Comparator | 8.68 × 10⁴ | 1.22 × 10⁻⁴ | 1.41 |
| Antibody 1 | 6.85 × 10⁴ | 4.66 × 10⁻⁴ | 6.79 |
| Antibody 2 | 5.64 × 10⁴ | 1.18 × 10⁻⁴ | 2.10 |

### Example 4. Binding to α4β7 Integrin or α4β1 Integrin on Cells

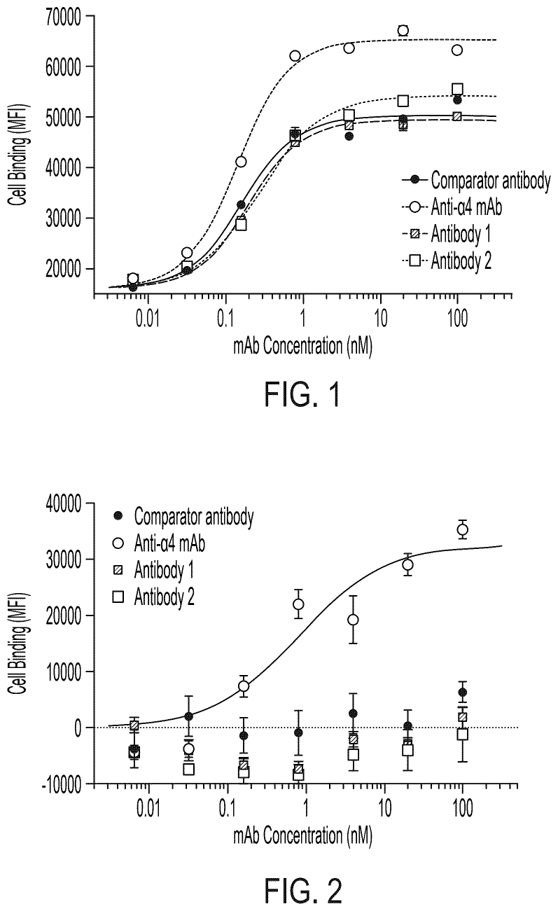

Antibody binding to α4β7 integrin or α4β1 integrin expressed on cells was determined using FACS and two cell lines. The first cell line was RPMI-8866 and is known to only express α4β7 integrin. The second cell line was Ramos and is known to only express α4β1. Briefly, cells were cultured and harvested according to standard vendor instructions. Cells were stained with purified antibodies at concentrations of 0 nM, 0.0064 nM, 0.032 nM, 0.16 nM, 0.8 nM, 4 nM, 20 nM, and 100 nM and incubated at 4° C. for 1 hour. Cells were subsequently stained with an Alexa Fluor 488-conjugated goat anti-human IgG secondary antibody at a 1:1000 dilution. Cells were incubated 4° C. for 1 hour, protected from light. Cells were then washed and the MFI of cells in each well were recorded by FACS using a flow cytometer. Subsequent data were analyzed using GraphPad Prism. EC50 values were determined as the concentration of antibody required to achieve 50% of the maximum plateau MFI. The anti-α4β7 integrin comparator antibody and Antibody 1 and Antibody 2 show specific binding to RPMI-8866 but not Ramos cells. On the other hand, an anti-α4 antibody tested binds to both cell lines. Results are summarized in Table 6 and shown in FIGS. 1 and 2.

TABLE 6

| | RPMI-8866 Binding | Ramos Binding |
|---|---|---|
| Antibody | EC₅₀ (nM) | EC₅₀ (nM) |
| Comparator | 0.17 | N.B. |
| Anti-α4 mAb | 0.14 | 0.81 |
| Antibody 1 | 0.20 | N.B. |
| Antibody 2 | 0.27 | N.B. |

N.B.—No binding observed within concentration range tested.

### Example 5. Inhibition of Cellular Adhesion Via MAdCAM-1 and α4β7 Integrin or VCAM-1 and α4ρ1 Integrin

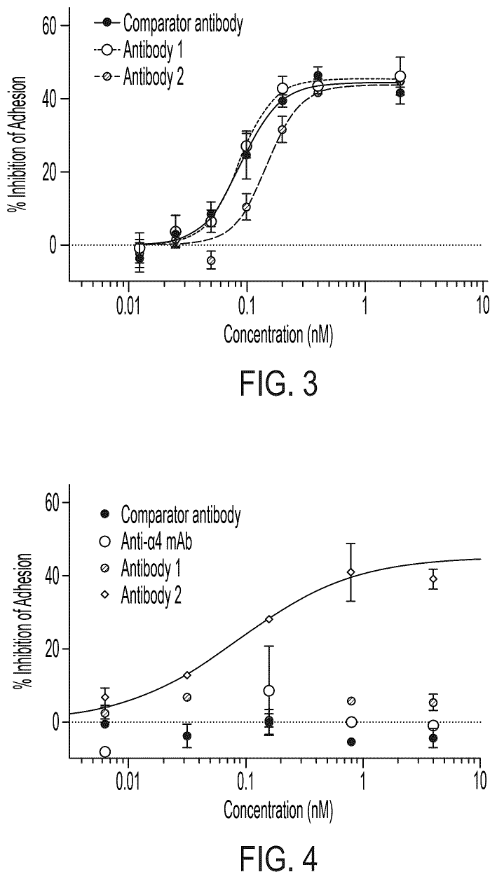

Integrins mediate cellular adhesion by binding to distinct cell adhesion molecules. Specifically, α4β7 integrin mediates adhesion through binding of MAdCAM-1 while α4β7 integrin mediates adhesion through binding of VCAM-1. To determine the ability of antibodies to block cellular adhesion mediated through either α4β7 integrin:MAdCAM-1 interaction or α4β1 integrin:VCAM-1, a cellular adhesion assay was conducted using HuT-78 cells, which have been shown to express both α4β7 and α4β1. Briefly, plates were prepared in advance by coating wells with either MAdCAM-1 diluted in PBS to 0.4 μg/mL or VCAM-1 diluted in PBS to 0.5 μg/mL. Plates were incubated at 4° C. overnight. The next day, cells were harvested according to standard vendor instructions. Cells were stained with Calcein AM, using a ratio of 1 μL of 1 mM Calcein AM per 1 mL of 1×10⁶ cells. Cells were incubated at 37° C. for 30 minutes. Subsequently cells were washed and resuspended in assay media consisting of DMEM, 0.1% BSA, 10 mM HEPES, and 0.5 mM MnCl₂ to a density of 800,000 cells/mL. Purified antibodies were mixed with cells at a 1:1 volume ratio, centrifuged gently at 10×g for 1 minute, and incubated at 37° C. for 30 minutes. Antibodies were used at final concentrations of 0 nM, 0.0125 nM, 0.025 nM, 0.05 nM, 0.1 nM, 0.2 nM, 0.4 nM, and 2 nM. Using a plate reader, individual well fluorescence values were read with a 485 nm excitation and 520 nm emission. Wells were then gently washed twice with washing buffer and plates were analyzed again in the same manner. Subsequent data were analyzed using GraphPad Prism. IC50 values were determined as the concentration of antibody required to inhibit 50% of the maximum cell adhesion observed. The comparator antibody and variant antibodies showed specific inhibition of MAdCAM-1 mediated cell adhesion but not VCAM-1 mediated. On the other hand, an anti-α4 antibody tested was able to inhibit cellular adhesion by VCAM-1. Results are summarized in Table 7 and shown in FIGS. 3 and 4.

TABLE 7

| | MAdCAM-1 Adhesion | VCAM-1 Adhesion |
|---|---|---|
| Antibody | Inhibition IC₅₀ (nM) | Inhibition IC₅₀ (nM) |
| Comparator | 0.090 | N.I. |
| Anti-α4 mAb | N.T. | 0.87 |
| Antibody 1 | 0.087 | N.I. |
| Antibody 2 | 0.15 | N.I. |

N.T.—Not tested.
N.I.—No inhibition observed within concentration range tested.

### EQUIVALENTS

The disclosure may be embodied in other specific forms without departing from the spirit or essential characteristics thereof. The foregoing embodiments are therefore to be considered in all respects illustrative rather than limiting the disclosure described herein. Scope of the disclosure is thus indicated by the appended claims rather than by the foregoing description, and all changes that come within the meaning and range of equivalency of the claims are intended to be embraced therein.

1. An α4β7 binding protein comprising a heavy chain variable domain (VH) comprising:
(a) a complementarity-determining region 1 (CDR1) comprising an amino acid sequence of SEQ ID NO: 3, a complementarity-determining region 1 (CDR2) comprising an amino acid sequence of SEQ ID NO: 109, and a complementarity-determining region 3 (CDR3) comprising an amino acid sequence of SEQ ID NO: 215; or
(b) a CDR1 comprising an amino acid sequence of SEQ ID NO: 4, a CDR2 comprising an amino acid sequence of SEQ ID NO: 110, and a CDR3 comprising an amino acid sequence of SEQ ID NO: 216; or
(c) a CDR1 comprising an amino acid sequence of SEQ ID NO: 5, a CDR2 comprising an amino acid sequence of SEQ ID NO: 111, and a CDR3 comprising an amino acid sequence of SEQ ID NO: 217; or
(d) a CDR1 comprising an amino acid sequence of SEQ ID NO: 6, a CDR2 comprising an amino acid sequence of SEQ ID NO: 112, and a CDR3 comprising an amino acid sequence of SEQ ID NO: 218.

2. The α4β7 binding protein of claim 1, further comprising a light chain variable region (VL), wherein
(a) the VH comprises the CDR1 having an amino acid sequence according to SEQ ID NO: 3, the CDR2 having an amino acid sequence according to SEQ ID NO: 109, and the CDR3 having an amino acid sequence according to SEQ ID NO: 215, and the VL comprises a CDR1 having an amino acid sequence according to SEQ ID NO: 321, a CDR2 having an amino acid sequence according to SEQ ID NO: 427, and a CDR3 having an amino acid sequence according to SEQ ID NO: 533; or
(b) the VH comprises the CDR1 having an amino acid sequence according to SEQ ID NO: 4, the CDR2 having an amino acid sequence according to SEQ ID NO: 110, and the CDR3 having an amino acid sequence according to SEQ ID NO: 216, and the VL comprises a CDR1 having an amino acid sequence according to SEQ ID NO: 322, a CDR2 having an amino acid sequence according to SEQ ID NO: 428, and a CDR3 having an amino acid sequence according to SEQ ID NO: 534; or
(c) the VH comprises the CDR1 having an amino acid sequence according to SEQ ID NO: 5, the CDR2 having an amino acid sequence according to SEQ ID NO: 111, and the CDR3 having an amino acid sequence according to SEQ ID NO: 217, and the VL comprises a CDR1 having an amino acid sequence according to SEQ ID NO: 323, a CDR2 having an amino acid sequence according to SEQ ID NO: 429, and a CDR3 having an amino acid sequence according to SEQ ID NO: 535; or
(d) the VH comprises the CDR1 having an amino acid sequence according to SEQ ID NO: 6, the CDR2 having an amino acid sequence according to SEQ ID NO: 112, and the CDR3 having an amino acid sequence according to SEQ ID NO: 218, and the VL comprises a CDR1 having an amino acid sequence according to SEQ ID NO: 324, a CDR2 having an amino acid sequence according to SEQ ID NO: 430, and a CDR3 having an amino acid sequence according to SEQ ID NO: 536.

3. The α4β7 binding protein of claim 2, wherein:
(a) the VH comprises the CDR1 having an amino acid sequence according to SEQ ID NO: 3, the CDR2 having an amino acid sequence according to SEQ ID NO: 109, and the CDR3 having an amino acid sequence according to SEQ ID NO: 215, the VL comprises the CDR1 having an amino acid sequence according to SEQ ID NO: 321, the CDR2 having an amino acid sequence according to SEQ ID NO: 427, and the CDR3 having an amino acid sequence according to SEQ ID NO: 533, and wherein the VH comprises a sequence having at least 80% sequence identity to the amino acid sequence of SEQ ID NO: 1911 and the VL comprises a sequence having at least 80% sequence identity to the amino acid sequence of SEQ ID NO: 2017; or
(b) the VH comprises the CDR1 having an amino acid sequence according to SEQ ID NO: 4, the CDR2 having an amino acid sequence according to SEQ ID NO: 110, and the CDR3 having an amino acid sequence according to SEQ ID NO: 216, the VL comprises the CDR1 having an amino acid sequence according to SEQ ID NO: 322, the CDR2 having an amino acid sequence according to SEQ ID NO: 428, and the CDR3 having an amino acid sequence according to SEQ ID NO: 534, and wherein the VH comprises a sequence having at least 80% sequence identity to the amino acid sequence of SEQ ID NO: 1912 and the VL comprises a sequence having at least 80% sequence identity to the amino acid sequence of SEQ ID NO: 2018; or
(c) the VH comprises the CDR1 having an amino acid sequence according to SEQ ID NO: 5, the CDR2 having an amino acid sequence according to SEQ ID NO: 111, and the CDR3 having an amino acid sequence according to SEQ ID NO: 217, the VL comprising the CDR1 having an amino acid sequence according to SEQ ID NO: 323, the CDR2 having an amino acid sequence according to SEQ ID NO: 429, and the CDR3 having an amino acid sequence according to SEQ ID NO: 535, and wherein the VH comprises a sequence having at least 80% sequence identity to the amino acid sequence of SEQ ID NO: 1913 and the VL comprises a sequence having at least 80% sequence identity to the amino acid sequence of SEQ ID NO: 2019; or
(d) the VH comprises the CDR1 having an amino acid sequence according to SEQ ID NO: 6, the CDR2 having an amino acid sequence according to SEQ ID NO: 112, and the CDR3 having an amino acid sequence according to SEQ ID NO: 218, the VL comprises the CDR1 having an amino acid sequence according to SEQ ID NO: 324, the CDR2 having an amino acid sequence according to SEQ ID NO: 430, and the CDR3 having an amino acid sequence according to SEQ ID NO: 536, and wherein the VH comprises a sequence having at least 80% sequence identity to the amino acid sequence of SEQ ID NO: 1914 and the VL comprises a sequence having at least 80% sequence identity to the amino acid sequence of SEQ ID NO: 2020.

4. The α4β7 binding protein of claim 1, comprising a modified Fc that extends half-life of the α4β7 binding protein as compared to an α4β7 binding protein that does not comprise the modified Fc.

5. The α4β7 binding protein of claim 4, wherein the modified Fc comprises amino acid modifications M252Y, S254T, and T256E (YTE) and/or L234A/G237A (LAGA).

6. A method of treating an inflammatory disease in a patient in need thereof, the method comprising administering subcutaneously or intravenously to the patient an effective amount of the α4β7 binding protein of claim 1.

7. A pharmaceutical composition comprising an effective amount of the α4β7 binding protein claim 1 and a pharmaceutically acceptable carrier.

8. An α4β7 binding protein comprising a heavy chain variable domain (VH) comprising:
(a) a CDR1 comprising an amino acid sequence of SEQ ID NO: 7, a CDR2 comprising an amino acid sequence of SEQ ID NO: 113, and a CDR3 comprising an amino acid sequence of SEQ ID NO: 219; or
(b) a CDR1 comprising an amino acid sequence of SEQ ID NO: 8, a CDR2 comprising an amino acid sequence of SEQ ID NO: 114, and a CDR3 comprising an amino acid sequence of SEQ ID NO: 220; or
(c) a CDR1 comprising an amino acid sequence of SEQ ID NO: 9, a CDR2 comprising an amino acid sequence of SEQ ID NO: 115, and a CDR3 comprising an amino acid sequence of SEQ ID NO: 221; or
(d) a CDR1 comprising an amino acid sequence of SEQ ID NO: 10, a CDR2 comprising an amino acid sequence of SEQ ID NO: 116, and a CDR3 comprising an amino acid sequence of SEQ ID NO: 222; or
(e) a CDR1 comprising an amino acid sequence of SEQ ID NO: 11, a CDR2 comprising an amino acid sequence of SEQ ID NO: 117, and a CDR3 comprising an amino acid sequence of SEQ ID NO: 223; or
(f) a CDR1 comprising an amino acid sequence of SEQ ID NO: 12, a CDR2 comprising an amino acid sequence of SEQ ID NO: 118, and a CDR3 comprising an amino acid sequence of SEQ ID NO: 224; or
(g) a CDR1 comprising an amino acid sequence of SEQ ID NO: 13, a CDR2 comprising an amino acid sequence of SEQ ID NO: 119, and a CDR3 comprising an amino acid sequence of SEQ ID NO: 225; or
(h) CDR1 comprising an amino acid sequence of SEQ ID NO: 14, a CDR2 comprising an amino acid sequence of SEQ ID NO: 120, and a CDR3 comprising an amino acid sequence of SEQ ID NO: 226; or
(i) a CDR1 comprising an amino acid sequence of SEQ ID NO: 15, a CDR2 comprising an amino acid sequence of SEQ ID NO: 121, and a CDR3 comprising an amino acid sequence of SEQ ID NO: 227; or
(j) a CDR1 comprising an amino acid sequence of SEQ ID NO: 16, a CDR2 comprising an amino acid sequence of SEQ ID NO: 122, and a CDR3 comprising an amino acid sequence of SEQ ID NO: 228; or
(k) a CDR1 comprising an amino acid sequence of SEQ ID NO: 17, a CDR2 comprising an amino acid sequence of SEQ ID NO: 123, and a CDR3 comprising an amino acid sequence of SEQ ID NO: 229; or
(l) a CDR1 comprising an amino acid sequence of SEQ ID NO: 18, a CDR2 comprising an amino acid sequence of SEQ ID NO: 124, and a CDR3 comprising an amino acid sequence of SEQ ID NO: 230.

9. The α4β7 binding protein of claim 8, further comprising a light chain variable region (VL), wherein
(a) the VH comprises the CDR1 having an amino acid sequence according to SEQ ID NO: 7, the CDR2 having an amino acid sequence according to SEQ ID NO: 113, and the CDR3 having an amino acid sequence according to SEQ ID NO: 219, and the VL comprises a CDR1 having an amino acid sequence according to SEQ ID NO: 325, a CDR2 having an amino acid sequence according to SEQ ID NO: 431, and a CDR3 having an amino acid sequence according to SEQ ID NO: 537; or
(b) the VH comprises the CDR1 having an amino acid sequence according to SEQ ID NO: 8, the CDR2 having an amino acid sequence according to SEQ ID NO: 114, and the vCDR3 having an amino acid sequence according to SEQ ID NO: 220, and the VL comprises a CDR1 having an amino acid sequence according to SEQ ID NO: 326, a CDR2 having an amino acid sequence according to SEQ ID NO: 432, and a CDR3 having an amino acid sequence according to SEQ ID NO: 538; or
(c) the VH comprises the CDR1 having an amino acid sequence according to SEQ ID NO: 9, the CDR2 having an amino acid sequence according to SEQ ID NO: 115, and the CDR3 having an amino acid sequence according to SEQ ID NO: 221, and the VL comprises a CDR1 having an amino acid sequence according to SEQ ID NO: 327, a CDR2 having an amino acid sequence according to SEQ ID NO: 433, and a CDR3 having an amino acid sequence according to SEQ ID NO: 539; or
(d) the VH comprises the CDR1 having an amino acid sequence according to SEQ ID NO: 10, the CDR2 having an amino acid sequence according to SEQ ID NO: 116, and the CDR3 having an amino acid sequence according to SEQ ID NO: 222, and the VL comprises a CDR1 having an amino acid sequence according to SEQ ID NO: 328, a CDR2 having an amino acid sequence according to SEQ ID NO: 434, and a CDR3 having an amino acid sequence according to SEQ ID NO: 540; or
(e) the VH comprises the CDR1 having an amino acid sequence according to SEQ ID NO: 11, the CDR2 having an amino acid sequence according to SEQ ID NO: 117, and the CDR3 having an amino acid sequence according to SEQ ID NO: 223, and the VL comprises a CDR1 having an amino acid sequence according to SEQ ID NO: 329, a CDR2 having an amino acid sequence according to SEQ ID NO: 435, and a CDR3 having an amino acid sequence according to SEQ ID NO: 541; or
(f) the VH comprises the CDR1 having an amino acid sequence according to SEQ ID NO: 12, the CDR2 having an amino acid sequence according to SEQ ID NO: 118, and the CDR3 having an amino acid sequence according to SEQ ID NO: 224, and the VL comprises a CDR1 having an amino acid sequence according to SEQ ID NO: 330, a CDR2 having an amino acid sequence according to SEQ ID NO: 436, and a CDR3 having an amino acid sequence according to SEQ ID NO: 542; or
(g) the VH comprises the CDR1 having an amino acid sequence according to SEQ ID NO: 13, the CDR2 having an amino acid sequence according to SEQ ID NO: 119, and the CDR3 having an amino acid sequence according to SEQ ID NO: 225, and the VL comprises a CDR1 having an amino acid sequence according to SEQ ID NO: 331, a CDR2 having an amino acid sequence according to SEQ ID NO: 437, and a CDR3 having an amino acid sequence according to SEQ ID NO: 543; or
(h) the VH comprises the CDR1 having an amino acid sequence according to SEQ ID NO: 14, the CDR2 having an amino acid sequence according to SEQ ID NO: 120, and the CDR3 having an amino acid sequence according to SEQ ID NO: 226, and the VL comprises a CDR1 having an amino acid sequence according to SEQ ID NO: 332, a CDR2 having an amino acid sequence according to SEQ ID NO: 438, and a CDR3 having an amino acid sequence according to SEQ ID NO: 544; or
(i) the VH comprises the CDR1 having an amino acid sequence according to SEQ ID NO: 15, the CDR2 having an amino acid sequence according to SEQ ID NO: 121, and the CDR3 having an amino acid sequence according to SEQ ID NO: 227, and the VL comprises a CDR1 having an amino acid sequence according to SEQ ID NO: 333, a CDR2 having an amino acid sequence according to SEQ ID NO: 439, and a CDR3 having an amino acid sequence according to SEQ ID NO: 545; or
(j) the VH comprises the CDR1 having an amino acid sequence according to SEQ ID NO: 16, the CDR2 having an amino acid sequence according to SEQ ID NO: 122, and the CDR3 having an amino acid sequence according to SEQ ID NO: 228, and the VL comprises a CDR1 having an amino acid sequence according to SEQ ID NO: 334, a CDR2 having an amino acid sequence according to SEQ ID NO: 440, and a CDR3 having an amino acid sequence according to SEQ ID NO: 546; or
(k) the VH comprises the CDR1 having an amino acid sequence according to SEQ ID NO: 17, the CDR2 having an amino acid sequence according to SEQ ID NO: 123, and the CDR3 having an amino acid sequence according to SEQ ID NO: 229, and the VL comprises a CDR1 having an amino acid sequence according to SEQ ID NO: 335, a CDR2 having an amino acid sequence according to SEQ ID NO: 441, and a CDR3 having an amino acid sequence according to SEQ ID NO: 547; or
(l) the VH comprises the CDR1 having an amino acid sequence according to SEQ ID NO: 18, the CDR2 having an amino acid sequence according to SEQ ID NO: 124, and the CDR3 having an amino acid sequence according to SEQ ID NO: 230, and the VL comprises a CDR1 having an amino acid sequence according to SEQ ID NO: 336, a CDR2 having an amino acid sequence according to SEQ ID NO: 442, and a CDR3 having an amino acid sequence according to SEQ ID NO: 548.

10. The α4β7 binding protein of claim 9, wherein:
(a) the VH comprises the CDR1 having an amino acid sequence according to SEQ ID NO: 7, the CDR2 having an amino acid sequence according to SEQ ID NO: 113, and the CDR3 having an amino acid sequence according to SEQ ID NO: 219, the VL comprises the CDR1 having an amino acid sequence according to SEQ ID NO: 325, the CDR2 having an amino acid sequence according to SEQ ID NO: 431, and the CDR3 having an amino acid sequence according to SEQ ID NO: 537, and wherein the VH comprises a sequence having at least 80% sequence identity to the amino acid sequence of SEQ ID NO: 1915 and the VL comprises a sequence having at least 80% sequence identity to the amino acid sequence of SEQ ID NO: 2021; or
(b) the VH comprises the CDR1 having an amino acid sequence according to SEQ ID NO: 8, the CDR2 having an amino acid sequence according to SEQ ID NO: 114, and the CDR3 having an amino acid sequence according to SEQ ID NO: 220, and the VL comprises the CDR1 having an amino acid sequence according to SEQ ID NO: 326 the CDR2 having an amino acid sequence according to SEQ ID NO: 432, and the CDR3 having an amino acid sequence according to SEQ ID NO: 538, and wherein the VH comprises a sequence having at least 80% sequence identity to the amino acid sequence of SEQ ID NO: 1916 and the VL comprises a sequence having at least 80% sequence identity to the amino acid sequence of SEQ ID NO: 2022; or
(c) the VH comprises the CDR1 having an amino acid sequence according to SEQ ID NO: 9, the CDR2 having an amino acid sequence according to SEQ ID NO: 115, and the CDR3 having an amino acid sequence according to SEQ ID NO: 221, and the VL comprises the CDR1 having an amino acid sequence according to SEQ ID NO: 327, the CDR2 having an amino acid sequence according to SEQ ID NO: 433, and the CDR3 having an amino acid sequence according to SEQ ID NO: 539, and wherein the VH comprises a sequence having at least 80% sequence identity to the amino acid sequence of SEQ ID NO: 1917 and the VL comprises a sequence having at least 80% sequence identity to the amino acid sequence of SEQ ID NO: 2023; or
(d) the VH comprises the CDR1 having an amino acid sequence according to SEQ ID NO: 10, the CDR2 having an amino acid sequence according to SEQ ID NO: 116, and the CDR3 having an amino acid sequence according to SEQ ID NO: 222, and the VL comprises the CDR1 having an amino acid sequence according to SEQ ID NO: 328, the CDR2 having an amino acid sequence according to SEQ ID NO: 434, and the CDR3 having an amino acid sequence according to SEQ ID NO: 540, and wherein the VH comprises a sequence having at least 80% sequence identity to the amino acid sequence of SEQ ID NO: 1918 and the VL comprises a sequence having at least 80% sequence identity to the amino acid sequence of SEQ ID NO: 2024; or
(e) the VH comprises the CDR1 having an amino acid sequence according to SEQ ID NO: 11, the CDR2 having an amino acid sequence according to SEQ ID NO: 117, and the CDR3 having an amino acid sequence according to SEQ ID NO: 223, and the VL comprises the CDR1 having an amino acid sequence according to SEQ ID NO: 329, the CDR2 having an amino acid sequence according to SEQ ID NO: 435, and the CDR3 having an amino acid sequence according to SEQ ID NO: 541, and wherein the VH comprises a sequence having at least 80% sequence identity to the amino acid sequence of SEQ ID NO: 1919 and the VL comprises a sequence having at least 80% sequence identity to the amino acid sequence of SEQ ID NO: 2025; or
(f) the VH comprises the CDR1 having an amino acid sequence according to SEQ ID NO: 12, the CDR2 having an amino acid sequence according to SEQ ID NO: 118, and the CDR3 having an amino acid sequence according to SEQ ID NO: 224, and the VL comprises the CDR1 having an amino acid sequence according to SEQ ID NO: 330, the CDR2 having an amino acid sequence according to SEQ ID NO: 436, and the CDR3 having an amino acid sequence according to SEQ ID NO: 542, and wherein the VH comprises a sequence having at least 80% sequence identity to the amino acid sequence of SEQ ID NO: 1920 and the VL comprises a sequence having at least 80% sequence identity to the amino acid sequence of SEQ ID NO: 2026; or
(g) the VH comprises the CDR1 having an amino acid sequence according to SEQ ID NO: 13, the CDR2 having an amino acid sequence according to SEQ ID NO: 119, and the CDR3 having an amino acid sequence according to SEQ ID NO: 225, and the VL comprises the CDR1 having an amino acid sequence according to SEQ ID NO: 331, the CDR2 having an amino acid sequence according to SEQ ID NO: 437, and the CDR3 having an amino acid sequence according to SEQ ID NO: 543, and wherein the VH comprises a sequence having at least 80% sequence identity to the amino acid sequence of SEQ ID NO: 1921 and the VL comprises a sequence having at least 80% sequence identity to the amino acid sequence of SEQ ID NO: 2027; or
(h) the VH comprises the CDR1 having an amino acid sequence according to SEQ ID NO: 14, the CDR2 having an amino acid sequence according to SEQ ID NO: 120, and the CDR3 having an amino acid sequence according to SEQ ID NO: 226, and the VL comprises the CDR1 having an amino acid sequence according to SEQ ID NO: 332, the CDR2 having an amino acid sequence according to SEQ ID NO: 438, and the CDR3 having an amino acid sequence according to SEQ ID NO: 544, and wherein the VH comprises a sequence having at least 80% sequence identity to the amino acid sequence of SEQ ID NO: 1922 and the VL comprises a sequence having at least 80% sequence identity to the amino acid sequence of SEQ ID NO: 2028; or
(i) the VH comprises the CDR1 having an amino acid sequence according to SEQ ID NO: 15, the CDR2 having an amino acid sequence according to SEQ ID NO: 121, and the CDR3 having an amino acid sequence according to SEQ ID NO: 227, and the VL comprises the CDR1 having an amino acid sequence according to SEQ ID NO: 333, the CDR2 having an amino acid sequence according to SEQ ID NO: 439, and the CDR3 having an amino acid sequence according to SEQ ID NO: 545, and wherein the VH comprises a sequence having at least 80% sequence identity to the amino acid sequence of SEQ ID NO: 1923 and the VL comprises a sequence having at least 80% sequence identity to the amino acid sequence of SEQ ID NO: 2029; or
(j) the VH comprises the CDR1 having an amino acid sequence according to SEQ ID NO: 16, the CDR2 having an amino acid sequence according to SEQ ID NO: 122, and the CDR3 having an amino acid sequence according to SEQ ID NO: 228, and the VL comprises the CDR1 having an amino acid sequence according to SEQ ID NO: 334, the CDR2 having an amino acid sequence according to SEQ ID NO: 440, and the CDR3 having an amino acid sequence according to SEQ ID NO: 546, and wherein the VH comprises a sequence having at least 80% sequence identity to the amino acid sequence of SEQ ID NO: 1924 and the VL comprises a sequence having at least 80% sequence identity to the amino acid sequence of SEQ ID NO: 2030; or
(k) the VH comprises the CDR1 having an amino acid sequence according to SEQ ID NO: 17, the CDR2 having an amino acid sequence according to SEQ ID NO: 123, and the CDR3 having an amino acid sequence according to SEQ ID NO: 229, and the VL comprises the CDR1 having an amino acid sequence according to SEQ ID NO: 335, the CDR2 having an amino acid sequence according to SEQ ID NO: 441, and the CDR3 having an amino acid sequence according to SEQ ID NO: 547, and wherein the VH comprises a sequence having at least 80% sequence identity to the amino acid sequence of SEQ ID NO: 1925 and the VL comprises a sequence having at least 80% sequence identity to the amino acid sequence of SEQ ID NO: 2031; or
(l) the VH comprises the CDR1 having an amino acid sequence according to SEQ ID NO: 18, the CDR2 having an amino acid sequence according to SEQ ID NO: 124, and the CDR3 having an amino acid sequence according to SEQ ID NO: 230, and the VL comprises the CDR1 having an amino acid sequence according to SEQ ID NO: 336, the CDR2 having an amino acid sequence according to SEQ ID NO: 442, and the CDR3 having an amino acid sequence according to SEQ ID NO: 548, and wherein the VH comprises a sequence having at least 80% sequence identity to the amino acid sequence of SEQ ID NO: 1926 and the VL comprises a sequence having at least 80% sequence identity to the amino acid sequence of SEQ ID NO: 2032.

11. The α4β7 binding protein of claim 8, comprising a modified Fc that extends half-life of the α4β7 binding protein as compared to an α4β7 binding protein that does not comprise the modified Fc.

12. The α4β7 binding protein of claim 11, wherein the modified Fc comprises amino acid modifications M252Y, S254T, and T256E (YTE) and/or L234A/G237A (LAGA).

13. A method of treating an inflammatory disease in a patient in need thereof, the method comprising administering subcutaneously or intravenously to the patient an effective amount of the α4β7 binding protein of claim 8.

14. A pharmaceutical composition comprising an effective amount of the α4β7 binding protein claim 8 and a pharmaceutically acceptable carrier.

15. An α4β7 binding protein comprising a heavy chain variable domain (VH) comprising:
(a) a CDR1 comprising an amino acid sequence of SEQ ID NO: 19, a CDR2 comprising an amino acid sequence of SEQ ID NO: 125, and a CDR3 comprising an amino acid sequence of SEQ ID NO: 231; or
(b) a CDR1 comprising an amino acid sequence of SEQ ID NO: 20, a CDR2 comprising an amino acid sequence of SEQ ID NO: 126, and a CDR3 comprising an amino acid sequence of SEQ ID NO: 232; or
(c) a CDR1 comprising an amino acid sequence of SEQ ID NO: 21, a CDR2 comprising an amino acid sequence of SEQ ID NO: 127, and a CDR3 comprising an amino acid sequence of SEQ ID NO: 233; or
(d) a CDR1 comprising an amino acid sequence of SEQ ID NO: 22, a CDR2 comprising an amino acid sequence of SEQ ID NO: 128, and a CDR3 comprising an amino acid sequence of SEQ ID NO: 234; or
(e) a CDR1 comprising an amino acid sequence of SEQ ID NO: 23, a CDR2 comprising an amino acid sequence of SEQ ID NO: 129, and a CDR3 comprising an amino acid sequence of SEQ ID NO: 235; or
(f) a CDR1 comprising an amino acid sequence of SEQ ID NO: 24, a CDR2 comprising an amino acid sequence of SEQ ID NO: 130, and a CDR3 comprising an amino acid sequence of SEQ ID NO: 236; or
(g) a CDR1 comprising an amino acid sequence of SEQ ID NO: 25, a CDR2 comprising an amino acid sequence of SEQ ID NO: 131 and a CDR3 comprising an amino acid sequence of SEQ ID NO: 237; or
(h) a CDR1 comprising an amino acid sequence of SEQ ID NO: 26, a CDR2 comprising an amino acid sequence of SEQ ID NO: 132, and a CDR3 comprising an amino acid sequence of SEQ ID NO: 238; or
(i) a CDR1 comprising an amino acid sequence of SEQ ID NO: 32, a CDR2 comprising an amino acid sequence of SEQ ID NO: 138, and a CDR3 comprising an amino acid sequence of SEQ ID NO: 244; or
(j) a CDR1 comprising an amino acid sequence of SEQ ID NO: 33, a CDR2 comprising an amino acid sequence of SEQ ID NO: 139, and a CDR3 comprising an amino acid sequence of SEQ ID NO: 245; or
(k) a CDR1 comprising an amino acid sequence of SEQ ID NO: 34, a CDR2 comprising an amino acid sequence of SEQ ID NO: 140, and a CDR3 comprising an amino acid sequence of SEQ ID NO: 246; or
(l) a CDR1 comprising an amino acid sequence of SEQ ID NO: 35, a CDR2 comprising an amino acid sequence of SEQ ID NO: 141, and a CDR3 comprising an amino acid sequence of SEQ ID NO: 247; or
(m) a CDR1 comprising an amino acid sequence of SEQ ID NO: 36, a CDR2 comprising an amino acid sequence of SEQ ID NO: 142, and a CDR3 comprising an amino acid sequence of SEQ ID NO: 248; or
(n) a CDR1 comprising an amino acid sequence of SEQ ID NO: 37, a CDR2 comprising an amino acid sequence of SEQ ID NO: 143, and a CDR3 comprising an amino acid sequence of SEQ ID NO: 249; or
(o) a CDR1 comprising an amino acid sequence of SEQ ID NO: 38, a CDR2 comprising an amino acid sequence of SEQ ID NO: 144, and a CDR3 comprising an amino acid sequence of SEQ ID NO: 250; or
(p) a CDR1 comprising an amino acid sequence of SEQ ID NO: 39, a CDR2 comprising an amino acid sequence of SEQ ID NO: 145, and a CDR3 comprising an amino acid sequence of SEQ ID NO: 251; or
(q) a CDR1 comprising an amino acid sequence of SEQ ID NO: 40, a CDR2 comprising an amino acid sequence of SEQ ID NO: 146, and a CDR3 comprising an amino acid sequence of SEQ ID NO: 252; or
(r) a CDR1 comprising an amino acid sequence of SEQ ID NO: 41, a CDR2 comprising an amino acid sequence of SEQ ID NO: 147, and a CDR3 comprising an amino acid sequence of SEQ ID NO: 253; or
(s) a CDR1 comprising an amino acid sequence of SEQ ID NO: 42, a CDR2 comprising an amino acid sequence of SEQ ID NO: 148, and a CDR3 comprising an amino acid sequence of SEQ ID NO: 254; or
(t) a CDR1 comprising an amino acid sequence of SEQ ID NO: 43, a CDR2 comprising an amino acid sequence of SEQ ID NO: 149, and a CDR3 comprising an amino acid sequence of SEQ ID NO: 255; or
(u) a CDR1 comprising an amino acid sequence of SEQ ID NO: 44, a CDR2 comprising an amino acid sequence of SEQ ID NO: 150, and a CDR3 comprising an amino acid sequence of SEQ ID NO: 256; or
(v) a CDR1 comprising an amino acid sequence of SEQ ID NO: 45, a CDR2 comprising an amino acid sequence of SEQ ID NO: 151, and a CDR3 comprising an amino acid sequence of SEQ ID NO: 257; or
(w) a CDR1 comprising an amino acid sequence of SEQ ID NO: 46, a CDR2 comprising an amino acid sequence of SEQ ID NO: 152, and a CDR3 comprising an amino acid sequence of SEQ ID NO: 258; or
(x) a CDR1 comprising an amino acid sequence of SEQ ID NO: 47, a CDR2 comprising an amino acid sequence of SEQ ID NO: 153, and a CDR3 comprising an amino acid sequence of SEQ ID NO: 259; or
(y) a CDR1 comprising an amino acid sequence of SEQ ID NO: 48, a CDR2 comprising an amino acid sequence of SEQ ID NO: 154; and a CDR3 comprising an amino acid sequence of SEQ ID NO: 260.

16. The α4β7 binding protein of claim 15, further comprising a light chain variable region (VL), wherein:
(a) the VH comprises the CDR1 having an amino acid sequence according to SEQ ID NO: 19, the CDR2 having an amino acid sequence according to SEQ ID NO: 125, and the CDR3 having an amino acid sequence according to SEQ ID NO: 231, and the VL comprises a CDR1 having an amino acid sequence according to SEQ ID NO: 337, a CDR2 having an amino acid sequence according to SEQ ID NO: 443, and a CDR3 having an amino acid sequence according to SEQ ID NO: 549; or
(b) the VH comprises the CDR1 having an amino acid sequence according to SEQ ID NO: 20, the CDR2 having an amino acid sequence according to SEQ ID NO: 126, and the CDR3 having an amino acid sequence according to SEQ ID NO: 232, and the VL comprises a CDR1 having an amino acid sequence according to SEQ ID NO: 338, a CDR2 having an amino acid sequence according to SEQ ID NO: 444, and a CDR3 having an amino acid sequence according to SEQ ID NO: 550; or
(c) the VH comprises the CDR1 having an amino acid sequence according to SEQ ID NO: 21, the CDR2 having an amino acid sequence according to SEQ ID NO: 127, and the CDR3 having an amino acid sequence according to SEQ ID NO: 233, and the VL comprises a CDR1 having an amino acid sequence according to SEQ ID NO: 339, a CDR2 having an amino acid sequence according to SEQ ID NO: 445, and a CDR3 having an amino acid sequence according to SEQ ID NO: 551; or
(d) the VH comprises the CDR1 having an amino acid sequence according to SEQ ID NO: 22, the CDR2 having an amino acid sequence according to SEQ ID NO: 128, and the CDR3 having an amino acid sequence according to SEQ ID NO: 234, and the VL comprises a CDR1 having an amino acid sequence according to SEQ ID NO: 340, a CDR2 having an amino acid sequence according to SEQ ID NO: 446, and a CDR3 having an amino acid sequence according to SEQ ID NO: 552; or
(e) the VH comprises the CDR1 having an amino acid sequence according to SEQ ID NO: 23, the CDR2 having an amino acid sequence according to SEQ ID NO: 129, and the CDR3 having an amino acid sequence according to SEQ ID NO: 235, and the VL comprises a CDR1 having an amino acid sequence according to SEQ ID NO: 341, a CDR2 having an amino acid sequence according to SEQ ID NO: 447, and a CDR3 having an amino acid sequence according to SEQ ID NO: 553; or
(f) the VH comprises the CDR1 having an amino acid sequence according to SEQ ID NO: 24, the CDR2 having an amino acid sequence according to SEQ ID NO: 130, and the CDR3 having an amino acid sequence according to SEQ ID NO: 236, and the VL comprises a CDR1 having an amino acid sequence according to SEQ ID NO: 342, a CDR2 having an amino acid sequence according to SEQ ID NO: 448, and a CDR3 having an amino acid sequence according to SEQ ID NO: 554; or
(g) the VH comprises the CDR1 having an amino acid sequence according to SEQ ID NO: 25, the CDR2 having an amino acid sequence according to SEQ ID NO: 131, and the CDR3 having an amino acid sequence according to SEQ ID NO: 237, and the VL comprises a CDR1 having an amino acid sequence according to SEQ ID NO: 343, a CDR2 having an amino acid sequence according to SEQ ID NO: 449, and a CDR3 having an amino acid sequence according to SEQ ID NO: 555; or
(h) the VH comprises the CDR1 having an amino acid sequence according to SEQ ID NO: 26, the CDR2 having an amino acid sequence according to SEQ ID NO: 132, and the CDR3 having an amino acid sequence according to SEQ ID NO: 238, and the VL comprises a CDR1 having an amino acid sequence according to SEQ ID NO: 344, a CDR2 having an amino acid sequence according to SEQ ID NO: 450, and a CDR3 having an amino acid sequence according to SEQ ID NO: 556; or
(i) the VH comprises the CDR1 having an amino acid sequence according to SEQ ID NO: 32, the CDR2 having an amino acid sequence according to SEQ ID NO: 138, and the CDR3 having an amino acid sequence according to SEQ ID NO: 244, and the VL comprises a CDR1 having an amino acid sequence according to SEQ ID NO: 350, a CDR2 having an amino acid sequence according to SEQ ID NO: 456, and a CDR3 having an amino acid sequence according to SEQ ID NO: 562; or
(j) the VH comprises the CDR1 having an amino acid sequence according to SEQ ID NO: 33, the CDR2 having an amino acid sequence according to SEQ ID NO: 139, and the CDR3 having an amino acid sequence according to SEQ ID NO: 245, and the VL comprises a CDR1 having an amino acid sequence according to SEQ ID NO: 351, a CDR2 having an amino acid sequence according to SEQ ID NO: 457, and a CDR3 having an amino acid sequence according to SEQ ID NO: 563; or
(k) the VH comprises the CDR1 having an amino acid sequence according to SEQ ID NO: 34, the CDR2 having an amino acid sequence according to SEQ ID NO: 140, and the CDR3 having an amino acid sequence according to SEQ ID NO: 246, and the VL comprises a CDR1 having an amino acid sequence according to SEQ ID NO: 352, a CDR2 having an amino acid sequence according to SEQ ID NO: 458, and a CDR3 having an amino acid sequence according to SEQ ID NO: 564; or
(l) the VH comprises the CDR1 having an amino acid sequence according to SEQ ID NO: 35, the CDR2 having an amino acid sequence according to SEQ ID NO: 141, and the CDR3 having an amino acid sequence according to SEQ ID NO: 247, and the VL comprises a CDR1 having an amino acid sequence according to SEQ ID NO: 353, a CDR2 having an amino acid sequence according to SEQ ID NO: 459, and a CDR3 having an amino acid sequence according to SEQ ID NO: 565; or
(m) the VH comprises the CDR1 having an amino acid sequence according to SEQ ID NO: 36, the CDR2 having an amino acid sequence according to SEQ ID NO: 142, and the CDR3 having an amino acid sequence according to SEQ ID NO: 248, and the VL comprises a CDR1 having an amino acid sequence according to SEQ ID NO: 354, a CDR2 having an amino acid sequence according to SEQ ID NO: 460, and a CDR3 having an amino acid sequence according to SEQ ID NO: 566; or
(n) the VH comprises the CDR1 having an amino acid sequence according to SEQ ID NO: 37, the CDR2 having an amino acid sequence according to SEQ ID NO: 143, and the CDR3 having an amino acid sequence according to SEQ ID NO: 249, and the VL comprises a CDR1 having an amino acid sequence according to SEQ ID NO: 355, a CDR2 having an amino acid sequence according to SEQ ID NO: 461, and a CDR3 having an amino acid sequence according to SEQ ID NO: 567; or
(o) the VH comprises the CDR1 having an amino acid sequence according to SEQ ID NO: 38, the CDR2 having an amino acid sequence according to SEQ ID NO: 144, and the CDR3 having an amino acid sequence according to SEQ ID NO: 250, and the VL comprises a CDR1 having an amino acid sequence according to SEQ ID NO: 356, a CDR2 having an amino acid sequence according to SEQ ID NO: 462, and a CDR3 having an amino acid sequence according to SEQ ID NO: 568; or
(p) the VH comprises the CDR1 having an amino acid sequence according to SEQ ID NO: 39, the CDR2 having an amino acid sequence according to SEQ ID NO: 145, and the CDR3 having an amino acid sequence according to SEQ ID NO: 251, and VL comprises a CDR1 having an amino acid sequence according to SEQ ID NO: 357, a CDR2 having an amino acid sequence according to SEQ ID NO: 463, and a CDR3 having an amino acid sequence according to SEQ ID NO: 569; or
(q) the VH comprises the CDR1 having an amino acid sequence according to SEQ ID NO: 40, the CDR2 having an amino acid sequence according to SEQ ID NO: 146, and the CDR3 having an amino acid sequence according to SEQ ID NO: 252, and the VL comprises a CDR1 having an amino acid sequence according to SEQ ID NO: 358, a CDR2 having an amino acid sequence according to SEQ ID NO: 464, and a CDR3 having an amino acid sequence according to SEQ ID NO: 570; or
(r) the VH comprises the CDR1 having an amino acid sequence according to SEQ ID NO: 41, the CDR2 having an amino acid sequence according to SEQ ID NO: 147, and the CDR3 having an amino acid sequence according to SEQ ID NO: 253, and the VL comprises a CDR1 having an amino acid sequence according to SEQ ID NO: 359, a CDR2 having an amino acid sequence according to SEQ ID NO: 465, and a CDR3 having an amino acid sequence according to SEQ ID NO: 571; or
(s) the VH comprises the CDR1 having an amino acid sequence according to SEQ ID NO: 42, the CDR2 having an amino acid sequence according to SEQ ID NO: 148, and the CDR3 having an amino acid sequence according to SEQ ID NO: 254, and the VL comprises a CDR1 having an amino acid sequence according to SEQ ID NO: 360, a CDR2 having an amino acid sequence according to SEQ ID NO: 466, and a CDR3 having an amino acid sequence according to SEQ ID NO: 572; or
(t) the VH comprises the CDR1 having an amino acid sequence according to SEQ ID NO: 43, the CDR2 having an amino acid sequence according to SEQ ID NO: 149, and the CDR3 having an amino acid sequence according to SEQ ID NO: 255, and the VL comprises a CDR1 having an amino acid sequence according to SEQ ID NO: 361, a CDR2 having an amino acid sequence according to SEQ ID NO: 467, and a CDR3 having an amino acid sequence according to SEQ ID NO: 573; or
(u) the VH comprises the CDR1 having an amino acid sequence according to SEQ ID NO: 44, the CDR2 having an amino acid sequence according to SEQ ID NO: 150, and the CDR3 having an amino acid sequence according to SEQ ID NO: 256, and the VL comprises a CDR1 having an amino acid sequence according to SEQ ID NO: 362, a CDR2 having an amino acid sequence according to SEQ ID NO: 468, and a CDR3 having an amino acid sequence according to SEQ ID NO: 574; or
(v) the VH comprises the CDR1 having an amino acid sequence according to SEQ ID NO: 45, the CDR2 having an amino acid sequence according to SEQ ID NO: 151, and the CDR3 having an amino acid sequence according to SEQ ID NO: 257, and the VL comprises a CDR1 having an amino acid sequence according to SEQ ID NO: 363, a CDR2 having an amino acid sequence according to SEQ ID NO: 469, and a CDR3 having an amino acid sequence according to SEQ ID NO: 575; or
(w) the VH comprises the CDR1 having an amino acid sequence according to SEQ ID NO: 46, the CDR2 having an amino acid sequence according to SEQ ID NO: 152, and the CDR3 having an amino acid sequence according to SEQ ID NO: 258, and the VL comprises a CDR1 having an amino acid sequence according to SEQ ID NO: 364, a CDR2 having an amino acid sequence according to SEQ ID NO: 470, and a CDR3 having an amino acid sequence according to SEQ ID NO: 576; or
(x) the VH comprises the CDR1 having an amino acid sequence according to SEQ ID NO: 47, the CDR2 having an amino acid sequence according to SEQ ID NO: 153, and the CDR3 having an amino acid sequence according to SEQ ID NO: 259, and the VL comprises a CDR1 having an amino acid sequence according to SEQ ID NO: 365, a CDR2 having an amino acid sequence according to SEQ ID NO: 471, and a CDR3 having an amino acid sequence according to SEQ ID NO: 577; or
(y) the VH comprises the CDR1 having an amino acid sequence according to SEQ ID NO: 48, the CDR2 having an amino acid sequence according to SEQ ID NO: 154, and the CDR3 having an amino acid sequence according to SEQ ID NO: 260, and the VL comprises a CDR1 having an amino acid sequence according to SEQ ID NO: 366, a CDR2 having an amino acid sequence according to SEQ ID NO: 472, and a CDR3 having an amino acid sequence according to SEQ ID NO: 578.

17. The α4β7 binding protein of claim 15, wherein:
(a) the VH comprises the CDR1 having an amino acid sequence according to SEQ ID NO: 19, the CDR2 having an amino acid sequence according to SEQ ID NO: 125, and the CDR3 having an amino acid sequence according to SEQ ID NO: 231, and the VL comprises the CDR1 having an amino acid sequence according to SEQ ID NO: 337, the CDR2 having an amino acid sequence according to SEQ ID NO: 443, and the CDR3 having an amino acid sequence according to SEQ ID NO: 549, and wherein the VH comprises a sequence having at least 80% sequence identity to the amino acid sequence of SEQ ID NO: 1927 and the VL comprises a sequence having at least 80% sequence identity to the amino acid sequence of SEQ ID NO: 2033; or
(b) the VH comprises the CDR1 having an amino acid sequence according to SEQ ID NO: 20, the CDR2 having an amino acid sequence according to SEQ ID NO: 126, and the CDR3 having an amino acid sequence according to SEQ ID NO: 232, and the VL comprises the CDR1 having an amino acid sequence according to SEQ ID NO: 338, the CDR2 having an amino acid sequence according to SEQ ID NO: 444, and the CDR3 having an amino acid sequence according to SEQ ID NO: 550, and wherein the VH comprises a sequence having at least 80% sequence identity to the amino acid sequence of SEQ ID NO: 1928 and the VL comprises a sequence having at least 80% sequence identity to the amino acid sequence of SEQ ID NO: 2034; or
(c) the VH comprises the CDR1 having an amino acid sequence according to SEQ ID NO: 21, the CDR2 having an amino acid sequence according to SEQ ID NO: 127, and the CDR3 having an amino acid sequence according to SEQ ID NO: 233, and the VL comprises the CDR1 having an amino acid sequence according to SEQ ID NO: 339, the CDR2 having an amino acid sequence according to SEQ ID NO: 445, and the CDR3 having an amino acid sequence according to SEQ ID NO: 551, and wherein the VH comprises a sequence having at least 80% sequence identity to the amino acid sequence of SEQ ID NO: 1929 and the VL comprises a sequence having at least 80% sequence identity to the amino acid sequence of SEQ ID NO: 2035; or
(d) the VH comprises the CDR1 having an amino acid sequence according to SEQ ID NO: 22, the CDR2 having an amino acid sequence according to SEQ ID NO: 128, and the CDR3 having an amino acid sequence according to SEQ ID NO: 234, and the VL comprises the CDR1 having an amino acid sequence according to SEQ ID NO: 340, the CDR2 having an amino acid sequence according to SEQ ID NO: 446, and the CDR3 having an amino acid sequence according to SEQ ID NO: 552, and wherein the VH comprises a sequence having at least 80% sequence identity to the amino acid sequence of SEQ ID NO: 1930 and the VL comprises a sequence having at least 80% sequence identity to the amino acid sequence of SEQ ID NO: 2036; or
(e) the VH comprises the CDR1 having an amino acid sequence according to SEQ ID NO: 23, the CDR2 having an amino acid sequence according to SEQ ID NO: 129, and the CDR3 having an amino acid sequence according to SEQ ID NO: 235, and the VL comprises the CDR1 having an amino acid sequence according to SEQ ID NO: 341, the CDR2 having an amino acid sequence according to SEQ ID NO: 447, and the CDR3 having an amino acid sequence according to SEQ ID NO: 553, and wherein the VH comprises a sequence having at least 80% sequence identity to the amino acid sequence of SEQ ID NO: 1931 and the VL comprises a sequence having at least 80% sequence identity to the amino acid sequence of SEQ ID NO: 2037; or
(f) the VH comprises the CDR1 having an amino acid sequence according to SEQ ID NO: 24, the CDR2 having an amino acid sequence according to SEQ ID NO: 130, and the CDR3 having an amino acid sequence according to SEQ ID NO: 236, and the VL comprises the CDR1 having an amino acid sequence according to SEQ ID NO: 342, the CDR2 having an amino acid sequence according to SEQ ID NO: 448, and the CDR3 having an amino acid sequence according to SEQ ID NO: 55, and wherein the VH comprises a sequence having at least 80% sequence identity to the amino acid sequence of SEQ ID NO: 1932 and the VL comprises a sequence having at least 80% sequence identity to the amino acid sequence of SEQ ID NO: 2038; or
(g) the VH comprises the CDR1 having an amino acid sequence according to SEQ ID NO: 25, the CDR2 having an amino acid sequence according to SEQ ID NO: 131, and the CDR3 having an amino acid sequence according to SEQ ID NO: 237, and the VL comprises the CDR1 having an amino acid sequence according to SEQ ID NO: 343, the CDR2 having an amino acid sequence according to SEQ ID NO: 449, and the CDR3 having an amino acid sequence according to SEQ ID NO: 555, and wherein the VH comprises a sequence having at least 80% sequence identity to the amino acid sequence of SEQ ID NO: 1933 and the VL comprises a sequence having at least 80% sequence identity to the amino acid sequence of SEQ ID NO: 2039; or
(h) the VH comprises the CDR1 having an amino acid sequence according to SEQ ID NO: 26, the CDR2 having an amino acid sequence according to SEQ ID NO: 132, and the CDR3 having an amino acid sequence according to SEQ ID NO: 238, and the VL comprises the CDR1 having an amino acid sequence according to SEQ ID NO: 344, the CDR2 having an amino acid sequence according to SEQ ID NO: 450, and the CDR3 having an amino acid sequence according to SEQ ID NO: 556, and wherein the VH comprises a sequence having at least 80% sequence identity to the amino acid sequence of SEQ ID NO: 1934 and the VL comprises a sequence having at least 80% sequence identity to the amino acid sequence of SEQ ID NO: 2040; or
(i) the VH comprises the CDR1 having an amino acid sequence according to SEQ ID NO: 32, the CDR2 having an amino acid sequence according to SEQ ID NO: 138, and the CDR3 having an amino acid sequence according to SEQ ID NO: 244, and the VL comprises the CDR1 having an amino acid sequence according to SEQ ID NO: 350, the CDR2 having an amino acid sequence according to SEQ ID NO: 456, and the CDR3 having an amino acid sequence according to SEQ ID NO: 562, and wherein the VH comprises a sequence having at least 80% sequence identity to the amino acid sequence of SEQ ID NO: 1940 and the VL comprises a sequence having at least 80% sequence identity to the amino acid sequence of SEQ ID NO: 2046; or
(j) the VH comprises the CDR1 having an amino acid sequence according to SEQ ID NO: 33, the CDR2 having an amino acid sequence according to SEQ ID NO: 139, and the CDR3 having an amino acid sequence according to SEQ ID NO: 245, and the VL comprises the CDR1 having an amino acid sequence according to SEQ ID NO: 351, the CDR2 having an amino acid sequence according to SEQ ID NO: 457, and the CDR3 having an amino acid sequence according to SEQ ID NO: 56, and wherein the VH comprises a sequence having at least 80% sequence identity to the amino acid sequence of SEQ ID NO: 1941 and the VL comprises a sequence having at least 80% sequence identity to the amino acid sequence of SEQ ID NO: 2047; or
(k) the VH comprises the CDR1 having an amino acid sequence according to SEQ ID NO: 34, the CDR2 having an amino acid sequence according to SEQ ID NO: 140, and the CDR3 having an amino acid sequence according to SEQ ID NO: 246, and the VL comprises the CDR1 having an amino acid sequence according to SEQ ID NO: 352, the CDR2 having an amino acid sequence according to SEQ ID NO: 458, and the CDR3 having an amino acid sequence according to SEQ ID NO: 564, and wherein the VH comprises a sequence having at least 80% sequence identity to the amino acid sequence of SEQ ID NO: 1942 and the VL comprises a sequence having at least 80% sequence identity to the amino acid sequence of SEQ ID NO: 2048; or
(l) the VH comprises the CDR1 having an amino acid sequence according to SEQ ID NO: 35, the CDR2 having an amino acid sequence according to SEQ ID NO: 141, and the CDR3 having an amino acid sequence according to SEQ ID NO: 247, and the VL comprises the CDR1 having an amino acid sequence according to SEQ ID NO: 353, the CDR2 having an amino acid sequence according to SEQ ID NO: 459, and the CDR3 having an amino acid sequence according to SEQ ID NO: 565, and wherein the VH comprises a sequence having at least 80% sequence identity to the amino acid sequence of SEQ ID NO: 1943 and the VL comprises a sequence having at least 80% sequence identity to the amino acid sequence of SEQ ID NO: 2049; or
(m) the VH comprises the CDR1 having an amino acid sequence according to SEQ ID NO: 36, the CDR2 having an amino acid sequence according to SEQ ID NO: 142, and the CDR3 having an amino acid sequence according to SEQ ID NO: 248, and the VL comprises the CDR1 having an amino acid sequence according to SEQ ID NO: 354, the CDR2 having an amino acid sequence according to SEQ ID NO: 460, and the CDR3 having an amino acid sequence according to SEQ ID NO: 566, and wherein the VH comprises a sequence having at least 80% sequence identity to the amino acid sequence of SEQ ID NO: 1944 and the VL comprises a sequence having at least 80% sequence identity to the amino acid sequence of SEQ ID NO: 2050; or
(n) the VH comprises the CDR1 having an amino acid sequence according to SEQ ID NO: 37, the CDR2 having an amino acid sequence according to SEQ ID NO: 143, and the CDR3 having an amino acid sequence according to SEQ ID NO: 249, and the VL comprises the CDR1 having an amino acid sequence according to SEQ ID NO: 355, the CDR2 having an amino acid sequence according to SEQ ID NO: 461, and the CDR3 having an amino acid sequence according to SEQ ID NO: 567, and wherein the VH comprises a sequence having at least 80% sequence identity to the amino acid sequence of SEQ ID NO: 1945 and the VL comprises a sequence having at least 80% sequence identity to the amino acid sequence of SEQ ID NO: 2051; or
(o) the VH comprises the CDR1 having an amino acid sequence according to SEQ ID NO: 38, the CDR2 having an amino acid sequence according to SEQ ID NO: 144, and the CDR3 having an amino acid sequence according to SEQ ID NO: 250, and the VL comprises the CDR1 having an amino acid sequence according to SEQ ID NO: 356, the CDR2 having an amino acid sequence according to SEQ ID NO: 462, and the CDR3 having an amino acid sequence according to SEQ ID NO: 568, and wherein the VH comprises a sequence having at least 80% sequence identity to the amino acid sequence of SEQ ID NO: 1946 and the VL comprises a sequence having at least 80% sequence identity to the amino acid sequence of SEQ ID NO: 2052; or
(p) the VH comprises the CDR1 having an amino acid sequence according to SEQ ID NO: 39, the CDR2 having an amino acid sequence according to SEQ ID NO: 145, and the CDR3 having an amino acid sequence according to SEQ ID NO: 251, and VL comprises the CDR1 having an amino acid sequence according to SEQ ID NO: 357, the CDR2 having an amino acid sequence according to SEQ ID NO: 463, and the CDR3 having an amino acid sequence according to SEQ ID NO: 569, and wherein the VH comprises a sequence having at least 80% sequence identity to the amino acid sequence of SEQ ID NO: 1947 and the VL comprises a sequence having at least 80% sequence identity to the amino acid sequence of SEQ ID NO: 2053; or
(q) the VH comprises the CDR1 having an amino acid sequence according to SEQ ID NO: 40, the CDR2 having an amino acid sequence according to SEQ ID NO: 146, and the CDR3 having an amino acid sequence according to SEQ ID NO: 252, and the VL comprises the CDR1 having an amino acid sequence according to SEQ ID NO: 358, the CDR2 having an amino acid sequence according to SEQ ID NO: 464, and the CDR3 having an amino acid sequence according to SEQ ID NO: 570, and wherein the VH comprises a sequence having at least 80% sequence identity to the amino acid sequence of SEQ ID NO: 1948 and the VL comprises a sequence having at least 80% sequence identity to the amino acid sequence of SEQ ID NO: 2054; or
(r) the VH comprises the CDR1 having an amino acid sequence according to SEQ ID NO: 41, the CDR2 having an amino acid sequence according to SEQ ID NO: 147, and the CDR3 having an amino acid sequence according to SEQ ID NO: 253, and the VL comprises the CDR1 having an amino acid sequence according to SEQ ID NO: 359, the CDR2 having an amino acid sequence according to SEQ ID NO: 465, and the CDR3 having an amino acid sequence according to SEQ ID NO: 571, and wherein the VH comprises a sequence having at least 80% sequence identity to the amino acid sequence of SEQ ID NO: 1949 and the VL comprises a sequence having at least 80% sequence identity to the amino acid sequence of SEQ ID NO: 2055; or
(s) the VH comprises the CDR1 having an amino acid sequence according to SEQ ID NO: 42, the CDR2 having an amino acid sequence according to SEQ ID NO: 148, and the CDR3 having an amino acid sequence according to SEQ ID NO: 254, and the VL comprises the CDR1 having an amino acid sequence according to SEQ ID NO: 360, the CDR2 having an amino acid sequence according to SEQ ID NO: 466, and the CDR3 having an amino acid sequence according to SEQ ID NO: 572, and wherein the VH comprises a sequence having at least 80% sequence identity to the amino acid sequence of SEQ ID NO: 1950 and the VL comprises a sequence having at least 80% sequence identity to the amino acid sequence of SEQ ID NO: 2056; or
(t) the VH comprises the CDR1 having an amino acid sequence according to SEQ ID NO: 43, the CDR2 having an amino acid sequence according to SEQ ID NO: 149, and the CDR3 having an amino acid sequence according to SEQ ID NO: 255, and the VL comprises the CDR1 having an amino acid sequence according to SEQ ID NO: 361, the CDR2 having an amino acid sequence according to SEQ ID NO: 467, and the CDR3 having an amino acid sequence according to SEQ ID NO: 573, and wherein the VH comprises a sequence having at least 80% sequence identity to the amino acid sequence of SEQ ID NO: 1951 and the VL comprises a sequence having at least 80% sequence identity to the amino acid sequence of SEQ ID NO: 2057; or
(u) the VH comprises the CDR1 having an amino acid sequence according to SEQ ID NO: 44, the CDR2 having an amino acid sequence according to SEQ ID NO: 150, and the CDR3 having an amino acid sequence according to SEQ ID NO: 256, and the VL comprises the CDR1 having an amino acid sequence according to SEQ ID NO: 362, the CDR2 having an amino acid sequence according to SEQ ID NO: 468, and the CDR3 having an amino acid sequence according to SEQ ID NO: 574, and wherein the VH comprises a sequence having at least 80% sequence identity to the amino acid sequence of SEQ ID NO: 1952 and the VL comprises a sequence having at least 80% sequence identity to the amino acid sequence of SEQ ID NO: 2058; or
(v) the VH comprises the CDR1 having an amino acid sequence according to SEQ ID NO: 45, the CDR2 having an amino acid sequence according to SEQ ID NO: 151, and the CDR3 having an amino acid sequence according to SEQ ID NO: 257, and the VL comprises the CDR1 having an amino acid sequence according to SEQ ID NO: 363, the CDR2 having an amino acid sequence according to SEQ ID NO: 469, and the CDR3 having an amino acid sequence according to SEQ ID NO: 575, and wherein the VH comprises a sequence having at least 80% sequence identity to the amino acid sequence of SEQ ID NO: 1953 and the VL comprises a sequence having at least 80% sequence identity to the amino acid sequence of SEQ ID NO: 2059; or
(w) the VH comprises the CDR1 having an amino acid sequence according to SEQ ID NO: 46, the CDR2 having an amino acid sequence according to SEQ ID NO: 152, and the CDR3 having an amino acid sequence according to SEQ ID NO: 258, and the VL comprises the CDR1 having an amino acid sequence according to SEQ ID NO: 364, the CDR2 having an amino acid sequence according to SEQ ID NO: 470, and the CDR3 having an amino acid sequence according to SEQ ID NO: 576, and wherein the VH comprises a sequence having at least 80% sequence identity to the amino acid sequence of SEQ ID NO: 1954 and the VL comprises a sequence having at least 80% sequence identity to the amino acid sequence of SEQ ID NO: 2060; or
(x) the VH comprises the CDR1 having an amino acid sequence according to SEQ ID NO: 47, the CDR2 having an amino acid sequence according to SEQ ID NO: 153, and the CDR3 having an amino acid sequence according to SEQ ID NO: 259, and the VL comprises the CDR1 having an amino acid sequence according to SEQ ID NO: 365, the CDR2 having an amino acid sequence according to SEQ ID NO: 471, and the CDR3 having an amino acid sequence according to SEQ ID NO: 577, and wherein the VH comprises a sequence having at least 80% sequence identity to the amino acid sequence of SEQ ID NO: 1955 and the VL comprises a sequence having at least 80% sequence identity to the amino acid sequence of SEQ ID NO: 2061; or
(y) the VH comprises the CDR1 having an amino acid sequence according to SEQ ID NO: 48, the CDR2 having an amino acid sequence according to SEQ ID NO: 154, and the CDR3 having an amino acid sequence according to SEQ ID NO: 260, and the VL comprises the CDR1 having an amino acid sequence according to SEQ ID NO: 366, the CDR2 having an amino acid sequence according to SEQ ID NO: 472, and the CDR3 having an amino acid sequence according to SEQ ID NO: 578, and wherein the VH comprises a sequence having at least 80% sequence identity to the amino acid sequence of SEQ ID NO: 1956 and the VL comprises a sequence having at least 80% sequence identity to the amino acid sequence of SEQ ID NO: 2062.

18. The α4β7 binding protein of claim 15, comprising a modified Fc that extends half-life of the α4β7 binding protein as compared to an α4β7 binding protein that does not comprise the modified Fc.

19. The α4β7 binding protein of claim 18, wherein the modified Fc comprises amino acid modifications M252Y, S254T, and T256E (YTE) and/or L234A/G237A (LAGA).

20. A method of treating an inflammatory disease in a patient in need thereof, the method comprising administering subcutaneously or intravenously to the patient an effective amount of the α4β7 binding protein of claim 15.

21. A pharmaceutical composition comprising an effective amount of the α4β7 binding protein claim 15 and a pharmaceutically acceptable carrier.
